(19) United States
(12) Patent Application Publication
(10) Pub. No.: US 2021/0145768 A1
(43) Pub. Date: May 20, 2021

(54) MODULATING IMMUNE RESPONSE VIA TARGETING OF OLFACTORY RECEPTOR ACTIVITY

(71) Applicant: La Jolla Institute for Allergy and Immunology, La Jolla, CA (US)

(72) Inventors: Klaus LEY, La Jolla, CA (US); Marco ORECCHIONI, La Jolla, CA (US)

(73) Assignee: La Jolla Institute for Allergy and Immunology, La Jolla, CA (US)

(21) Appl. No.: 17/048,059

(22) PCT Filed: Apr. 15, 2019
(86) PCT No.: PCT/US2019/027558

§ 371 Date: Oct. 15, 2020

Related U.S. Application Data
(60) Provisional application No. 62/658,581, filed on Apr. 16, 2018.

(51) Int. Cl. A61K 31/11; A61P 37/02; C12N 15/113

(52) U.S. Cl. CPC A61K 31/11; C12N 2310/14; C12N 15/1138; A61P 37/02

(57) ABSTRACT

This disclosure provides agents that are useful for modulating an immune response in subject and for treating diseases, such as autoimmune diseases, cardiovascular diseases, infectious diseases, and cancer. These agents may comprise an olfactory receptor (OLFR), an OLFR ligand, or a protein involved in the trafficking of an OLFR to the plasma membrane of a cell. Alternatively, the agents may modulate the expression or activity of an OLFR, OLFR ligand, or protein involved in the trafficking of the OLFR to the plasma membrane of a cell.

### CROSS-REFERENCE TO RELATED APPLICATIONS

This application claims priority under 35 U.S.C. § 119(e) to U.S. Provisional Application No. 62/658,581, filed Apr. 16, 2018, the content of which is hereby incorporated by reference in its entirety.

### GOVERNMENT SUPPORT

This invention was made with support under grants R01 HL115232 and HL 088093 awarded by the National Institutes of Health. The government has certain rights in this invention.

### SUMMARY OF INVENTION

There is presently provided novel regulators of immune response and methods of modulating the novel regulators to modulate an immune response. The present invention identifies olfactory receptors in macrophages, monocytes and dendritic cells that can be targeted to modulate immune responses, including innate immune responses. In particular embodiments of the present invention, the olfactory receptors identified are present on macrophages (e.g. vascular macrophages) and their expression, activity, or binding can be targeted to modulate cardiovascular disease, atherosclerosis, infection, or cancer.

Thus, in one aspect, there is provided a method of modulating an immune response, the method comprising modulating expression or activity of one or more olfactory receptors. In another aspect, there is provided a method of modulating an adverse cardiovascular event or cardiovascular disease, the method comprising modulating expression or activity of one or more olfactory receptors. In an additional aspect, there is provided a method of modulating atherosclerosis, the method comprising modulating expression or activity of one or more olfactory receptors. In another aspect, there is provided a method of modulating a viral, bacterial or fungal infection, neoplasia, neoplastic disorder, tumor, cancer or malignancy, metastasis of a neoplasia, tumor, cancer or malignancy to other sites, or formation or establishment of a metastatic neoplasia, neoplastic disorder, tumor, cancer or malignancy to other sites distal from a primary neoplasia, neoplastic disorder, tumor, cancer or malignancy, the method comprising modulating expression or activity of one or more olfactory receptors. In certain embodiments, the method comprises decreasing, reducing, inhibiting, suppressing, limiting or controlling expression or activity of one or more olfactory receptors. In other embodiments, the method comprises enhancing, stimulating, inducing or increasing expression or activity of one or more olfactory receptors.

In an additional aspect, there is provided a method of modulating an immune response, the method comprising modulating expression or activity of one or more olfactory receptors listed in any of Table 1, Table 2, Table 3, Table 4, Table 5, Table 6, Table 7, Table 8, and Table 9. In specific embodiments, the one or more olfactory receptors includes Olfr2. In certain embodiments, the method comprises decreasing, reducing, inhibiting, suppressing, limiting or controlling expression or activity of one or more olfactory receptor listed in any of Table 1, Table 2, Table 3, Table 4, Table 5, Table 6, Table 7, Table 8, and Table 9. In certain embodiments, the method comprises decreasing, reducing, inhibiting, suppressing, limiting or controlling expression or activity of one or more olfactory receptor listed in any of FIGS. 2A and 2B. In other embodiments, the method comprises enhancing, stimulating, inducing or increasing expression or activity of one or more olfactory receptor listed in any of Table 1, Table 2, Table 3, Table 4, Table 5, Table 6, Table 7, Table 8, and Table 9. In other embodiments, the method comprises enhancing, stimulating, inducing or increasing expression or activity of one or more olfactory receptor listed in any of FIGS. 2A and 2B.

In another aspect, there is provided a method of decreasing, reducing, inhibiting, suppressing, limiting or controlling an undesirable or aberrant immune response, immune disorder, inflammatory response or inflammation in a subject, comprising modulating expression or activity of one or more olfactory receptor listed in any of Table 1, Table 2, Table 3, Table 4, Table 5, Table 6, Table 7, Table 8, and Table 9. In yet another aspect, there is provided a method of decreasing, reducing, inhibiting, suppressing, limiting or controlling an undesirable or aberrant immune response, immune disorder, inflammatory response or inflammation in a subject, comprising modulating expression or activity of one or more olfactory receptor listed in any of FIGS. 2A and 2B. In yet another aspect, there is provided a method of decreasing, reducing, inhibiting, suppressing, limiting or controlling an autoimmune response, disorder or disease in a subject, comprising modulating expression or activity of one or more olfactory receptor listed in any of Table 1, Table 2, Table 3, Table 4, Table 5, Table 6, Table 7, Table 8, and Table 9. In yet another aspect, there is provided a method of decreasing, reducing, inhibiting, suppressing, limiting or controlling an autoimmune response, disorder or disease in a subject, comprising modulating expression or activity of one or more olfactory receptor listed in any of FIGS. 2A and 2B.

In certain embodiments, the methods comprise decreasing, reducing, inhibiting, suppressing, limiting or controlling an undesirable or aberrant immune response, immune disorder, inflammatory response or inflammation, comprising modulating expression or activity of one or more olfactory receptors. In different embodiments, the methods comprise decreasing, reducing, inhibiting, suppressing, limiting or controlling an autoimmune response, disorder or disease in a subject, comprising modulating expression or activity of one or more olfactory receptors.

In some embodiments, the method comprises one or more of reducing, inhibiting, suppressing, limiting, controlling, enhancing, stimulating, inducing or increasing expression or activity of one or more olfactory receptors. In certain embodiments, the reducing, inhibiting, suppressing, limiting, controlling, enhancing, stimulating, inducing or increasing expression or activity of one or more olfactory receptors comprises reducing, inhibiting, suppressing, limiting, controlling, enhancing, stimulating, or inducing signaling through olfactory receptors and/or the respective downstream signaling effects of reducing, inhibiting, suppressing, limiting, controlling, enhancing, stimulating, inducing or increasing expression or activity of olfactory receptors. Certain alternative embodiments comprise decreasing, reducing, inhibiting, suppressing, limiting or controlling an adverse symptom of the undesirable or aberrant immune response, immune disorder, inflammatory response or inflammation, or an adverse symptom of the autoimmune response, disorder or disease. In certain embodiments, the one or more olfactory receptors are selected from the olfactory receptors listed in any of Table 1, Table 2, Table 3, Table 4, Table 5, Table 6, Table 7, Table 8 and Table 9. In certain embodiments, the one or more olfactory receptors are selected from the olfactory receptors listed in any of FIGS. 2A and 2B. In some embodiments, such adverse symptoms of the undesirable or aberrant immune response, immune disorder, inflammatory response or inflammation or an adverse symptom of the autoimmune response, disorder or disease include but are not limited to swelling, pain, rash, headache, fever, nausea, diarrhea, bloat, lethargy, skeletal joint stiffness or tissue or cell damage. In different embodiments, the adverse symptom of the undesirable or aberrant immune response, immune disorder, inflammatory response or inflammation or the adverse symptom of the autoimmune response, disorder or disease is chronic or acute. In certain embodiments, the subject is in need of treatment for or is at risk of an undesirable or aberrant immune response, immune disorder, inflammatory response or inflammation or an autoimmune response, disorder or disease. The immune disorder, inflammatory response, inflammation, autoimmune response disorder or autoimmune disease of the present methods include but are not limited rheumatoid arthritis, juvenile rheumatoid arthritis, osteoarthritis, psoriatic arthritis, diabetes mellitus, multiple sclerosis, encephalomyelitis, myasthenia gravis, systemic lupus erythematosus (SLE), autoimmune thyroiditis, atopic dermatitis, eczematous dermatitis, psoriasis, Sjogren's Syndrome, Crohn's disease, aphthous ulcer, iritis, conjunctivitis, keratoconjunctivitis, ulcerative colitis, inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common g chain (c) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (g5/14.1) deficiency, Hyper-IgM syndrome: X-linked (CD40 ligand deficiency) or non-X-linked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon g receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1 A/SAP deficiency), or hyper IgE syndrome.

In another aspect, there is provided a method of decreasing, reducing, inhibiting, suppressing, limiting or controlling an adverse cardiovascular event or cardiovascular disease, comprising modulating expression or activity of one or more olfactory receptors. In some embodiments, the adverse cardiovascular event or cardiovascular disease includes but is not limited to coronary artery disease, peripheral artery disease, cerebrovascular disease, renal artery disease, stroke, myocardial infarction (heart attack), ischemic heart failure, transient ischemic attack or brain trauma, atherosclerotic plaque formation or elevated blood cholesterol. In another aspect, there is provided a method decreasing, reducing, inhibiting, suppressing, limiting or controlling atherosclerosis, comprising modulating expression or activity of one or more olfactory receptors. In some embodiments, the one or more olfactory receptors are selected from the olfactory receptors listed in any of Table 1, Table 2, Table 3, Table 4, Table 5, Table 6, Table 7, Table 8, and Table 9. In some embodiments, the one or more olfactory receptors are selected from the olfactory receptors listed in any of FIGS. 2A and 2B.

In another aspect, there is provided a method of reducing or inhibiting in a subject a neoplasia, neoplastic disorder, tumor, cancer or malignancy, metastasis of a neoplasia, tumor, cancer or malignancy to other sites, or formation or establishment of a metastatic neoplasia, neoplastic disorder, tumor, cancer or malignancy to other sites distal from a primary neoplasia, neoplastic disorder, tumor, cancer or malignancy, the method comprising modulating expression or activity of one or more olfactory receptors. In some embodiments, modulating expression or activity of one or more olfactory receptors comprises administering an agonist of one or more olfactory receptors. In another aspect, there is provided a method of reducing or inhibiting in a subject a neoplasia, neoplastic disorder, tumor, cancer or malignancy, metastasis of a neoplasia, tumor, cancer or malignancy to other sites, or formation or establishment of a metastatic neoplasia, neoplastic disorder, tumor, cancer or malignancy to other sites distal from a primary neoplasia, neoplastic disorder, tumor, cancer or malignancy, the method comprising increasing expression or activity of one or more olfactory receptors. In some embodiments, increasing expression or activity of one or more olfactory receptors comprises administering an agonist of one or more olfactory receptors. In yet another aspect, there is provided a method of reducing or inhibiting in a subject viral, bacterial or fungal infection, the method comprising modulating expression or activity of one or more olfactory receptors. In different embodiments, these methods comprise one or more of reducing, inhibiting, suppressing, limiting, controlling enhancing, stimulating, inducing or increasing expression or activity of one or more olfactory receptors. In some embodiments, the one or more olfactory receptors are selected from the olfactory receptors listed in any of Table 1, Table 2, Table 3, Table 4, Table 5, Table 6, Table 7, Table 8, and Table 9. In some embodiments, the one or more olfactory receptors are selected from the olfactory receptors listed in any of FIGS. 2A and 2B. In some embodiments, the agonist is one or more of the agonists listed in Table 8.

In certain embodiments, the methods comprise decreasing, reducing, inhibiting, suppressing, limiting or controlling an adverse symptom of the neoplasia, neoplastic disorder, tumor, cancer or malignancy, metastasis of a neoplasia, tumor, cancer or malignancy to other sites, or formation or establishment of a metastatic neoplasia, neoplastic disorder, tumor, cancer or malignancy to other sites distal from a primary neoplasia, neoplastic disorder, tumor, cancer or malignancy, or viral, bacterial or fungal infection. The neoplasia, neoplastic disorder, tumor, cancer or malignancy of the methods provided herein include but are not limited to a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, lymphosarcoma, liposarcoma, osteosarcoma, chondrosarcoma, leiomyosarcoma, rhabdomyosarcoma, fibrosarcoma or melanoma; or a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, neoplastic disorder, tumor, cancer or malignancy.

In certain embodiments of the present invention, the methods comprise modulating an innate immune response in the subject. In some embodiments, the methods comprise modulating the activity of a macrophage. In alternative embodiments, the macrophage is a vascular macrophage.

In another aspect, there is provided a method for modulating expression or activity of one or more olfactory receptors comprising modulating the binding of the olfactory receptor to a ligand. In some embodiments, the modulating of the binding of the olfactory receptor to a ligand comprises inhibiting binding of the ligand to the olfactory receptor. In certain alternative embodiments, the modulating of the binding of the olfactory receptor to a ligand comprises agonizing binding of the ligand to the olfactory receptor.

In particular embodiments, the methods of the present invention comprise administering to the subject a therapeutically effective amount of an agent that modulates expression or activity of one or more olfactory receptors listed in any of Table 1, Table 2, Table 3, Table 4, Table 5, Table 6, Table 7, Table 8, and Table 9. Alternatively, the methods of the present invention comprise administering to the subject a therapeutically effective amount of an agent that modulates expression or activity of one or more olfactory receptors listed in any of FIGS. 2A and 2B. In different embodiments, the agent is an antagonist or agonist of expression or activity of one or more olfactory receptor listed in any of Table 1, Table 2, Table 3, Table 4, Table 5, Table 6, Table 7, Table 8, and Table 9. In certain alternative embodiments, the antagonist or agonist is one or more of the agonists listed in Table 8 or the antagonists listed in Table 9. In different embodiments, the agent is an antagonist or agonist of expression or activity of one or more olfactory receptor listed in any of FIGS. 2A and 2B. In certain embodiments, the agent is a protein, a peptide, an antibody, an siRNA, a small molecule or a ligand mimetic.

In some embodiment of the present methods, the subject is a mammal, including but not limited to a human.

In another aspect, there is provided a method of modulating an immune response in a subject, comprising modulating expression or activity of one or more olfactory receptors (OLFR).

In some embodiments, the OLFR is expressed by a cell in vivo. In some embodiments, the cell is an animal cell, with the proviso that the cell is not an olfactory cell. In some embodiments, the cell is a macrophage. In some embodiments, the macrophage is a vascular macrophage.

In some embodiments, the modulation comprises one or more of inhibiting, decreasing, reducing, suppressing, limiting or controlling the immune response by a method comprising administering to the subject an effective amount of an agent that inhibits the expression of or deactivates the OLFR.

In some embodiments, the modulation comprises one or more of decreasing, reducing, inhibiting, suppressing, limiting or controlling an undesirable or aberrant immune response, immune disorder, inflammatory response or inflammation, by a method comprising administering to the subject an effective amount of an agent that inhibits the expression of or deactivates the OLFR.

In some embodiments, the modulation comprises one or more of decreasing, reducing, inhibiting, suppressing, limiting or controlling an autoimmune response, disorder or disease in a subject, by a method comprising administering to the subject an effective amount of an agent that inhibits the expression of or deactivates the OLFR.

In some embodiments, the modulation comprises one or more of decreasing, reducing, inhibiting, suppressing, limiting or controlling an adverse symptom of the undesirable or aberrant immune response, immune disorder, inflammatory response or inflammation, or an adverse symptom of the autoimmune response, disorder or disease in the subject.

In some embodiments, the adverse symptom of the undesirable or aberrant immune response, immune disorder, inflammatory response or inflammation or an adverse symptom of the autoimmune response, disorder or disease is swelling, pain, rash, headache, fever, nausea, diarrhea, bloat, lethargy, skeletal joint stiffness or tissue or cell damage. In some embodiments, the adverse symptom of the undesirable or aberrant immune response, immune disorder, inflammatory response or inflammation or the adverse symptom of the autoimmune response, disorder or disease is chronic or acute.

In some embodiments, immune disorder, inflammatory response, inflammation, autoimmune response disorder or autoimmune disease comprises rheumatoid arthritis, juvenile rheumatoid arthritis, osteoarthritis, psoriatic arthritis, diabetes mellitus, multiple sclerosis, encephalomyelitis, myasthenia gravis, systemic lupus erythematosus (SLE), autoimmune thyroiditis, atopic dermatitis, eczematous dermatitis, psoriasis, Sjogren's Syndrome, Crohn's disease, aphthous ulcer, iritis, conjunctivitis, keratoconjunctivitis, ulcerative colitis, inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common g chain (c) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (g5/14.1) deficiency, Hyper-IgM syndrome: X-linked (CD40 ligand deficiency) or non-X-linked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon g receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1 A/SAP deficiency), or hyper IgE syndrome.

In some embodiments, the modulation comprises one or more of decreasing, reducing, inhibiting, suppressing, limiting or controlling an adverse cardiovascular event or cardiovascular disease by a method comprising administering to the subject an effective amount of an agent that inhibits the expression of or deactivates the OLFR. In some embodiments, the adverse cardiovascular event or cardiovascular disease comprises coronary artery disease, peripheral artery disease, cerebrovascular disease, renal artery disease, stroke, myocardial infarction (heart attack), ischemic heart failure, transient ischemic attack or brain trauma, atherosclerosis, atherosclerotic plaque formation or elevated blood cholesterol.

In some embodiments, the modulation comprises one or more of decreasing, reducing, inhibiting, suppressing, limiting or controlling atherosclerosis, by a method comprising administering to the subject an effective amount of an agent that inhibits the expression of or deactivates the OLFR.

In some embodiments, the method comprises one or more of reducing or inhibiting in a subject viral, bacterial or fungal infection, by a method comprising administering to the subject an effective amount of an agent that increases the expression of or activates the OLFR.

In some embodiments, the modulation comprises one or more of comprising decreasing, reducing, inhibiting, suppressing, limiting or controlling an adverse symptom of the neoplasia, neoplastic disorder, tumor, cancer or malignancy, metastasis of a neoplasia, tumor, cancer or malignancy to other sites, or formation or establishment of a metastatic neoplasia, neoplastic disorder, tumor, cancer or malignancy to other sites distal from a primary neoplasia, neoplastic disorder, tumor, cancer or malignancy, or viral, bacterial or fungal infection by a method comprising administering to the subject an effective amount of an agent that increases the expression of or activates the OLFR. In some embodiments, the neoplasia, neoplastic disorder, tumor, cancer or malignancy treated is a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, lymphosarcoma, liposarcoma, osteosarcoma, chondrosarcoma, leiomyosarcoma, rhabdomyosarcoma, fibrosarcoma or melanoma; or a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, neoplastic disorder, tumor, cancer or malignancy.

In some embodiments, the OLFR is an OLFR listed in any one of Tables 1-9, and FIGS. 2A and 2B. In some embodiments, the OLFR is an OLFR listed in Table 7. In some embodiments, the OLFR is selected from the group consisting of OR7C1, OR7D4, OR10A6, OR11H6, OR4E2, OR10H1, and OR6A2.

In some embodiments, the agent is selected from the group of: a ligand or small molecule that binds to the OLFR or blocks the binding of the OLFR to the ligand or an agent that inhibits the expression of the OLFR by the cell. In some embodiments, the agent is selected from the group of an antibody, fragment or mimetic that binds to OLFR or an OLFR ligand, an anti-OLFR gene silencing agent, octanal, heptanal, or a prodrug or solvate thereof.

In some embodiments, the agent modulates the OLFR by modulating the trafficking of the OLFR to a plasma membrane of a cell. In some embodiments, the agent is a protein selected from the group of receptor transporting protein 1 (RTP1), RTP2, receptor expression enhancing protein 1 (REEP1), aminoacylase 3 (Acy3), or guanine nucleotide-binding protein G(olf) subunit alpha (Gnal). In some embodiments, the agent is a nucleotide encoding a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal).

In some embodiments, the immune response is stimulated by a method comprising administering an agent that increases expression or secretion of an inflammatory cytokine. In some embodiments, the inflammatory cytokine is selected from the group consisting of tumor necrosis factor (TNF), C-C motif chemokine ligand 2 (CCL2), CCL4, CCL5, interleukin 6 (IL-6), IL-1B, IL-18, and nitric oxide synthase 2 (NOS2).

In another aspect, there is provided a method of suppressing an immune response in a subject in need thereof, the method comprising administering to the subject an agent that decreases the expression of or activity of an olfactory receptor (OLFR), thereby suppressing an immune response in the subject.

In some embodiments, the OLFR is an OLFR listed in any one of Tables 1-9. In some embodiments, the OLFR is an OLFR listed in Table 7. In some embodiments, the OLFR is selected from the group consisting of OR7C1, OR7D4, OR10A6, OR11H6, OR4E2, OR10H1, and OR6A2.

In some embodiments, the agent is a gene silencing agent. In some embodiments, the gene silencing agent is selected from the group consisting of a RNA interference (RNAi) molecule, zinc finger nuclease, transcription activator-like effector nuclease (TALEN), and Clustered Regulatory Interspaced Short Palindromic Repeats (CRISPR) enzyme. In some embodiments, the RNAi molecule is selected from the group consisting of a small interference RNA (siRNA), short hairpin RNA (shRNA) and microRNA (miRNA).

In some embodiments, the agent decreases the activity of the OLFR by inhibiting the binding of the OLFR with its ligand. In some embodiments, the ligand is an OLFR ligand listed in Table 8.

In some embodiments, the agent is an OLFR antagonist. In some embodiments, the OLFR antagonist is citral, undecanal, oxyphenylon, phenirat, methyl cinnamaldehyde, hydrocinamaldehyde, bourgeonal, ethylhexanoic acid, α-ionone, octanoic acid, a solvate or prodrug thereof. In some embodiments, the OLFR antagonist is an antagonist listed in Table 9.

In some embodiments, the agent decreases the activity of the OLFR by inhibiting the trafficking of the OLFR to a plasma membrane of a cell.

In some embodiments, the agent is an antibody, fragment or mimetic thereof that binds to an OLFR.

In another aspect, there is provided a method of suppressing an immune response in a subject in need thereof, the method comprising administering to the subject an agent that inhibits the trafficking of an olfactory receptor (OLFR) to a plasma membrane of a cell, thereby suppressing an immune response in the subject.

In another aspect, there is provided a method of treating an autoimmune disease in a subject in need thereof, the method comprising administering to the subject an agent that decreases the expression of or activity of an olfactory receptor (OLFR). In another aspect, there is provided a method of treating an autoimmune disease in a subject in need thereof, the method comprising administering to the subject an agent that inhibits the trafficking of an olfactory receptor (OLFR) to a plasma membrane of a cell.

In another aspect, there is provided a method of treating a cardiovascular disease in a subject in need thereof, the method comprising administering to the subject an agent that decreases the expression of or activity of an olfactory receptor (OLFR). In another aspect, there is provided a method of a cardiovascular disease in a subject in need thereof, the method comprising administering to the subject an agent that inhibits the trafficking of an olfactory receptor (OLFR) to a plasma membrane of a cell.

In another aspect, there is provided a method of increasing an immune response in a subject in need thereof, the method comprising administering to the subject an agent that increases or promotes the trafficking of an olfactory receptor (OLFR) to a plasma membrane of a cell, thereby increasing an immune response in the subject.

In another aspect, there is provided a method of treating an infection in a subject in need thereof, the method comprising administering to the subject an agent that increases the expression of or activates an olfactory receptor (OLFR). In another aspect, there is provided a method of treating an infection in a subject in need thereof, the method comprising administering to the subject an agent that increases or promotes the trafficking of an olfactory receptor (OLFR) to a plasma membrane of a cell. In some embodiments, the infection is selected from the group consisting of a viral infection, bacterial infection, or fungal infection.

In another aspect, there is provided a method of treating cancer in a subject in need thereof, the method comprising administering to the subject an agent that increases the expression of or activates an olfactory receptor (OLFR). In another aspect, there is provided a method of cancer in a subject in need thereof, the method comprising administering to the subject an agent that increases or promotes the trafficking of an olfactory receptor (OLFR) to a plasma membrane of a cell.

In another aspect, there is provided a method of modulating the expression or activity of one or more proteins involved in an immune response, the method comprising contacting a cell with an agent that modulates the expression or activity of an olfactory receptor (OLFR). In some embodiments, the agent increases the expression of or activates the OLFR. In other embodiments, the agent decreases the expression or activity of the OLFR.

In another aspect, there is provided a method of increasing the expression or activity of one or more proteins involved in an immune response, the method comprising contacting a cell with an agent that increases the expression of or activates an olfactory receptor (OLFR).

In another aspect, there is provided a method of decreasing the expression or activity of one or more proteins involved in an immune response, the method comprising contacting a cell with an agent that decreases the expression of or inhibits an olfactory receptor (OLFR).

In another aspect, there is provided a method of modulating the expression or activity of one or more proteins involved in an immune response, the method comprising contacting a cell with an agent that modulates the trafficking of an olfactory receptor (OLFR) to a plasma membrane of the cell. In some embodiments, the agent increases or promotes the trafficking of the OLFR to the plasma membrane of the cell. In some embodiments, the agent decreases or inhibits the trafficking of the OLFR to the plasma membrane of a cell.

In another aspect, there is provided a method of increasing the expression or activity of one or more proteins involved in an immune response, the method comprising contacting a cell with an agent that increases or promotes the trafficking of an olfactory receptor (OLFR) to a plasma membrane of the cell.

In another aspect, there is provided a method of decreasing the expression or activity of one or more proteins involved in an immune response, the method comprising contacting a cell with an agent that decreases or inhibits the trafficking of an olfactory receptor (OLFR) to a plasma membrane of a cell.

In some embodiments, the methods comprise the use of one or more agents. In some embodiments, the agent is a gene silencing agent. In some embodiments, the gene silencing agent is selected from the group consisting of a RNA interference (RNAi) molecule, zinc finger nuclease, transcription activator-like effector nuclease (TALEN), and Clustered Regulatory Interspaced Short Palindromic Repeats (CRISPR) enzyme. In some embodiments, the RNAi molecule is selected from the group consisting of a small interference RNA (siRNA), short hairpin RNA (shRNA) and microRNA (miRNA).

In some embodiments, the agent decreases the activity of the OLFR by inhibiting the binding of the OLFR with its ligand. In some embodiments, the ligand is an OLFR ligand listed in Table 8.

In some embodiments, the agent is an OLFR antagonist. In some embodiments, the OLFR antagonist is citral, undecanal, oxyphenylon, phenirat, methyl cinnamaldehyde, hydrocinamaldehyde, bourgeonal, ethylhexanoic acid, α-ionone, octanoic acid, a solvate or prodrug thereof. In some embodiments, the OLFR antagonist is an antagonist listed in Table 9.

In some embodiments, the agent decreases the activity of the OLFR by inhibiting the trafficking of the OLFR to a plasma membrane of a cell.

In some embodiments, the agent is an antibody, fragment or mimetic thereof that binds to an OLFR.

In some embodiments, the agent is an OLFR agonist. In some embodiments, the OLFR agonist is an agonist listed in Table 8. In some embodiments, the OLFR agonist is selected from the group consisting of octanal, coumarin, helional, lilial, b-ionone, androstenone, androstadienone, caramel furanone, 3-phenyl propyl propionate, eugenol, ethil vanillin, 2-ethyl-fencol, isovaleric acid, nonanoic acid, butyl butyryllactate, butyric acid, isovaleric acid, propionic acid, N-amyl acetate, eugenol acetate, sandalwood, S-(−)-citronellol, S-(−)-citronellal, (+)-carvine, (−) carvone, (+) carvone, linalool, bourgeonal, acetophenone, amyl butyrate, nonanethiol, allyl phenyl acetate, N-amyl acetate, muscone, isoeugenol, eugenol methyl ether, 1-hexanol, 1-heptanole, 1-octanol, celery ketone, anis aldehyde, (+)-menthol, vanillin, guaiacol, lyral, ethyl heptanoate, methyl octanoate, nonanal 1-nonanol, 2-nonanol, 3-octanone, 3-nonanone, decyl aldehyde. In some embodiments, the agent is octanal, heptanal, or a prodrug or solvate thereof.

In some embodiments, the agent is a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal). In some embodiments, the agent comprises a nucleotide encoding a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal).

In some embodiments, the OLFR is an OLFR listed in any one of Tables 1-9 and FIGS. 2A-2B. In some embodiments, the OLFR is an OLFR listed in Table 7. In some embodiments, the OLFR is selected from the group consisting of OR7C1, OR7D4, OR10A6, OR11H6, OR4E2, OR10H1, and OR6A2.

In some embodiments, the OLFR is expressed by a cell in vivo. In some embodiments, the cell is an animal cell, with the proviso that the cell is not an olfactory cell. In some embodiments, the cell is a macrophage. In some embodiments, the macrophage is a vascular macrophage.

In some embodiments, the one or more proteins involved in an immune response is any of the proteins shown in FIGS. 16 and 17. In some embodiments, the one or more proteins involved in an immune response is a protein selected from CCL5 (C-C Motif Chemokine Ligand 5), Tnfrsf12a (Osteoprotegerin), Axin 1, Nadk, Ahr (Aryl hydrocarbon receptor), QDPR (quinoid dihydropteridine reductase), HGF (Hepatocyte Growth Factor), ADAM23, or Snap29.

In some embodiments, the administration is local or systemic. In some embodiments, the administration comprises by a method comprising intravenously.

In some embodiments, the subject is a mammal. In some embodiments, the subject has or is predisposed to a disease or disorder involving an immune dysregulation. In some embodiments, the subject is a human patient.

In another aspect, there is provided a kit comprising an agent that modulates the activity of an OLFR and instructions for use.

In some embodiments, the agent is a gene silencing agent. In some embodiments, the gene silencing agent is selected from the group consisting of a RNA interference (RNAi) molecule, zinc finger nuclease, transcription activator-like effector nuclease (TALEN), and Clustered Regulatory Interspaced Short Palindromic Repeats (CRISPR) enzyme. In some embodiments, the RNAi molecule is selected from the group consisting of a small interference RNA (siRNA), short hairpin RNA (shRNA) and microRNA (miRNA).

In some embodiments, the agent modulates the activity of the OLFR by inhibiting the binding of the OLFR with its ligand. In some embodiments, the ligand is an OLFR ligand listed in Table 8.

In some embodiments, the agent is an OLFR antagonist. In some embodiments, the OLFR antagonist is citral, undecanal, oxyphenylon, phenirat, methyl cinnamaldehyde, hydrocinamaldehyde, bourgeonal, ethylhexanoic acid, α-ionone, octanoic acid, a solvate or prodrug thereof. In some embodiments, the OLFR antagonist is an antagonist listed in Table 9.

In some embodiments, the agent modulates the activity of the OLFR by inhibiting the trafficking of the OLFR to a plasma membrane of a cell.

In some embodiments, the agent is an antibody, fragment or mimetic thereof that binds to an OLFR.

In some embodiments, the agent is an OLFR agonist. In some embodiments, the OLFR agonist is an agonist listed in Table 8. In some embodiments, the OLFR agonist is selected from the group consisting of octanal, coumarin, helional, lilial, b-ionone, androstenone, androstadienone, caramel furanone, 3-phenyl propyl propionate, eugenol, ethil vanillin, 2-ethyl-fencol, isovaleric acid, nonanoic acid, butyl butyryllactate, butyric acid, isovaleric acid, propionic acid, N-amyl acetate, eugenol acetate, sandalwood, S-(−)-citronellol, S-(−)-citronellal, (+)-carvine, (−) carvone, (+) carvone, linalool, bourgeonal, acetophenone, amyl butyrate, nonanethiol, allyl phenyl acetate, N-amyl acetate, muscone, isoeugenol, eugenol methyl ether, 1-hexanol, 1-heptanole, 1-octanol, celery ketone, anis aldehyde, (+)-menthol, vanillin, guaiacol, lyral, ethyl heptanoate, methyl octanoate, nonanal 1-nonanol, 2-nonanol, 3-octanone, 3-nonanone, decyl aldehyde. In some embodiments, the agent is octanal, heptanal, or a prodrug or solvate thereof.

In some embodiments, the agent is a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal). In some embodiments, the agent comprises a nucleotide encoding a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal).

In some embodiments, wherein the OLFR is an OLFR listed in any one of Tables 1-9 and FIGS. 2A-2B. In some embodiments, the OLFR is an OLFR listed in Table 7. In some embodiments, the OLFR is selected from the group consisting of OR7C1, OR7D4, OR10A6, OR11H6, OR4E2, OR10H1, and OR6A2.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF THE DRAWINGS

FIGS. 1A-C show a non-limiting example of Olfr2 inhibition with Citral and the resulting decrease in inflammatory gene expression and atherosclerosis progression in vivo. FIG. 1A shows the results of a gene expression analysis of CCL2, CCL4, TNF and iNOS after treatment with the Olfr2 inhibitor citral alone or in combination with octanal and LPS. FIG. 1B shows the results of an atherosclerosis lesion evaluation (% and number of pixels with lesions) after the treatment with OLFR2 inhibitor citral. FIG. 1C shows pictures of pinned aortas of mice treated with citral or saline (untreated).

FIG. 2A depicts a heat map of Olfr genes (total 1123) expressed in mouse vascular macrophages and several other tissue macrophages, DCs and blood monocytes.

FIG. 2B displays a heat map of expressed Olfrs with known human orthologues.

FIG. 3A shows the relative expression of Olfr2 quantified by real time PCR analysis of Olfr2 expression normalized to expression of Gapdh and untreated control.

FIG. 3B shows Olfr2 protein expression in BMDM lysate.

FIGS. 3C-D show flow cytometric analysis of extracellular Olfr2 expression. Median Fluorescence Intensity (MFI) of live F4/80⁺ BMDMs is reported.

FIG. 3E shows Airyscan high resolution imaging of previously treated BMDMs stained for Olfr2 (AF488). Hoechst was used for the nuclear staining Scale bar=200 mm.

FIG. 3F shows Olfr2 expression was determined by real-time PCR.

FIGS. 3G-H show flow cytometric analysis of Olfr2 by aortic macrophages. Olfr2 Median Fluorescence Intensity (MFI) of vascular macrophages (CD45⁺, Live, TCRβ⁻, CD19⁻, F4/80⁺) is reported. (n=5-6/group).

FIG. 3I shows microphotographs depicting Olfr2, CD68 and Hoechst immunoreactivity in whole mount aorta from Apoe^{-/-} mouse fed WD for 2 weeks. Right: Zoom in of Olfr2⁺ CD68⁺ macrophages. Data are presented as mean±SEM. *p<0.05, ** p<0.01, ***p<0.005, ****p<0.001. p calculated by One-Way ANOVA test, Tukey's multiple comparisons test.

FIGS. 4A-B show Rtp1 (FIG. 4A) and Rtp2 (FIG. 4B) expression in aorta as measured by qPCR.

FIGS. 5A-B show Olfr2 expression in T cells and B cells from whole digested aorta.

FIG. 6A show Olfr2 expression on blood mononuclear cells.

FIG. 6B shows Olfr2 function on blood mononuclear cells.

FIGS. 7A-G show that Olfr2 ligation induces an inflammatory response by vascular macrophages. FIG. 7A) Ca²⁺ signals of Ctrl and Oct are displayed as a dot plot. FIG. 7B) Time point analyzed Fluo-4 Median Fluorescence Intensity (MFI). SD is calculated among the cells for each time point. (n=20-50). FIG. 7C) Oct induces pro-inflammatory gene expression in aorta. FIG. 7D) Cit (100 μM) reduced octanal induced expression of Ccl2, Ccl4, Tnf, and Nos2 in aortas. (n=5-6/group). FIG. 7E) CCL2, CCL4, and TNF protein quantification by cytokine bead arrays and flow cytometry in supernatants of stimulated aortas. FIG. 7F) Intracellular TNF expression in F4/80⁺ vascular macrophages, displayed as MFI. FIG. 7G) BMDMs were treated with LPS (50 ng/mL) for 18 h and subsequently stimulated with Oct and Cit alone or both combined.

FIGS. 8A-B show calcium (Ca2+) signal detection displayed as a dot plot. FIG. 8A: Dump channel+ cells (TCRb+, and CD19+), loaded with 2 μM Fluo-4 before and after addition of Olfr2 agonist octanal (Oct 10 μM), and antagonist Citral (Cit 100 μM) alone or combined (Oct+Cit). Oct, and Cit injected at 60 seconds. Whole aortas were digested, stained with Fluo4 (2 μM) at 37° C. for 30 min, then washed and incubated with cell surface markers at RT for 20 min CD45+, live, dump channel+(TCRb+, and CD19+) cells were analyzed by flow cytometry. FIG. 8B: The time average Median Fluorescence Intensity (MFI) of Fluo-4 is reported. SD is calculated among the cells for each time point (n 20-50). The analysis is made out one of three experiments.

FIGS. 9A-B shows the results of evaluating octanal dose response. Octanal (Oct) dose response analysis on representative pro inflammatory genes (Ccl4 and Nos2). Explanted aortas of Apoe−/− mice kept in WD, were treated for 12h with three Octanal concentrations (3 μM, 10 μM and 30 μM) with (FIG. 9A) and without (FIG. 9B) LPS (500 ng/mL). After incubation whole aortas were transferred into Qiazol (500 μl; Qiagen) and digested with gentleMACS™ Dissociator (Miltenyi). RNA extraction was performed using a Qiazol/RNAeasy micro kit hybrid protocol (Qiagen). Total RNA was quantified using a nanodrop spectrophotometer (Peqlab) and 500 ng were reverse transcribed into cDNA using Omniscript reverse transcriptase (Qiagen). Real time PCR reactions were performed according to the RT² SYBR green gene expression assay protocol (Qiagen). RT2 SYBR Green qPCR master mix and premade RT² qPCR Primer Assays for mouse Ccl2, Ccl4, and Gapdh were used. Relative expression was normalized to Gapdh expression and the untreated control. Data are presented as means±SEM. n=3 per group *p<0.05, **p<0.01, ***p<0.005, ****p<0.001. p calculated by One-Way ANOVA test, Tukey's multiple comparisons test.

FIGS. 10A-J shows that octanal concentration increases in blood plasma of western diet fed mice and exacerbates atherosclerosis. FIGS. 10A-C: Murine blood plasma was analyzed for octanal by derivatization and stable isotope dilution LC/MS/MS. [²H₁₆]octanal was spiked to plasma before derivatization reaction as internal standard. Male WT C57BL/6J (B) and Apoe^{-/-} (C) mice were fed chow diet (CD) or western diet (WD) and plasma was analyzed for octanal concentrations (n=9/10/group). FIGS. 10D-G) En face atherosclerosis quantification in vehicle-, octanal- or citral-treated mice. Apoe^{-/-} mice (n=9-11/group) were fed WD for 8 weeks and injected every three days intraperitoneally with vehicle (PBS+0.1% DMSO), octanal (Oct, 10 mg/kg), or citral (Cit, 10 mg/kg) for the last 4 weeks for a total of 12 injections. D) Representative pictures of pinned aortic arches of vehicle and Oct-treated mice. Scale bar=0.5 cm. E) En face atherosclerotic lesion size evaluation in aortic arches as percentage of total area analyzed. F) Representative pictures of pinned aortic arches of mice vehicle- or Cit-treated mice. Scale bar=0.5 cm. G) Aortic arch lesion size quantification of citral treated mice displayed as percentage of total area analyzed. FIGS. 10H-J) Lldr^{-/-} male mice (n=8-10/group) were lethally irradiated and reconstituted with Rtp1/Rtp2^{+/+}, Rtp1/Rtp2^{+/-}, Rtp1/Rtp2^{-/-} bone marrow cells and fed high cholesterol diet for 12 weeks. Olfr2 expression of BMDMs from BM transferred mice by (H) western blot or (I) flow cytometry. J) Aortic arch lesion size quantification as percent of analyzed arch area. Data are presented as mean±SEM. *p<0.05, **p<0.01, ***p<0.005, ****p<0.001. p calculated by One-Way ANOVA test, non-parametric Kruskal-Wallis test with Dunn's test for multiple comparisons and unpaired t-test.

FIGS. 11A-B shows body weight and hematological parameters, in octanal (FIG. 11A) and citral (FIG. 11B) treated mice. Neutrophils (NE), Lymphocytes (LY), Monocytes (MO), Eosinophils (EO) and Basophils (BA) % among total withe blood cells is reported.

FIG. 12 shows body weight and hematological parameters, in Lldr−/− male mice (n 8/10 per group) transplanted with Rtp1/Rtp2+/+, Rtp1/Rtp2+/−, Rtp1/Rtp2−/− derived Bone Marrow cells and treated 12 wks with high cholesterol diet. Neutrophils (NE), Lymphocytes (LY), Monocytes (MO), Eosinophils (EO) and Basophils (BA) % among total with blood cells is reported.

FIG. 13 shows that human macrophages express the Olfr2 orthologue OR6A2 and ligation activates a pro-inflammatory response. FIG. 13A) Olfactory receptor and accessory gene expression in human atherosclerotic plaques and PBMCs shown as a heat map (red=high, blue=low, by row). Data was extracted from the BIKE database (GSE21545). Olfactory receptors highly expressed in plaques including OR6A2 (green) are highlighted. FIGS. 13B-E) Monocytes from healthy human donors were differentiated into macrophages (hMDMs). hMDMs were untreated or incubated with octanal (Oct, 10 μM) and LPS, (500 ng/mL) alone or combined (LPS+Oct) for 12 h. Bi) Quantitative real time PCR analysis of OR6A2 expression normalized to Gapdh expression and untreated control. Bii and iii) Flow cytometric analysis of OR6A2 expression on living hMDMs reported as MFI. FIG. 13C) High resolution imaging of hMDMs stained for OR6A2 (AF488) and hoechst dye (nuclei). FIG. 13D) Real time PCR quantification for human IL6, TNF, and NOS2 gene expression normalized to Gapdh and untreated control. FIG. 13E) IL6 and TNF protein quantification by cytokine bead arrays and flow cytometry in supernatants of untreated and stimulated hMDMs (n=4/group). FIG. 13F) hMDMs were treated with LPS (50 ng/mL) for 18 h and left untreated or further incubated with Oct and Cit alone or combined. IL-1β protein in supernatants was quantified by a human cytokine bead array and flow cytometry. FIG. 13G) hMDMs were transfected with a OR6A2 small interfering RNAs (SiORA6A2) or scramble control siRNAs (SiCtrl). After 48 h, hMDMs were treated with LPS (500 ng/mL) alone or with LPS+Oct (10 μM) for 12 h. OR6A2, TNF, IL6 and NOS2 gene expression analysis was analyzed by real time PCR and normalized to Gapdh and LPS siCtrl treated controls. All experiments were replicated at least three times. Data are presented as mean±SEM. *p<0.05, **p<0.01, ***p<0.005, ****p<0.001. p calculated by One-Way ANOVA test, Tukey's multiple comparisons test and unpaired t-test.

FIG. 14 shows an analysis of pro inflammatory genes CCL2, CCL4, and Tnf in aortas of Apoe^{-/-} mice. Explanted aortas of Apoe^{-/-} mice kept in WD (2 wks), were treated for 12h with LPS (500 ng/mL) and different odorants (10 μM) or left untreated. Relative expression of Ccl2, Ccl4, and Tnf was quantified by real time PCR analysis and normalized to Gapdh expression and untreated control applying the 2-ΔΔCT method. N=3 per group.

FIG. 15 shows a statistical analysis of the relative expression of pro inflammatory genes Ccl2, Ccl4 and Tnf in FIG. 14. Average relative expression for Ccl2, Ccl4, and Tnf was quantified by real time PCR analysis and normalized to Gapdh expression and untreated control applying the 2-ΔΔCT method. N=3 per group. p calculated by unpaired T Test. A p value<0.05 was considered significative.

FIG. 16 shows significantly modulated proteins in plasma from octanal treated mice. EDTA plasma samples were collected from octanal treated and vehicle treated mice at the end of the 8 weeks and were sent for analysis of 92 proteins. Analysis of 92 proteins comprising the Olink Mouse exploratory panel (https://www.olink.com/products/mouse-exploratory/) was performed using PEA (Proximity Extension Assay) technology. The PEA technique allows simultaneous assessment of proteins using oligonucleotide-labeled antibody probe pairs that bind to each protein within the sample. The PEA technique also permits accurate assessment of protein levels with repeated measurements by requiring both dual recognition of correctly matched antibody pairs and DNA-barcoding from sequence-specific oligonucleotides to generate a signal (PLoS ONE. 2014; 9(4)). The assay measures fold change in log 2 units. For this analysis, the raw data are converted into a t-statistic which can be compared across assays. Data are presented as means±SEM. P value<0.05 calculated by unpaired t-test.

FIG. 17 shows significantly modulated proteins in plasma from citral treated mice. EDTA plasma samples were collected from citral treated and vehicle treated mice at the end of the 8 weeks and were sent for analysis of 92 proteins. Analysis of 92 proteins comprising the Olink Mouse exploratory panel (https://www.olink.com/products/mouse-exploratory/) was performed using PEA (Proximity Extension Assay) technology, discussed above. Data are presented as means±SEM. P value<0.05. p calculated by unpaired t-test.

FIG. 18 shows “germ free” (no microbiome) (GF) and conventional C57BL/6J (Ctrl) mice plasma analysed for octanal concentrations (n=5/group).

### DETAILED DESCRIPTION

In the present invention, a number of olfactory receptors (Olfrs), as well as the essential Olfr accessory transport proteins RTP1 (Receptor Transporter Protein 1) and RTP2 (Receptor Transporter Protein 2), are shown to be expressed in vascular macrophages isolated from ApoE−/− murine aortas. Furthermore, stimulation of the olfactory receptor Olfr2 by a small molecule odorant (octanal) induces expression of inflammatory mediators, CCL2 (C-C motif chemokine ligand 2), iNOS (inducible Nitric Oxide Synthase) and TNF (tumor necrosis factor alpha); exposure to both octanal and LPS (lipopolysaccharide) exacerbate the inflammatory response, indicating that the inflammation may be caused by microbiota-derived odorants.

Olfactory receptors (Olfrs) are class A, G protein coupled receptors (GPCRs) that are bound by volatile chemical ligands, or odorants. Olfrs were first described in olfactory epithelial cells in the nasal cavity of rats^{1,2}. Olfrs have an average length of 320±25 amino acid residues and share characteristic conserved motifs³. Olfr genes constitute the largest family GPCRs with ˜1100 genes in mice and ˜400 in humans^{4,5}. Many Olfrs require the transporters Rtp1 and 2 and other molecules such as receptor expression enhancing protein 3 (Reep3) for expression⁶. Olfrs couple through a specialized G protein (Gαolf, Gnal)⁷ to adenylate cyclase 3 (Acy3)⁸⁻¹¹¹. Activation of Gα(olf) and Acy3 induces an increase in the intracellular concentration of cyclic adenosine monophosphate (cAMP) followed by the activation of cAMP-gated channels, influx of extracellular Na⁺ and Ca²⁺, and membrane depolarization.

Applicants have discovered that the Olfr2 ligand octanal significantly enhances LPS-induced inflammatory cytokines, which is blocked by the Olfr2 antagonist citral. Octanal naturally occurred in citrus oils and is used as a flavor food additive. It is in a class of odorants (aldheydes) that may have effects on atherosclerosis. Treating Apoe−/− mice with citral reduced their atherosclerotic lesions by ˜50%. Olfr2 is highly expressed in vascular macrophage. qRT-PCR showed that Olfr2 expression is increased fourfold by LPS and further increased by adding its ligand octanal, but octanal alone had no effect on Olfr2 expression. This increased expression of Olfr2 was confirmed at the protein level. Without being bound by theory, Applicants believe that it may be caused by increased expression of the transporters Rpt 1 and 2, which are both induced by LPS. LPS treatment of mice also increased Olfr2 expression on Ly6C+ blood monocytes and, to a lesser extent, on Ly6C-patrolling monocytes, but not on any other immune cells in the aorta or in blood (data not shown).

To begin to address the possible function of Olfr2 in vascular macrophages, inflammatory cytokines, chemokines and the M1-defining enzyme iNOS were surveyed by qRT-PCR. The known induction by LPS of CCL2 (=MCP-1) and CCL4 was significantly enhanced by octanal, and even octanal alone (without LPS) resulted in a measurable increase of both chemokines. iNOS expression induced by LPS was greatly enhanced by octanal. Octanal had the greatest effect on TNF, where ligation of Olfr2 was required to reveal an LPS-induced increase. The enhanced production of CCL2, CCL4 and TNF was confirmed at the protein level by cytometric bead array. Octanal also boosted the expression of TNF in both Ly6C+ and Ly6C− blood monocytes.

Some microbiota-derived molecules are known to promote atherosclerosis, e.g., TMAO. TMAO is produced by intestinal bacteria from choline. It has various effects, including enhanced platelet activation. Other microbiota-derived odorants have been identified. By way of example, and not by way of limitation, some of the identified microbiota-derived odorants include TMA and its liver derivative TMAO, p-cresol and its liver derivatives p-cresol-sulfate and p-cresol-glucuronide, indole and its liver derivatives indoxyl-sulfate and indoxyl-glucuronide, tryptamine, serotonin (also endogenous), SCFA (acetic, propionic, butyric, valeric, isovaleric, succinic, lactic acid), Phenylacetic acid and its liver derivatives phenylacetyl-glutamine and phenylacetyl-glycine, Indole-acetic acid, Indole-propionic acid, Phenyl-acetic acid, Phenyl-propionic acid, Benzoic acid and its liver derivative hippuric acid (also 2-hydroxy benzoic, 3-hydroxy benzoic and 4-hydroxybenzoic and their liver derivatives).

Thus, in certain embodiments, the olfactory receptors identified herein are modulated for treatment of an undesirable or aberrant immune response, immune disorder, inflammatory response or inflammation or an autoimmune response, disorder or disease including but not limited to rheumatoid arthritis, juvenile rheumatoid arthritis, osteoarthritis, psoriatic arthritis, diabetes mellitus, multiple sclerosis, encephalomyelitis, myasthenia gravis, systemic lupus erythematosus (SLE), autoimmune thyroiditis, atopic dermatitis, eczematous dermatitis, psoriasis, Sjogren's Syndrome, Crohn's disease, aphthous ulcer, iritis, conjunctivitis, keratoconjunctivitis, ulcerative colitis, inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common g chain (c) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (g5/14.1) deficiency, Hyper-IgM syndrome: X-linked (CD40 ligand deficiency) or non-X-linked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon g receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1 A/SAP deficiency), or hyper IgE syndrome.

In certain embodiments, the undesirable or aberrant immune response, immune disorder, inflammatory response or inflammation or an autoimmune response, disorder or disease is a symptom, side-effect or result of is of cancer or a pathogen infection.

In other embodiments, the olfactory receptors identified herein are modulated for treatment of a neoplastic disorder, tumor, cancer or malignancy, metastasis of a neoplasia, tumor, cancer or malignancy to other sites, or formation or establishment of a metastatic neoplasia, neoplastic disorder, tumor, cancer or malignancy to other sites distal from a primary neoplasia, neoplastic disorder, tumor, cancer or malignancy.

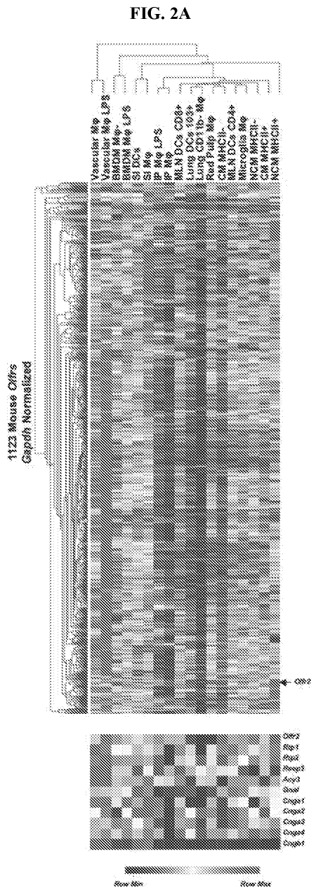

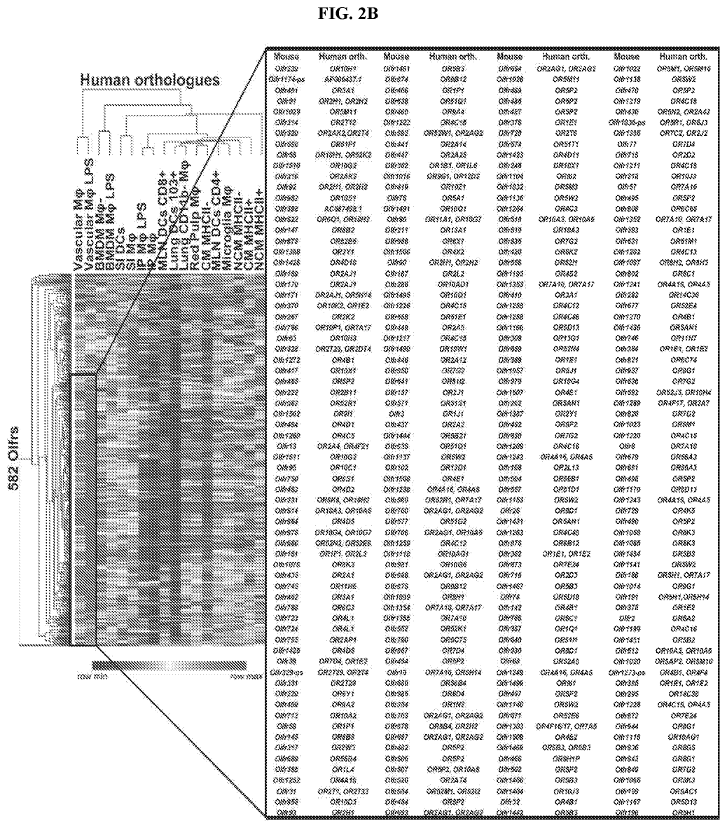

In some embodiment, the present invention comprises modulating one or more olfactory receptor listed in any of Table 1, Table 2, Table 3, Table 4, Table 5, Table 6, Table 7, Table 8, Table 9, FIGS. 2A and 2B. In certain embodiments, the present invention comprises decreasing, reducing, inhibiting, suppressing, limiting or controlling expression or activity of one or more olfactory receptors listed in any of Table 1, Table 2, Table 3, Table 4, Table 5, Table 6, Table 7, Table 8, Table 9, FIGS. 2A and 2B. In other embodiments, the present invention comprises enhancing, stimulating, inducing or increasing expression or activity of one or more olfactory receptors listed in any of Table 1, Table 2, Table 3, Table 4, Table 5, Table 6, Table 7, Table 8, Table 9, FIGS. 2A and 2B. In certain embodiments, the present invention comprises a combination of decreasing, reducing, inhibiting, suppressing, limiting or controlling expression or activity of one or olfactory receptors listed in any of Table 1, Table 2, Table 3, Table 4, Table 5, Table 6, Table 7, Table 8, Table 9, FIGS. 2A and 2B, and enhancing, stimulating, inducing or increasing expression or activity of one or more different olfactory receptor listed in any of Table 1, Table 2, Table 3, Table 4, Table 5, Table 6, Table 7, Table 8, Table 9, FIGS. 2A and 2B.

Non-limiting examples of an undesirable or aberrant immune response, immune disorder, inflammatory response or inflammation or an autoimmune response, disorder or disease include rheumatoid arthritis, juvenile rheumatoid arthritis, osteoarthritis, psoriatic arthritis, diabetes mellitus, multiple sclerosis, encephalomyelitis, myasthenia gravis, systemic lupus erythematosus (SLE), autoimmune thyroiditis, atopic dermatitis, eczematous dermatitis, psoriasis, Sjogren's Syndrome, Crohn's disease, aphthous ulcer, iritis, conjunctivitis, keratoconjunctivitis, ulcerative colitis, inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common g chain (c) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (g5/14.1) deficiency, Hyper-IgM syndrome: X-linked (CD40 ligand deficiency) or non-X-linked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon g receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1 A/SAP deficiency), or hyper IgE syndrome.

Non-limiting examples of neoplasia, neoplastic disorders, tumors, cancers or malignancies treated by the present methods include, a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, lymphosarcoma, liposarcoma, osteosarcoma, chondrosarcoma, leiomyosarcoma, rhabdomyosarcoma, fibrosarcoma or melanoma as well as a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, neoplastic disorder, tumor, cancer or malignancy.

Disclosed herein are methods of modulating an immune response in a subject in need thereof. In some embodiments, the method of modulating an immune response in a subject, comprises modulating expression or activity of one or more olfactory receptors (OLFR). The OLFR may be expressed by a cell in vivo. The cell may be an animal cell, with the proviso that the cell is not an olfactory cell. The cell may be a macrophage. The macrophage may be a vascular macrophage.

The modulation may comprise one or more of inhibiting, decreasing, reducing, suppressing, limiting or controlling the immune response by a method comprising administering to the subject an effective amount of an agent that inhibits the expression of or deactivates the (OLFR). The modulation may comprise one or more of decreasing, reducing, inhibiting, suppressing, limiting or controlling an undesirable or aberrant immune response, immune disorder, inflammatory response or inflammation, by a method comprising administering to the subject an effective amount of an agent that inhibits the expression of or deactivates the (OLFR). The modulation may comprise one or more of decreasing, reducing, inhibiting, suppressing, limiting or controlling an autoimmune response, disorder or disease in a subject, by a method comprising administering to the subject an effective amount of an agent that inhibits the expression of or deactivates the (OLFR). The modulation may comprise one or more of decreasing, reducing, inhibiting, suppressing, limiting or controlling an adverse symptom of the undesirable or aberrant immune response, immune disorder, inflammatory response or inflammation, or an adverse symptom of the autoimmune response, disorder or disease in the subject.

The methods or uses disclosed herein may comprise modulating an adverse symptom. The adverse symptom of the undesirable or aberrant immune response, immune disorder, inflammatory response or inflammation or an adverse symptom of the autoimmune response, disorder or disease may be swelling, pain, rash, headache, fever, nausea, diarrhea, bloat, lethargy, skeletal joint stiffness or tissue or cell damage. The adverse symptom of the undesirable or aberrant immune response, immune disorder, inflammatory response or inflammation or the adverse symptom of the autoimmune response, disorder or disease may be chronic or acute.

The immune disorder, inflammatory response, inflammation, autoimmune response disorder or autoimmune disease may comprise rheumatoid arthritis, juvenile rheumatoid arthritis, osteoarthritis, psoriatic arthritis, diabetes mellitus, multiple sclerosis, encephalomyelitis, myasthenia gravis, systemic lupus erythematosus (SLE), autoimmune thyroiditis, atopic dermatitis, eczematous dermatitis, psoriasis, Sjogren's Syndrome, Crohn's disease, aphthous ulcer, iritis, conjunctivitis, keratoconjunctivitis, ulcerative colitis, inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common g chain (c) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (g5/14.1) deficiency, Hyper-IgM syndrome: X-linked (CD40 ligand deficiency) or non-X-linked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon g receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1 A/SAP deficiency), or hyper IgE syndrome.

The modulation may comprise one or more of decreasing, reducing, inhibiting, suppressing, limiting or controlling an adverse cardiovascular event or cardiovascular disease by a method comprising administering to the subject an effective amount of an agent that inhibits the expression of or deactivates the OLFR. The adverse cardiovascular event or cardiovascular disease may comprise coronary artery disease, peripheral artery disease, cerebrovascular disease, renal artery disease, stroke, myocardial infarction (heart attack), ischemic heart failure, transient ischemic attack or brain trauma, atherosclerosis, atherosclerotic plaque formation or elevated blood cholesterol.

The modulation may comprise one or more of decreasing, reducing, inhibiting, suppressing, limiting or controlling atherosclerosis, by a method comprising administering to the subject an effective amount of an agent that inhibits the expression of or deactivates the OLFR.

The method may comprise one or more of reducing or inhibiting in a subject viral, bacterial or fungal infection or a neoplasia, neoplastic disorder, tumor, cancer or malignancy, by a method comprising administering to the subject an effective amount of an agent that increases the expression of or activates the OLFR.

The modulation may comprise one or more of comprising decreasing, reducing, inhibiting, suppressing, limiting or controlling an adverse symptom of the neoplasia, neoplastic disorder, tumor, cancer or malignancy, metastasis of a neoplasia, tumor, cancer or malignancy to other sites, or formation or establishment of a metastatic neoplasia, neoplastic disorder, tumor, cancer or malignancy to other sites distal from a primary neoplasia, neoplastic disorder, tumor, cancer or malignancy, or viral, bacterial or fungal infection by a method comprising administering to the subject an effective amount of an agent that modulates the expression of or activity of the OLFR.

The OLFR may be enhanced or stimulated in the subject by administration of an effective amount of an agent that stimulates or enhances the activity of the OLFR. The OLFR may be enhanced or stimulated in the subject by administration of an OLFR. The OLFR may be enhanced or stimulated in the subject by administration of a nucleotide that encodes an OLFR. The OLFR may be enhanced or stimulated in the subject by administration of an OLFR ligand. The OLFR may be enhanced or stimulated in the subject by administration of a receptor transporting protein 1 (RTP1) or a nucleotide encoding RTP1. The OLFR may be enhanced or stimulated in the subject by administration of a RTP2 or a nucleotide encoding RTP2. The OLFR may be enhanced or stimulated in the subject by administration of a receptor expression enhancing protein 1 (REEP1) or a nucleotide encoding REEP1. The OLFR may be enhanced or stimulated in the subject by administration of an aminoacylase 3 (Acy3) or a nucleotide encoding Acy3. The OLFR may be enhanced or stimulated in the subject by administration of a guanine nucleotide-binding protein G(olf) subunit alpha (Gnal) or a nucleotide encoding Gnal.

The methods disclosed herein may be used to treat a neoplasia, neoplastic disorder, tumor, cancer or malignancy. Further disclosed herein is the use of an agent that modulates the activity or expression of an OLFR in the preparation of a medicament for the treatment of a neoplasia, neoplastic disorder, tumor, cancer or malignancy. Further disclosed herein is the use of an agent that increases the activity or expression of an OLFR in the preparation of a medicament for the treatment of a neoplasia, neoplastic disorder, tumor, cancer or malignancy. Further disclosed herein is the use of an OLFR ligand in the preparation of a medicament for the treatment of a neoplasia, neoplastic disorder, tumor, cancer or malignancy. Disclosed herein is the use of octanal in the preparation of a medicament for the treatment of a neoplasia, neoplastic disorder, tumor, cancer or malignancy. Disclosed herein is the use of an OLFR ligand for the treatment of a neoplasia, neoplastic disorder, tumor, cancer or malignancy. Further disclosed herein is the use of octanal for the treatment of a neoplasia, neoplastic disorder, tumor, cancer or malignancy.

The neoplasia, neoplastic disorder, tumor, cancer or malignancy treated may be a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, lymphosarcoma, liposarcoma, osteosarcoma, chondrosarcoma, leiomyosarcoma, rhabdomyosarcoma, fibrosarcoma or melanoma; or a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, neoplastic disorder, tumor, cancer or malignancy.

The methods and uses disclosed herein may comprise modulating the activity or expression of an OLFR. The OLFR may be an OLFR listed in any one of Tables 1-9 Tables 2A and 2B. The OLFR may be an OLFR listed in Table 1. The OLFR may be an OLFR listed in Table 2. The OLFR may be an OLFR listed in Table 3. The OLFR may be an OLFR listed in Table 4. The OLFR may be an OLFR listed in Table 5. The OLFR may be an OLFR listed in Table 6. The OLFR may be an OLFR listed in Table 7. The OLFR may be an OLFR listed in Table 8. The OLFR may be an OLFR listed in Table 9. The OLFR may be an OLFR listed in FIG. 2A. The OLFR may be an OLFR listed in FIG. 2B. The OLFR may be selected from the group consisting of OR7C1, OR7D4, OR10A6, OR11H6, OR4E2, OR10H1, and OR6A2. The OLFR may be OR7C1. The OLFR may be OR7D4. The OLFR may be OR10A6. The OLFR may be OR11H6. The OLFR may be OR4E2. The OLFR may be OR10H1. The OLFR may be OR6A2.

The agent may be selected from the group of: a ligand or small molecule that binds to the OLFR or blocks the binding of the OLFR to the ligand or an agent that inhibits the expression of the OLFR by the cell. The agent may be a ligand or small molecule that binds to the OLFR. The agent may be a ligand or small molecule that blocks the binding of the OLFR to the ligand. The agent may inhibit the expression of the OLFR by the cell. The agent may be selected from the group of an antibody, fragment or mimetic that binds to OLFR or an OLFR ligand, an anti-OLFR gene silencing agent, or octanal or a prodrug or solvate thereof. The agent may be an antibody, fragment or mimetic that binds to OLFR. The agent may be an antibody, fragment or mimetic that binds to an OLFR ligand. The agent may be an anti-OLFR gene silencing agent. The agent may be octanal, heptanal, or a prodrug or solvate thereof.

The agent may activate the OLFR by increasing the trafficking of the OLFR to a plasma membrane of a cell. The agent may be a protein selected from the group of receptor transporting protein 1 (RTP1), RTP2, receptor expression enhancing protein 1 (REEP1), aminoacylase 3 (Acy3), or guanine nucleotide-binding protein G(olf) subunit alpha (Gnal).

The agent may be a nucleotide encoding a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal).

The immune response may be stimulated by a method comprising administering an agent that increases expression or secretion of an inflammatory cytokine. The inflammatory cytokine may be selected from the group consisting of tumor necrosis factor (TNF), C-C motif chemokine ligand 2 (CCL2), CCL4, CCL5, interleukin 6 (IL-6), interleukin 1-beta (IL-1B), interleukin 18 (IL-18), and nitric oxide synthase 2 (NOS2). The inflammatory cytokine may be TNF. The inflammatory cytokine may be CCL2. The inflammatory cytokine may be CCL4. The inflammatory cytokine may be CCL5. The inflammatory cytokine may be IL-6. The inflammatory cytokine may be IL-1B. The inflammatory cytokine may be IL-18. The inflammatory cytokine may be NOS2.

Increasing or Stimulating an Immune Response

Disclosed herein are methods of increasing or stimulating an immune response in a subject in need thereof. In some embodiments, the method comprises administering to the subject an olfactory receptor (OLFR). Alternatively, or additionally, the method comprises administering to the subject an agent that increases the expression of or activity of an olfactory receptor (OLFR), thereby increasing or stimulating an immune response in the subject. In some embodiments, the method comprises administering to the subject an agent that increases or promotes the trafficking of an olfactory receptor (OLFR) to a plasma membrane of a cell, thereby increasing an immune response in the subject. Further disclosed herein is the use of an olfactory receptor (OLFR) in the manufacture of a medicament for increasing or stimulating an immune response. Disclosed herein is the use of an agent that increases the expression of or activity of an olfactory receptor (OLFR) in the manufacture of a medicament for increasing or stimulating an immune response. Disclosed herein is the use of an agent that increases or promotes the trafficking of an olfactory receptor (OLFR) to a plasma membrane of a cell in the manufacture of a medicament for increasing or stimulating an immune response. The OLFR may be an OLFR listed in Table 1. The OLFR may be an OLFR listed in Table 2. The OLFR may be an OLFR listed in Table 3. The OLFR may be an OLFR listed in Table 4. The OLFR may be an OLFR listed in Table 5. The OLFR may be an OLFR listed in Table 6. The OLFR may be an OLFR listed in Table 7. The OLFR may be an OLFR listed in Table 8. The OLFR may be an OLFR listed in Table 9. The OLFR may be an OLFR listed in FIG. 2A. The OLFR may be an OLFR listed in FIG. 2B. The OLFR may be selected from the group consisting of OR7C1, OR7D4, OR10A6, OR11H6, OR4E2, OR10H1, and OR6A2. The OLFR may be OR7C1. The OLFR may be OR7D4. The OLFR may be OR10A6. The OLFR may be OR11H6. The OLFR may be OR4E2. The OLFR may be OR10H1. The OLFR may be OR6A2. The agent may be a ligand that binds to the OLFR. The agent may be an OLFR agonist. The agonist may be selected from the group consisting of octanal, coumarin, helional, lilial, b-ionone, androstenone, androstadienone, caramel furanone, 3-phenyl propyl propionate, eugenol, ethil vanillin, 2-ethyl-fencol, isovaleric acid, nonanoic acid, butyl butyryllactate, butyric acid, isovaleric acid, propionic acid, N-amyl acetate, eugenol acetate, sandalwood, S-(−)-citronellol, S-(−)-citronellal, (+)-carvine, (−) carvone, (+) carvone, linalool, bourgeonal, acetophenone, amyl butyrate, nonanethiol, allyl phenyl acetate, N-amyl acetate, muscone, isoeugenol, eugenol methyl ether, 1-hexanol, 1-heptanole, 1-octanol, celery ketone, anis aldehyde, (+)-menthol, vanillin, guaiacol, lyral, ethyl heptanoate, methyl octanoate, nonanal, 1-nonanol, 2-nonanol, 3-octanone, 3-nonanone, decyl aldehyde. the agonist may be selected from the list of agonists provided in Table 8. The agent may be octanal, heptanal, or a prodrug or solvate thereof. The agent may activate the OLFR by increasing the trafficking of the OLFR to a plasma membrane of a cell. The agent may be a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal). The agent may comprise a nucleotide encoding a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal). Stimulating an immune response may comprise increasing expression or secretion of an inflammatory cytokine. The inflammatory cytokine may be selected from the group consisting of tumor necrosis factor (TNF), C-C motif chemokine ligand 2 (CCL2), CCL4, CCL5, interleukin 6 (IL-6), IL-1B, IL-18, and nitric oxide synthase 2 (NOS2).

In some embodiments, the immune response is increased or stimulated by at least 10%, 20%, 30%, 40%, 50%, 60%, 70%, 80%, 90%, 95%, 97%, or 100% as compared to the immune response in a subject who is not treated with the agent. The immune response may be increased or stimulated by at least 1.5, 2, 2.5, 3, 3.5, 4, 4.5, 5, 5.5, 6, 6.5, 7, 7.5, 8, 8.5, 9, 9.5, or 10-fold or more as compared to the immune response in a subject who is not treated with the agent. The immune response may be increased or stimulated by at least 15, 20, 25, 30, 35, 40, 45, 50, 55, 60, 65, 70, 75, 80, 85, 90, 95, or 100-fold or more as compared to the immune response in a subject who is not treated with the agent. The immune response may be measured by detecting the level of one or more inflammatory cytokines. Alternatively, or additionally, the immune response is measured by detecting the expression of one or more inflammatory cytokines. In some embodiments, the immune response is measured by detecting one or more circulating cytokines and/or chemokines. In some embodiments, the immune response is measured by detecting one or more cells selected from activated CD8 T cells, Th1 cells, Th2 cells, Th17 cells, TFH cells, TIP-DCs, and M1 polarized macrophages. In some embodiments, the immune response is measured by detecting one or more markers of T cell activation. In some embodiments, the immune response is measured by detecting the levels of antibody titers to one or more tumor antigens. In some embodiments, an increase in the expression level of or more inflammatory cytokines is indicative of an increase in the immune response. In some embodiments, an increase in the quantity of circulating cytokines and/or chemokines is indicative of an increase in the immune response. In some embodiments, an increase in the quantity of one or more cells is indicative of an increase in the immune response. In some embodiments, an increase in quantity of one or more markers of T cell activation that is detected is indicative of an increase in the immune response. In some embodiments, an increase in the level of antibody titers detected is indicative of an increase in the immune response. Alternatively, or additionally, the immune response is measured by a method such as ELISA, cytometric bead array, Olink proteomic, and flow cytometry.

Suppressing an Immune Response

Disclosed herein are methods of suppressing an immune response in a subject in need thereof. In some embodiments, the method comprises administering to the subject an agent that decreases the expression of or activity of an olfactory receptor (OLFR), thereby suppressing an immune response in the subject. In some embodiments, the method comprises administering to the subject an agent that inhibits the trafficking of an olfactory receptor (OLFR) to a plasma membrane of a cell, thereby suppressing an immune response in the subject. Further disclosed herein are uses of an agent that decreases the expression or activity of an OLFR in the manufacture of a medicament for suppressing an immune response. Further disclosed herein is the use of an agent that inhibits the trafficking of an OLFR to a plasma membrane of a cell in the manufacture of a medicament for suppressing an immune response. The OLFR may be an OLFR listed in Table 1. The OLFR may be an OLFR listed in Table 2. The OLFR may be an OLFR listed in Table 3. The OLFR may be an OLFR listed in Table 4. The OLFR may be an OLFR listed in Table 5. The OLFR may be an OLFR listed in Table 6. The OLFR may be an OLFR listed in Table 7. The OLFR may be an OLFR listed in Table 8. The OLFR may be an OLFR listed in Table 9. The OLFR may be an OLFR listed in FIG. 2A. The OLFR may be an OLFR listed in FIG. 2B. The OLFR may be selected from the group consisting of OR7C1, OR7D4, OR10A6, OR11H6, OR4E2, OR10H1, and OR6A2. The OLFR may be OR7C1. The OLFR may be OR7D4. The OLFR may be OR10A6. The OLFR may be OR11H6. The OLFR may be OR4E2. The OLFR may be OR10H1. The OLFR may be OR6A2. The agent may be a gene silencing agent. The gene silencing agent may be selected from the group consisting of a RNA interference (RNAi) molecule, zinc finger nuclease, transcription activator-like effector nuclease (TALEN), and Clustered Regulatory Interspaced Short Palindromic Repeats (CRISPR) enzyme. The RNAi molecule may be selected from the group consisting of a small interference RNA (siRNA), short hairpin RNA (shRNA) and microRNA (miRNA). The agent may decrease the activity of the OLFR by inhibiting the binding of the OLFR with its ligand. The agent may be an OLFR antagonist. The OLFR antagonist may be citral, a solvate or prodrug thereof. The OLFR antagonist may be methylcinnamaldehyde, hydrocinnamaldehyde, bourgeonal, undecanal, methyl isoeugenol a solvate or prodrug thereof. The antagonist may be selected from the list of antagonists provided in Table 8, or a solvate or prodrug thereof. The agent may decrease the activity of the OLFR by inhibiting the trafficking of the OLFR to a plasma membrane of a cell. The agent may be an antibody, fragment or mimetic thereof that binds to an OLFR.

In some embodiments, the immune response is suppressed or decreased by at least 10%, 20%, 30%, 40%, 50%, 60%, 70%, 80%, 90%, 95%, 97%, or 100% as compared to the immune response in a subject who is not treated with the agent. The immune response may be suppressed or decreased by at least 1.5, 2, 2.5, 3, 3.5, 4, 4.5, 5, 5.5, 6, 6.5, 7, 7.5, 8, 8.5, 9, 9.5, or 10-fold or more as compared to the immune response in a subject who is not treated with the agent. The immune response may be suppressed or decreased by at least 15, 20, 25, 30, 35, 40, 45, 50, 55, 60, 65, 70, 75, 80, 85, 90, 95, or 100-fold or more as compared to the immune response in a subject who is not treated with the agent. The immune response may be measured by detecting the level of one or more inflammatory cytokines. Alternatively, or additionally, the immune response is measured by detecting the expression of one or more inflammatory cytokines. In some embodiments, the immune response is measured by detecting one or more circulating cytokines and/or chemokines. In some embodiments, the immune response is measured by detecting one or more cells selected from activated CD8 T cells, Th1 cells, Th2 cells, Th17 cells, TFH cells, TIP-DCs, and M1 polarized macrophages. In some embodiments, the immune response is measured by detecting one or more markers of T cell activation. In some embodiments, the immune response is measured by detecting the levels of antibody titers to one or more tumor antigens. In some embodiments, a decrease in the expression level of or more inflammatory cytokines is indicative of a decrease or suppression in the immune response. In some embodiments, a decrease in the quantity of circulating cytokines and/or chemokines is indicative of a decrease or suppression in the immune response. In some embodiments, a decrease in the quantity of one or more cells is indicative of a decrease or suppression in the immune response. In some embodiments, a decrease in quantity of one or more markers of T cell activation that is detected is indicative of a decrease or suppression in the immune response. In some embodiments, a decrease in the level of antibody titers detected is indicative of a decrease or suppression in the immune response. Alternatively, or additionally, the immune response is measured by a method such as ELISA, cytometric bead array, Olink proteomic, and flow cytometry.

Autoimmune Disease

Further disclosed herein are methods of treating an autoimmune disease in a subject in need thereof. In some embodiments, the method comprises administering to the subject an agent that decreases the expression of or activity of an olfactory receptor (OLFR). Alternatively, or additionally, the method of treating an autoimmune disease in a subject in need thereof, comprises administering to the subject an agent that inhibits the trafficking of an olfactory receptor (OLFR) to a plasma membrane of a cell. Further disclosed herein is the use of an agent that decreases the expression of or activity of an olfactory receptor (OLFR) in the manufacture of a medicament for treating an autoimmune disease. Also disclosed herein is the use of an agent that decreases the expression of or activity of an olfactory receptor (OLFR) for the treatment of an autoimmune disease. Further disclosed herein is the use of an agent that inhibits the trafficking of an olfactory receptor (OLFR) to a plasma membrane of a cell in the manufacture of a medicament for treating an autoimmune disease. Also disclosed herein is the use of an agent that inhibits the trafficking of an olfactory receptor (OLFR) to a plasma membrane of a cell for the treatment of an autoimmune disease. The OLFR may be an OLFR listed in Table 1. The OLFR may be an OLFR listed in Table 2. The OLFR may be an OLFR listed in Table 3. The OLFR may be an OLFR listed in Table 4. The OLFR may be an OLFR listed in Table 5. The OLFR may be an OLFR listed in Table 6. The OLFR may be an OLFR listed in Table 7. The OLFR may be an OLFR listed in Table 8. The OLFR may be an OLFR listed in Table 9. The OLFR may be an OLFR listed in FIG. 2A. The OLFR may be an OLFR listed in FIG. 2B. The OLFR may be selected from the group consisting of OR7C1, OR7D4, OR10A6, OR11H6, OR4E2, OR10H1, and OR6A2. The OLFR may be OR7C1. The OLFR may be OR7D4. The OLFR may be OR10A6. The OLFR may be OR11H6. The OLFR may be OR4E2. The OLFR may be OR10H1. The OLFR may be OR6A2. The agent may be a gene silencing agent. The gene silencing agent may be selected from the group consisting of a RNA interference (RNAi) molecule, zinc finger nuclease, transcription activator-like effector nuclease (TALEN), and Clustered Regulatory Interspaced Short Palindromic Repeats (CRISPR) enzyme. The RNAi molecule may be selected from the group consisting of a small interference RNA (siRNA), short hairpin RNA (shRNA) and microRNA (miRNA). The agent may decrease the activity of the OLFR by inhibiting the binding of the OLFR with its ligand. The agent may be an OLFR antagonist. The OLFR antagonist may be citral, a solvate or prodrug thereof. The OLFR antagonist may be methylcinnamaldehyde, hydrocinnamaldehyde, bourgeonal, undecanal, methyl isoeugenol a solvate or prodrug thereof. The antagonist may be selected from the list of antagonists provided in Table 9, or a solvate or prodrug thereof.

The agent may decrease the activity of the OLFR by inhibiting the trafficking of the OLFR to a plasma membrane of a cell. The agent may be an antibody, fragment or mimetic thereof that binds to an OLFR.

In some embodiments, the autoimmune disease is selected from the group consisting of rheumatoid arthritis, juvenile rheumatoid arthritis, osteoarthritis, psoriatic arthritis, diabetes mellitus, multiple sclerosis, encephalomyelitis, myasthenia gravis, systemic lupus erythematosus (SLE), autoimmune thyroiditis, atopic dermatitis, eczematous dermatitis, psoriasis, Sjogren's Syndrome, Crohn's disease, aphthous ulcer, iritis, conjunctivitis, keratoconjunctivitis, ulcerative colitis, inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common g chain (c) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (g5/14.1) deficiency, Hyper-IgM syndrome: X-linked (CD40 ligand deficiency) or non-X-linked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon g receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1 A/SAP deficiency), or hyper IgE syndrome.

Cardiovascular Disease

Disclosed herein are methods of treating a cardiovascular disease in a subject in need thereof. In some embodiments, the method comprises administering to the subject an agent that decreases the expression of or activity of an olfactory receptor (OLFR). Alternatively, or additionally, the method comprises administering to the subject an agent that inhibits the trafficking of an olfactory receptor (OLFR) to a plasma membrane of a cell. Further disclosed herein is the use of an agent that decreases the expression of or activity of an olfactory receptor (OLFR) in the manufacture of a medicament for treating a cardiovascular disease. Also disclosed herein is the use of an agent that decreases the expression of or activity of an olfactory receptor (OLFR) for the treatment of a cardiovascular disease. Further disclosed herein is the use of an agent that inhibits the trafficking of an olfactory receptor (OLFR) to a plasma membrane of a cell in the manufacture of a medicament for treating a cardiovascular disease. Also disclosed herein is the use of an agent that inhibits the trafficking of an olfactory receptor (OLFR) to a plasma membrane of a cell for the treatment of a cardiovascular disease. The OLFR may be an OLFR listed in Table 1. The OLFR may be an OLFR listed in Table 2. The OLFR may be an OLFR listed in Table 3. The OLFR may be an OLFR listed in Table 4. The OLFR may be an OLFR listed in Table 5. The OLFR may be an OLFR listed in Table 6. The OLFR may be an OLFR listed in Table 7. The OLFR may be an OLFR listed in Table 8. The OLFR may be an OLFR listed in Table 9. The OLFR may be an OLFR listed in FIG. 2A. The OLFR may be an OLFR listed in FIG. 2B. The OLFR may be selected from the group consisting of OR7C1, OR7D4, OR10A6, OR11H6, OR4E2, OR10H1, and OR6A2. The OLFR may be OR7C1. The OLFR may be OR7D4. The OLFR may be OR10A6. The OLFR may be OR11H6. The OLFR may be OR4E2. The OLFR may be OR10H1. The OLFR may be OR6A2. The agent may be a gene silencing agent. The gene silencing agent may be selected from the group consisting of a RNA interference (RNAi) molecule, zinc finger nuclease, transcription activator-like effector nuclease (TALEN), and Clustered Regulatory Interspaced Short Palindromic Repeats (CRISPR) enzyme. The RNAi molecule may be selected from the group consisting of a small interference RNA (siRNA), short hairpin RNA (shRNA) and microRNA (miRNA). The agent may decrease the activity of the OLFR by inhibiting the binding of the OLFR with its ligand. The agent may be an OLFR antagonist. The OLFR antagonist may be citral, a solvate or prodrug thereof. The OLFR antagonist may be methylcinnamaldehyde, hydrocinnamaldehyde, bourgeonal, undecanal, methyl isoeugenol a solvate or prodrug thereof. The antagonist may be selected from the list of antagonists provided in Table 9 or a solvate or prodrug thereof. The agent may decrease the activity of the OLFR by inhibiting the trafficking of the OLFR to a plasma membrane of a cell. The agent may be an antibody, fragment or mimetic thereof that binds to an OLFR.

In some embodiments, the adverse cardiovascular event or cardiovascular disease is selected from the group consisting of coronary artery disease, peripheral artery disease, cerebrovascular disease, renal artery disease, stroke, myocardial infarction (heart attack), ischemic heart failure, transient ischemic attack or brain trauma, atherosclerosis, atherosclerotic plaque formation and elevated blood cholesterol.

Infections or Infectious Diseases

Disclosed herein are methods of treating an infection or infectious disease in a subject in need thereof. In some embodiments, the method comprises administering to the subject an olfactory receptor (OLFR). Alternatively, or additionally, the method comprises administering to the subject an agent that increases the expression of or activates an olfactory receptor (OLFR). Alternatively, or additionally, the method comprises administering to the subject an agent that increases or promotes the trafficking of an olfactory receptor (OLFR) to a plasma membrane of a cell. Further disclosed herein is the use of an olfactory receptor (OLFR) in the manufacture of a medicament for the treatment of an infection. Further disclosed herein is the use of an agent that increases the expression of or activates an olfactory receptor (OLFR) in the manufacture of a medicament for the treatment of an infection. Disclosed herein is the use of an agent that increases or promotes the trafficking of an olfactory receptor (OLFR) to a plasma membrane of a cell in the manufacture of a medicament for the treatment of an infection. Disclosed herein is the use of an olfactory receptor (OLFR) for the treatment of an infection or infectious disease. Disclosed herein is the use of an agent that increases the expression of or activates an olfactory receptor (OLFR) for the treatment of an infection or infectious disease. Disclosed herein is the use of an agent that increases or promotes the trafficking of an olfactory receptor (OLFR) to a plasma membrane of a cell for the treatment of an infection or infectious disease. The OLFR may be an OLFR listed in Table 1. The OLFR may be an OLFR listed in Table 2. The OLFR may be an OLFR listed in Table 3. The OLFR may be an OLFR listed in Table 4. The OLFR may be an OLFR listed in Table 5. The OLFR may be an OLFR listed in Table 6. The OLFR may be an OLFR listed in Table 7. The OLFR may be an OLFR listed in Table 8. The OLFR may be an OLFR listed in Table 9. The OLFR may be an OLFR listed in FIG. 2A. The OLFR may be an OLFR listed in FIG. 2B. The OLFR may be selected from the group consisting of OR7C1, OR7D4, OR10A6, OR11H6, OR4E2, OR10H1, and OR6A2. The OLFR may be OR7C1. The OLFR may be OR7D4. The OLFR may be OR10A6. The OLFR may be OR11H6. The OLFR may be OR4E2. The OLFR may be OR10H1. The OLFR may be OR6A2. The agent may be a ligand that binds to the OLFR. The agent may be an OLFR agonist. The agonist may be selected from the group consisting of octanal, coumarin, helional, lilial, b-ionone, androstenone, androstadienone, caramel furanone, 3-phenyl propyl propionate, eugenol, ethil vanillin, 2-ethyl-fencol, isovaleric acid, nonanoic acid, butyl butyryllactate, butyric acid, isovaleric acid, propionic acid, N-amyl acetate, eugenol acetate, sandalwood, S-(−)-citronellol, S-(−)-citronellal, (+)-carvine, (−) carvone, (+) carvone, linalool, bourgeonal, acetophenone, amyl butyrate, nonanethiol, allyl phenyl acetate, N-amyl acetate, muscone, isoeugenol, eugenol methyl ether, 1-hexanol, 1-heptanole, 1-octanol, celery ketone, anis aldehyde, (+)-menthol, vanillin, guaiacol, lyral, ethyl heptanoate, methyl octanoate, nonanal, 1-nonanol, 2-nonanol, 3-octanone, 3-nonanone, and decyl aldehyde. The agonist may be selected from the list of agonists provided in Table 8. The agent may be octanal, heptanal, or a prodrug or solvate thereof. The agent may activate the OLFR by increasing the trafficking of the OLFR to a plasma membrane of a cell. The agent may be a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal). The agent may comprise a nucleotide encoding a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal). Stimulating an immune response may comprise increasing expression or secretion of an inflammatory cytokine. The inflammatory cytokine may be selected from the group consisting of tumor necrosis factor (TNF), C-C motif chemokine ligand 2 (CCL2), CCL4, CCL5, interleukin 6 (IL-6), IL-1B, IL-18, and nitric oxide synthase 2 (NOS2). The infection may be a viral infection. The infection may be a bacterial infection. The infection may be a fungal infection.

In some embodiments, the infection is a viral infection. In some embodiments, the viral infection is caused by a virus selected from the group consisting of influenza virus, enterovirus (such as coxsackievirus and echovirus), cytomegalovirus, Zika virus, rabies virus, West Nile virus, rubella virus, polio virus, rotavirus, norovirus, herpes simplex virus, varicella-zoster virus, lymphocytic choriomeningitis virus, human immunodeficiency virus, Chikungunya virus, Crimean-Congo hemorrhagic fever virus, Japanese encephalitis virus, Rift Valley Fever virus, Ross River virus, and louping ill virus.

In some embodiments, the infection is a bacterial infection. In some embodiments, the bacterial infection is caused by a bacteria selected from the group consisting of streptococcus, staphylococcus, Escherichia coli, Campylobacter jejuni, Clostridium botulinum, Listeria monocytogenes, salmonella, and vibrio.

In some embodiments, the infection is a fungal infection. In some embodiments, the fungal infection is caused by a fungus selected from the group consisting aspergillus, candida, coccidioides, sphorotrichosis, Pneumocystis jirovecii, Blastomyces, Histoplasma, Cryptococcus neoformans, and Talaromyces. Examples of fungal infections include, but are not limited to aspergillosis, candidiasis, C. gattii infection, fungal nail infection, mucormycosis, pneumocystis pneumonia, blastomycosis, sporotrichosis, histoplasmosis, mycetoma, ringworm, talaromycosis, athlete's foot and yeast infection.

In some embodiments, the infection is selected from the group consisting of a respiratory infection, gastrointestinal infection, liver infection, nervous system infection, ear infection, throat infection, bladder infection, kidney infection, urinary tract infection, sexually transmitted diseases, and skin infection.

In some embodiments, the infection is a respiratory infection. Examples of respiratory infections include, but are not limited to, sore throat, bronchitis, sinusitis, tuberculosis, and pneumonia.

In some embodiments, the infection is a sexually transmitted disease. Examples of sexually transmitted diseases include, but are not limited to, chlamydia, gonorrhea, syphilis, and bacterial vaginosis.

In some embodiments, the infection is a skin infection. Examples of skin infections include, but are not limited to, cellulitis, folliculitis, impetigo, and boils.

Cancer

Disclosed herein are methods of treating cancer in a subject in need thereof. In some embodiments, the method comprises administering to the subject an olfactory receptor (OLFR). Alternatively, or additionally, the method comprises administering to the subject an agent that increases the expression of or activates an olfactory receptor (OLFR). Alternatively, or additionally, the method comprises administering to the subject an agent that increases or promotes the trafficking of an olfactory receptor (OLFR) to a plasma membrane of a cell. Further disclosed herein is the use of an agent that increases the expression of or activates an olfactory receptor (OLFR) in the manufacture of a medicament for the treatment of cancer. Disclosed herein is the use of an agent that increases or promotes the trafficking of an olfactory receptor (OLFR) to a plasma membrane of a cell in the manufacture of a medicament for the treatment of cancer. Disclosed herein is the use of an agent that increases the expression of or activates an olfactory receptor (OLFR) for the treatment of cancer. Disclosed herein is the use of an agent that increases or promotes the trafficking of an olfactory receptor (OLFR) to a plasma membrane of a cell for the treatment of cancer. The OLFR may be an OLFR listed in Table 1. The OLFR may be an OLFR listed in Table 2. The OLFR may be an OLFR listed in Table 3. The OLFR may be an OLFR listed in Table 4. The OLFR may be an OLFR listed in Table 5. The OLFR may be an OLFR listed in Table 6. The OLFR may be an OLFR listed in Table 7. The OLFR may be an OLFR listed in Table 8. The OLFR may be an OLFR listed in Table 9. The OLFR may be an OLFR listed in FIG. 2A. The OLFR may be an OLFR listed in FIG. 2B. The OLFR may be selected from the group consisting of OR7C1, OR7D4, OR10A6, OR11H6, OR4E2, OR10H1, and OR6A2. The OLFR may be OR7C1. The OLFR may be OR7D4. The OLFR may be OR10A6. The OLFR may be OR11H6. The OLFR may be OR4E2. The OLFR may be OR10H1. The OLFR may be OR6A2. The agent may be a ligand that binds to the OLFR. The agent may be an OLFR agonist. The agonist may be selected from the group consisting of octanal, coumarin, helional, lilial, b-ionone, androstenone, androstadienone, caramel furanone, 3-phenyl propyl propionate, eugenol, ethil vanillin, 2-ethyl-fencol, isovaleric acid, nonanoic acid, butyl butyryllactate, butyric acid, isovaleric acid, propionic acid, N-amyl acetate, eugenol acetate, sandalwood, S-(−)-citronellol, S-(−)-citronellal, (+)-carvine, (−) carvone, (+) carvone, linalool, bourgeonal, acetophenone, amyl butyrate, nonanethiol, allyl phenyl acetate, N-amyl acetate, muscone, isoeugenol, eugenol methyl ether, 1-hexanol, 1-heptanole, 1-octanol, celery ketone, anis aldehyde, (+)-menthol, vanillin, guaiacol, lyral, ethyl heptanoate, methyl octanoate, nonanal, 1-nonanol, 2-nonanol, 3-octanone, 3-nonanone, and decyl aldehyde. The agonist may be selected from the list of agonists provided in Table 8. The agent may be octanal, heptanal, or a prodrug or solvate thereof. The agent may activate the OLFR by increasing the trafficking of the OLFR to a plasma membrane of a cell. The agent may be a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal). The agent may comprise a nucleotide encoding a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal). Stimulating an immune response may comprise increasing expression or secretion of an inflammatory cytokine. The inflammatory cytokine may be selected from the group consisting of tumor necrosis factor (TNF), C-C motif chemokine ligand 2 (CCL2), CCL4, CCL5 interleukin 6 (IL-6), IL-1B, IL-18, and nitric oxide synthase 2 (NOS2). The administration may be local. The administration may be systemic. The administration may comprise an intravenous method. The subject may be a mammal. The subject may have or may be predisposed to a disease or disorder involving an immune dysregulation. The subject may be a human patient.

In some embodiments, the neoplasia, neoplastic disorder, tumor, cancer or malignancy treated is selected from the group consisting of a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, lymphosarcoma, liposarcoma, osteosarcoma, chondrosarcoma, leiomyosarcoma, rhabdomyosarcoma, fibrosarcoma or melanoma; or a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, neoplastic disorder, tumor, cancer or malignancy.

Agents

An “agent” as used herein refers to any molecule (e.g. antibody, Fab fragment, protein, peptide, nucleic acid, synthetic chemical, small chemical molecule, ligand mimetic) that can be administered to a subject. An agent that modulates expression or activity of an olfactory receptor provided in any of Table 1, Table 2, Table 3, Table 4, Table 5, Table 6, Table 7, Table 8, Table 9, FIG. 2A, and FIG. 2B is a molecule or composition that when administered to a subject modulates expression or activity of an olfactory receptor provided in any of Table 1, Table 2, Table 3, Table 4, Table 5, Table 6, Table 7, Table 8, Table 9, FIG. 2A, and FIG. 2B. In certain embodiments, the agent modulates, inhibits, blocks, decreases, increases, enhances, promotes or elicits expression or activity of an olfactory receptor provided in any of Table 1, Table 2, Table 3, Table 4, Table 5, Table 6, Table 7, Table 8, Table 9, FIG. 2A, and FIG. 2B. In some embodiments, the agent modulates, enhances, stimulates, induces or increases expression or activity of an olfactory receptor provided in any of Table 1, Table 2, Table 3, Table 4, Table 5, Table 6, Table 7, Table 8, Table 9, FIG. 2A, and FIG. 2B. In certain embodiments, the agent modulates, inhibits, blocks, decreases, increases, enhances, promotes or elicits an immune response. In some embodiments, the agent modulates, enhances, stimulates, induces or increases an immune response. In certain embodiments, modulation of an immune response includes modulating the innate immune response (e.g. endothelial cells, macrophages, monocytes, dendritic cells, cytokines).

The methods, uses, and kits disclosed herein may comprise one or more agents that modulate the activity of an olfactory receptor (OLFR). The agent may be selected from the group of: a ligand or small molecule that binds to the OLFR or blocks the binding of the OLFR to the ligand or an agent that inhibits the expression of the OLFR by the cell. The agent may be selected from the group of an antibody, fragment or mimetic that binds to OLFR or an OLFR ligand, an anti-OLFR gene silencing agent, or octanal or a prodrug or solvate thereof.

The methods, uses, and kits disclosed herein may comprise one or more agents that increase the expression of or activity of an OLFR. The agent may be a ligand that binds to an OLFR listed in any one of Tables 1-9 and FIGS. 2A-2B. The agent may be a ligand that binds to an OLFR listed in Table 7. The agent may be a ligand that binds to an OLFR selected from the group consisting of OR7C1, OR7D4, OR10A6, OR11H6, OR4E2, OR10H1, and OR6A2. The agent may be octanal, heptanal, or a prodrug or solvate thereof. In some embodiments, the expression of the OLFR is increased by at least about 5%, 10%, 15%, 20%, 25%, 30%, 35%, 40%, 45%, 50%, 55%, 60%, 65%, 70%, 75%, 80%, 85%, 90%, 95%, 97%, or 100% or more. In some embodiments, the expression of the OLFR is increased by at least about 1, 1.5, 2, 2.5, 3, 3.5, 4, 4.5, 4, 5.5, 6, 6.5, 7, 7.5, 8, 8.5, 9, 9.5, or 10-fold or more. In some embodiments, the activity of the OLFR is increased by at least about 5%, 10%, 15%, 20%, 25%, 30%, 35%, 40%, 45%, 50%, 55%, 60%, 65%, 70%, 75%, 80%, 85%, 90%, 95%, 97%, or 100% or more. In some embodiments, the activity of the OLFR is increased by at least about 1, 1.5, 2, 2.5, 3, 3.5, 4, 4.5, 4, 5.5, 6, 6.5, 7, 7.5, 8, 8.5, 9, 9.5, or 10-fold or more. In some embodiments, the activity of the OLFR is determined by measuring the level of expression of one or more inflammatory cytokines. In some embodiments, the activity of the OLFR is determined by measuring the level of secretion of one or more inflammatory cytokines. In some embodiments, the increase in the expression of OLFR is based on a comparison to the subject who has not been administered the agent. Alternatively, or additionally, the expression of the OLFR is measured over a time period and the increase in the level of expression of the OLFR is based on a comparison of samples from the subject over two or more time points. In some embodiments, the increase in the activity of OLFR is based on a comparison to the subject who has not been administered the agent. Alternatively, or additionally, the activity of the OLFR is measured over a time period and the increase in the activity of the OLFR is based on a comparison of samples from the subject over two or more time points.

The methods, uses, and kits disclosed herein may comprise one or more agents that increase or promote the trafficking of an olfactory receptor (OLFR) to a plasma membrane of a cell. The agent may be a protein selected from the group of receptor transporting protein 1 (RTP1), RTP2, receptor expression enhancing protein 1 (REEP1), aminoacylase 3 (Acy3), or guanine nucleotide-binding protein G(olf) subunit alpha (Gnal). The agent may comprise a nucleotide encoding a protein selected from the group of receptor transporting protein 1 (RTP1), RTP2, receptor expression enhancing protein 1 (REEP1), aminoacylase 3 (Acy3), or guanine nucleotide-binding protein G(olf) subunit alpha (Gnal). In some embodiments, the level of trafficking of the OLFR to the plasma membrane is increased by at least about 5%, 10%, 15%, 20%, 25%, 30%, 35%, 40%, 45%, 50%, 55%, 60%, 65%, 70%, 75%, 80%, 85%, 90%, 95%, 97%, or 100% or more. In some embodiments, the level of trafficking of the OLFR to the plasma membrane is increased by at least about 1, 1.5, 2, 2.5, 3, 3.5, 4, 4.5, 4, 5.5, 6, 6.5, 7, 7.5, 8, 8.5, 9, 9.5, or 10-fold or more. In some embodiments, the level of trafficking of the OLFR to the plasma membrane is determined by detecting the amount of OLFR on the surface of the cell. In some embodiments, the increase in the level of trafficking of the OLFR to the plasma membrane is based on a comparison to the subject who has not been administered the agent. Alternatively, or additionally, the level of trafficking of the OLFR to the plasma membrane is measured over a time period and the increase in the level of trafficking is based on a comparison of samples from the subject over two or more time points.

The methods, uses, and kits disclosed herein may comprise one or more agents that increase expression or secretion of an inflammatory cytokine. The inflammatory cytokine may be selected from the group consisting of tumor necrosis factor (TNF), C-C motif chemokine ligand 2 (CCL2), CCL4, CCL5, interleukin 6 (IL-6), IL-1B, IL-18, and nitric oxide synthase 2 (NOS2).

The methods, uses, and kits disclosed herein may comprise one or more agents that decrease or inhibit the expression of or activity of an OLFR. The agent may be a gene silencing agent. The gene silencing agent may be selected from the group consisting of a RNA interference (RNAi) molecule, zinc finger nuclease, transcription activator-like effector nuclease (TALEN), and Clustered Regulatory Interspaced Short Palindromic Repeats (CRISPR) enzyme. The RNAi molecule may be selected from the group consisting of a small interference RNA (siRNA), short hairpin RNA (shRNA) and microRNA (miRNA). The gene silencing agent may target an OLFR listed in any one of Tables 1-9 and FIGS. 2A-2B. The gene silencing agent may target an OLFR listed in Table 7. The gene silencing agent may target an OLFR selected from the group consisting of OR7C1, OR7D4, OR10A6, OR11H6, OR4E2, OR10H1, and OR6A2. In some embodiments, the expression of the OLFR is decreased by at least about 5%, 10%, 15%, 20%, 25%, 30%, 35%, 40%, 45%, 50%, 55%, 60%, 65%, 70%, 75%, 80%, 85%, 90%, 95%, 97%, or 100% or more. In some embodiments, the expression of the OLFR is decreased by at least about 1, 1.5, 2, 2.5, 3, 3.5, 4, 4.5, 4, 5.5, 6, 6.5, 7, 7.5, 8, 8.5, 9, 9.5, or 10-fold or more. In some embodiments, the activity of the OLFR is decreased by at least about 5%, 10%, 15%, 20%, 25%, 30%, 35%, 40%, 45%, 50%, 55%, 60%, 65%, 70%, 75%, 80%, 85%, 90%, 95%, 97%, or 100% or more. In some embodiments, the activity of the OLFR is decreased by at least about 1, 1.5, 2, 2.5, 3, 3.5, 4, 4.5, 4, 5.5, 6, 6.5, 7, 7.5, 8, 8.5, 9, 9.5, or 10-fold or more. In some embodiments, the activity of the OLFR is determined by measuring the level of expression of one or more inflammatory cytokines. In some embodiments, the activity of the OLFR is determined by measuring the level of secretion of one or more inflammatory cytokines. In some embodiments, the decrease in the expression of OLFR is based on a comparison to the subject who has not been administered the agent. Alternatively, or additionally, the expression of the OLFR is measured over a time period and the decrease in the level of expression of the OLFR is based on a comparison of samples from the subject over two or more time points. In some embodiments, the decrease in the activity of OLFR is based on a comparison to the subject who has not been administered the agent. Alternatively, or additionally, the activity of the OLFR is measured over a time period and the decrease in the activity of the OLFR is based on a comparison of samples from the subject over two or more time points.

One of skill in the art can use methods such as RNA interference (RNAi), CRISPR, TALEN, ZFN or other methods that target specific sequences to reduce or eliminate expression and/or function of proteins, such as an OLFR, OLFR ligand, or a protein involved in trafficking of OLFR to the plasma membrane of a cell. CRISPR, TALEN, ZFN or other genome editing tools can also be used to modulate expression and/or function of genes.

As used herein, “RNAi” (RNA interference) refers to the method of reducing or eliminating gene expression in a cell by targeting specific mRNA sequences for degradation via introduction of short pieces of double stranded RNA (dsRNA) and small interfering RNA (such as siRNA, shRNA or miRNA etc.) (Agrawal, N. et al.; Microbiol Mol Biol Rev. 2003; 67:657-685, Arenz, C. et al.; Naturwissenschaften. 2003; 90:345-359, Hannon G J.; Nature. 2002; 418:244-251).

As used herein, the term “CRISPR” refers to a technique of sequence specific genetic manipulation relying on the clustered regularly interspaced short palindromic repeats pathway. CRISPR can be used to perform gene editing and/or gene regulation, as well as to simply target proteins to a specific genomic location. “Gene editing” refers to a type of genetic engineering in which the nucleotide sequence of a target polynucleotide is changed through introduction of deletions, insertions, single stranded or double stranded breaks, or base substitutions to the polynucleotide sequence. In some aspects, CRISPR-mediated gene editing utilizes the pathways of non-homologous end-joining (NHEJ) or homologous recombination to perform the edits. Gene regulation refers to increasing or decreasing the production of specific gene products such as protein or RNA.

The term “gRNA” or “guide RNA” as used herein refers to guide RNA sequences used to target specific polynucleotide sequences for gene editing employing the CRISPR technique. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, M. et al. (2016) J of Biotechnology 233 (2016) 74-83).

The term “Cas9” refers to a CRISPR associated endonuclease referred to by this name Non-limiting exemplary Cas9s include Staphylococcus aureus Cas9, nuclease dead Cas9, and orthologs and biological equivalents each thereof. Orthologs include but are not limited to Streptococcus pyogenes Cas9 (“spCas9”), Cas9 from Streptococcus thermophiles, Legionella pneumophilia, Neisseria lactamica, Neisseria meningitides, Francisella novicida; and Cpf1 (which performs cutting functions analogous to Cas9) from various bacterial species including Acidaminococcus spp. and Francisella novicida U112.

As used herein, “TALEN” (transcription activator-like effector nucleases) refers to engineered nucleases that comprise a non-specific DNA-cleaving nuclease fused to a TALE DNA-binding domain, which can target DNA sequences and be used for genome editing. Boch (2011) Nature Biotech. 29: 135-6; and Boch et al. (2009) Science 326: 1509-12; Moscou et al. (2009) Science 326: 3501. TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence. To produce a TALEN, a TALE protein is fused to a nuclease (N), which is a wild-type or mutated Fold endonuclease. Several mutations to Fold have been made for its use in TALENs; these, for example, improve cleavage specificity or activity. Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Bio. 200: 96. The Fold domain functions as a dimer, requiring two constructs with unique DNA binding domains for sites in the target genome with proper orientation and spacing. Both the number of amino acid residues between the TALE DNA binding domain and the Fold cleavage domain and the number of bases between the two individual TALEN binding sites appear to be important parameters for achieving high levels of activity. Miller et al. (2011) Nature Biotech. 29: 143-8. TALENs specific to sequences in immune cells can be constructed using any method known in the art, including various schemes using modular components. Zhang et al. (2011) Nature Biotech. 29: 149-53; Geibler et al. (2011) PLoS ONE 6: e19509.

As used herein, “ZFN” (Zinc Finger Nuclease) refers to engineered nucleases that comprise a non-specific DNA-cleaving nuclease fused to a zinc finger DNA binding domain, which can target DNA sequences and be used for genome editing. Like a TALEN, a ZFN comprises a Fold nuclease domain (or derivative thereof) fused to a DNA-binding domain. In the case of a ZFN, the DNA-binding domain comprises one or more zinc fingers. Carroll et al. (2011) Genetics Society of America 188: 773-782; and Kim et al. (1996) Proc. Natl. Acad. Sci. USA 93: 1156-1160. A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Like a TALEN, a ZFN must dimerize to cleave DNA. Thus, a pair of ZFNs are required to target non-palindromic DNA sites. The two individual ZFNs must bind opposite strands of the DNA with their nucleases properly spaced apart. Bitinaite et al. (1998) Proc. Natl. Acad. Sci. USA 95: 10570-5. ZFNs specific to sequences in immune cells can be constructed using any method known in the art. See, e.g., Provasi (2011) Nature Med. 18: 807-815; Torikai (2013) Blood 122: 1341-1349; Cathomen et al. (2008) Mol. Ther. 16: 1200-7; Guo et al. (2010) J. Mol. Bioi. 400: 96; U.S. Patent Publication 201110158957; and U.S. Patent Publication 2012/0060230.

The agent may decrease the activity of the OLFR by inhibiting the binding of the OLFR with its ligand. The agent may be an OLFR antagonist. The OLFR antagonist may be citral, a solvate or prodrug thereof. The OLFR antagonist may be methylcinnamaldehyde, hydrocinnamaldehyde, bourgeonal, undecanal, methyl isoeugenol a solvate or prodrug thereof. The antagonist may be selected from the list of antagonists provided in Table 9 or a solvate or prodrug thereof.

The agent may decrease the activity of the OLFR by inhibiting the trafficking of the OLFR to a plasma membrane of a cell. The methods, uses, and kits disclosed herein may comprise one or more agents that decrease or inhibit the trafficking of an olfactory receptor (OLFR) to a plasma membrane of a cell. The agent may be a gene silencing agent that inhibits the expression of a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal).

The agent may be an antibody, fragment, derivative, or mimetic thereof. The antibody, fragment, derivative, or mimetic thereof may bind to an OLFR. The agent may be an antibody that binds to an OLFR listed in any one of Tables 1-9 and FIGS. 2A-2B. The agent may be an antibody that binds to an OLFR listed in Table 7. The agent may be an antibody that binds to an OLFR selected from the group consisting of OR7C1, OR7D4, OR10A6, OR11H6, OR4E2, OR10H1, and OR6A2. The antibody, fragment, derivative, or mimetic thereof may bind to a ligand of an OLFR. The antibody, fragment, derivative, or mimetic thereof may bind to a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal).

The antibody may be polyclonal, monoclonal, multispecific (e.g., bispecific antibodies), and antibody fragments, so long as they exhibit the desired biological activity. Antibodies can be isolated from any suitable biological source, e.g., murine, rat, sheep and canine. The antibody may be a human antibody, humanized antibody, or chimeric antibody. The antibody may be a human monoclonal antibody. The antibody may be a recombinant human antibody.

As used herein, the terms “antibody,” “antibodies” and “immunoglobulin” includes whole antibodies and any antigen binding fragment or a single chain thereof. Thus the term “antibody” includes any protein or peptide containing molecule that comprises at least a portion of an immunoglobulin molecule. The terms “antibody,” “antibodies” and “immunoglobulin” also include immunoglobulins of any isotype, fragments of antibodies which retain specific binding to antigen, including, but not limited to, Fab, Fab′, F(ab)₂, Fv, scFv, dsFv, Fd fragments, dAb, VH, VL, VhH, and V-NAR domains; minibodies, diabodies, triabodies, tetrabodies and kappa bodies; multispecific antibody fragments formed from antibody fragments and one or more isolated. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, at least one portion of a binding protein, chimeric antibodies, humanized antibodies, single-chain antibodies, and fusion proteins comprising an antigen-binding portion of an antibody and a non-antibody protein. The variable regions of the heavy and light chains of the immunoglobulin molecule contain a binding domain that interacts with an antigen. The constant regions of the antibodies (Abs) may mediate the binding of the immunoglobulin to host tissues. The term “anti-” when used before a protein name, anti-DNABII, anti-IHF, anti-HU, anti-OMP P5, for example, refers to a monoclonal or polyclonal antibody that binds and/or has an affinity to a particular protein. For example, “anti-IHF” refers to an antibody that binds to the IHF protein. The specific antibody may have affinity or bind to proteins other than the protein it was raised against. For example, anti-IHF, while specifically raised against the IHF protein, may also bind other proteins that are related either through sequence homology or through structure homology.

As used herein, “monoclonal antibody” refers to an antibody obtained from a substantially homogeneous antibody population. Monoclonal antibodies are highly specific, as each monoclonal antibody is directed against a single determinant on the antigen. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

Monoclonal antibodies may be generated using hybridoma techniques or recombinant DNA methods known in the art. A hybridoma is a cell that is produced in the laboratory from the fusion of an antibody-producing lymphocyte and a non-antibody producing cancer cell, usually a myeloma or lymphoma. A hybridoma proliferates and produces a continuous sample of a specific monoclonal antibody. Alternative techniques for generating or selecting antibodies include in vitro exposure of lymphocytes to antigens of interest, and screening of antibody display libraries in cells, phage, or similar systems.

The term “human antibody” as used herein, is intended to include antibodies having variable and constant regions derived from human germline immunoglobulin sequences. The human antibodies disclosed herein may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, (VL, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

A “human monoclonal antibody” refers to antibodies displaying a single binding specificity which have variable and constant regions derived from human germline immunoglobulin sequences. The term also intends recombinant human antibodies. Methods to making these antibodies are described herein.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, e.g., from a transfectoma, antibodies isolated from a recombinant, combinatorial human antibody library, and antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. Methods to making these antibodies are described herein.

As used herein, chimeric antibodies are antibodies whose light and heavy chain genes have been constructed, typically by genetic engineering, from antibody variable and constant region genes belonging to different species.

As used herein, the term “humanized antibody” or “humanized immunoglobulin” refers to a human/non-human chimeric antibody that contains a minimal sequence derived from non-human immunoglobulin. For the most part, humanized antibodies are human immunoglobulins (recipient antibody) in which residues from a variable region of the recipient are replaced by residues from a variable region of a non-human species (donor antibody) such as mouse, rat, rabbit, or non-human primate having the desired specificity, affinity and capacity. Humanized antibodies may comprise residues that are not found in the recipient antibody or in the donor antibody. The humanized antibody can optionally also comprise at least a portion of an immunoglobulin constant region (Fc), typically that of a human immunoglobulin, a non-human antibody containing one or more amino acids in a framework region, a constant region or a CDR, that have been substituted with a correspondingly positioned amino acid from a human antibody. In general, humanized antibodies are expected to produce a reduced immune response in a human host, as compared to a non-humanized version of the same antibody. The humanized antibodies may have conservative amino acid substitutions which have substantially no effect on antigen binding or other antibody functions. Conservative substitutions groupings include: glycine-alanine, valine-leucine-isoleucine, phenylalanine-tyrosine, lysine-arginine, alanine-valine, serine-threonine and asparagine-glutamine.

The terms “polyclonal antibody” or “polyclonal antibody composition” as used herein refer to a preparation of antibodies that are derived from different B-cell lines. They are a mixture of immunoglobulin molecules secreted against a specific antigen, each recognizing a different epitope.

As used herein, the term “antibody derivative”, comprises a full-length antibody or a fragment of an antibody, wherein one or more of the amino acids are chemically modified by alkylation, pegylation, acylation, ester formation or amide formation or the like, e.g., for linking the antibody to a second molecule. This includes, but is not limited to, pegylated antibodies, cysteine-pegylated antibodies, and variants thereof.

The methods, uses, and kits disclosed herein may comprise one or more agents that decrease or inhibit expression or secretion of an inflammatory cytokine. The inflammatory cytokine may be selected from the group consisting of tumor necrosis factor (TNF), C-C motif chemokine ligand 2 (CCL2), CCL4, CCL5, interleukin 6 (IL-6), IL-1B, IL-18, and nitric oxide synthase 2 (NOS2).

Kits

Further disclosed herein are kits comprising any of the agents disclosed herein. In some embodiments, the kit comprises an agent that modulates the activity of an OLFR and instructions for use. Alternatively, or additionally, the kit comprises an agent that increases the activity of an OLFR and instructions for use. The kit may comprise an agent that increases the expression of an OLFR and instructions for use. The kit may comprise an agent that increases or promotes the trafficking of an olfactory receptor (OLFR) to a plasma membrane of a cell and instructions for use. The kit may comprise an agent that decreases the activity of an OLFR and instructions for use. The kit may comprise an agent that decreases the expression of an OLFR and instructions for use. The kit may comprise an agent that decreases or inhibits the trafficking of an olfactory receptor (OLFR) to a plasma membrane of a cell and instructions for use. The kit may comprise a ligand of an olfactory receptor (OLFR) and instructions for use. The kit may comprise an olfactory receptor (OLFR) agonist and instructions for use. The kit may comprise an olfactory receptor (OLFR) antagonist and instructions for use. The kit may comprise octanal and instructions for use. The kit may comprise citral and instructions for use. The kit may comprise a gene silencing agent that targets an olfactory receptor (OLFR) and instructions for use. The kit may comprise a gene silencing agent that targets a ligand of an olfactory receptor (OLFR) and instructions for use. The kit may comprise a gene silencing agent that targets one or more proteins selected from the group consisting of a receptor transporting protein 1 (RTP1), RTP2, receptor expression enhancing protein 1 (REEP1), aminoacylase 3 (Acy3), and guanine nucleotide-binding protein G(olf) subunit alpha (Gnal) and instructions for use.

The kits disclosed herein may further comprise one or more pharmaceutically acceptable carriers. “Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

Administration

The methods, uses, and kits disclosed herein may comprise administering an agent or instructions to administer an agent to a subject in need thereof.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, and topical application.

An agent of the present disclosure can be administered for therapy by any suitable route of administration. It will also be appreciated that the optimal route will vary with the condition and age of the recipient, and the disease being treated. The term “effective amount” refers to a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of an immunogenic composition, in some embodiments the effective amount is the amount sufficient to result in a protective response against a pathogen. In other embodiments, the effective amount of an immunogenic composition is the amount sufficient to result in antibody generation against the antigen. In some embodiments, the effective amount is the amount required to confer passive immunity on a subject in need thereof. With respect to immunogenic compositions, in some embodiments the effective amount will depend on the intended use, the degree of immunogenicity of a particular antigenic compound, and the health/responsiveness of the subject's immune system, in addition to the factors described above. The skilled artisan will be able to determine appropriate amounts depending on these and other factors.

As used herein, the terms “treat” or “treatment” may refer to any prevention or delay in onset, reduction in the frequency or severity of symptoms, amelioration of symptoms, improvement in patient comfort or function, decrease in severity of the condition or disease state, decrease in the occurrence of a given condition or disease or condition or disease symptoms in a patient etc. The effect of treatment can be compared to an individual or pool of individuals not receiving a given treatment, or to the same patient prior to, or after cessation of, treatment. As indicated above, the treatment may be complete (no detectable symptoms) or partial, such that fewer symptoms are observed than would likely occur absent treatment.

In some embodiments, a method of treatment results in partial or complete destruction of the neoplastic, tumor, cancer or malignant cell mass; a reduction in volume, size or numbers of cells of the neoplastic, tumor, cancer or malignant cell mass; stimulating, inducing or increasing neoplastic, tumor, cancer or malignant cell necrosis, lysis or apoptosis; reducing neoplasia, tumor, cancer or malignancy cell mass; inhibiting or preventing progression or an increase in neoplasia, tumor, cancer or malignancy volume, mass, size or cell numbers; or prolonging lifespan. In some embodiments, a method of treatment results in reducing or decreasing severity, duration or frequency of an adverse symptom or complication associated with or caused by the neoplasia, tumor, cancer or malignancy. In some embodiments, a method of treatment results in reducing or decreasing pain, discomfort, nausea, weakness or lethargy. In some embodiments, a method of treatment results in increased energy, appetite, improved mobility or psychological well-being.

In certain embodiments, a method of treatment comprises administering a therapeutically effective amount of an agent to a subject, wherein the agent modulates expression or activity of one or more olfactory receptor listed in any of Table 1, Table 2, Table 3, Table 4, Table 5, Table 6, Table 7, Table 8, Table 9, FIG. 2A, and FIG. 2B. By “therapeutically effective amount” as used herein is meant a dose that produces effects for which it is administered (e.g. treating or preventing a disease or disorder). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

The present inventions further provide novel agents identified by the assays described herein and use of such agent in the methods of treatment described herein.

Unless otherwise defined, all technical and scientific terms used herein have the same meaning as commonly understood by one of ordinary skill in the art to which this invention belongs.

All applications, publications, patents and other references, GenBank citations and ATCC citations cited herein are incorporated by reference in their entirety. In case of conflict, the specification, including definitions, will control.

All of the features disclosed herein may be combined in any combination. Each feature disclosed in the specification may be replaced by an alternative feature serving a same, equivalent, or similar purpose. Thus, unless expressly stated otherwise, disclosed features (e.g., compound structures) are an example of a genus of equivalent or similar features.

As used herein, the singular forms “a”, “and,” and “the” include plural referents unless the context clearly indicates otherwise. Thus, for example, reference to “a first, second, third, fourth, fifth, etc. predictor gene” or a “positive or negative predictor gene” includes a plurality of such first, second, third, fourth, fifth, etc., genes, or a plurality of positive and/or negative predictor genes.

As used herein, all numerical values or numerical ranges include integers within such ranges and fractions of the values or the integers within ranges unless the context clearly indicates otherwise. Thus, to illustrate, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

Reference to a number with more (greater) or less than includes any number greater or less than the reference number, respectively. Thus, for example, a reference to less than 30,000, includes 29,999, 29,998, 29,997, etc. all the way down to the number one (1); and less than 20,000, includes 19,999, 19,998, 19,997, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as a percentage range, 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. Reference to a range of 1-5 fold therefore includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

Reference to a series of ranges includes ranges which combine the values of the boundaries of different ranges within the series. Thus, to illustrate reference to a series of ranges of 2-72 hours, 2-48 hours, 4-24 hours, 4-18 hours and 6-12 hours, includes ranges of 2-6 hours, 2-18 hours, 2-24 hours, etc., and 4-27 hours, 4-48 hours, 4-6 hours, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed.

### EXAMPLES

### Example 1: Effect of Citral on Expression of Inflammatory Genes (CCL2, CCL4, TNF and iNOS)

In this example, the effects of citral on CCL2, CCL4, TNF and iNOS expression in vascular macrophages in Apoe−/− mice was investigated. The effect of citral on atherosclerosis in Apoe−/− mice fed a high fat western diet, a widely used model of atherosclerosis, was also investigated in this example

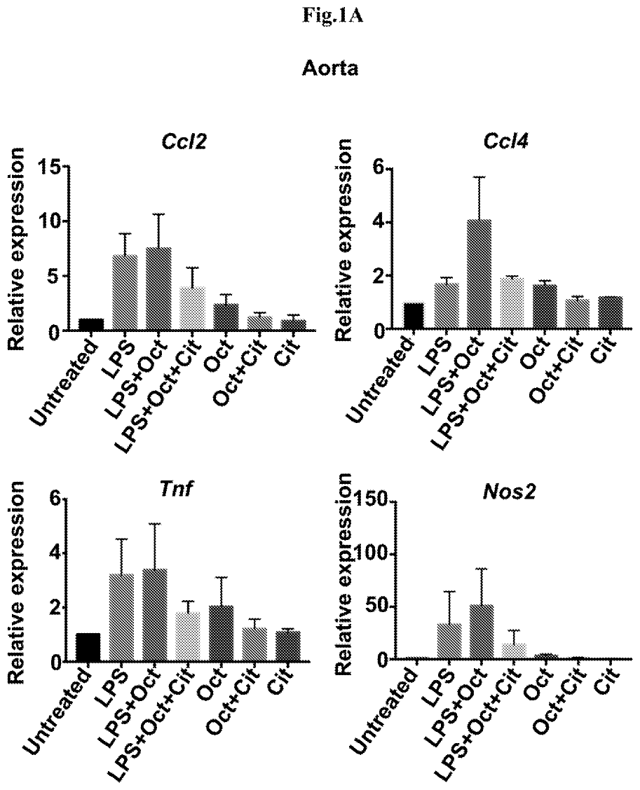

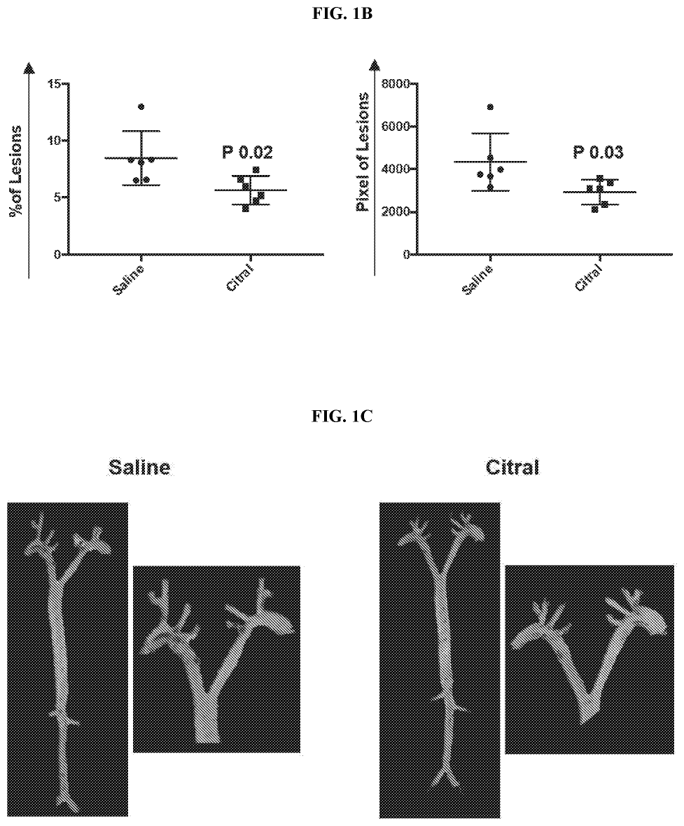

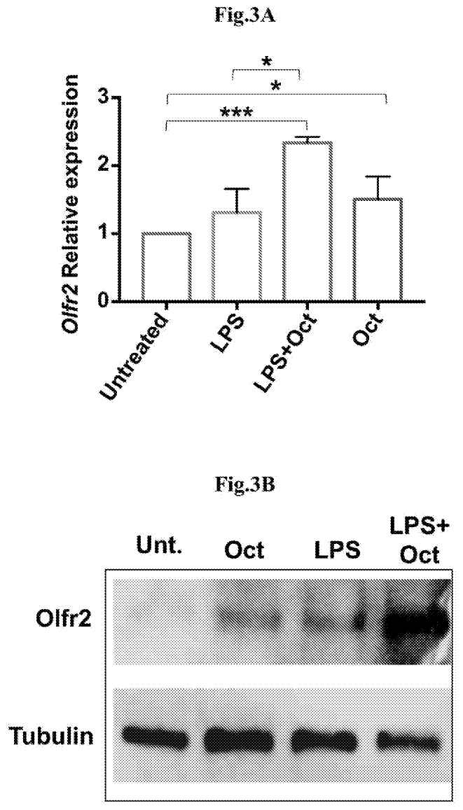

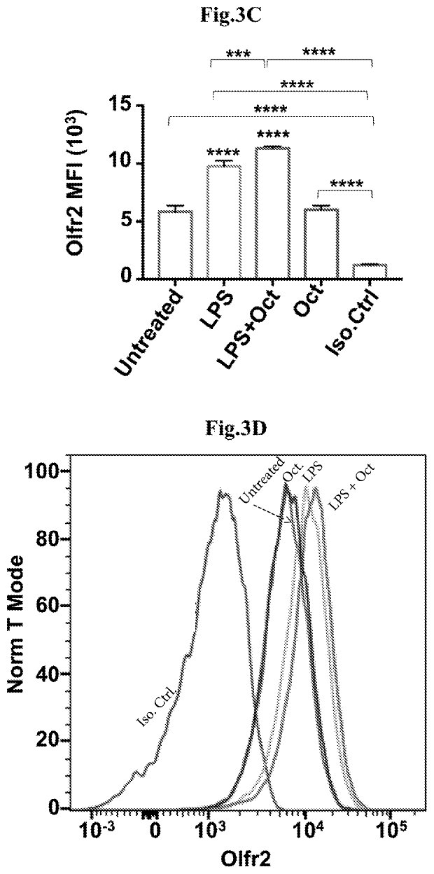

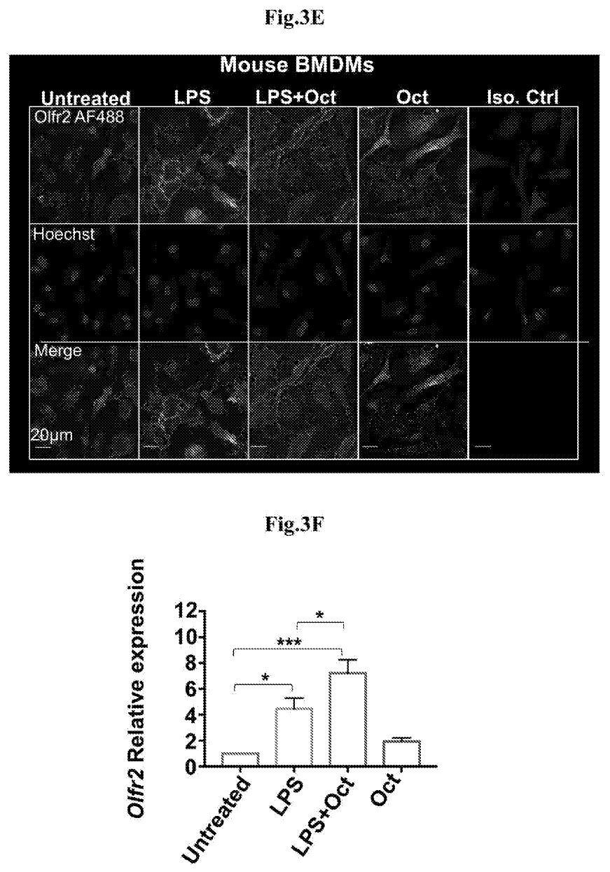

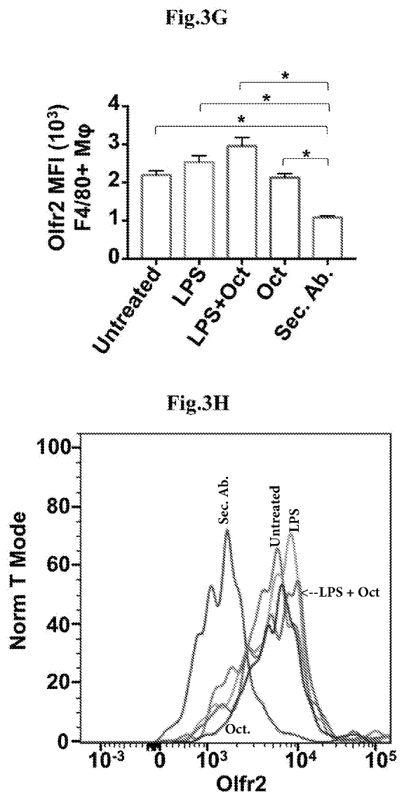

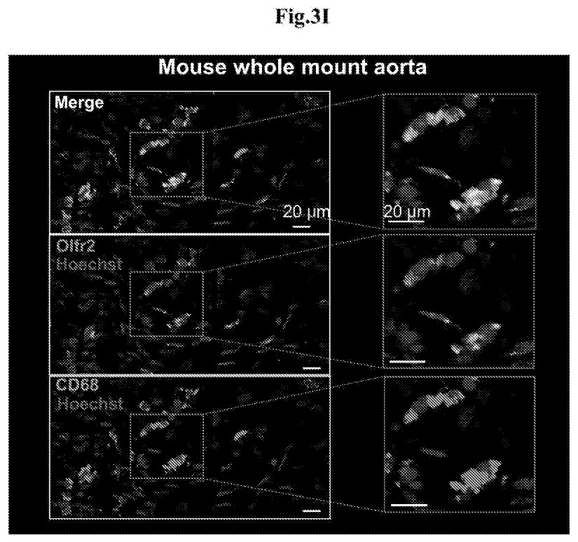

Gene expression analysis was performed by harvesting whole aortas from apoE^{-/-} mice kept on western diet (WD) for 2 weeks and treating for 12h with DMSO (Ctrl), citral (Cit, 100 μM), octanal (Oct, 10 μM in DMSO) or lipopolysaccharide (lps, 0.5 μg/mL in DMSO) alone or in combination. After the incubation, aortas were transferred in Trizol reagent (1 ml) and digested with gentleMACS™ Dissociator (Millteny). Total RNA was then purified with RNAse Micro kit (Qiagen) and reverse transcribed using the Omniscript reverse transcriptase kit (Qiagen). Real-time PCR reaction was performed following the RT² Sybr green gene expression assay protocol (Qiagen). RT2 SYBR Green qPCR mastermix mix and premade RT² qPCR Primer Assay for mouse GAPDH, CCL2, CCL4, TNF, and iNOS were used. Data were normalized to the expression levels of the housekeeping gene GAPDH. FIG. 1A: shows the results of a gene expression analysis of CCL2, CCL4, TNF and iNOS after treatment with the Olfr2 inhibitor citral alone or in combination with octanal and LPS. As shown in FIG. 3A, citral completely blocked the increase in CCL2, CCL4, TNF and iNOS, both in the presence and absence of LPS.

The effect of citral on atherosclerosis in Apoe−/− mice fed a high fat western diet, a widely used model of atherosclerosis, was investigated. Briefly, ApoE^{-/-} Mice (10 weeks old) were fed WD for 4 weeks before the inhibitor treatment, citral, was started. Citral at 10 mg/kg was diluted in 100 uL of PBS and injected I.P. every 3 days for 4 weeks for a total of 12 injections. During this time, mice continue to eat WD for a total of 8 weeks. After that, the mice were sacrificed and harvested for aorta explant and pinning FIG. 1B shows the results of an atherosclerosis lesion evaluation (% and number of pixels with lesions) after the treatment with OLFR2 inhibitor citral. FIG. 1C shows pictures of pinned aortas of mice treated with citral or saline (untreated). As shown in FIG. 1B, the citral-treated Apoe−/− mice showed significantly reduced atherosclerotic lesions, from ˜8% of the aortic area to ˜5% (P<0.02). Thus, blocking Olfr2 is anti-atherosclerotic, showing that Olfr2 contributes to atherosclerosis.

### Example 2: Transcriptomic Study of Vascular Macrophages

Atherosclerosis is an inflammatory disease of the arterial wall driven by macrophages and other immune cells. Olfactory receptors (Olfrs) are G-protein coupled receptors expressed in olfactory epithelial cells and are responsible for the sense of smell. To better understanding atherosclerosis, a transcriptomic study of vascular macrophages was performed.

In a transcriptomic study of vascular macrophages sorted from aortas of atherosclerosis-prone Apoe^{-/-} mice, hundreds of Olfrs, Gαolf (Gnal), Acy3, Reep3, Rtp1, Rtp2 and the cyclic nucleotide-gated ion channel subunits Cnga1-4 and Cngb1 were unexpectedly found to be expressed (Table 2). Some of these genes were detectable even in aortic macrophages from mice without atherosclerosis, and many were upregulated in response to LPS (FIG. 2A, Table 1).

FIG. 2A depicts a heat map of Olfr genes (total 1123) expressed in mouse vascular macrophages and several other tissue macrophages, DCs and blood monocytes [data were normalized through Gapdh expression ranking (=1), by row]. Several microarray datasets (Affymetrix mouse gene 1.0 ST array type) were used for the comparison: GSE68968 Vascular Mφ; GSE77104 BMDMs, GSE43075 peritoneal (PT) Mφ; GSE100393 Small Intestine (SI) Mφ; GSE15907 Lung Mφ; GSE15907 Spleen (red pulp) Mφ; GSE15907 Microglia; GSE100393 SI DCs; GSE15907 Lung DCs; GSE15907 mesenteric LN (MLN) DCs; GSE15907 blood classical (CM) and non-classical monocyte (NCM) subpopulations. The one minus Pearson correlation clustering of rows and columns is displayed. Olfr2, Rtp1, Rtp2, Reep3, Acy3, Gnal, Cnga1-4 and Cngb1 are highlighted.

In published transcriptomes of other tissue macrophages (small intestinal (SI) macrophages, untreated intraperitoneal (IP) macrophages or stimulated with LPS, lung CD11b-macrophages, splenic red pulp macrophages, microglia), dendritic cells (DCs) (small intestinal DCs, mesenteric lymph node (MLN) DCs, lung DCs) and monocytes (classical (CM), non-classical (NCM), both MHC-II+ and −) much fewer Olfrs were expressed. The list of normalized Olfrs expression and accessory molecules can be found in Table 1.

FIG. 2B displays a heat map of expressed Olfrs with known human orthologues. Of the 1123 known mouse Olfrs, 582 have known human orthologues (Olfrs sharing the same human orthologue are included), 280 of which (48%) were expressed in vascular macrophages from LPS treated mice (FIG. 2B).

This experiment demonstrates that vascular macrophages express hundreds of olfactory receptors and accessory molecules needed for Olfr signalling and trafficking.

### Example 3: Olfr2 and Atherosclerosis

As shown in Example 2, vascular macrophages express hundreds of olfactory receptors including Olfr2, a receptor for octanal. They also express Rtp1, Rtp2, Reep3, Acy3 and Gnal, accessory molecules needed for Olfr signaling and trafficking. Ligation of the human orthologue of Olfr2 (OR6A2), expressed in human atherosclerotic plaque and in human monocyte-derived macrophages, is strongly pro-inflammatory. In this example, the role of Olfr2 in atherosclerosis is investigated.

Olfr2 (human orthologue: OR6A2) is investigated in this example because (1) its expression is increased in LPS treated vascular macrophages, (2) is expressed in bone marrow-derived macrophages (BMDMs), (3) has known ligands (aliphatic aldehydes including octanal), (4) has a known inhibitor (citral), (5) has a human orthologue (OR2A6), which (6) is expressed in human carotid endarteriectomy samples and human monocyte-derived macrophages.

Olfr2 binds medium-chain aliphatic aldehydes such as octanal with an EC50 of ˜10 μM^{15,16}. Octanal is a 8-carbon aldehyde also known as fatty aldehyde that is produced by reduction of the carboxy group of caprylic acid (octanoic acid) or via lipid peroxidation during oxidative stress¹⁷.

Olfr2 expression in BMDMs was investigated. Briefly, bone marrow cells from Apoe^{-/-} mice were stimulated with MCSF (20 ng/ml) for 7 d to generate BMDMs, which were left untreated or incubated with the Olfr2 agonist octanal (Oct, 10 μM), LPS, (500 ng/mL), or with both for 12 h.

In mouse BMDMs, Olfr2 mRNA (FIG. 3A) and protein expression was found by western blot (FIG. 3B). Olfr2 mRNA and protein expression was enhanced by bacterial lipopolysaccharide (LPS), octanal and potentiated by both combined. Flow cytometry (FIGS. 3C-D) demonstrated surface expression of Olfr2. Cell surface and intracellular Olfr2 expression was confirmed by immunofluorescence (FIG. 3E).

Next, Olfr2 expression in aortas harvested from western-type diet (WD)-fed male mice (n=5) was investigated¹⁸. Briefly, whole aortas were harvested from Apoe^{-/-} mice fed western-like diet (WD) for 2 weeks and treated as described above. Relative expression of Olfr2 quantified by real time PCR analysis of Olfr2 expression normalized to expression of Gapdh and untreated control.

In addition, Rtp1 and Rtp2 expression in aortas harvested from Apoe−/− mice was investigated. Briefly, explanted aortas of Apoe−/− mice treated for 12h with Oct (10 μM) and/or lipopolysaccharide (LPS, 500 ng/mL). Rtp1 and Rtp2 expression was assessed by qPCR as described above. N=3 independent experiments (relative expression calculated by 2^{-ΔΔCt} method). Data are presented as means±SEM. *p<0.05, **p<0.01, ***p<0.005, ****p<0.001. p calculated by One-Way ANOVA test, Tukey's multiple comparisons test.

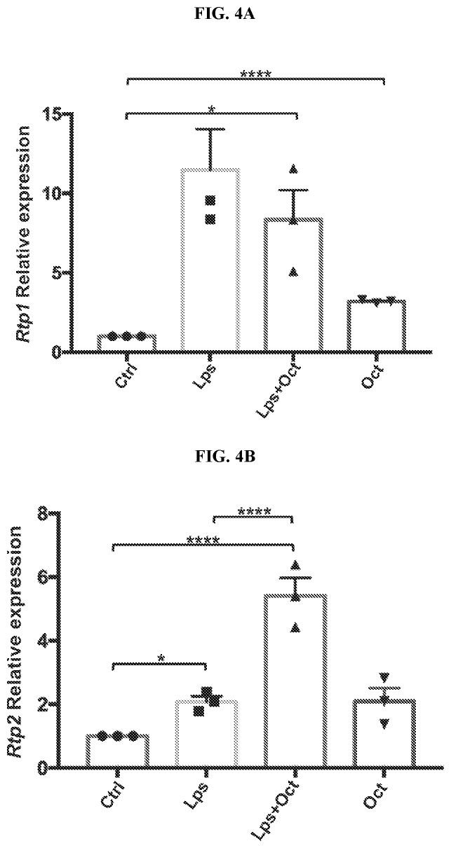

As shown in FIGS. 3F and 4A-B, aortic Olfr2 mRNA, Rtp1 and Rtp2 mRNA were found to be induced by LPS treatment ex vivo and significantly enhanced by adding octanal. Olfr2 expression was confirmed at the protein level on F4/80⁺ vascular macrophages purified form aortas as elsewhere described¹⁹ (FIG. 3G-H).

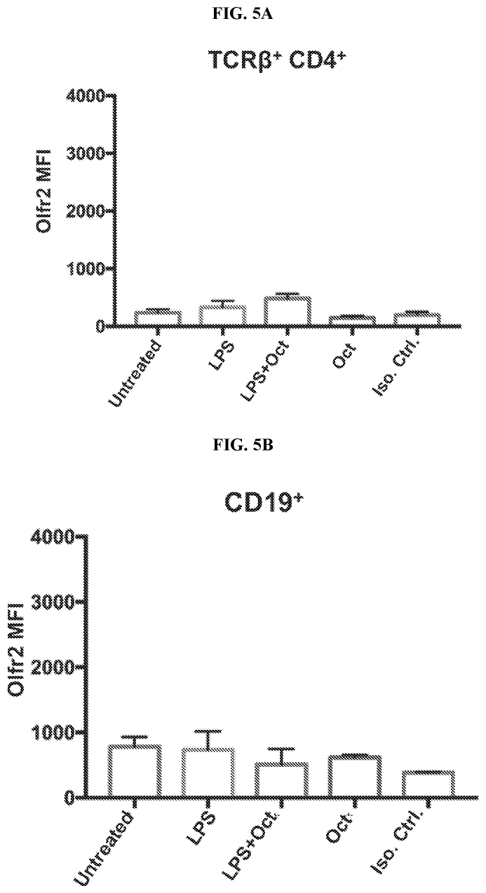

Olfr2 expression in T cells and B cells was also investigated. Briefly, whole aortas were harvested from Apoe−/− mice kept on western diet (WD) for at least 2 weeks incubated with Octanal (Oct, 10 μM) and lipopolysaccharide (LPS, 500 ng/mL) for 12h. Cells were harvested and stained for Olfr2 detection (as described in FIG. 3E) and analyzed by flow cytometry (LSRII, BD Biosciences). Olfr2-AF488 Median Fluorescence Intensity (MFI) of B cells (CD45+, Live, TCRβ−, CD19+) and CD4+ T cells (CD45+, Live, TCRβ+, CD4+) is reported. N=5 per group. Notably, other aortic immune cell populations such as CD4⁺ T cells and CD19⁺ B cells, did not show significant Olfr2 expression (FIGS. 5A-B). Immunofluorescence experiments in aortic whole mounts of WD-fed mice confirmed the Olfr2 expression in many, but not all CD68⁺ macrophages (FIG. 3I).

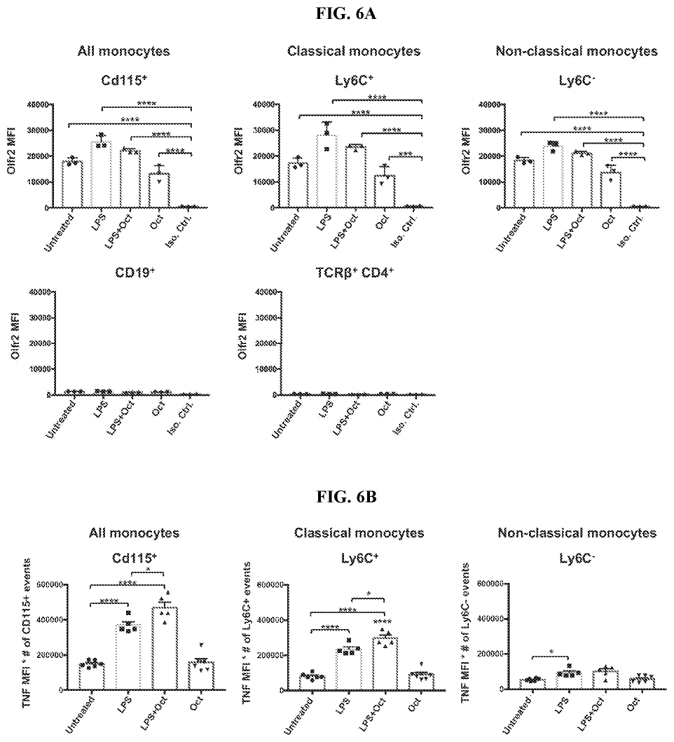

In addition, Olfr2 expression and function on blood mononuclear cells was investigated. Briefly, Apoe−/− mice blood harvested by cardiac puncture was red blood cells lysed, and incubated with Octanal (Oct, 10 μM) and lipopolysaccharide (LPS, 500 ng/mL) for 12h. Cells were harvested and stained for Olfr2 detection (as described in FIG. 3E) and analyzed by flow cytometry (LSRII, BD Biosciences). Olfr2-AF488 Median Fluorescence Intensity (MFI) of total monocytes (CD45+, Live, TCRβ−, CD19−, CD115+), classical monocytes (CD45+, Live, TCRβ−, CD19−, CD115+, Ly6C+), non classical monocytes (CD45+, Live, TCRβ−, CD19−, CD115+, Ly6C−), B cells (CD45+, Live, TCRβ−, CD19+) and CD4+ T cells (CD45+, Live, TCRβ+, CD4+) is reported. n 5-6 per group. Data are presented as means±SEM. *p<0.05, **p<0.01, ***p<0.005, ****p<0.001. p calculated by One-Way ANOVA test, Tukey's multiple comparisons test. Olfr2 protein was found to be expressed on circulating classical (CD115⁺Ly6C⁺) and non-classical (Cd115⁺Ly6C⁻) blood monocytes (FIG. 6A).

To assess the functional properties of Olfr2 in monocytes, cells were enriched form Apoe−/− mice blood by the negative selection. Purified monocytes were then incubated as described above. TNF expression (TNF-PE) was evaluate by intracellular staining protocol by flow cytometry. Classical and non-classical monocytes were divided as previously described (Live, CD115+, LyC6+, and LyC6− respectively). FIG. 6B shows Olfr2 function in monocytes reported as TNF MFI multiplied to the cell events. n 5 per group. Data are presented as means±SEM. *p<0.05, **p<0.01, ***p<0.005, ****p<0.001. p calculated by One-Way ANOVA test, Tukey's multiple comparisons test.

The role of Olfr2 ligation on inflammatory responses was investigated by measuring intracellular free calcium (Can in vascular macrophages. Briefly, mouse aortic cell suspensions were loaded with 2 μM Fluo-4 at 37° C. for 30 min, then washed and incubated with cell surface markers at RT for 20 min CD45⁺, live, dump channel⁻ (TCRb⁻, CD19⁻), F4/80⁺ macrophages were analyzed by flow cytometry. Olfr2 agonist Oct (10 μM), and antagonist Cit (100 μM) alone or combined (Oct+Cit) were added 60 seconds (s) after the acquisition started. DMSO (0.1%) was injected as a control (Ctrl).

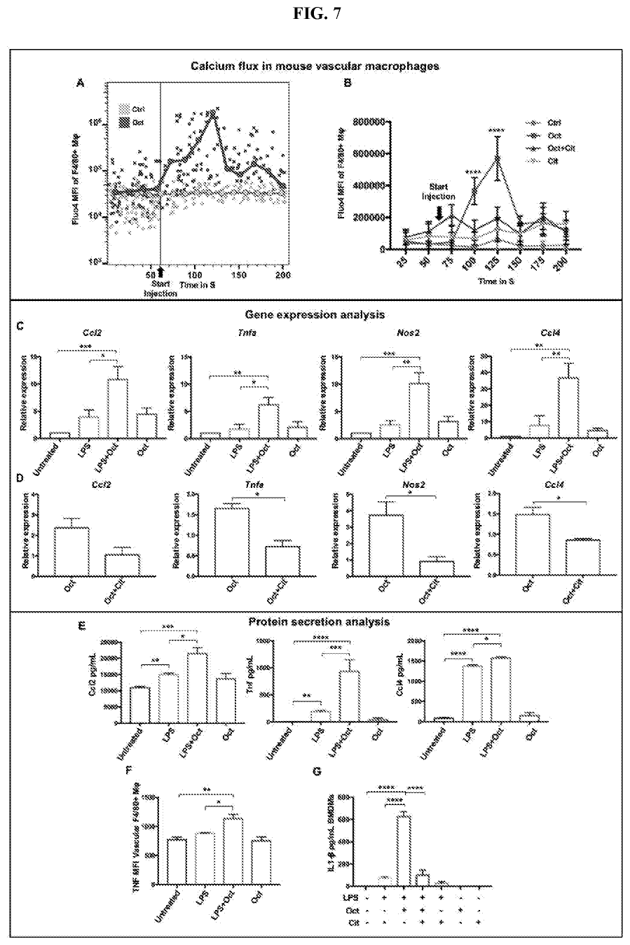

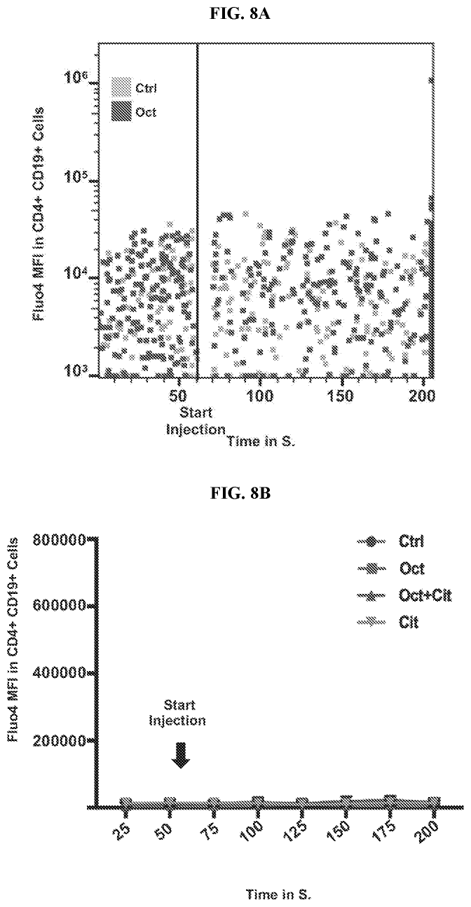

To test the functionality of Olfr2, time-resolved calcium flux in mouse vascular macrophages by flow cytometry was studied (FIG. 7A). Octanal induced a highly significant increase in intracellular free calcium at 100 and 125 s after injection, consistent with the known calcium dynamics of Olfrs¹⁵ (FIG. 7B). This calcium flux was completely blocked by the Olfr2 inhibitor citral^{9,16} (FIG. 7B). No calcium signal was detected in other immune cells (FIGS. 8A-B). FIG. 8A shows dump channel+ cells (TCRb+, and CD19+), loaded with 2 μM Fluo-4 before and after addition of Olfr2 agonist octanal (Oct 10 μM), and antagonist Citral (Cit 100 μM) alone or combined (Oct+Cit). Oct, and Cit injected at 60 seconds. Whole aortas were digested, stained with Fluo4 (2 μM) at 37° C. for 30 min, then washed and incubated with cell surface markers at RT for 20 min CD45+, live, dump channel+(TCRb+, and CD19+) cells were analyzed by flow cytometry. FIG. 8B shows the time average Median Fluorescence Intensity (MFI) of Fluo-4 is reported. SD is calculated among the cells for each time point (n 20-50). The analysis is made out one of three experiments.

An early step in atherosclerosis is the subendothelial retention of lipoproteins, which then triggers a non-resolving inflammatory process that over time drives plaque progression^{20,21}. Macrophages contribute to the inflammatory response and plaque formation by producing pro-inflammatory cytokines, such as interleukin-1β (IL-1β) and tumor necrosis factor (TNF), chemokines including CCL2 and CCL4, and inducible nitric oxide synthase (iNOS)²². Most vascular macrophages derive from CX3CR1⁺ embryonic precursors with a postnatal contribution from bone marrow-derived monocytes that colonize the tissue immediately after birth^{23,24}. During adulthood, vascular macrophages proliferate locally²⁵. In atherosclerotic mice, plaques contain several phenotypically distinct macrophage subsets²⁶⁻²⁸. Specifically, four major macrophage populations were found in atherosclerotic aortas: a resident-like, an inflammatory and a TREM2⁺ subset²⁶ and foam cells²⁸.

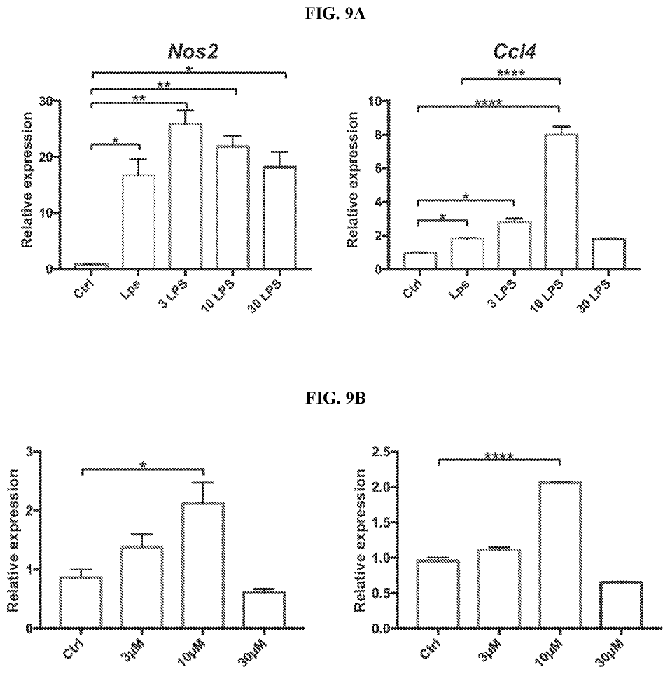

To test physiologic effects of Olfr2 activation in situ, explanted whole aortas of mice (n=5 per group) were treated with octanal with or without LPS. Briefly, whole aortas were left untreated or incubated with the Olfr2 agonist octanal (Oct, 10 μM), LPS, (500 ng/mL), or with both for 12 h. Quantitative Real time PCR reaction was performed to detect Ccl2, Ccl4, Tnf, and Nos2 gene expression. Relative expression was normalized to Gapdh expression and the untreated control. As shown in FIG. 7C, octanal treatment induced mRNA expression of the inflammatory chemokines Ccl2 (MCP-1) and Ccl4 (MIP-1β), the inflammatory cytokine Tnf and the inflammatory nitric oxide synthase iNOS (Nos2). In all cases, octanal enhanced the LPS-induced expression of these inflammatory molecules, peaking at 3-10 μM (FIG. 7C, FIGS. 9A-B). Conversely, the Olfr2 inhibitor citral was effective at curbing inflammatory mediator expression in explanted aortas incubated with octanal (FIG. 7D). Citral did not affect Olfr2 mRNA expression (data not shown). Octanal and LPS also enhanced the secretion of TNF, CCL2 and CCL4 protein into the supernatant of treated aortas (FIG. 7E). TNF secretion by vascular macrophages (F4/80+) was confirmed by intracellular staining using flow cytometry (FIG. 7F). Similar results were observed in classical Ly6C⁺ monocytes and not in non-classical Ly6C⁻ monocytes (FIG. 6B).

Macrophages are known producers of one of the most prominent cytokines driving atherosclerosis, IL-1β²⁹. IL-1β production and secretion requires assembly of the inflammasome, a cytoplasmic complex containing several proteins that serves as a molecular platform for activation of the cysteine protease caspase^{30,31}. To test whether octanal induced IL-1β secretion in macrophages, BMDMs were primed with low-dose LPS (50 ng/mL) for 18 h followed by exposure to octanal alone or together with citral. IL-1β protein in the supernatant was quantified by a cytokine bead array. Data are presented as mean±SEM. *p<0.05, **p<0.01, ***p<0.005, ****p<0.001. p calculated by One-Way ANOVA test, Tukey's multiple comparisons test and unpaired t-test. As shown in FIG. 7G, octanal (10 μM) strongly increased the release of IL-1β into the supernatant, which was inhibited by citral. These data suggest a possible enhancement of the inflammasome activation by octanal.

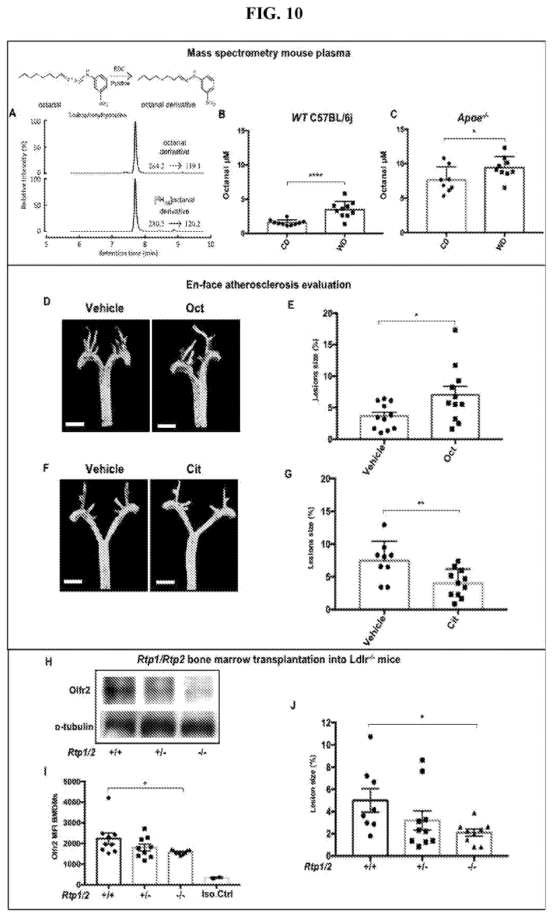

Next, mass spectrometry was used to measure octanal in blood plasma of wild-type and Apoe−/− mice on chow (CD) or western (WD) diet. Briefly, murine blood plasma was analyzed for octanal by derivatization and stable isotope dilution LC/MS/MS. [²H₁₆]octanal was spiked to plasma before derivatization reaction as internal standard. Stable isotope dilution HPLC with on-line tandem mass spectrometry (LC/MS/MS) (FIG. 10A) showed detectable levels of octanal in all mice. In plasma of C57BL/6J mice on CD, octanal ranged between 1.5 and 2.5 μM (FIG. 10B). The octanal level was significantly increased to an average of 3.5 μM in mice fed WD (FIG. 10B). Octanal was also elevated in Apoe^{-/-} mice receiving CD and WD (FIG. 10C). Plasma of healthy human donors also contained octanal at an average concentration of 1.5 μM (±0.82, n=5).

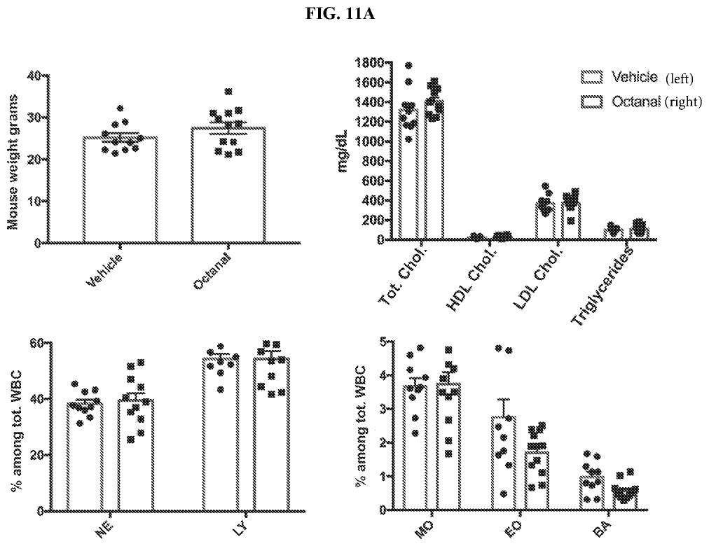

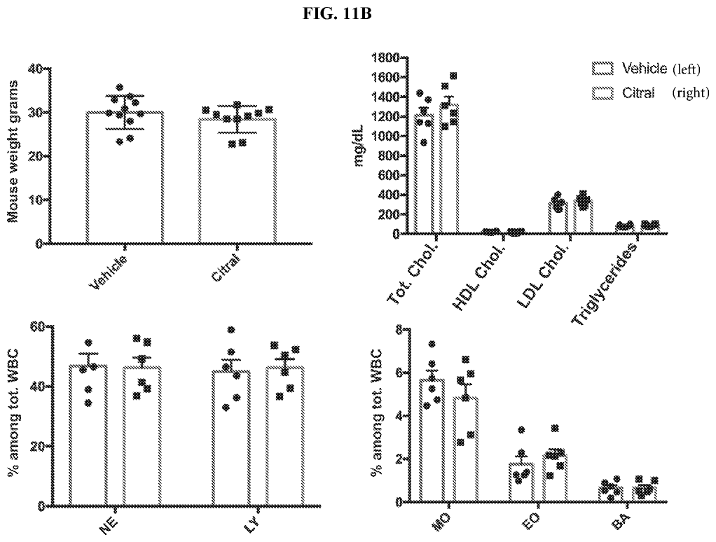

To test a possible impact of Olfr2 on atherosclerosis in vivo, male Apoe^{-/-} mice were fed WD for 8 weeks and injected octanal (10 μg/g) intraperitoneally for the last 4 weeks every three days for a total of 12 injections. En face staining of aortic arches showed that octanal treatment more than doubled lesion size (FIGS. 10D and E). Octanal had no effect on total cholesterol, LDL cholesterol, HDL cholesterol or triglycerides (FIG. 11A). Similarly, in a loss-of-function experiment, male Apoe^{-/-} mice were treated with citral (10 μg/g, 12 injections over 4 weeks), which reduced the size of atherosclerotic lesions by ˜40% (FIGS. 10F and G). Citral treatment like octanal had no effect on other parameters (FIG. 11A). This finding suggests that endogenous levels of volatile octanal are sufficient to induce a pathophysiologically relevant activation of Olfr2.

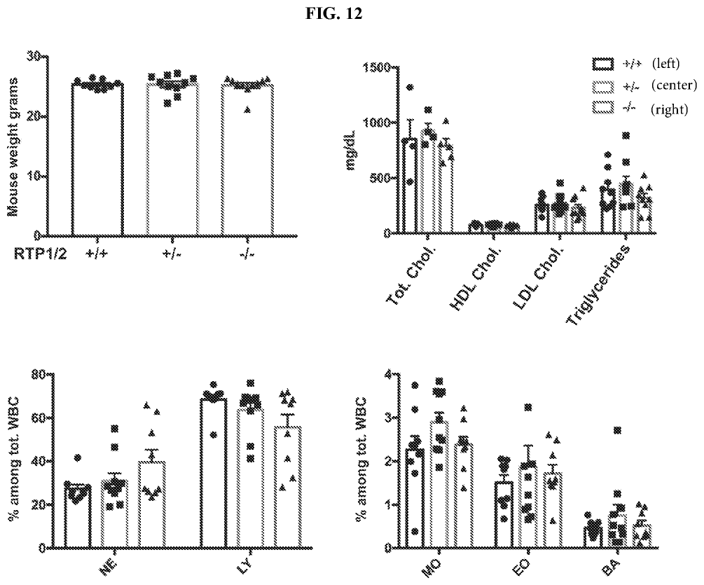

Since the expression and the trafficking of most olfactory receptors and specifically Olfr2 is regulated by Rtp1 and Rtp2¹⁹, whether their deletion would influence atherosclerotic lesion size was investigated. Male Ldlr^{-/-} mice were lethally irradiated and reconstituted with bone marrow cells from Rtp1/Rtp2^{-/-}, Rtp1/Rtp2^{+/-} or Rtp1/Rtp2^{+/+} mice. Western blot and flow cytometry analysis confirmed that Olfr2 expression was significantly reduced in BMDMs of Rtp1/Rtp2^{-/-} mice (FIGS. 10H and I; FIG. 12). After 2 weeks recovery, mice were fed a high-fat diet for 12 weeks. Circulating metabolic parameters (total cholesterol, high-density lipoprotein-cholesterol, and triglycerides), blood leukocyte counts and animal weights were similar between the 3 groups (FIG. 12). Aortic arch lesions in Rtp1/Rtp2^{-/-} chimeric mice were significantly reduced compared to Rtp1/Rtp2^{+/+} recipient mice (FIG. 10J). Taken together, Olfr2 and possibly other Olfrs regulated by Rtp1 and 2 promote atherosclerosis in mice. Inhibition of Olfr2 limits atherosclerosis in mice.

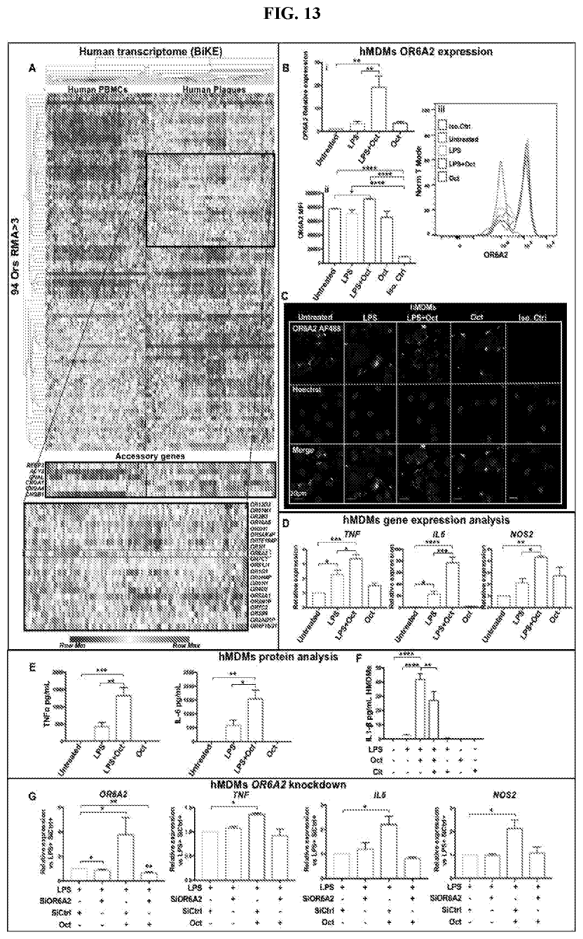

Having shown that human plasma contains octanal, the applicability of the mouse to human atherosclerosis was investigated. To this end, the transcriptomes of carotid artery plaques deposited in the Biobank of Karolinska Endarterectomy (BiKE) (GSE21545) were interrogated. OR6A2 mRNA, the human orthologue of mouse Olfr2, was expressed in all 126 plaque samples (RMA>3) and in 83 of 97 (85%) of peripheral blood mononuclear cell (PBMC) samples (FIG. 13A).

Next, whether human monocyte-derived macrophages (hMDMs) express OR6A2 was investigated. OR6A2 mRNA expression was detectable, increased by LPS and further augmented by adding octanal (FIG. 13Bi). OR6A2 protein surface expression in hMDMs was confirmed by flow cytometry (FIG. 13Bii and iii). Immunostaining suggests that OR6A2 expression in LPS+octanal-treated hMDMs may be polarized (FIG. 13C).

Octanal treatment of hMDMs induced mRNA expression of pro-inflammatory molecules including TNF, IL6 and NOS2 (FIG. 13D). In all cases, octanal enhanced the LPS-induced expression of these inflammatory molecules and their secretion as detected in the supernatant (FIG. 13E). Like in mouse macrophages, inflammasome activation was potentiated by octanal, resulting in a highly significant increase of IL-1β in the supernatant, which was partially but significantly inhibited by citral (FIG. 13F).

OR6A2 is not the only receptor for octanal (Araneda, R. C., Kini, A. D. & Firestein, S. The molecular receptive range of an odorant receptor. Nat Neurosci 3, 1248-1255, doi:10.1038/81774 (2000); Li, Y. et al. Aldehyde recognition and discrimination by mammalian odorant receptors via functional group-specific hydration chemistry. ACS Chem Biol 9, 2563-2571, doi:10.1021/cb400290u (2014)). To test whether OR6A2 is relevant for octanal-mediated hMDM activation, OR6A2 was silenced in hMDMs by small interference RNAs (SiOR6A2). LPS- and ocatanal-treated hMDMs exhibited reduced expression of IL6, NOS2 and TNF compared to hMDMs treated with scrambled siRNAs (SiCtrl) (FIG. 13G).

The discovery of a new class of GPCRs, Olfrs, that enhance atherosclerosis suggest a new class of drug targets. The octanal receptor Olfr2 and its human orthologue OR6A2 are expressed on aortic vascular macrophages, mouse BMDMs, human hMDMs and human atherosclerotic lesions. Olfr2/OR6A2 expression is increased by LPS and further elevated in the presence of octanal at the mRNA and protein level. Olfr2 ligation by octanal induces inflammatory cytokines and chemokines in mouse and human macrophages. Injecting mice with the known Olfr2 agonist octanal exacerbated atherosclerosis, while blocking Olfr2 by citral or impairing Olfr trafficking by Rtp1/Rtp2^{-/-} protected mice from atherosclerosis. Since blocking Olfr2 or impairing the trafficking of Olfrs suggests that octanal and perhaps other natural endogenous volatile compounds significantly drive atherosclerosis in vivo. Relevance to human atherosclerosis is suggested by the decreased inflammatory activation of octanal-stimulated hMDMs depleted of OR6A2, the observation of octanal in human blood serum and the presence of OR6A2 in all 126 human atherosclerotic plaques tested.

Since Olfrs have no known role yet in host defense, the examples demonstrate that drug-like small molecules targeting Olfr2, other Olfrs, and downstream signaling pathways might be viable therapeutic targets for the treatment, prevention and reversal of atherosclerosis.

Methods

Animals

Apoe^{-/-}, Ldlr^{-/-} and wt mice on C57BL/6J background were purchased from Jackson Laboratories. Rtp1/Rtp2^{-/+} breeding pairs were kindly provided by Dr. Hiroaki Matsunami from the Duke University Medical Center. Gender-matched mice (8-10 weeks old) were used in all experiments unless specified. Mice were housed in a conventional environment and had access to chow diet ad libitum. From 8-10 weeks of age on, Apoe^{-/-} and C57BL/6J mice were fed a western-like diet (WD) with 42% from fat (Harlan Labs Cat #: TD.88137) and remained on WD until sacrifice. Ldlr^{-/-} male mice (8-10 weeks of age) were used for bone marrow transplantation experiments as specify in detail below.

All animal experiments were conducted in accordance with the institutional guideline at the La Jolla Institute for Immunology animal facility.

Mouse Aorta Ex Vivo Culture

Each aorta was surgically removed and carefully cleaned of adipose tissue in situ. Aortas were either kept whole or enzymatically digested as previously described¹⁸. In brief, aortas were cut into small pieces and individually digested 1h at 37° C. in HBSS containing 450U/mL Collagenase I (Sigma Aldrich), 250 U/mL Collagenase XI (Sigma Aldrich), 120 U/mL Hyaluronidase (Sigma Aldrich), and 120 U/mL DNase I (Worthington). The digested aortic cell suspension was filtered through a 50 μm cell strainer (Partec) and washed with warm 37° C. RPMI-1640 medium (Gibco-ThermoFischer) supplemented with 10% fetal bovine serum (FBS) and 1% Penicillin/Streptomycin solution (Gibco-ThermoFischer) (from here on referred to as culture medium).

Whole aortas or digested aortic cells were cultured in culture medium for further stimulation as described below.

Mouse Blood Collection and Leukocyte Isolation

Mouse blood was collected in ethylenediamine tetraacetic acid (EDTA)-coated tubes (Sarstedt) by cardiac heart puncture during organ harvest.

Circulating leukocytes were obtained from full mouse blood incubated with 10 mL of 1× Red Blood Cell (RBC) Lysis Buffer (eBioscience) for 10 min at room temperature (RT) in the dark. Subsequently, cells were washed twice with culture medium and used for further flow cytometry studies.

Bone Marrow Cell Isolation and Generation of BMDMs

Mouse bone marrow was isolated from femurs and tibiae flushed with 5 ml ice cold RPMI-1640 medium. Bone marrow cells were passed through a 70 μm cell strainer (BioPioneer) and resuspended at 1×10⁶ cells/ml in culture medium supplemented with 20 ng/mL of mouse recombinant macrophage-colony stimulating factor (M-CSF; Peprotech). Cells were cultured at 37° C. and 5% CO₂ in a 3.8 cm² (Thermo Fischer Scientific) dish. After 3 days, 5 ml of fresh medium were added to the culture. On day 7, floating cells were discarded and adherent cells used for further studies.

Human Monocyte Derived Macrophages (hMDMs)

Peripheral blood mononuclear cells (PBMCs) were isolated from EDTA-venous blood of informed healthy donors (25-50 years old) from La Jolla Institute for Immunology's in-house Normal Blood Donor Program (NBDP), using a Ficoll-Paque (GE Healthcare) standard separation protocol. Human monocytes were enriched from PBMCs by the EasySep™ Human Monocyte Isolation Kit (StemCell) according to the manufacturer's instructions. Enriched monocytes were incubated with culture medium supplemented with 50 ng/ml rhM-CSF at 37° C. and 5% CO₂ for 7 days. Differentiated macrophages at day 7 were used for further studies.

Olfr2 Functional Ex Vivo and In Vivo Experimental Procedures

Ex Vivo Analysis

Octanal, citral, and dimethylsulfoxide (DMSO), were purchased from Sigma. All chemicals used were of analytical grade or the highest grade available. Citral and octanal were reconstituted/diluted in DMSO at 10 mM conc.

Apoe^{-/-} mice were fed WD for 2 weeks before sacrifice for aorta explant culture. Whole aortas were incubated in culture medium with DMSO (0.1% as a vehicle), octanal (Oct, 10 μM), LPS (500 ng/mL) alone or octanal and LPS combined (LPS+Oct) for 12 h at 37° C. and 5% CO₂. Subsequently, aortas were collected and further processed as below discussed for RNA extraction, and flow cytometry analysis. Culture supernatants were collected, aliquoted, and frozen at −80° C. until further analysis.

In Vivo Studies

Male Apoe^{-/-} mice (9 to 10 wks old) were fed WD for 4 weeks. Oct or Cit were diluted in sterile phosphate buffered saline (PBS) (Gibco) at a final concentration of 10 μg/g. 100 μl were injected intraperitoneally (i.p.) every 3 days for 4 weeks (12 injections total) while maintaining WD administration. PBS+0.1% DMSO (Sigma Aldrich) was injected as a vehicle control following the same protocol. Mice were sacrificed, perfused with PBS implemented with 2% heparin (Fresenius Kabi USA, LLC) and aortas removed for en face atherosclerotic lesion assessment. Mouse whole blood (heart puncture) was collected in EDTA tubes. Leukocyte blood counts were determined with HemaVet 950 (Drew Scientific). Plasma was collected from blood centrifuged at 3000 g for 15 min at 4° C. and stored at −80° C. until further analysis.

Bone Marrow Transplantation

Ten-week-old Ldlr^{-/-} male mice were irradiated twice 2-4h apart with 550 rads X-ray irradiation to induce systemic medullar aplasia. Irradiated mice were repopulated intravenously (tail vein) with bone marrow cells (5×10⁶ cells) isolated from femurs and tibias of 10-week-old Rtp1/Rtp2^{+/+}, Rtp1/Rtp2^{+/-}, Rtp1/Rtp2^{-/-} mice, respectively. After a 2-week recovery period, mice were fed a high cholesterol diet (product number D12108C, Research Diets Inc.) for additional 12 wks. At the end of the 12 wks mice were sacrificed, perfused with PBS implemented with 2% heparin (Fresenius Kabi USA, LLC) and aortas removed for en face atherosclerotic lesion assessment. Blood was collected in EDTA tubes by cardiac heart puncture. Leukocyte blood counts were determined with HemaVet 950 (Drew Scientific). Plasma was collected from blood centrifuged at 3000 g for 15 min at 4° C. and stored −80° C. until further analysis.

Femurs and tibias were also collected for BMDMs differentiation flowing the same protocol already described, and furtherly used for flow cytometry and western blot analysis of Olfr2 expression.

Atherosclerosis Quantification in Whole Aortas

After explant, each aorta was pinned out after 4% paraformaldehyde incubation at room temperature for at least 2 hours Staining for atherosclerotic plaque was performed by incubating samples in Sudan IV. Microphotographs of the luminal (endothelial) aspect were of the aortic surface were analyzed using Photoshop CS (Adobe Systems). Atherosclerotic lesion size measurement was performed by blinded investigators using Image-Pro Premier software (Media Cybernetics).

Lipid Analysis

Lipid analysis of collected plasma was performed by IDEXX BioResearch.

Quantitative Real Time PCR

BMDMs and hMDMs were incubated in culture media for 12 h at 37° C. and 5% CO₂ with Oct, (10 μM), LPS (500 ng/mL) alone or combined with Oct (LPS+Oct) and DMSO (0.1%) as a control and lysed with Qiazol (500 μL; Qiagen) Similarly, whole aortas incubated as previously described were transferred into Qiazol (500 μl; Qiagen) and digested with gentleMACS™ Dissociator (Miltenyi).

RNA extraction was performed using a Qiazol/RNAeasy micro kit hybrid protocol (Qiagen).

Total RNA was quantified using a nanodrop spectrophotometer (Peqlab) and 500 ng were reverse transcribed into cDNA using Omniscript reverse transcriptase (Qiagen). Real time PCR reactions were performed according to the RT² SYBR green gene expression assay protocol (Qiagen). RT2 SYBR Green qPCR master mix and premade RT² qPCR Primer Assays for mouse Olfr2, Rtp1, Rtp2, Ccl2, Ccl4, Tnf, Nos2 and Gapdh were used. Similarly, real time PCR analysis of hMDMs samples was performed following the RT² SYBR green gene expression assay protocol (Qiagen), applying RT² SYBR Green qPCR master mix and premade RT² qPCR Primer Assays for human OR6A2, TNF, IL-6, NOS2 and GAPDH. The results were calculated applying the 2^{-ΔΔCT} method³² using Gapdh/GAPDH as housekeeping gene.

OR6A2 Silencing in hMDMs

Human MDM cells (2×10⁶) were resuspended in 100 μl RT Nucleofector® Solution A mixture (Amaxa® Cell Line Nucleofector® Kit V; Lonza) containing 5 pmol non-silencing siRNA duplex (siCtrl, Thermo Fischer ientific) or silencing siRNA duplex targeted against the OR6A2 gene (assay ID 148343, Thermo Fischer Scientific). The suspension was transferred to an aluminum electroporation cuvette (Lonza) and pulsed in a Nucleofector 2b Device using (Lonza) using the premade BMDMs program. After electroporation, cells were transferred to RPMI-1640 medium supplemented with 20% of fetal calf serum, 100 U/mL penicillin and 100 μg/mL streptomycin, and 50 ng/mL rhMCSF (Peprotech) and cultured in 48-well plates at 37° C. and 5% CO₂ for 48 h. Cell viability was determined by a trypan blue exclusion assay. Cells were further treated in culture medium for 12 h at 37° C. and 5% CO₂ with Oct, (10 μM), LPS (500 ng/mL) alone or combined with Oct (LPS+Oct), and DMSO (0.1%) as a control and subsequently lysed in Qiazol (500 μl) for RNA purification. OR6A2, TNF, HA and NOS2 expression levels were analyzed by Real Time PCR as described before.

Flow Cytometric Analysis

BMDMs, hMDMs, mouse blood leukocytes and whole mount aortas were incubated in culture medium with Oct, (10 μM), LPS, (500 ng/mL) alone or combined (LPS+Oct) for 12h at 37° C. and 5% CO₂. After the incubation aortas were cut into small pieces and digested for cell suspension as previously described¹⁸.

Subsequently, aortic cell suspensions, BMDMs, blood leukocytes and hMDMs were incubated for 1 h with mouse or human Fc block solution, respectively, and stained with primary antibodies detecting Olfr2 (1:500, Thermo Fischer Scientific) or OR6A2 (1:500, Sigma Aldrich) for 1 h at RT. Normal rabbit IgG (1:500 Sigma Aldrich) incubated cells were used as a control.

All cells were then washed 3 times and incubated with an AlexaFluor488-conjugated goat anti-rabbit IgG secondary antibody (1:500, Thermo Fischer Scientific) for 30 min at RT. After 3 washing steps with PBS, BMDMs were stained in the dark for 20 min at RT with LIVE/DEAD (LD) Yellow fixable dye (Invitrogen) and F4/80-BV421. Aortic cells were stained in the dark for 20 min at RT with a mixture of CD45-PerCP, CD11b-PECy7, CD11c-BV605, TCRβ-BV711, CD4-PE-AlexaFluor610, CD19-APCCy7, GR1-BV786, F4/80-BV421 (Biolegend) antibodies and LD Yellow fixable dye. Mouse blood leukocytes were stained in the dark for 20 min at RT with a mixture of CD45-PerCP, CD115-PECy7, Ly6C-BV421, TCRβ-BV711, CD4-PE-AlexaFluor610, CD19-APCCy7, (Biolegend) antibodies and LD Yellow fixable dye. hMDMs were stained in the dark for 20 min at RT with LD Yellow fixable dye and anti-human CD45-AF700.

Samples were analyzed with the LSR-II flow cytometer (BD Biosciences). Data was acquired with the FACSDiva software (BD Biosciences) and analyzed with FlowJo software (Treestar Inc).

Intracellular Staining

Whole aortas and mouse blood leukocytes were incubated in culture medium with Oct, (10 μM), LPS, (500 ng/mL) alone or combined (LPS+Oct) for 12h at 37° C. and 5% CO₂. The protein transport inhibitor cocktail 1× (eBioscience) was added for whole 12h in the whole aorta samples and for the last 6 hours in the mouse blood leukocyte samples. Aortic cell suspensions were obtained as mentioned above, stained with CD45-PerCP, CD11b-PECy7, CD11c-BV605, TCRβ-BV711, CD4-PE-AlexaFluor610, CD19-APCCy7, GR1-BV786, F4/80-BV421 and LD Yellow fixable dye (Invitrogen). Blood leukocytes were stained with CD45-PerCP, CD115-PECy7, Ly6C-BV421, TCRβ−/CD4/CD19-APCCy7 (dump channel), and LD Yellow fixable dye.

Cells were then washed, fixed, permeabilized applying the Intracellular Fixation and Permeabilization Buffer Set (eBioscience), and stained with mouse anti-TNF-PE (eBioscience). Samples were analyzed with LSR-II (BD Biosciences). Data was acquired with FACSDiva software (BD Biosciences) and analyzed with FlowJo software (Treestar Inc).

IL-1β Production

Mouse BMDMs and human MDMs were pretreated with LPS (50 ng/mL) or left untreated for 18 h at 37° C. and 5% CO₂. After this period LPS pretreated and untreated cells were further incubated with Oct (10 μM) and Cit (100 μM) alone (LPS+Oct; LPS+Cit; Oct; Cit, respectively) or combined (LPS+Oct+Cit) for further 8 h. Supernatants were collected by centrifugation at 2000 g for 15 minutes at 4° C., collected and frozen at −80° C. until further IL-1β analysis by CBA (BD Biosciences).

Secreted Cytokines

Supernatants of stimulated cells and aortic tissue were analyzed by BD CBA Flex Sets (BD Biosciences) for mouse TNFα, CCL2, CCL4, IL-1β, or human TNF, IL6, and IL-1β according to the manufacturer's protocol. Samples were analyzed with LSR-II (BD Biosciences). Data was acquired with FACSDiva software (BD Biosciences) and analyzed with FCAP array software 3.0 (BD Biosciences).

Calcium Signalling

Aortic cell suspensions obtained as previously described¹⁸ were pre-incubated with extracellular markers for vascular macrophages including CD45-PerCP, TCRβ/CD4-APCCy7 (Dump Channel), F4/80-BV421 and LD Yellow fixable dye (Invitrogen) for 20 min in Fc block solution at RT in the dark. Aortic cell suspensions were than washed twice and loaded with 2 μM of Fluo4 dye (Thermo Fischer Scientific) for 30 min at 37° C.

Subsequently, cells were washed and resuspended in 37° C. warm HBSS buffer containing Ca²⁺ and Mg²⁺ and acquired with the LSRII flow cytometer. After 60 seconds, Oct (10 μM), Cit (100 μM) or vehicle (DMSO 0.1%) was added and acquisition continued for additional 140 seconds. Data was acquired with FACSDiva software (BD Biosciences) and analyzed with FlowJo software (Treestar Inc). Mean fluorescence intensity of Fluo4 was integrated over 25 seconds form the start of the acquisition (tot. 8 intervals).

Immunoblotting

For Olfr2 protein detection in BMDMs, cells were incubated in culture medium with Oct, (10 μM), LPS, (500 ng/mL) alone or combined (LPS+Oct) for 12h at 37° C. and 5% CO₂. Cells were then washed and lysed in RIPA buffer containing protease inhibitors. Total protein content was quantified using DC Protein Assay (Bio-Rad) and solubilized in a volume ratio of 1:1 with Laemmli Buffer (Bio-Rad). SDS-Page analysis was conducted by heating the samples to 95° C. for 10 min and resolved on 12% Mini-PROTEAN TGX gels (Bio-Rad) under denaturing conditions. Pre-stained protein molecular weight standards (Thermo Fischer Scientific) were used as markers. Proteins were transferred onto Nitrocellulose membranes (Amersham Protran 0.2 μm NC) using the Mini Trans-blot electrophoretic transfer cell (Bio-Rad). Membranes were blocked in 5% bovine serum albumin (Sigma) prepared in tris-buffered saline with 0.1% Tween-20 (TBST). Membranes were incubated overnight at 4° C. with 1:1000 diluted Olfr2 (Thermo Fischer Scientific) or tubulin (CST) primary antibodies. Membranes were washed thrice with TBST and incubated with a HRP-conjugated secondary antibody (Promega) for 1 h at RT. Blots were again washed thrice with TBST and developed using a chemiluminescent substrate (Thermo Fischer Scientific). Protein bands were visualized with the ChemiDoc MP Imaging System (Bio-Rad) using Image Lab software.

Microarray Analysis and Dataset Comparison.

Published microarray datasets (Affymetrix mouse gene 1.0 ST array type) were used for the Olfr comparison: GSE68968 Vascular Mc); GSE77104 BMDMs, GSE43075 intra-peritoneal (IP) Mc); GSE100393 Small Intestine (SI) Mc); GSE15907 Lung Mc); GSE15907 Spleen (Red pulp) Mc); GSE15907 Microglia; GSE100393 SI DCs; GSE15907 Lung DCs; GSE15907 Mesenteric LN (MLN) DCs; GSE15907 Blood classical (CM) and non-classical monocyte (NCM) subpopulations. Affymetrix gene chip data were normalized using the robust multi-array average (RMA) method (log 2, background-corrected, quantile normalized). Correlation analyses were based on Pearson's correlation. Gene expression among dataset were normalized based on Gapdh expression (Xmax) using the ranking strategy: X−Xmin/Xmax−Xmin. Heatmap analysis and visualization was performed applying Morpheus, software.broadinstitute.org/morpheus.

RNAseq Analysis

For bulk RNA-seq, living CD45⁺, TCRβ⁻, CD19⁻, F4/80⁺ macrophages from aortas of Apoe^{-/-} mice fed a WD for 20 weeks were FACS-sorted with the FACSAriaIII (BD Biosciences).

Sequencing libraries were prepared from purified RNA using the RNeasy Mini Kit (Qiagen).

Double stranded (ds)-cDNA was prepared using the SMART-seq v4 Ultra Low RNA Kit for Illumina Sequencing (Takara-Clontech) according to the manufacturer's instructions. cDNA was amplified using 11 cycles and eluted in 12 μL. 5 μL of resulting cDNA was processed using a NexteraXT kit (Illumina, Calif., USA) following manufacturer's instructions. Samples were pooled into two batches, loaded on an Illumina HiSeq4000 and sequenced with 50 base pair single end reads (SR50). Transcriptomes were mapped to the mm10 mouse genome. Post-mapping quality control checks were used to exclude poor quality samples.

Immunofluorescence and Whole Mount Aorta Imaging

BMDMs and hMDMs were cultured on μ-Slide 8 well glass bottom (Ibidi) and treated as described above. Subsequent to incubation, cells were fixed with 2% paraformaldehyde (PFA) for 30 min at 37° C. and incubated with 5% BSA for 30 min at RT. BMDMs and hMDMs were than stained with Olfr2 and OR6A2 primary antibodies [1:500, Thermo Fischer Scientific (mouse Olfr2) and 1:500 Sigma-Aldrich Co (human OR6A2)], respectively, for 1 h in Fc blocking solution. Cells were then washed 5 times for 5 minutes with cold PBS and stained with secondary anti-rabbit IgG-AF488 (1:400, Thermo Fischer Scientific) for 30 min at 4° C. Nuclear counterstaining with Hoechst (1:2500, Sigma) was performed for 10 min Cells were then washed 5 times and imaged with a Zeiss LSM880 confocal scanning microscope. Image acquisition settings were adjusted with control samples (unstained sample, and isotypes control) and kept throughout the experiment.

For whole mount imaging, aortae form Apoe^{-/-} mice fed WD for 2 weeks were carefully cleaned in situ and fixed in 4% paraformaldehyde at 4° C. for 24 h. Subsequently, aortae were washed with PBS and transferred to 5 mL incubation buffer (2% FCS, 0.5% saponin, 0.1% sodium azide) containing Olfr2 primary antibody (1:1000, Thermo Fischer Scientific) for and agitated for 24 h at 37° C. Aortas were then washed 5 times with PBS and stained while agitation in 5 mL incubation buffer containing secondary anti-rabbit IgG-AF555 (1:500, Thermo Fischer Scientific), anti-mouse CD68-AF647 (1:200, Biolegend) and Hoechst (1:10000, Sigma) for 24 h at 37° C. Stained aortas were washed with PBS, opened longitudinally, mounted between glass slides, and imaged form the adventitial side with a Zeiss LSM880 confocal scanning microscope. Image acquisition settings were set with control samples (Unstained samples, CD68-AF647 FMO, and secondary anti-rabbit IgG-AF555 FMO) and maintained throughout the experiment. Image and scan processing were performed with Zen microscope software (Zeiss) and Imaris analysis software (Bitplane).

Plasma Octanal Quantification

Mouse blood plasma was collected reto-orbitally from 8-10 weeks old Apoe^{-/-} and WT mice fed WD or chow diet for 1 day.

Plasma octanal was derivatized with 3-nitrophenylhydrazine in the presence of 1-ethyl-3-(3-dimethylaminopropyl)carbodiimide (EDC) and detected by LC/MS/MS after injection onto a Titan™ C18 UHPLC Column (1.9 μm particle size, 10 cm×2.1 mm, Supelco) with elution solvent generated by two pumps from solvent A, water+0.01% formic acid and B, acetonitrile+0.01% formic acid. A typical ion extracted LC chromatogram of octanal derivative in positive MRM mode with parent to daughter transition, m/z 264.2→119.1 was provided. [²H₁₆]octanal was spiked to plasma before derivatization reaction as internal standard and monitored in positive MRM mode with parent to daughter transition, m/z 280.2→120.2. Series concentrations of Oct were mixed with fixed amount of [²H₁₆]octanal followed by derivatization reaction were used to prepare calibration curve.

Statistics

All statistics in this study were performed using Graphpad Prism 6.0.

Analysis were done with ordinary one-way ANOVA followed by Tukey's test for multiple comparisons or the non-parametric Kruskal-Wallis test with Dunn's test for multiple comparisons. Where appropriate, statistical significance was tested with unpaired t-Test. The respective statistical tests are indicated in each figure legend. Data are presented as mean±SEM. P-values below 0.05 were considered significant.

### Example 4: Identification of OLFRs that Modulate Immune Responses

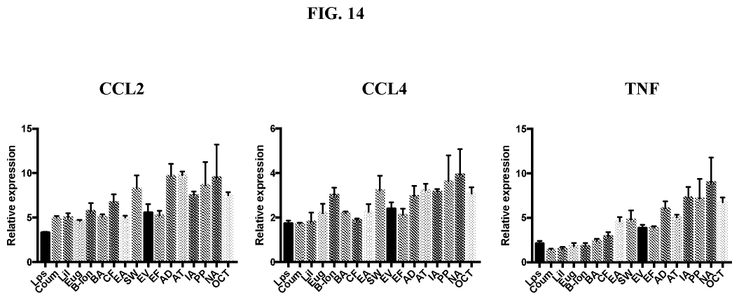

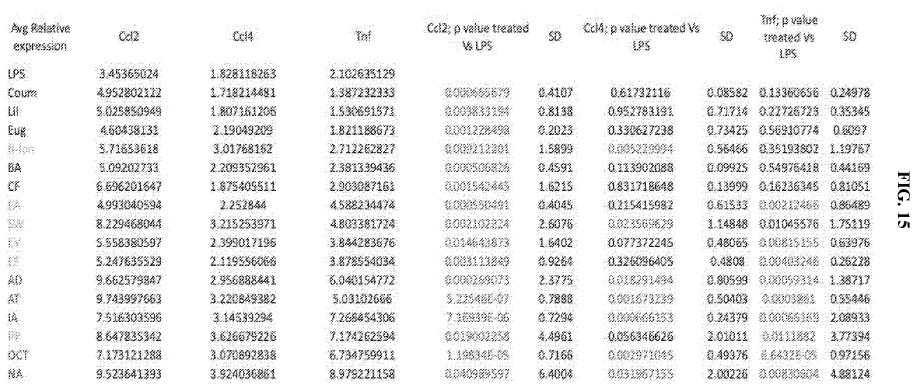

An analysis on representative pro inflammatory genes (Ccl2, Ccl4 and Tnf) was performed (FIG. 14). Briefly, explanted aortas of Apoe^{-/-} mice kept in WD (2 wks), were treated for 12h with LPS (500 ng/mL) and different odorants (10 μM) or left untreated. Relative expression of Ccl2, Ccl4, and Tnf was quantified by real time PCR analysis and normalized to Gapdh expression and untreated control applying the 2-ΔΔCT method. N=3 per group.

Legend for odorants screened: Coumarin (Coum), Lilial (LIL), b-ionone (b-iono), Eugenol (EU), Butyric Acid (BA), Caramel Furanone (CF), Eugenol acetate (EA), Sandalwood (SW), Ethil Vanillin (EV), Ethil Fencol (EF), Androstadienone (AD), Androstenone (AT), Isovaleric Acid (IA), N-Amil Acetate (NA), Propyl propionate (PP), Octanal (OCT).

A statistical analysis of the representative pro inflammatory genes (Ccl2, Ccl4 and Tnf) was then performed (FIG. 15). The average relative expression for Ccl2, Ccl4, and Tnf was quantified by real time PCR analysis and normalized to Gapdh expression and untreated control applying the 2-ΔΔCT method. N=3 per group. p calculated by unpaired T Test. A p value<0.05 is considered significant. This analysis led to the identification of olfactory receptors for which the modulation of the expression or activity of one or more of these olfactory receptors, without being bound to a particular theory, results in modulation of an immune response corresponding to the embodiments provided herein. By way of example, and not by way of limitation, these olfactory receptors may include those olfactory receptors listed in Table 7 or one or more of OR7C1, OR7D4, OR10A6, OR11H6, OR4E2, OR10H1, or OR6A2 or an ortholog, homologue, variant or derivative thereof.

### Example 5: Plasma Proteome Analysis

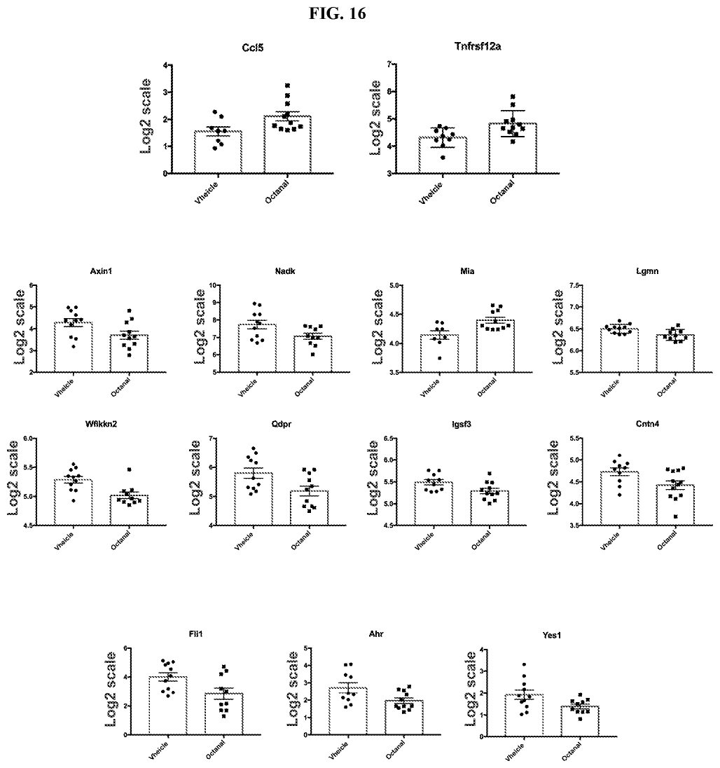

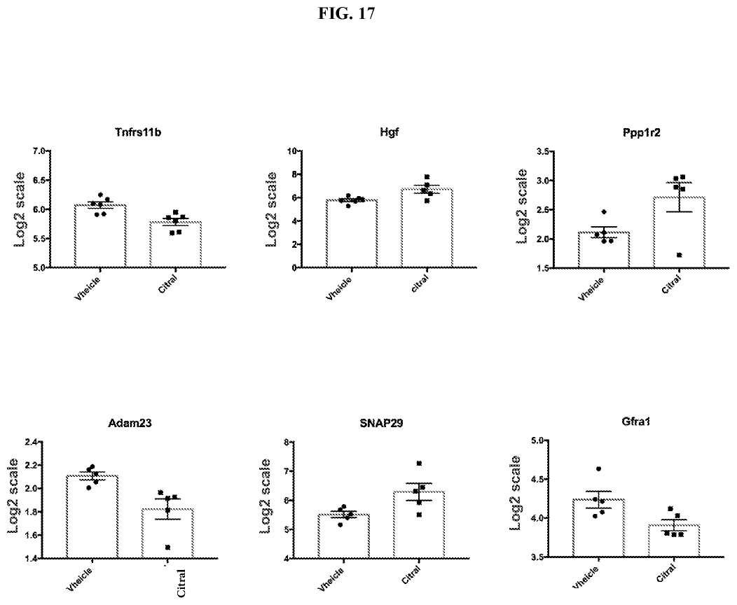

In this example, significantly modulated proteins in plasma from octanal treated mice (FIG. 16) and citral treated mice (FIG. 17) were examined Briefly, EDTA plasma samples were collected from octanal treated, citral treated, and vehicle treated mice at the end of the 8 weeks and analysis of 92 proteins therein were examined. The analysis of the 92 proteins comprising the Olink Mouse exploratory panel (https://www.olink.com/products/mouse-exploratory/) was performed using PEA (Proximity Extension Assay) technology. The PEA technique allows simultaneous assessment of proteins using oligonucleotide-labeled antibody probe pairs that bind to each protein within the sample. The PEA technique also permits accurate assessment of protein levels with repeated measurements by requiring both dual recognition of correctly matched antibody pairs and DNA-barcoding from sequence-specific oligonucleotides to generate a signal (PLoS ONE. 2014; 9(4)). The assay measures fold change in log 2 units. For this analysis, the raw data are converted into a t-statistic which can be compared across assays. Data are presented as means±SEM. P value<0.05 calculated by unpaired t-test. This analysis confirmed that modulation of olfactory receptor expression or activity may increase or decrease the expression or activity of proteins involved with immune response, and as such increasing or decreasing expression or activity of olfactory receptors may be utilized to increase or decrease expression or activity of certain immune response-related proteins. By way of example, and not by way of limitation, immune response-related proteins may include those genes listed in FIG. 16 and/or FIG. 17, or one or more of CCL5 (C-C Motif Chemokine Ligand 5), Tnfrsf12a (Osteoprotegerin), Axin 1, Nadk, Ahr (Aryl hydrocarbon receptor), QDPR (quinoid dihydropteridine reductase), HGF (Hepatocyte Growth Factor), ADAM23, or Snap29.

### Example 6: Plasma Proteome Analysis

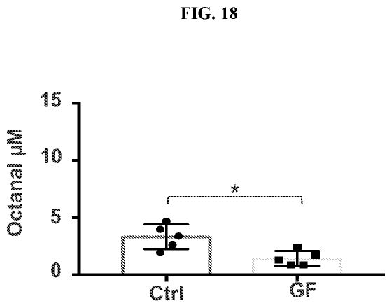

There are different sources of OLFR ligands, including diet, bacterial microbiota metabolism of various precursors (including from diet), and naturally occurring octanal in the subject. The role of gut microbiota in the production of ligands is demonstrated by generation of mice lacking gut microbiota which are found to have less concentrations of OLFR ligands than wild-type mice. For example, mice lacking gut microbiota are found to have 3-fold less concentration of octanal than wild-type mice (FIG. 18).

### Example 7: Role of OLFRs in Immune Modulation

In order to determine an olfactory receptors' role in immune modulation, an Olfr knockout mouse is prepared. These mice are treated with a ligand or agonist of the OLFR and show reduced atherosclerosis when compared to Apoe−/− mice and WT mice also treated with the ligand or agonist. In one example, an Olfr2 knockout mouse is prepared and treated with octanal. These mice show reduced atherosclerosis when compared to Apoe−/− mice and WT mice also treated with octanal. As such, reduction of Olfr2 expression or activity reduces or inhibits inflammatory response.

To further define the role of olfactory receptors in modulating immune response, olfactory receptor involvement in inflammasome activation/signaling is demonstrated. For example, Olfr2 binding to its ligand represents a Signal 2 or activation signal, promoting the maturation and secretion of pro-inflammatory cytokines interleukin 1-beta (IL-1B) and interleukin 18 (IL-18). Without being bound to any particular theory, olfactory receptor activation may result in the activation of nucleotide-binding oligomerization domain and leucine-rich repeat-containing receptors (NLRs) in the inflammasome. By way of example, and not by way of limitation, NLRs, including NALP3 (NACHT, LRR and PYD domains-containing protein 3, also known as cryopyrin) or NLRC4 (NLR family CARD domain-containing protein 4) are activated by OLFR2.

### REFERENCES

The references cited throughout this application are listed below. Each reference is incorporated by reference in their entirety.
1 Buck, L. & Axel, R. A novel multigene family may encode odorant receptors: a molecular basis for odor recognition. Cell 65, 175-187 (1991).
2 Jacoby, E., Bouhelal, R., Gerspacher, M. & Seuwen, K. The 7 TM G-protein-coupled receptor target family. ChemMedChem 1, 761-782, doi:10.1002/cmdc.200600134 (2006).
3 Kato, A. & Touhara, K. Mammalian olfactory receptors: pharmacology, G protein coupling and desensitization. Cell Mol Life Sci 66, 3743-3753, doi:10.1007/s00018-009-0111-6 (2009).
4 Rouquier, S., Blancher, A. & Giorgi, D. The olfactory receptor gene repertoire in primates and mouse: evidence for reduction of the functional fraction in primates. Proc Natl Acad Sci USA 97, 2870-2874, doi:10.1073/pnas.040580197 (2000).
5 Go, Y. & Niimura, Y. Similar numbers but different repertoires of olfactory receptor genes in humans and chimpanzees. Mol Biol Evol 25, 1897-1907, doi:10.1093/molbev/msn135 (2008).
6 Saito, H., Kubota, M., Roberts, R. W., Chi, Q. & Matsunami, H. RTP family members induce functional expression of mammalian odorant receptors. Cell 119, 679-691, doi:10.1016/j.cell.2004.11.021 (2004).
7 Li, F. et al. Heterotrimeric G protein subunit Ggamma13 is critical to olfaction. J Neurosci 33, 7975-7984, doi:10.1523/JNEUROSCI.5563-12.2013 (2013).
8 Silva Teixeira, C. S., Cerqueira, N. M. & Silva Ferreira, A. C. Unravelling the Olfactory Sense: From the Gene to Odor Perception. Chem Senses 41, 105-121, doi:10.1093/chemse/bjv075 (2016).
9 Ukhanov, K., Brunert, D., Corey, E. A. & Ache, B. W. Phosphoinositide 3-kinase-dependent antagonism in mammalian olfactory receptor neurons. J Neurosci 31, 273-280, doi:10.1523/JNEUROSCI.3698-10.2011 (2011).
10 Ukhanov, K., Corey, E. A., Brunert, D., Klasen, K. & Ache, B. W. Inhibitory odorant signaling in Mammalian olfactory receptor neurons. J Neurophysiol 103, 1114-1122, doi:10.1152/jn.00980.2009 (2010).
11 Ferrer, I. et al. Olfactory Receptors in Non-Chemosensory Organs: The Nervous System in Health and Disease. Front Aging Neurosci 8, 163, doi:10.3389/fnagi.2016.00163 (2016).
12 Lee, S. J., Depoortere, I. & Hatt, H. Therapeutic potential of ectopic olfactory and taste receptors. Nat Rev Drug Discov 18, 116-138, doi:10.1038/s41573-018-0002-3 (2019).
13 Li, J. J. et al. Activation of olfactory receptors on mouse pulmonary macrophages promotes monocyte chemotactic protein-1 production. PLoS One 8, e80148, doi:10.1371/journal.pone.0080148 (2013).
14 Pluznick, J. L. et al. Functional expression of the olfactory signaling system in the kidney. Proc Natl Acad Sci USA 106, 2059-2064, doi:10.1073/pnas.0812859106 (2009).
15 Bozza, T., Feinstein, P., Zheng, C. & Mombaerts, P. Odorant receptor expression defines functional units in the mouse olfactory system. J Neurosci 22, 3033-3043, doi:20026321 (2002).
16 Araneda, R. C., Peterlin, Z., Zhang, X., Chesler, A. & Firestein, S. A pharmacological profile of the aldehyde receptor repertoire in rat olfactory epithelium. J Physiol 555, 743-756, doi:10.1113/jphysiol.2003.058040 (2004).
17 Rizzo, W. B. Fatty aldehyde and fatty alcohol metabolism: review and importance for epidermal structure and function. Biochim Biophys Acta 1841, 377-389, doi:10.1016/j.bbalip.2013.09.001 (2014).
18 Butcher, M. J., Herre, M., Ley, K. & Galkina, E. Flow cytometry analysis of immune cells within murine aortas. J Vis Exp, doi:10.3791/2848 (2011).
19 Sharma, R. et al. Olfactory receptor accessory proteins play crucial roles in receptor function and gene choice. Elife 6, doi:10.7554/eLife.21895 (2017).
20 Ross, R. Atherosclerosis—an inflammatory disease. N Engl J Med 340, 115-126, doi:10.1056/NEJM199901143400207 (1999).
21 Wolf, D., Zirlik, A. & Ley, K. Beyond vascular inflammation—recent advances in understanding atherosclerosis. Cell Mol Life Sci 72, 3853-3869, doi:10.1007/s00018-015-1971-6 (2015).
22 Moore, K. J., Sheedy, F. J. & Fisher, E. A. Macrophages in atherosclerosis: a dynamic balance. Nat Rev Immunol 13, 709-721, doi:10.1038/nri3520 (2013).
23 Gomez Perdiguero, E. et al. Tissue-resident macrophages originate from yolk-sac-derived erythro-myeloid progenitors. Nature 518, 547-551, doi:10.1038/nature13989 (2015).
24 Ginhoux, F. & Jung, S. Monocytes and macrophages: developmental pathways and tissue homeostasis. Nat Rev Immunol 14, 392-404, doi:10.1038/nri3671 (2014).
25 Ensan, S. et al. Self-renewing resident arterial macrophages arise from embryonic CX3CR1(+) precursors and circulating monocytes immediately after birth. Nat Immunol 17, 159-168, doi:10.1038/ni.3343 (2016).
26 Cochain, C. et al. Single-Cell RNA-Seq Reveals the Transcriptional Landscape and Heterogeneity of Aortic Macrophages in Murine Atherosclerosis. Circ Res 122, 1661-1674, doi:10.1161/CIRCRESAHA.117.312509 (2018).
27 Winkels, H. et al. Atlas of the Immune Cell Repertoire in Mouse Atherosclerosis Defined by Single-Cell RNA-Sequencing and Mass Cytometry. Circ Res 122, 1675-1688, doi:10.1161/CIRCRESAHA.117.312513 (2018).
28 Kim, K. et al. Transcriptome Analysis Reveals Nonfoamy Rather Than Foamy Plaque Macrophages Are Proinflammatory in Atherosclerotic Murine Models. Circ Res 123, 1127-1142, doi:10.1161/CIRCRESAHA.118.312804 (2018).
29 Ridker, P. M. et al. Antiinflammatory Therapy with Canakinumab for Atherosclerotic Disease. N Engl J Med 377, 1119-1131, doi:10.1056/NEJMoa1707914 (2017).
30 Martinon, F., Burns, K. & Tschopp, J. The inflammasome: a molecular platform triggering activation of inflammatory caspases and processing of proIL-beta. Mol Cell 10, 417-426 (2002).
31 Karasawa, T. & Takahashi, M. Role of NLRP3 Inflammasomes in Atherosclerosis. J Atheroscler Thromb 24, 443-451, doi:10.5551/jat.RV17001 (2017).
32 Pfaffl, M. W. A new mathematical model for relative quantification in real-time RT-PCR. Nucleic Acids Res 29, e45 (2001).

### EXEMPLARY EMBODIMENTS

1. A method of modulating an immune response in a subject, comprising modulating expression or activity of one or more olfactory receptors (OLFR).
2. The method of embodiment 1, wherein the OLFR is expressed by a cell in vivo.
3. The method of embodiment 2, wherein the cell is an animal cell, with the proviso that the cell is not an olfactory cell.
4. The method of any one of embodiments 1-3, wherein the cell is a macrophage.
5. The method of claim 4, wherein the macrophage is a vascular macrophage.
6. The method of any one of embodiments 1-5, wherein the modulation comprises one or more of inhibiting, decreasing, reducing, suppressing, limiting or controlling the immune response by a method comprising administering to the subject an effective amount of an agent that inhibits the expression of or deactivates the OLFR.
7. The method of any one of embodiments 1-5, wherein the modulation comprises one or more of decreasing, reducing, inhibiting, suppressing, limiting or controlling an undesirable or aberrant immune response, immune disorder, inflammatory response or inflammation, by a method comprising administering to the subject an effective amount of an agent that inhibits the expression of or deactivates the OLFR.
8. The method of any one of embodiments 1-5, wherein the modulation comprises one or more of decreasing, reducing, inhibiting, suppressing, limiting or controlling an autoimmune response, disorder or disease in a subject, by a method comprising administering to the subject an effective amount of an agent that inhibits the expression of or deactivates the OLFR.
9. The method of embodiment 8, wherein the modulation comprises one or more of decreasing, reducing, inhibiting, suppressing, limiting or controlling an adverse symptom of the undesirable or aberrant immune response, immune disorder, inflammatory response or inflammation, or an adverse symptom of the autoimmune response, disorder or disease in the subject.
10. The method of embodiment 9, wherein the adverse symptom of the undesirable or aberrant immune response, immune disorder, inflammatory response or inflammation or an adverse symptom of the autoimmune response, disorder or disease is swelling, pain, rash, headache, fever, nausea, diarrhea, bloat, lethargy, skeletal joint stiffness or tissue or cell damage.
11. The method of embodiment 9 or 10, wherein the adverse symptom of the undesirable or aberrant immune response, immune disorder, inflammatory response or inflammation or the adverse symptom of the autoimmune response, disorder or disease is chronic or acute.
12. The method of any one of embodiments 8 to 11, wherein the immune disorder, inflammatory response, inflammation, autoimmune response disorder or autoimmune disease comprises rheumatoid arthritis, juvenile rheumatoid arthritis, osteoarthritis, psoriatic arthritis, diabetes mellitus, multiple sclerosis, encephalomyelitis, myasthenia gravis, systemic lupus erythematosus (SLE), autoimmune thyroiditis, atopic dermatitis, eczematous dermatitis, psoriasis, Sjogren's Syndrome, Crohn's disease, aphthous ulcer, iritis, conjunctivitis, keratoconjunctivitis, ulcerative colitis, inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common g chain (c) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (g5/14.1) deficiency, Hyper-IgM syndrome: X-linked (CD40 ligand deficiency) or non-X-linked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon g receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1 A/SAP deficiency), or hyper IgE syndrome.
13. The method of any one of embodiments 1-5, wherein the modulation comprises one or more of decreasing, reducing, inhibiting, suppressing, limiting or controlling an adverse cardiovascular event or cardiovascular disease by a method comprising administering to the subject an effective amount of an agent that inhibits the expression of or deactivates the OLFR.
14. The method of embodiment 13, wherein the adverse cardiovascular event or cardiovascular disease comprises coronary artery disease, peripheral artery disease, cerebrovascular disease, renal artery disease, stroke, myocardial infarction (heart attack), ischemic heart failure, transient ischemic attack or brain trauma, atherosclerosis, atherosclerotic plaque formation or elevated blood cholesterol.
15. The method of any one of embodiments 1-5, wherein the modulation comprises one or more of decreasing, reducing, inhibiting, suppressing, limiting or controlling atherosclerosis, by a method comprising administering to the subject an effective amount of an agent that inhibits the expression of or deactivates the OLFR.
16. The method of any one of embodiments 1-5, wherein the method comprises one or more of reducing or inhibiting in a subject viral, bacterial or fungal infection, by a method comprising administering to the subject an effective amount of an agent that increases the expression of or activates the OLFR.
17. The method of any one of embodiments 1-5, wherein the modulation comprises one or more of comprising decreasing, reducing, inhibiting, suppressing, limiting or controlling an adverse symptom of the neoplasia, neoplastic disorder, tumor, cancer or malignancy, metastasis of a neoplasia, tumor, cancer or malignancy to other sites, or formation or establishment of a metastatic neoplasia, neoplastic disorder, tumor, cancer or malignancy to other sites distal from a primary neoplasia, neoplastic disorder, tumor, cancer or malignancy, or viral, bacterial or fungal infection by a method comprising administering to the subject an effective amount of an agent that increases the expression of or activates the OLFR.
18. The method of embodiment 17, wherein the neoplasia, neoplastic disorder, tumor, cancer or malignancy treated is a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, lymphosarcoma, liposarcoma, osteosarcoma, chondrosarcoma, leiomyosarcoma, rhabdomyosarcoma, fibrosarcoma or melanoma; or a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, neoplastic disorder, tumor, cancer or malignancy.
19. The method of any one of embodiments 1-18, wherein the OLFR is an OLFR listed in any one of Tables 1-9, and FIGS. 2A and 2B.
20. The method of any one of embodiments 1-18, wherein the OLFR is an OLFR listed in Table 7.
21. The method of any one of embodiments 1-18, wherein the OLFR is selected from the group consisting of OR7C1, OR7D4, OR10A6, OR11H6, OR4E2, OR10H1, and OR6A2.
22. The method of any one of embodiments 6 to 21, wherein the agent is selected from the group of: a ligand or small molecule that binds to the OLFR or blocks the binding of the OLFR to the ligand or an agent that inhibits the expression of the OLFR by the cell.
23. The method of any preceding claim, wherein the agent is selected from the group of an antibody, fragment or mimetic that binds to OLFR or an OLFR ligand, an anti-OLFR gene silencing agent, octanal, heptanal, or a prodrug or solvate thereof.
24. The method of any one of embodiments 6-21, wherein the agent modulates the OLFR by modulating the trafficking of the OLFR to a plasma membrane of a cell.
25. The method of embodiment 24, wherein the agent is a protein selected from the group of receptor transporting protein 1 (RTP1), RTP2, receptor expression enhancing protein 1 (REEP1), aminoacylase 3 (Acy3), or guanine nucleotide-binding protein G(olf) subunit alpha (Gnal).
26. The method of embodiment 24, wherein the agent is a nucleotide encoding a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal).
27. The method of any of embodiments 16-26, wherein the immune response is stimulated by a method comprising administering an agent that increases expression or secretion of an inflammatory cytokine.
28. The method of embodiment 28, wherein the inflammatory cytokine is selected from the group consisting of tumor necrosis factor (TNF), C-C motif chemokine ligand 2 (CCL2), CCL4, CCL5, interleukin 6 (IL-6), IL-1B, IL-18, and nitric oxide synthase 2 (NOS2).
29. A method of suppressing an immune response in a subject in need thereof, the method comprising administering to the subject an agent that decreases the expression of or activity of an olfactory receptor (OLFR), thereby suppressing an immune response in the subject.
30. The method of embodiment 29, wherein the OLFR is an OLFR listed in any one of Tables 1-9.
31. The method of embodiment 29 or 30, wherein the OLFR is an OLFR listed in Table 7.
32. The method of any of embodiments 29-31, wherein the OLFR is selected from the group consisting of OR7C1, OR7D4, OR10A6, OR11H6, OR4E2, OR10H1, and OR6A2.
33. The method of any of embodiments 29-32, wherein the agent is a gene silencing agent.
34. The method of embodiment 23 or 33, wherein the gene silencing agent is selected from the group consisting of a RNA interference (RNAi) molecule, zinc finger nuclease, transcription activator-like effector nuclease (TALEN), and Clustered Regulatory Interspaced Short Palindromic Repeats (CRISPR) enzyme.
35. The method of embodiment 34, wherein the RNAi molecule is selected from the group consisting of a small interference RNA (siRNA), short hairpin RNA (shRNA) and microRNA (miRNA).
36. The method of any of embodiments 29-32, wherein the agent decreases the activity of the OLFR by inhibiting the binding of the OLFR with its ligand.
37. The method of embodiment 36, wherein the ligand is an OLFR ligand listed in Table 8.
38. The method of any of embodiments 29-32, wherein the agent is an OLFR antagonist.
39. The method of embodiment 38, wherein the OLFR antagonist is citral, undecanal, oxyphenylon, phenirat, methyl cinnamaldehyde, hydrocinamaldehyde, bourgeonal, ethylhexanoic acid, α-ionone, octanoic acid, a solvate or prodrug thereof. 40. The method of embodiment 38, wherein the OLFR antagonist is an antagonist listed in Table 9.
41. The method of any of embodiments 29-32, wherein the agent decreases the activity of the OLFR by inhibiting the trafficking of the OLFR to a plasma membrane of a cell.
42. The method of any of embodiments 29-32, wherein the agent is an antibody, fragment or mimetic thereof that binds to an OLFR.
43. A method of increasing an immune response in a subject in need thereof, the method comprising administering to the subject an agent that increases or promotes the trafficking of an olfactory receptor (OLFR) to a plasma membrane of a cell, thereby increasing an immune response in the subject.
44. A method of suppressing an immune response in a subject in need thereof, the method comprising administering to the subject an agent that inhibits the trafficking of an olfactory receptor (OLFR) to a plasma membrane of a cell, thereby suppressing an immune response in the subject.
45. A method of treating an autoimmune disease in a subject in need thereof, the method comprising administering to the subject an agent that decreases the expression of or activity of an olfactory receptor (OLFR).
46. A method of treating an autoimmune disease in a subject in need thereof, the method comprising administering to the subject an agent that inhibits the trafficking of an olfactory receptor (OLFR) to a plasma membrane of a cell.
47. A method of treating a cardiovascular disease in a subject in need thereof, the method comprising administering to the subject an agent that decreases the expression of or activity of an olfactory receptor (OLFR).
48. A method of a cardiovascular disease in a subject in need thereof, the method comprising administering to the subject an agent that inhibits the trafficking of an olfactory receptor (OLFR) to a plasma membrane of a cell.
49. A method of treating an infection in a subject in need thereof, the method comprising administering to the subject an agent that increases the expression of or activates an olfactory receptor (OLFR).
50. A method of treating an infection in a subject in need thereof, the method comprising administering to the subject an agent that increases or promotes the trafficking of an olfactory receptor (OLFR) to a plasma membrane of a cell.
51. The method of embodiment 49 or 50, wherein the infection is selected from the group consisting of a viral infection, bacterial infection, or fungal infection.
52. A method of treating cancer in a subject in need thereof, the method comprising administering to the subject an agent that increases the expression of or activates an olfactory receptor (OLFR).
53. A method of cancer in a subject in need thereof, the method comprising administering to the subject an agent that increases or promotes the trafficking of an olfactory receptor (OLFR) to a plasma membrane of a cell.
54. A method of modulating the expression or activity of one or more proteins involved in an immune response, the method comprising contacting a cell with an agent that modulates the expression or activity of an olfactory receptor (OLFR).
55. The method of embodiment 54, wherein the agent increases the expression of or activates the OLFR.
56. The method of embodiment 54, wherein the agent decreases the expression or activity of the OLFR.
57. A method of increasing the expression or activity of one or more proteins involved in an immune response, the method comprising contacting a cell with an agent that increases the expression of or activates an olfactory receptor (OLFR).
58. A method of decreasing the expression or activity of one or more proteins involved in an immune response, the method comprising contacting a cell with an agent that decreases the expression of or inhibits an olfactory receptor (OLFR).
59. A method of modulating the expression or activity of one or more proteins involved in an immune response, the method comprising contacting a cell with an agent that modulates the trafficking of an olfactory receptor (OLFR) to a plasma membrane of the cell.
60. The method of embodiment 59, wherein agent increases or promotes the trafficking of the OLFR to the plasma membrane of the cell.
61. The method of embodiment 59, wherein the agent decreases or inhibits the trafficking of the OLFR to the plasma membrane of a cell.
62. A method of increasing the expression or activity of one or more proteins involved in an immune response, the method comprising contacting a cell with an agent that increases or promotes the trafficking of an olfactory receptor (OLFR) to a plasma membrane of the cell.
63. A method of decreasing the expression or activity of one or more proteins involved in an immune response, the method comprising contacting a cell with an agent that decreases or inhibits the trafficking of an olfactory receptor (OLFR) to a plasma membrane of a cell.
64. The method of any of embodiments 44-48, 54, 56, 58, 59, 61, and 63, wherein the agent is a gene silencing agent.
65. The method of embodiment 64, wherein the gene silencing agent is selected from the group consisting of a RNA interference (RNAi) molecule, zinc finger nuclease, transcription activator-like effector nuclease (TALEN), and Clustered Regulatory Interspaced Short Palindromic Repeats (CRISPR) enzyme.
66. The method of embodiment 65, wherein the RNAi molecule is selected from the group consisting of a small interference RNA (siRNA), short hairpin RNA (shRNA) and microRNA (miRNA).
67. The method of any of embodiments 45, 47, 56, and 58, wherein the agent decreases the activity of the OLFR by inhibiting the binding of the OLFR with its ligand.
68. The method of embodiment 67, wherein the ligand is an OLFR ligand listed in Table 8.
69. The method of any of embodiments 44-48, 54, 56, 58, 59, 61, and 63, wherein the agent is an OLFR antagonist.
70. The method of embodiment 69, wherein the OLFR antagonist is citral, undecanal, oxyphenylon, phenirat, methyl cinnamaldehyde, hydrocinamaldehyde, bourgeonal, ethylhexanoic acid, α-ionone, octanoic acid, a solvate or prodrug thereof. 71. The method of embodiment 69, wherein the OLFR antagonist is an antagonist listed in Table 9.
72. The method of any of embodiments 45, 47, 56, and 58, wherein the agent decreases the activity of the OLFR by inhibiting the trafficking of the OLFR to a plasma membrane of a cell.
73. The method of any of embodiments 44-48, 54, 56, 58, 59, 61, and 63, wherein the agent is an antibody, fragment or mimetic thereof that binds to an OLFR.
74. The method of any of embodiments 43, 49-55, 57, 59, and 60, wherein the agent is an OLFR agonist.
75. The method of embodiment 74, wherein the OLFR agonist is an agonist listed in Table 8.
76. The method of embodiment 74, wherein the OLFR agonist is selected from the group consisting of octanal, coumarin, helional, lilial, b-ionone, androstenone, androstadienone, caramel furanone, 3-phenyl propyl propionate, eugenol, ethil vanillin, 2-ethyl-fencol, isovaleric acid, nonanoic acid, butyl butyryllactate, butyric acid, isovaleric acid, propionic acid, N-amyl acetate, eugenol acetate, sandalwood, S-(−)-citronellol, S-(−)-citronellal, (+)-carvine, (−) carvone, (+) carvone, linalool, bourgeonal, acetophenone, amyl butyrate, nonanethiol, allyl phenyl acetate, N-amyl acetate, muscone, isoeugenol, eugenol methyl ether, 1-hexanol, 1-heptanole, 1-octanol, celery ketone, anis aldehyde, (+)-menthol, vanillin, guaiacol, lyral, ethyl heptanoate, methyl octanoate, nonanal 1-nonanol, 2-nonanol, 3-octanone, 3-nonanone, decyl aldehyde.
77. The method of any of embodiments 43, 49-55, 57, 59, and 60, wherein the agent is octanal, heptanal, or a prodrug or solvate thereof.
78. The method of any of embodiments 43, 49-55, 57, 59, and 60, wherein the agent is a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal).
79. The method of any of embodiments 43, 49-55, 57, 59, and 60, wherein the agent comprises a nucleotide encoding a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal).
80. The method of any of embodiments 43-79, wherein the OLFR is an OLFR listed in any one of Tables 1-9 and FIGS. 2A-2B.
81. The method of any of embodiments 43-80, wherein the OLFR is an OLFR listed in Table 7.
82. The method of any of embodiments 43-81, wherein the OLFR is selected from the group consisting of OR7C1, OR7D4, OR10A6, OR11H6, OR4E2, OR10H1, and OR6A2.
83. The method of any of embodiments 43-82, wherein the OLFR is expressed by a cell in vivo.
84. The method of embodiment 83, wherein the cell is an animal cell, with the proviso that the cell is not an olfactory cell.
85. The method of embodiment 83 or 84, wherein the cell is a macrophage. 86. The method of claim 85, wherein the macrophage is a vascular macrophage. 87. The method of any one of embodiments 54-63, wherein the one or more proteins involved in an immune response is any of the proteins shown in FIGS. 16 and 17.
88. The method of any one of embodiments 54-63, wherein the one or more proteins involved in an immune response is a protein selected from CCL5 (C-C Motif Chemokine Ligand 5), Tnfrsf12a (Osteoprotegerin), Axin 1, Nadk, Ahr (Aryl hydrocarbon receptor), QDPR (quinoid dihydropteridine reductase), HGF (Hepatocyte Growth Factor), ADAM23, or Snap29.
89. The method of any of embodiments 6 to 53, wherein the administration is local or systemic.
90. The method of embodiment 89, wherein the administration comprises by a method comprising intravenously.
91. The method of any one of embodiments 6 to 53, 89, and 90, wherein the subject is a mammal.
92. The method of any one of embodiments 6 to 53, and 89-91, wherein the subject has or is predisposed to a disease or disorder involving an immune dysregulation.
93. The method of embodiment 91 or 92, wherein the subject is a human patient.
94. A kit comprising an agent that modulates the activity of an OLFR and instructions for use.
95. The kit of any embodiment 94, wherein the agent is a gene silencing agent.
96. The kit of embodiment 95, wherein the gene silencing agent is selected from the group consisting of a RNA interference (RNAi) molecule, zinc finger nuclease, transcription activator-like effector nuclease (TALEN), and Clustered Regulatory Interspaced Short Palindromic Repeats (CRISPR) enzyme.
97. The kit of embodiment 96, wherein the RNAi molecule is selected from the group consisting of a small interference RNA (siRNA), short hairpin RNA (shRNA) and microRNA (miRNA).
98. The kit of any of embodiments 94-97, wherein the agent modulates the activity of the OLFR by inhibiting the binding of the OLFR with its ligand.
99. The kit of embodiment 98, wherein the ligand is an OLFR ligand listed in Table 8.
100. The kit of embodiment 94, wherein the agent is an OLFR antagonist.
101. The kit of embodiment 100, wherein the OLFR antagonist is citral, undecanal, oxyphenylon, phenirat, methyl cinnamaldehyde, hydrocinamaldehyde, bourgeonal, ethylhexanoic acid, α-ionone, octanoic acid, a solvate or prodrug thereof.
102. The kit of embodiment 100, wherein the OLFR antagonist is an antagonist listed in Table 9.
103. The kit of embodiment 94, wherein the agent modulates the activity of the OLFR by inhibiting the trafficking of the OLFR to a plasma membrane of a cell.
104. The kit of embodiment 94, wherein the agent is an antibody, fragment or mimetic thereof that binds to an OLFR.
105. The kit of embodiment 94, wherein the agent is an OLFR agonist.
106. The kit of embodiment 105, wherein the OLFR agonist is an agonist listed in Table 8.
107. The kit of embodiment 105, wherein the OLFR agonist is selected from the group consisting of octanal, coumarin, helional, lilial, b-ionone, androstenone, androstadienone, caramel furanone, 3-phenyl propyl propionate, eugenol, ethil vanillin, 2-ethyl-fencol, isovaleric acid, nonanoic acid, butyl butyryllactate, butyric acid, isovaleric acid, propionic acid, N-amyl acetate, eugenol acetate, sandalwood, S-(−)-citronellol, S-(−)-citronellal, (+)-carvine, (−) carvone, (+) carvone, linalool, bourgeonal, acetophenone, amyl butyrate, nonanethiol, allyl phenyl acetate, N-amyl acetate, muscone, isoeugenol, eugenol methyl ether, 1-hexanol, 1-heptanole, 1-octanol, celery ketone, anis aldehyde, (+)-menthol, vanillin, guaiacol, lyral, ethyl heptanoate, methyl octanoate, nonanal 1-nonanol, 2-nonanol, 3-octanone, 3-nonanone, decyl aldehyde. 108. The kit of embodiment 94, wherein the agent is octanal, heptanal, or a prodrug or solvate thereof.
109. The kit of embodiment 94, wherein the agent is a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal).
110. The kit of embodiment 94, wherein the agent comprises a nucleotide encoding a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal).
111. The kit of any of embodiments 94-110, wherein the OLFR is an OLFR listed in any one of Tables 1-9 and FIGS. 2A-2B.
112. The kit of any of embodiments 94-111, wherein the OLFR is an OLFR listed in Table 7.
113. The kit of any of embodiments 94-112, wherein the OLFR is selected from the group consisting of OR7C1, OR7D4, OR10A6, OR11H6, OR4E2, OR10H1, and OR6A2.

TABLE 1
Olfactory receptors and accessory genes GAPDH ranked
expression as displayed in the heatmap of FIG. 1

| | Aortic | Aortic | BMDM | BMDM | | | IP | | MLN | Lung |
|---|---|---|---|---|---|---|---|---|---|---|
| | Mφ | Mφ | Mφ − | Mφ | SI | SI | Mφ | IP | DCs | DCs |
| | Naive | LPS | M0 | LPS | DCs | Mφ | LPS | Mφ | CD8+ | 103+ |
| Gapdh | 1.00 | 1.00 | 1.00 | 1.00 | 1.00 | 1.00 | 1.00 | 1.00 | 1.00 | 1.00 |
| Rtp1 | 0.35 | 0.33 | 0.30 | 0.30 | 0.28 | 0.30 | 0.28 | 0.25 | 0.32 | 0.29 |
| Rtp2 | 0.39 | 0.41 | 0.33 | 0.38 | 0.38 | 0.37 | 0.31 | 0.29 | 0.34 | 0.35 |
| Reep3 | 0.894 | 0.921 | 0.694 | 0.765 | 0.599 | 0.718 | 0.603 | 0.675 | 0.616 | 0.678 |
| Cnga1 | 0.16 | 0.163 | 0.136 | 0.162 | 0.149 | 0.169 | 0.123 | 0.109 | 0.133 | 0.107 |
| Cnga2 | 0.169 | 0.156 | 0.142 | 0.169 | 0.201 | 0.199 | 0.156 | 0.138 | 0.185 | 0.169 |
| Cnga3 | 0.291 | 0.286 | 0.249 | 0.254 | 0.273 | 0.297 | 0.235 | 0.216 | 0.295 | 0.253 |
| Cnga4 | 0.235 | 0.206 | 0.23 | 0.245 | 0.232 | 0.249 | 0.213 | 0.181 | 0.256 | 0.225 |
| Cngb1 | 0.914 | 0.929 | 0.314 | 0.31 | 0.393 | 0.412 | 0.26 | 0.256 | 0.27 | 0.247 |
| Acy3 | 0.529 | 0.535 | 0.451 | 0.465 | 0.395 | 0.502 | 0.435 | 0.443 | 0.379 | 0.342 |
| Gna1 | 0.24 | 0.24 | 0.23 | 0.22 | 0.29 | 0.26 | 0.15 | 0.16 | 0.17 | 0.17 |
| Olfr1 | 0.23 | 0.25 | 0.22 | 0.25 | 0.23 | 0.22 | 0.21 | 0.20 | 0.23 | 0.20 |
| Olfr10 | 0.47 | 0.48 | 0.23 | 0.25 | 0.24 | 0.27 | 0.21 | 0.19 | 0.22 | 0.22 |
| Olfr100 | 0.28 | 0.26 | 0.18 | 0.16 | 0.14 | 0.14 | 0.16 | 0.14 | 0.19 | 0.15 |
| Olfr1000 | 0.08 | 0.11 | 0.07 | 0.09 | 0.17 | 0.16 | 0.08 | 0.08 | 0.10 | 0.09 |
| Olfr1002 | 0.02 | 0.02 | 0.04 | 0.06 | 0.05 | 0.06 | 0.07 | 0.05 | 0.04 | 0.03 |
| Olfr1006 | 0.22 | 0.23 | 0.21 | 0.22 | 0.22 | 0.25 | 0.21 | 0.17 | 0.19 | 0.20 |
| Olfr1008 | 0.12 | 0.17 | 0.13 | 0.14 | 0.17 | 0.18 | 0.08 | 0.08 | 0.13 | 0.10 |
| Olfr1009 | 0.27 | 0.27 | 0.16 | 0.18 | 0.18 | 0.20 | 0.17 | 0.16 | 0.18 | 0.16 |
| Olfr101 | 0.26 | 0.26 | 0.21 | 0.26 | 0.19 | 0.24 | 0.17 | 0.16 | 0.18 | 0.17 |
| Olfr1010 | 0.33 | 0.31 | 0.21 | 0.23 | 0.25 | 0.26 | 0.21 | 0.19 | 0.25 | 0.19 |
| Olfr1012 | 0.31 | 0.35 | 0.25 | 0.31 | 0.25 | 0.26 | 0.22 | 0.21 | 0.24 | 0.22 |
| Olfr1013 | 0.08 | 0.11 | 0.09 | 0.12 | 0.12 | 0.12 | 0.09 | 0.09 | 0.06 | 0.06 |
| Olfr1014 | 0.11 | 0.16 | 0.07 | 0.09 | 0.10 | 0.13 | 0.08 | 0.09 | 0.05 | 0.07 |
| Olfr1015 | 0.26 | 0.30 | 0.22 | 0.26 | 0.24 | 0.27 | 0.20 | 0.20 | 0.21 | 0.18 |
| Olfr1016 | 0.31 | 0.30 | 0.22 | 0.27 | 0.23 | 0.28 | 0.24 | 0.21 | 0.26 | 0.22 |
| Olfr1018 | 0.16 | 0.15 | 0.14 | 0.16 | 0.18 | 0.19 | 0.15 | 0.14 | 0.16 | 0.14 |
| Olfr1019 | 0.18 | 0.22 | 0.19 | 0.22 | 0.21 | 0.21 | 0.20 | 0.17 | 0.20 | 0.18 |
| Olfr102 | 0.28 | 0.26 | 0.18 | 0.16 | 0.14 | 0.14 | 0.16 | 0.14 | 0.19 | 0.15 |
| Olfr1020 | 0.10 | 0.15 | 0.10 | 0.09 | 0.10 | 0.09 | 0.09 | 0.07 | 0.06 | 0.05 |
| Olfr1022 | 0.17 | 0.19 | 0.12 | 0.08 | 0.12 | 0.15 | 0.09 | 0.08 | 0.10 | 0.08 |
| Olfr1023 | 0.13 | 0.17 | 0.13 | 0.14 | 0.13 | 0.15 | 0.12 | 0.10 | 0.13 | 0.10 |
| Olfr1024 | 0.21 | 0.23 | 0.17 | 0.20 | 0.22 | 0.28 | 0.16 | 0.15 | 0.19 | 0.19 |
| Olfr1026 | 0.12 | 0.11 | 0.12 | 0.12 | 0.14 | 0.15 | 0.11 | 0.09 | 0.11 | 0.10 |
| Olfr1028 | 0.24 | 0.31 | 0.16 | 0.17 | 0.18 | 0.19 | 0.08 | 0.08 | 0.09 | 0.09 |
| Olfr1029 | 0.58 | 0.59 | 0.24 | 0.25 | 0.26 | 0.27 | 0.15 | 0.18 | 0.19 | 0.14 |
| Olfr103 | 0.22 | 0.20 | 0.17 | 0.20 | 0.15 | 0.15 | 0.15 | 0.12 | 0.15 | 0.15 |
| Olfr1030 | 0.21 | 0.23 | 0.11 | 0.13 | 0.11 | 0.12 | 0.12 | 0.11 | 0.09 | 0.09 |
| Olfr1031 | 0.12 | 0.13 | 0.11 | 0.11 | 0.08 | 0.09 | 0.08 | 0.09 | 0.07 | 0.07 |
| Olfr1032 | 0.24 | 0.22 | 0.16 | 0.16 | 0.16 | 0.16 | 0.13 | 0.13 | 0.15 | 0.12 |
| Olfr1033 | 0.07 | 0.09 | 0.06 | 0.06 | 0.04 | 0.07 | 0.03 | 0.05 | 0.03 | 0.03 |
| Olfr1034 | 0.20 | 0.18 | 0.16 | 0.16 | 0.17 | 0.21 | 0.19 | 0.17 | 0.20 | 0.17 |
| Olfr1036 | 0.20 | 0.18 | 0.16 | 0.16 | 0.17 | 0.21 | 0.19 | 0.17 | 0.20 | 0.17 |
| Olfr1037 | 0.04 | 0.04 | 0.05 | 0.08 | 0.08 | 0.07 | 0.04 | 0.04 | 0.04 | 0.04 |
| Olfr1038-ps | 0.16 | 0.27 | 0.11 | 0.11 | 0.13 | 0.12 | 0.07 | 0.06 | 0.11 | 0.07 |
| Olfr1039 | 0.05 | 0.02 | 0.02 | 0.04 | 0.02 | 0.04 | NA | NA | 0.02 | 0.02 |
| Olfr1040 | 0.24 | 0.28 | 0.20 | 0.22 | 0.23 | 0.24 | 0.18 | 0.18 | 0.19 | 0.20 |
| Olfr1042 | 0.30 | 0.31 | 0.32 | 0.36 | 0.30 | 0.36 | 0.24 | 0.24 | 0.24 | 0.23 |
| Olfr1043 | 0.26 | 0.30 | 0.21 | 0.24 | 0.17 | 0.19 | 0.18 | 0.17 | 0.18 | 0.16 |
| Olfr1044 | 0.12 | 0.14 | 0.14 | 0.15 | 0.14 | 0.15 | 0.14 | 0.12 | 0.10 | 0.09 |
| Olfr1045 | 0.20 | 0.25 | 0.10 | 0.09 | 0.17 | 0.17 | 0.13 | 0.11 | 0.15 | 0.14 |
| Olfr1046 | 0.02 | 0.03 | 0.03 | 0.06 | 0.03 | 0.06 | 0.03 | 0.01 | 0.02 | 0.01 |
| Olfr1047 | 0.00 | 0.00 | NA | NA | NA | 0.01 | NA | NA | 0.01 | 0.00 |
| Olfr1048 | 0.16 | 0.16 | 0.10 | 0.14 | 0.19 | 0.19 | 0.12 | 0.11 | 0.12 | 0.12 |
| Olfr1049 | NA | NA | 0.04 | 0.05 | 0.08 | 0.02 | 0.00 | NA | 0.01 | 0.00 |
| Olfr1051 | NA | NA | NA | 0.07 | 0.02 | 0.02 | NA | NA | NA | NA |
| Olfr1052 | 0.07 | 0.08 | 0.11 | 0.09 | 0.11 | 0.12 | 0.08 | 0.06 | 0.10 | 0.08 |
| Olfr1053 | 0.16 | 0.21 | 0.18 | 0.20 | 0.22 | 0.24 | 0.16 | 0.12 | 0.18 | 0.22 |
| Olfr1054 | 0.24 | 0.28 | 0.17 | 0.18 | 0.23 | 0.21 | 0.15 | 0.14 | 0.16 | 0.18 |
| Olfr1055 | 0.08 | 0.10 | 0.09 | 0.10 | 0.09 | 0.10 | 0.09 | 0.06 | 0.09 | 0.07 |
| Olfr1056 | 0.17 | 0.22 | 0.11 | 0.13 | 0.18 | 0.17 | 0.15 | 0.12 | 0.15 | 0.15 |
| Olfr1057 | 0.22 | 0.24 | 0.18 | 0.20 | 0.18 | 0.19 | 0.13 | 0.12 | 0.13 | 0.13 |
| Olfr1058 | 0.12 | 0.18 | 0.10 | 0.10 | 0.10 | 0.12 | 0.08 | 0.07 | 0.07 | 0.06 |
| Olfr106-ps | 0.02 | 0.02 | 0.02 | 0.13 | 0.04 | 0.06 | 0.02 | 0.01 | 0.01 | 0.02 |
| Olfr1061 | NA | 0.01 | 0.01 | 0.02 | 0.04 | 0.03 | NA | 0.00 | NA | 0.01 |
| Olfr1062 | 0.17 | 0.16 | 0.13 | 0.18 | 0.16 | 0.19 | 0.16 | 0.14 | 0.14 | 0.16 |
| Olfr1065 | 0.12 | 0.18 | 0.10 | 0.10 | 0.10 | 0.12 | 0.08 | 0.07 | 0.07 | 0.06 |
| Olfr1066 | 0.07 | 0.10 | 0.02 | 0.04 | 0.04 | 0.03 | 0.01 | 0.00 | NA | 0.00 |
| Olfr107 | 0.04 | 0.06 | 0.13 | 0.24 | 0.11 | 0.14 | 0.05 | 0.05 | 0.08 | 0.05 |
| Olfr1076 | 0.36 | 0.39 | 0.09 | 0.14 | 0.14 | 0.18 | 0.06 | 0.06 | 0.07 | 0.05 |
| Olfr1077-ps1 | 0.28 | 0.32 | 0.21 | 0.22 | 0.24 | 0.30 | 0.18 | 0.18 | 0.23 | 0.20 |
| Olfr1079 | 0.09 | 0.13 | 0.06 | 0.09 | 0.10 | 0.08 | 0.05 | 0.05 | 0.09 | 0.05 |
| Olfr108 | 0.12 | 0.16 | 0.10 | 0.27 | 0.12 | 0.09 | 0.05 | 0.07 | 0.07 | 0.05 |
| Olfr1080 | 0.01 | 0.03 | 0.03 | 0.05 | 0.10 | 0.09 | 0.00 | NA | 0.02 | NA |
| Olfr1082 | 0.02 | 0.06 | NA | 0.02 | 0.02 | 0.05 | 0.00 | NA | NA | NA |
| Olfr1084 | 0.03 | 0.10 | 0.02 | 0.02 | 0.01 | NA | NA | NA | NA | NA |
| Olfr1085 | 0.19 | 0.28 | 0.17 | 0.23 | 0.25 | 0.31 | 0.18 | 0.15 | 0.16 | 0.16 |
| Olfr1086 | 0.27 | 0.24 | 0.26 | 0.29 | 0.25 | 0.28 | 0.21 | 0.20 | 0.22 | 0.21 |
| Olfr1087 | 0.14 | 0.17 | 0.11 | 0.14 | 0.14 | 0.16 | 0.08 | 0.08 | 0.10 | 0.11 |
| Olfr1089 | 0.17 | 0.18 | 0.12 | 0.16 | 0.12 | 0.13 | 0.12 | 0.09 | 0.11 | 0.08 |
| Olfr109 | 0.31 | 0.28 | 0.27 | 0.48 | 0.24 | 0.25 | 0.26 | 0.25 | 0.27 | 0.25 |
| Olfr1090 | 0.11 | 0.13 | 0.12 | 0.10 | 0.12 | 0.14 | 0.08 | 0.08 | 0.10 | 0.09 |
| Olfr1093 | 0.20 | 0.20 | 0.21 | 0.20 | 0.22 | 0.23 | 0.16 | 0.15 | 0.19 | 0.16 |
| Olfr1094 | 0.07 | 0.06 | 0.08 | 0.10 | 0.13 | 0.13 | 0.08 | 0.07 | 0.09 | 0.07 |
| Olfr1095 | 0.00 | 0.02 | 0.01 | 0.12 | 0.04 | 0.10 | 0.04 | 0.02 | 0.05 | 0.03 |
| Olfr1097 | 0.15 | 0.17 | 0.08 | 0.10 | 0.13 | 0.13 | 0.10 | 0.07 | 0.04 | 0.07 |
| Olfr1098 | 0.09 | 0.12 | 0.10 | 0.09 | 0.09 | 0.12 | 0.08 | 0.07 | 0.05 | 0.06 |
| Olfr1099 | 0.26 | 0.31 | 0.21 | 0.21 | 0.24 | 0.25 | 0.17 | 0.13 | 0.16 | 0.18 |
| Olfr11 | 0.31 | 0.30 | 0.23 | 0.30 | 0.30 | 0.33 | 0.23 | 0.20 | 0.25 | 0.23 |
| Olfr110 | 0.22 | 0.24 | 0.52 | 0.45 | 0.22 | 0.23 | 0.17 | 0.15 | 0.19 | 0.18 |
| Olfr1100 | 0.10 | 0.12 | 0.05 | 0.11 | 0.09 | 0.12 | 0.03 | 0.03 | 0.04 | 0.02 |
| Olfr1101 | 0.17 | 0.17 | 0.14 | 0.13 | 0.16 | 0.16 | 0.09 | 0.09 | 0.10 | 0.12 |
| Olfr1102 | 0.04 | 0.07 | 0.03 | 0.03 | 0.04 | 0.05 | 0.03 | 0.02 | NA | 0.00 |
| Olfr1104 | 0.24 | 0.24 | 0.16 | 0.17 | 0.16 | 0.19 | 0.13 | 0.12 | 0.14 | 0.14 |
| Olfr1105 | 0.23 | 0.24 | 0.20 | 0.21 | 0.19 | 0.26 | 0.18 | 0.16 | 0.22 | 0.17 |
| Olfr1106 | 0.17 | 0.16 | 0.10 | 0.10 | 0.11 | 0.11 | 0.12 | 0.11 | 0.11 | 0.08 |
| Olfr1107 | 0.09 | 0.10 | 0.06 | 0.07 | 0.10 | 0.12 | 0.04 | 0.02 | 0.03 | 0.02 |
| Olfr1109 | 0.08 | 0.10 | 0.06 | 0.10 | 0.12 | 0.12 | 0.06 | 0.05 | 0.07 | 0.04 |
| Olfr111 | 0.26 | 0.27 | 0.72 | 0.68 | 0.18 | 0.23 | 0.20 | 0.21 | 0.14 | 0.14 |
| Olfr1110 | 0.16 | 0.19 | 0.13 | 0.10 | 0.13 | 0.13 | 0.09 | 0.07 | 0.11 | 0.11 |
| Olfr1111 | 0.20 | 0.20 | 0.15 | 0.16 | 0.18 | 0.15 | 0.12 | 0.10 | 0.13 | 0.11 |
| Olfr1112 | 0.08 | 0.09 | 0.07 | 0.08 | 0.10 | 0.09 | 0.05 | 0.04 | 0.05 | 0.05 |
| Olfr1113 | 0.21 | 0.27 | 0.15 | 0.16 | 0.15 | 0.17 | 0.10 | 0.10 | 0.12 | 0.10 |
| Olfr1115 | 0.29 | 0.30 | 0.19 | 0.22 | 0.23 | 0.26 | 0.19 | 0.18 | 0.21 | 0.17 |
| Olfr1116 | 0.08 | 0.16 | 0.05 | 0.05 | 0.07 | 0.06 | 0.04 | 0.04 | 0.02 | 0.00 |
| Olfr1118 | 0.27 | 0.28 | 0.15 | 0.20 | 0.17 | 0.16 | 0.13 | 0.12 | 0.18 | 0.13 |
| Olfr112 | 0.07 | 0.11 | 0.46 | 0.49 | 0.10 | 0.11 | 0.05 | 0.05 | 0.03 | 0.04 |
| Olfr1120 | 0.21 | 0.21 | 0.13 | 0.16 | 0.16 | 0.14 | 0.13 | 0.13 | 0.14 | 0.12 |
| Olfr1121 | 0.10 | 0.11 | 0.10 | 0.12 | 0.14 | 0.16 | 0.09 | 0.08 | 0.09 | 0.09 |
| Olfr1122 | 0.25 | 0.25 | 0.21 | 0.24 | 0.24 | 0.28 | 0.17 | 0.16 | 0.19 | 0.17 |
| Olfr1123 | 0.03 | 0.05 | 0.05 | 0.07 | 0.04 | 0.03 | 0.08 | 0.06 | 0.07 | 0.06 |
| Olfr1124 | 0.13 | 0.15 | 0.02 | 0.06 | 0.07 | 0.09 | 0.02 | 0.03 | 0.01 | NA |
| Olfr1126 | 0.30 | 0.33 | 0.16 | 0.25 | 0.20 | 0.22 | 0.18 | 0.17 | 0.20 | 0.18 |
| Olfr1128 | 0.33 | 0.32 | 0.24 | 0.24 | 0.25 | 0.27 | 0.22 | 0.19 | 0.22 | 0.19 |
| Olfr1129 | 0.14 | 0.18 | 0.09 | 0.13 | 0.11 | 0.10 | 0.09 | 0.09 | 0.08 | 0.08 |
| Olfr113 | 0.40 | 0.42 | 0.49 | 0.54 | 0.27 | 0.26 | 0.21 | 0.21 | 0.21 | 0.18 |
| Olfr1130 | 0.10 | 0.14 | 0.12 | 0.17 | 0.15 | 0.18 | 0.07 | 0.05 | 0.07 | 0.07 |
| Olfr1131 | 0.18 | 0.18 | 0.11 | 0.13 | 0.15 | 0.15 | 0.13 | 0.12 | 0.11 | 0.09 |
| Olfr1132 | 0.22 | 0.21 | 0.15 | 0.21 | 0.22 | 0.25 | 0.17 | 0.14 | 0.14 | 0.16 |
| Olfr1133 | 0.31 | 0.31 | 0.21 | 0.27 | 0.27 | 0.29 | 0.18 | 0.17 | 0.21 | 0.19 |
| Olfr1134 | 0.06 | 0.04 | 0.05 | 0.07 | 0.09 | 0.10 | 0.07 | 0.10 | 0.12 | 0.06 |
| Olfr1135 | 0.08 | 0.12 | 0.06 | 0.14 | 0.16 | 0.19 | 0.07 | 0.06 | 0.05 | 0.04 |
| Olfr1136 | 0.24 | 0.22 | 0.15 | 0.19 | 0.15 | 0.14 | 0.14 | 0.11 | 0.15 | 0.13 |
| Olfr1137 | 0.28 | 0.29 | 0.19 | 0.18 | 0.23 | 0.22 | 0.16 | 0.14 | 0.15 | 0.15 |
| Olfr1138 | 0.17 | 0.19 | 0.15 | 0.13 | 0.18 | 0.16 | 0.13 | 0.12 | 0.16 | 0.13 |
| Olfr114 | 0.24 | 0.26 | 0.41 | 0.37 | 0.19 | 0.17 | 0.12 | 0.10 | 0.12 | 0.11 |
| Olfr1140 | 0.18 | 0.23 | 0.10 | 0.11 | 0.14 | 0.17 | 0.10 | 0.10 | 0.11 | 0.12 |
| Olfr1141 | 0.12 | 0.15 | 0.07 | 0.10 | 0.12 | 0.13 | 0.08 | 0.08 | 0.09 | 0.07 |
| Olfr1143 | 0.12 | 0.16 | 0.08 | 0.09 | 0.11 | 0.13 | 0.09 | 0.07 | 0.08 | 0.07 |
| Olfr1145 | 0.12 | 0.17 | 0.13 | 0.14 | 0.13 | 0.15 | 0.11 | 0.09 | 0.11 | 0.10 |
| Olfr1148 | 0.42 | 0.44 | 0.31 | 0.31 | 0.37 | 0.37 | 0.40 | 0.40 | 0.27 | 0.31 |
| Olfr115 | 0.24 | 0.25 | 0.35 | 0.35 | 0.23 | 0.23 | 0.23 | 0.21 | 0.21 | 0.19 |
| Olfr1151 | 0.19 | 0.19 | 0.14 | 0.16 | 0.16 | 0.16 | 0.12 | 0.10 | 0.14 | 0.11 |
| Olfr1152 | 0.48 | 0.49 | 0.37 | 0.42 | 0.44 | 0.50 | 0.32 | 0.33 | 0.37 | 0.31 |
| Olfr1153 | 0.21 | 0.23 | 0.18 | 0.16 | 0.16 | 0.19 | 0.16 | 0.14 | 0.15 | 0.16 |
| Olfr1154 | 0.17 | 0.17 | 0.11 | 0.14 | 0.14 | 0.13 | 0.09 | 0.08 | 0.12 | 0.09 |
| Olfr1155 | 0.35 | 0.38 | 0.30 | 0.32 | 0.32 | 0.34 | 0.27 | 0.25 | 0.28 | 0.26 |
| Olfr1156 | 0.28 | 0.25 | 0.22 | 0.27 | 0.24 | 0.27 | 0.18 | 0.18 | 0.22 | 0.20 |
| Olfr1157 | 0.12 | 0.14 | 0.13 | 0.15 | 0.13 | 0.15 | 0.14 | 0.12 | 0.14 | 0.16 |
| Olfr1158 | 0.19 | 0.21 | 0.10 | 0.14 | 0.13 | 0.13 | 0.13 | 0.12 | 0.09 | 0.11 |
| Olfr116 | 0.12 | 0.16 | 0.15 | 0.18 | 0.10 | 0.11 | 0.09 | 0.08 | 0.10 | 0.09 |
| Olfr1160 | 0.22 | 0.28 | NA | 0.03 | 0.02 | 0.01 | 0.03 | 0.01 | NA | NA |
| Olfr1161 | 0.10 | 0.13 | 0.11 | 0.12 | 0.15 | 0.18 | 0.11 | 0.10 | 0.11 | 0.09 |
| Olfr1162 | 0.26 | 0.29 | 0.22 | 0.23 | 0.26 | 0.27 | 0.26 | 0.24 | 0.25 | 0.21 |
| Olfr1163 | 0.26 | 0.29 | 0.22 | 0.23 | 0.26 | 0.27 | 0.26 | 0.24 | 0.25 | 0.21 |
| Olfr1164 | 0.09 | 0.08 | 0.10 | 0.11 | 0.16 | 0.18 | 0.08 | 0.08 | 0.08 | 0.08 |
| Olfr1166 | 0.22 | 0.29 | 0.12 | 0.08 | 0.11 | 0.14 | 0.12 | 0.12 | 0.10 | 0.07 |
| Olfr1167 | 0.06 | 0.11 | 0.01 | 0.05 | 0.05 | 0.08 | 0.06 | 0.02 | 0.01 | 0.01 |
| Olfr1168 | 0.14 | 0.14 | 0.15 | 0.15 | 0.17 | 0.18 | 0.12 | 0.13 | 0.13 | 0.09 |
| Olfr117 | 0.15 | 0.13 | 0.12 | 0.15 | 0.14 | 0.13 | 0.11 | 0.12 | 0.13 | 0.12 |
| Olfr1170 | 0.13 | 0.15 | 0.10 | 0.12 | 0.12 | 0.14 | 0.15 | 0.12 | 0.08 | 0.09 |
| Olfr1173 | 0.11 | 0.12 | 0.09 | 0.09 | 0.13 | 0.15 | 0.08 | 0.09 | 0.07 | 0.06 |
| Olfr1174-ps | 0.79 | 0.80 | 0.62 | 0.67 | 0.61 | 0.63 | 0.57 | 0.52 | 0.66 | 0.63 |
| Olfr1176 | 0.09 | 0.09 | 0.07 | 0.11 | 0.11 | 0.13 | 0.09 | 0.07 | 0.06 | 0.07 |
| Olfr1178 | 0.18 | 0.20 | 0.10 | 0.10 | 0.11 | 0.12 | 0.08 | 0.06 | 0.09 | 0.07 |
| Olfr1179 | 0.28 | 0.27 | 0.22 | 0.21 | 0.21 | 0.27 | 0.16 | 0.18 | 0.18 | 0.16 |
| Olfr118 | 0.15 | 0.15 | 0.14 | 0.18 | 0.21 | 0.24 | 0.11 | 0.11 | 0.15 | 0.15 |
| Olfr1180 | 0.13 | 0.16 | 0.08 | 0.11 | 0.09 | 0.08 | 0.09 | 0.07 | 0.07 | 0.07 |
| Olfr1181 | 0.30 | 0.31 | 0.05 | 0.07 | 0.12 | 0.13 | 0.04 | 0.04 | 0.04 | 0.02 |
| Olfr1182 | 0.36 | 0.40 | 0.15 | 0.17 | 0.17 | 0.19 | 0.13 | 0.11 | 0.09 | 0.13 |
| Olfr1183 | 0.11 | 0.20 | 0.05 | 0.07 | 0.10 | 0.08 | 0.03 | 0.01 | 0.02 | 0.02 |
| Olfr1184 | 0.08 | 0.10 | 0.08 | 0.07 | 0.08 | 0.12 | 0.10 | 0.07 | 0.05 | 0.06 |
| Olfr1186 | 0.07 | 0.07 | 0.03 | 0.07 | 0.04 | 0.06 | 0.05 | 0.05 | 0.05 | 0.03 |
| Olfr1188 | 0.20 | 0.21 | 0.12 | 0.16 | 0.17 | 0.17 | 0.14 | 0.14 | 0.14 | 0.10 |
| Olfr1189 | 0.10 | 0.10 | 0.07 | 0.11 | 0.16 | 0.16 | 0.08 | 0.07 | 0.06 | 0.06 |
| Olfr119 | 0.19 | 0.21 | 0.12 | 0.13 | 0.23 | 0.20 | 0.08 | 0.06 | 0.14 | 0.11 |
| Olfr1193 | 0.23 | 0.25 | 0.15 | 0.11 | 0.11 | 0.12 | 0.08 | 0.08 | 0.11 | 0.09 |
| Olfr1195 | 0.06 | 0.04 | 0.04 | 0.08 | 0.10 | 0.11 | 0.05 | 0.03 | 0.04 | 0.04 |
| Olfr1196 | 0.25 | 0.30 | 0.17 | 0.19 | 0.18 | 0.17 | 0.17 | 0.15 | 0.17 | 0.14 |
| Olfr1197 | 0.25 | 0.30 | 0.17 | 0.19 | 0.18 | 0.17 | 0.17 | 0.15 | 0.17 | 0.14 |
| Olfr1198 | 0.05 | 0.07 | 0.06 | 0.06 | 0.10 | 0.11 | 0.03 | 0.02 | 0.04 | 0.04 |
| Olfr1199 | 0.10 | 0.18 | 0.05 | 0.07 | 0.08 | 0.12 | 0.02 | 0.01 | 0.02 | 0.01 |
| Olfr12 | 0.38 | 0.35 | 0.28 | 0.29 | 0.28 | 0.31 | 0.25 | 0.23 | 0.27 | 0.28 |
| Olfr120 | 0.15 | 0.16 | 0.07 | 0.12 | 0.13 | 0.13 | 0.11 | 0.10 | 0.11 | 0.13 |
| Olfr1200 | 0.36 | 0.48 | 0.25 | 0.21 | 0.22 | 0.25 | 0.24 | 0.22 | 0.25 | 0.21 |
| Olfr1201 | 0.18 | 0.24 | 0.14 | 0.15 | 0.14 | 0.18 | 0.18 | 0.16 | 0.13 | 0.12 |
| Olfr1202 | 0.15 | 0.18 | 0.08 | 0.11 | 0.11 | 0.09 | 0.07 | 0.07 | 0.09 | 0.06 |
| Olfr1204 | 0.07 | 0.08 | 0.07 | 0.12 | 0.07 | 0.10 | 0.07 | 0.05 | 0.08 | 0.08 |
| Olfr1205 | 0.18 | 0.24 | 0.14 | 0.15 | 0.14 | 0.18 | 0.18 | 0.16 | 0.13 | 0.12 |
| Olfr1206 | 0.18 | 0.24 | 0.14 | 0.15 | 0.14 | 0.18 | 0.18 | 0.16 | 0.13 | 0.12 |
| Olfr1208 | 0.08 | 0.14 | 0.11 | 0.08 | 0.13 | 0.14 | 0.09 | 0.09 | 0.06 | 0.08 |
| Olfr1209 | 0.21 | 0.26 | 0.15 | 0.17 | 0.15 | 0.19 | 0.15 | 0.16 | 0.16 | 0.14 |
| Olfr121 | 0.23 | 0.29 | 0.19 | 0.20 | 0.20 | 0.21 | 0.12 | 0.14 | 0.13 | 0.09 |
| Olfr1211 | 0.16 | 0.18 | 0.11 | 0.10 | 0.12 | 0.10 | 0.08 | 0.08 | 0.09 | 0.08 |
| Olfr1212 | 0.04 | 0.06 | 0.03 | 0.01 | 0.04 | 0.05 | 0.02 | 0.02 | 0.03 | 0.03 |
| Olfr1213 | 0.13 | 0.18 | 0.10 | 0.13 | 0.16 | 0.18 | 0.09 | 0.07 | 0.08 | 0.08 |
| Olfr1214 | 0.04 | 0.10 | 0.05 | 0.06 | 0.12 | 0.12 | 0.02 | 0.04 | 0.07 | 0.06 |
| Olfr1215 | 0.09 | 0.11 | 0.10 | 0.13 | 0.12 | 0.16 | 0.09 | 0.07 | 0.06 | 0.06 |
| Olfr1216 | 0.06 | 0.07 | 0.07 | 0.08 | 0.06 | 0.03 | 0.04 | 0.04 | 0.05 | 0.04 |
| Olfr1217 | 0.29 | 0.28 | 0.07 | 0.05 | 0.09 | 0.07 | 0.01 | 0.02 | 0.02 | 0.02 |
| Olfr1218 | 0.07 | 0.09 | 0.08 | 0.08 | 0.10 | 0.13 | 0.06 | 0.05 | 0.06 | 0.06 |
| Olfr1219 | 0.17 | 0.18 | 0.10 | 0.13 | 0.12 | 0.11 | 0.09 | 0.08 | 0.09 | 0.08 |
| Olfr122 | 0.30 | 0.34 | 0.26 | 0.32 | 0.32 | 0.32 | 0.20 | 0.18 | 0.24 | 0.20 |
| Olfr1220 | 0.13 | 0.16 | 0.07 | 0.07 | 0.11 | 0.12 | 0.08 | 0.06 | 0.06 | 0.07 |
| Olfr1221 | 0.03 | 0.06 | 0.04 | 0.08 | 0.09 | 0.06 | NA | 0.00 | 0.02 | NA |
| Olfr1222 | 0.31 | 0.34 | 0.23 | 0.26 | 0.28 | 0.32 | 0.26 | 0.24 | 0.20 | 0.21 |
| Olfr1223 | 0.14 | 0.14 | 0.11 | 0.12 | 0.14 | 0.12 | 0.09 | 0.08 | 0.08 | 0.05 |
| Olfr1225 | 0.18 | 0.20 | 0.15 | 0.16 | 0.16 | 0.18 | 0.13 | 0.12 | 0.13 | 0.12 |
| Olfr1226 | 0.29 | 0.32 | 0.18 | 0.18 | 0.22 | 0.20 | 0.18 | 0.14 | 0.16 | 0.14 |
| Olfr1228 | 0.09 | 0.11 | 0.05 | 0.08 | 0.07 | 0.06 | 0.04 | 0.04 | 0.04 | 0.04 |
| Olfr1229 | 0.43 | 0.44 | 0.38 | 0.40 | 0.38 | 0.39 | 0.31 | 0.28 | 0.36 | 0.30 |
| Olfr123 | 0.31 | 0.30 | 0.22 | 0.27 | 0.25 | 0.28 | 0.20 | 0.19 | 0.24 | 0.21 |
| Olfr1230 | 0.19 | 0.22 | 0.21 | 0.23 | 0.19 | 0.21 | 0.18 | 0.16 | 0.18 | 0.16 |
| Olfr1231 | 0.21 | 0.22 | 0.15 | 0.15 | 0.12 | 0.15 | 0.14 | 0.15 | 0.16 | 0.14 |
| Olfr1232 | 0.06 | 0.05 | 0.08 | 0.08 | 0.09 | 0.07 | 0.03 | 0.03 | 0.04 | 0.03 |
| Olfr1233 | 0.26 | 0.27 | 0.16 | 0.16 | 0.16 | 0.15 | 0.17 | 0.15 | 0.19 | 0.15 |
| Olfr1234 | 0.15 | 0.15 | 0.11 | 0.13 | 0.16 | 0.17 | 0.11 | 0.09 | 0.12 | 0.10 |
| Olfr1238 | 0.27 | 0.32 | 0.21 | 0.18 | 0.19 | 0.24 | 0.15 | 0.14 | 0.20 | 0.15 |
| Olfr1239 | 0.26 | 0.29 | 0.18 | 0.22 | 0.18 | 0.23 | 0.20 | 0.16 | 0.17 | 0.19 |
| Olfr124 | 0.17 | 0.17 | 0.18 | 0.21 | 0.19 | 0.21 | 0.13 | 0.12 | 0.21 | 0.15 |
| Olfr1240 | 0.13 | 0.13 | 0.08 | 0.13 | 0.13 | 0.15 | 0.10 | 0.10 | 0.10 | 0.08 |
| Olfr1241 | 0.14 | 0.20 | 0.10 | 0.10 | 0.13 | 0.16 | 0.09 | 0.08 | 0.12 | 0.10 |
| Olfr1242 | 0.21 | 0.26 | 0.14 | 0.18 | 0.17 | 0.19 | 0.17 | 0.15 | 0.15 | 0.13 |
| Olfr1243 | 0.13 | 0.14 | 0.07 | 0.09 | 0.08 | 0.08 | 0.07 | 0.07 | 0.07 | 0.07 |
| Olfr1245 | 0.04 | 0.08 | 0.04 | 0.07 | 0.08 | 0.07 | 0.07 | 0.04 | 0.06 | 0.04 |
| Olfr1246 | 0.06 | 0.03 | 0.02 | 0.09 | 0.06 | 0.04 | 0.01 | 0.00 | NA | NA |
| Olfr1247 | 0.06 | 0.10 | 0.08 | 0.08 | 0.12 | 0.10 | 0.05 | 0.04 | 0.08 | 0.07 |
| Olfr1248 | 0.02 | 0.03 | 0.02 | 0.06 | 0.10 | 0.10 | 0.02 | 0.00 | NA | NA |
| Olfr1249 | 0.18 | 0.26 | 0.20 | 0.22 | 0.20 | 0.22 | 0.14 | 0.13 | 0.13 | 0.08 |
| Olfr125 | 0.03 | 0.03 | 0.06 | 0.06 | 0.10 | 0.12 | 0.05 | 0.03 | 0.05 | 0.04 |
| Olfr1250 | 0.02 | 0.03 | 0.04 | 0.04 | 0.05 | 0.07 | 0.07 | 0.05 | 0.02 | 0.03 |
| Olfr1251 | NA | NA | NA | 0.01 | 0.02 | NA | 0.00 | 0.01 | NA | NA |
| Olfr1252 | 0.34 | 0.30 | 0.10 | 0.19 | 0.13 | 0.13 | 0.14 | 0.10 | 0.11 | 0.12 |
| Olfr1253 | 0.00 | 0.00 | 0.03 | 0.07 | 0.06 | 0.07 | 0.03 | 0.02 | 0.04 | 0.01 |
| Olfr1254 | 0.01 | 0.00 | 0.01 | 0.03 | 0.06 | 0.08 | NA | NA | NA | NA |
| Olfr1255 | 0.23 | 0.22 | 0.15 | 0.17 | 0.16 | 0.19 | 0.12 | 0.11 | 0.12 | 0.12 |
| Olfr1256 | 0.20 | 0.17 | 0.17 | 0.19 | 0.18 | 0.20 | 0.18 | 0.15 | 0.12 | 0.14 |
| Olfr1257 | 0.10 | 0.09 | 0.06 | 0.10 | 0.08 | 0.06 | 0.04 | 0.04 | 0.06 | 0.05 |
| Olfr1258 | 0.22 | 0.29 | 0.17 | 0.20 | 0.16 | 0.17 | 0.15 | 0.15 | 0.16 | 0.16 |
| Olfr1259 | 0.27 | 0.29 | 0.18 | 0.18 | 0.20 | 0.20 | 0.13 | 0.12 | 0.16 | 0.12 |
| Olfr126 | 0.15 | 0.22 | 0.15 | 0.17 | 0.15 | 0.15 | 0.10 | 0.10 | 0.11 | 0.09 |
| Olfr1260 | 0.39 | 0.42 | 0.25 | 0.27 | 0.26 | 0.28 | 0.21 | 0.20 | 0.23 | 0.21 |
| Olfr1261 | 0.12 | 0.10 | 0.07 | 0.08 | 0.05 | 0.04 | 0.03 | 0.02 | 0.04 | 0.04 |
| Olfr1262 | 0.34 | 0.29 | 0.20 | 0.16 | 0.23 | 0.20 | 0.16 | 0.16 | 0.20 | 0.20 |
| Olfr1263 | 0.20 | 0.23 | 0.14 | 0.17 | 0.16 | 0.20 | 0.14 | 0.14 | 0.17 | 0.12 |
| Olfr1264 | 0.23 | 0.30 | 0.17 | 0.21 | 0.17 | 0.19 | 0.16 | 0.14 | 0.16 | 0.14 |
| Olfr1265 | 0.13 | 0.13 | 0.12 | 0.14 | 0.15 | 0.17 | 0.10 | 0.10 | 0.10 | 0.10 |
| Olfr1269 | 0.13 | 0.13 | 0.12 | 0.14 | 0.15 | 0.17 | 0.10 | 0.10 | 0.10 | 0.10 |
| Olfr127 | 0.15 | 0.22 | 0.15 | 0.17 | 0.15 | 0.15 | 0.10 | 0.10 | 0.11 | 0.09 |
| Olfr1270 | 0.14 | 0.18 | 0.09 | 0.10 | 0.12 | 0.12 | 0.09 | 0.10 | 0.11 | 0.10 |
| Olfr1271 | 0.13 | 0.14 | 0.14 | 0.17 | 0.20 | 0.23 | 0.13 | 0.10 | 0.15 | 0.07 |
| Olfr1272 | 0.42 | 0.41 | 0.24 | 0.24 | 0.27 | 0.30 | 0.24 | 0.22 | 0.28 | 0.26 |
| Olfr1273-ps | 0.10 | 0.15 | 0.03 | 0.06 | 0.05 | 0.11 | 0.03 | 0.04 | 0.03 | 0.01 |
| Olfr1274-ps | 0.31 | 0.22 | 0.20 | 0.23 | 0.20 | 0.19 | 0.20 | 0.18 | 0.18 | 0.17 |
| Olfr1275 | 0.11 | 0.16 | 0.12 | 0.12 | 0.12 | 0.15 | 0.09 | 0.08 | 0.07 | 0.09 |
| Olfr1276 | 0.27 | 0.24 | 0.23 | 0.23 | 0.22 | 0.22 | 0.19 | 0.16 | 0.20 | 0.18 |
| Olfr1277 | 0.31 | 0.32 | 0.23 | 0.24 | 0.26 | 0.31 | 0.24 | 0.21 | 0.23 | 0.22 |
| Olfr1278 | 0.12 | 0.19 | 0.15 | 0.14 | 0.16 | 0.16 | 0.12 | 0.11 | 0.11 | 0.10 |
| Olfr1279 | 0.38 | 0.37 | 0.28 | 0.29 | 0.29 | 0.32 | 0.23 | 0.22 | 0.23 | 0.22 |
| Olfr128 | 0.15 | 0.22 | 0.15 | 0.17 | 0.15 | 0.15 | 0.10 | 0.10 | 0.11 | 0.09 |
| Olfr1280 | 0.08 | 0.10 | 0.09 | 0.11 | 0.11 | 0.13 | 0.15 | 0.14 | 0.06 | 0.07 |
| Olfr1281 | 0.14 | 0.16 | 0.08 | 0.10 | 0.09 | 0.11 | 0.03 | 0.03 | 0.04 | 0.02 |
| Olfr1282 | 0.08 | 0.10 | 0.09 | 0.11 | 0.11 | 0.13 | 0.15 | 0.14 | 0.06 | 0.07 |
| Olfr1283 | 0.08 | 0.10 | 0.09 | 0.11 | 0.11 | 0.13 | 0.15 | 0.14 | 0.06 | 0.07 |
| Olfr1284 | 0.38 | 0.42 | 0.30 | 0.34 | 0.32 | 0.34 | 0.23 | 0.23 | 0.26 | 0.25 |
| Olfr1286 | 0.08 | 0.10 | 0.09 | 0.11 | 0.11 | 0.13 | 0.15 | 0.14 | 0.06 | 0.07 |
| Olfr1287 | 0.08 | 0.10 | 0.09 | 0.11 | 0.11 | 0.13 | 0.15 | 0.14 | 0.06 | 0.07 |
| Olfr1288 | 0.08 | 0.11 | 0.06 | 0.08 | 0.08 | 0.08 | 0.06 | 0.05 | 0.05 | 0.05 |
| Olfr1289 | 0.13 | 0.17 | 0.08 | 0.10 | 0.12 | 0.13 | 0.05 | 0.05 | 0.05 | 0.05 |
| Olfr129 | 0.37 | 0.36 | 0.28 | 0.30 | 0.27 | 0.29 | 0.25 | 0.23 | 0.27 | 0.23 |
| Olfr1290 | 0.09 | 0.10 | 0.14 | 0.13 | 0.19 | 0.15 | 0.05 | 0.05 | 0.08 | 0.09 |
| Olfr1294 | 0.13 | 0.17 | 0.11 | 0.14 | 0.14 | 0.14 | 0.12 | 0.10 | 0.11 | 0.11 |
| Olfr1295 | 0.17 | 0.16 | 0.09 | 0.16 | 0.14 | 0.16 | 0.13 | 0.10 | 0.07 | 0.09 |
| Olfr1297 | 0.08 | 0.04 | 0.09 | 0.17 | 0.10 | 0.12 | 0.08 | 0.06 | 0.06 | 0.06 |
| Olfr1298 | 0.16 | 0.33 | 0.07 | 0.10 | 0.11 | 0.12 | 0.06 | 0.04 | 0.04 | 0.05 |
| Olfr1299 | 0.18 | 0.21 | 0.09 | 0.11 | 0.12 | 0.14 | 0.09 | 0.08 | 0.10 | 0.07 |
| Olfr13 | 0.39 | 0.40 | 0.29 | 0.33 | 0.31 | 0.34 | 0.27 | 0.25 | 0.30 | 0.27 |
| Olfr130 | 0.13 | 0.16 | 0.10 | 0.15 | 0.14 | 0.13 | 0.09 | 0.09 | 0.10 | 0.08 |
| Olfr1300-ps1 | 0.07 | 0.10 | 0.06 | 0.07 | 0.06 | 0.11 | 0.06 | 0.03 | 0.04 | 0.04 |
| Olfr1301 | 0.18 | 0.16 | 0.16 | 0.15 | 0.16 | 0.15 | 0.13 | 0.15 | 0.18 | 0.15 |
| Olfr1302 | 0.08 | 0.12 | 0.07 | 0.03 | 0.07 | 0.07 | 0.03 | 0.04 | 0.02 | 0.02 |
| Olfr1303 | 0.18 | 0.22 | 0.08 | 0.11 | 0.08 | 0.10 | 0.07 | 0.07 | 0.10 | 0.08 |
| Olfr1305 | 0.15 | 0.12 | 0.13 | 0.12 | 0.14 | 0.16 | 0.12 | 0.11 | 0.13 | 0.11 |
| Olfr1306 | NA | 0.02 | 0.01 | 0.02 | 0.04 | 0.03 | NA | 0.01 | NA | NA |
| Olfr1307 | 0.26 | 0.26 | 0.13 | 0.15 | 0.18 | 0.16 | 0.13 | 0.13 | 0.16 | 0.12 |
| Olfr1308 | 0.05 | 0.10 | 0.00 | 0.04 | 0.05 | 0.03 | 0.03 | 0.01 | 0.03 | 0.01 |
| Olfr1309 | 0.19 | 0.22 | 0.18 | 0.23 | 0.18 | 0.18 | 0.16 | 0.14 | 0.17 | 0.14 |
| Olfr131 | 0.18 | 0.25 | 0.14 | 0.16 | 0.17 | 0.18 | 0.14 | 0.13 | 0.14 | 0.13 |
| Olfr1310 | 0.16 | 0.19 | 0.13 | 0.15 | 0.16 | 0.18 | 0.11 | 0.10 | 0.12 | 0.11 |
| Olfr1311 | 0.23 | 0.21 | 0.15 | 0.15 | 0.17 | 0.18 | 0.15 | 0.13 | 0.16 | 0.14 |
| Olfr1312 | 0.23 | 0.25 | 0.08 | 0.07 | 0.06 | 0.08 | 0.04 | 0.03 | 0.03 | 0.02 |
| Olfr1313 | 0.17 | 0.23 | 0.11 | 0.12 | 0.16 | 0.17 | 0.14 | 0.09 | 0.12 | 0.11 |
| Olfr1314 | 0.18 | 0.15 | 0.10 | 0.13 | 0.13 | 0.14 | 0.11 | 0.08 | 0.11 | 0.10 |
| Olfr1316 | 0.13 | 0.15 | 0.11 | 0.14 | 0.15 | 0.15 | 0.10 | 0.08 | 0.11 | 0.09 |
| Olfr1317 | 0.06 | 0.13 | 0.09 | 0.06 | 0.10 | 0.09 | 0.06 | 0.05 | 0.08 | 0.05 |
| Olfr1318 | 0.12 | 0.19 | 0.11 | 0.10 | 0.10 | 0.14 | 0.08 | 0.08 | 0.13 | 0.08 |
| Olfr132 | 0.23 | 0.18 | 0.18 | 0.16 | 0.19 | 0.21 | 0.13 | 0.13 | 0.15 | 0.15 |
| Olfr1320 | 0.22 | 0.23 | 0.19 | 0.18 | 0.19 | 0.21 | 0.20 | 0.19 | 0.18 | 0.17 |
| Olfr1321 | 0.23 | 0.30 | 0.16 | 0.23 | 0.19 | 0.22 | 0.13 | 0.13 | 0.16 | 0.14 |
| Olfr1322 | 0.23 | 0.25 | 0.22 | 0.23 | 0.23 | 0.20 | 0.20 | 0.21 | 0.24 | 0.22 |
| Olfr1323 | 0.15 | 0.17 | 0.12 | 0.11 | 0.15 | 0.16 | 0.11 | 0.11 | 0.16 | 0.11 |
| Olfr1324 | 0.29 | 0.27 | 0.22 | 0.25 | 0.23 | 0.24 | 0.19 | 0.19 | 0.20 | 0.17 |
| Olfr1325 | 0.11 | 0.14 | 0.08 | 0.13 | 0.12 | 0.09 | 0.07 | 0.07 | 0.08 | 0.09 |
| Olfr1328 | 0.24 | 0.23 | 0.23 | 0.23 | 0.23 | 0.28 | 0.26 | 0.25 | 0.23 | 0.19 |
| Olfr1329 | 0.24 | 0.23 | 0.23 | 0.23 | 0.23 | 0.28 | 0.26 | 0.25 | 0.23 | 0.19 |
| Olfr133 | 0.05 | 0.05 | 0.04 | 0.08 | 0.05 | 0.06 | 0.02 | 0.02 | 0.03 | 0.02 |
| Olfr1330 | 0.24 | 0.23 | 0.23 | 0.23 | 0.23 | 0.28 | 0.26 | 0.25 | 0.23 | 0.19 |
| Olfr1331 | 0.34 | 0.37 | 0.18 | 0.24 | 0.24 | 0.29 | 0.20 | 0.18 | 0.18 | 0.18 |
| Olfr1333 | 0.24 | 0.23 | 0.23 | 0.23 | 0.23 | 0.28 | 0.26 | 0.25 | 0.23 | 0.19 |
| Olfr1335 | 0.24 | 0.23 | 0.23 | 0.23 | 0.23 | 0.28 | 0.26 | 0.25 | 0.23 | 0.19 |
| Olfr1336 | 0.23 | 0.27 | 0.27 | 0.27 | 0.23 | 0.22 | 0.21 | 0.20 | 0.26 | 0.24 |
| Olfr1337 | 0.24 | 0.23 | 0.23 | 0.23 | 0.23 | 0.28 | 0.26 | 0.25 | 0.23 | 0.19 |
| Olfr1338 | 0.24 | 0.23 | 0.23 | 0.23 | 0.23 | 0.28 | 0.26 | 0.25 | 0.23 | 0.19 |
| Olfr1339 | 0.39 | 0.40 | 0.35 | 0.34 | 0.32 | 0.35 | 0.29 | 0.29 | 0.34 | 0.30 |
| Olfr134 | 0.06 | 0.04 | 0.05 | 0.08 | 0.06 | 0.03 | NA | 0.01 | 0.01 | 0.01 |
| Olfr1340 | 0.38 | 0.35 | 0.31 | 0.33 | 0.31 | 0.33 | 0.24 | 0.23 | 0.33 | 0.30 |
| Olfr1341 | 0.20 | 0.23 | 0.16 | 0.19 | 0.18 | 0.21 | 0.13 | 0.11 | 0.15 | 0.16 |
| Olfr1342 | 0.29 | 0.30 | 0.25 | 0.27 | 0.29 | 0.32 | 0.24 | 0.21 | 0.26 | 0.23 |
| Olfr1344 | 0.30 | 0.31 | 0.27 | 0.29 | 0.31 | 0.33 | 0.28 | 0.25 | 0.32 | 0.29 |
| Olfr1346 | 0.45 | 0.48 | 0.37 | 0.41 | 0.40 | 0.42 | 0.31 | 0.29 | 0.41 | 0.36 |
| Olfr1347 | 0.56 | 0.59 | 0.40 | 0.41 | 0.43 | 0.44 | 0.40 | 0.40 | 0.45 | 0.42 |
| Olfr1348 | 0.28 | 0.22 | 0.24 | 0.25 | 0.25 | 0.26 | 0.23 | 0.22 | 0.30 | 0.30 |
| Olfr1349 | 0.43 | 0.43 | 0.39 | 0.40 | 0.36 | 0.38 | 0.37 | 0.34 | 0.42 | 0.40 |
| Olfr135 | 0.04 | 0.05 | 0.06 | 0.06 | 0.08 | 0.08 | 0.05 | 0.04 | 0.03 | 0.04 |
| Olfr1350 | 0.28 | 0.29 | 0.26 | 0.26 | 0.30 | 0.32 | 0.25 | 0.19 | 0.25 | 0.23 |
| Olfr1351 | 0.18 | 0.20 | 0.15 | 0.17 | 0.19 | 0.24 | 0.13 | 0.13 | 0.15 | 0.14 |
| Olfr1352 | 0.15 | 0.18 | 0.11 | 0.10 | 0.13 | 0.11 | 0.10 | 0.10 | 0.09 | 0.10 |
| Olfr1353 | 0.23 | 0.25 | 0.13 | 0.14 | 0.16 | 0.16 | 0.09 | 0.08 | 0.11 | 0.11 |
| Olfr1354 | 0.26 | 0.30 | 0.17 | 0.20 | 0.22 | 0.23 | 0.19 | 0.17 | 0.21 | 0.17 |
| Olfr1355 | 0.26 | 0.30 | 0.17 | 0.20 | 0.22 | 0.23 | 0.19 | 0.17 | 0.21 | 0.17 |
| Olfr1356 | 0.16 | 0.21 | 0.12 | 0.11 | 0.14 | 0.18 | 0.11 | 0.08 | 0.11 | 0.07 |
| Olfr1357 | 0.27 | 0.30 | 0.22 | 0.24 | 0.23 | 0.26 | 0.20 | 0.19 | 0.21 | 0.20 |
| Olfr1359 | 0.40 | 0.40 | 0.27 | 0.28 | 0.29 | 0.29 | 0.26 | 0.24 | 0.28 | 0.26 |
| Olfr136 | 0.10 | 0.13 | 0.03 | 0.04 | 0.06 | 0.06 | 0.03 | 0.03 | 0.06 | 0.02 |
| Olfr1360 | 0.40 | 0.40 | 0.27 | 0.28 | 0.29 | 0.29 | 0.26 | 0.24 | 0.28 | 0.26 |
| Olfr1361 | 0.37 | 0.35 | 0.27 | 0.24 | 0.24 | 0.26 | 0.22 | 0.20 | 0.26 | 0.29 |
| Olfr1362 | 0.41 | 0.43 | 0.32 | 0.35 | 0.34 | 0.40 | 0.29 | 0.28 | 0.32 | 0.31 |
| Olfr1364 | 0.37 | 0.39 | 0.33 | 0.36 | 0.30 | 0.32 | 0.30 | 0.28 | 0.30 | 0.29 |
| Olfr1366 | 0.37 | 0.42 | 0.31 | 0.34 | 0.29 | 0.31 | 0.27 | 0.24 | 0.29 | 0.26 |
| Olfr1367 | 0.13 | 0.17 | 0.12 | 0.16 | 0.13 | 0.15 | 0.11 | 0.09 | 0.15 | 0.13 |
| Olfr1368 | 0.16 | 0.14 | 0.08 | 0.10 | 0.10 | 0.10 | 0.07 | 0.07 | 0.12 | 0.07 |
| Olfr137 | 0.28 | 0.34 | 0.26 | 0.24 | 0.28 | 0.31 | 0.23 | 0.22 | 0.24 | 0.20 |
| Olfr1370 | 0.23 | 0.31 | 0.20 | 0.19 | 0.18 | 0.19 | 0.18 | 0.16 | 0.16 | 0.19 |
| Olfr1371 | 0.34 | 0.29 | 0.15 | 0.22 | 0.41 | 0.27 | 0.24 | 0.22 | 0.23 | 0.23 |
| Olfr1372-ps1 | 0.11 | 0.08 | 0.09 | 0.11 | 0.15 | 0.13 | 0.12 | 0.13 | 0.16 | 0.11 |
| Olfr1373 | 0.34 | 0.29 | 0.15 | 0.22 | 0.41 | 0.27 | 0.24 | 0.22 | 0.23 | 0.23 |
| Olfr1377 | 0.20 | 0.22 | 0.22 | 0.22 | 0.27 | 0.25 | 0.17 | 0.17 | 0.23 | 0.18 |
| Olfr1378 | 0.30 | 0.32 | 0.27 | 0.31 | 0.28 | 0.34 | 0.32 | 0.30 | 0.30 | 0.24 |
| Olfr138 | 0.04 | 0.04 | 0.02 | 0.04 | 0.03 | 0.03 | NA | NA | NA | 0.01 |
| Olfr1380 | 0.34 | 0.29 | 0.15 | 0.22 | 0.41 | 0.27 | 0.24 | 0.22 | 0.23 | 0.23 |
| Olfr1381 | 0.34 | 0.29 | 0.15 | 0.22 | 0.41 | 0.27 | 0.24 | 0.22 | 0.23 | 0.23 |
| Olfr1382 | 0.34 | 0.29 | 0.15 | 0.22 | 0.41 | 0.27 | 0.24 | 0.22 | 0.23 | 0.23 |
| Olfr1383 | 0.32 | 0.41 | 0.22 | 0.23 | 0.29 | 0.29 | 0.25 | 0.23 | 0.25 | 0.23 |
| Olfr1384 | 0.34 | 0.34 | 0.20 | 0.20 | 0.24 | 0.25 | 0.19 | 0.18 | 0.20 | 0.18 |
| Olfr1385 | 0.26 | 0.25 | 0.24 | 0.25 | 0.22 | 0.23 | 0.20 | 0.20 | 0.21 | 0.20 |
| Olfr1386 | 0.26 | 0.25 | 0.24 | 0.25 | 0.22 | 0.23 | 0.20 | 0.20 | 0.21 | 0.20 |
| Olfr1387 | 0.22 | 0.22 | 0.15 | 0.17 | 0.20 | 0.22 | 0.15 | 0.15 | 0.18 | 0.16 |
| Olfr1388 | 0.44 | 0.44 | 0.29 | 0.34 | 0.33 | 0.36 | 0.27 | 0.26 | 0.31 | 0.28 |
| Olfr1389 | 0.34 | 0.31 | 0.26 | 0.28 | 0.28 | 0.32 | 0.22 | 0.23 | 0.25 | 0.25 |
| Olfr139 | 0.36 | 0.35 | 0.31 | 0.33 | 0.33 | 0.36 | 0.29 | 0.28 | 0.34 | 0.31 |
| Olfr1390 | 0.28 | 0.28 | 0.28 | 0.30 | 0.30 | 0.29 | 0.24 | 0.26 | 0.26 | 0.26 |
| Olfr1391 | 0.17 | 0.15 | 0.17 | 0.20 | 0.22 | 0.23 | 0.15 | 0.16 | 0.19 | 0.18 |
| Olfr1392 | 0.37 | 0.36 | 0.29 | 0.28 | 0.31 | 0.34 | 0.23 | 0.23 | 0.32 | 0.25 |
| Olfr1393 | 0.59 | 0.61 | 0.38 | 0.45 | 0.40 | 0.40 | 0.33 | 0.33 | 0.35 | 0.32 |
| Olfr1394 | 0.51 | 0.52 | 0.45 | 0.46 | 0.46 | 0.50 | 0.37 | 0.35 | 0.46 | 0.42 |
| Olfr1395 | 0.54 | 0.55 | 0.43 | 0.49 | 0.42 | 0.45 | 0.41 | 0.39 | 0.46 | 0.45 |
| Olfr1396 | 0.30 | 0.34 | 0.25 | 0.54 | 0.23 | 0.24 | 0.23 | 0.21 | 0.25 | 0.24 |
| Olfr140 | 0.36 | 0.36 | 0.28 | 0.29 | 0.28 | 0.33 | 0.24 | 0.25 | 0.28 | 0.26 |
| Olfr1402 | 0.40 | 0.40 | 0.29 | 0.30 | 0.31 | 0.32 | 0.26 | 0.25 | 0.34 | 0.29 |
| Olfr1404 | 0.18 | 0.17 | 0.13 | 0.11 | 0.16 | 0.15 | 0.11 | 0.09 | 0.12 | 0.10 |
| Olfr1406 | 0.14 | 0.16 | 0.17 | 0.21 | 0.19 | 0.19 | 0.13 | 0.12 | 0.13 | 0.12 |
| Olfr1408 | 0.20 | 0.22 | 0.20 | 0.20 | 0.23 | 0.25 | 0.15 | 0.16 | 0.17 | 0.14 |
| Olfr141 | 0.15 | 0.15 | 0.15 | 0.15 | 0.13 | 0.16 | 0.13 | 0.11 | 0.12 | 0.09 |
| Olfr1410 | 0.54 | 0.56 | 0.41 | 0.47 | 0.40 | 0.42 | 0.37 | 0.35 | 0.42 | 0.40 |
| Olfr1411 | 0.35 | 0.31 | 0.27 | 0.27 | 0.23 | 0.22 | 0.24 | 0.23 | 0.27 | 0.24 |
| Olfr1412 | 0.40 | 0.41 | 0.32 | 0.35 | 0.33 | 0.32 | 0.26 | 0.25 | 0.31 | 0.31 |
| Olfr1413 | 0.35 | 0.39 | 0.35 | 0.36 | 0.36 | 0.37 | 0.32 | 0.31 | 0.36 | 0.34 |
| Olfr1414 | 0.37 | 0.38 | 0.33 | 0.35 | 0.33 | 0.35 | 0.37 | 0.33 | 0.37 | 0.34 |
| Olfr1415 | 0.37 | 0.38 | 0.33 | 0.35 | 0.33 | 0.35 | 0.37 | 0.33 | 0.37 | 0.34 |
| Olfr1416 | 0.37 | 0.38 | 0.33 | 0.35 | 0.33 | 0.35 | 0.37 | 0.33 | 0.37 | 0.34 |
| Olfr1417 | 0.36 | 0.37 | 0.30 | 0.34 | 0.30 | 0.33 | 0.25 | 0.24 | 0.28 | 0.26 |
| Olfr1418 | 0.42 | 0.47 | 0.23 | 0.29 | 0.27 | 0.30 | 0.18 | 0.19 | 0.19 | 0.19 |
| Olfr1419 | 0.24 | 0.27 | 0.22 | 0.24 | 0.21 | 0.23 | 0.21 | 0.20 | 0.26 | 0.24 |
| Olfr142 | 0.20 | 0.19 | 0.09 | 0.12 | 0.12 | 0.14 | 0.10 | 0.09 | 0.11 | 0.09 |
| Olfr1420 | 0.26 | 0.25 | 0.28 | 0.27 | 0.25 | 0.25 | 0.20 | 0.19 | 0.22 | 0.21 |
| Olfr1423 | 0.24 | 0.26 | 0.24 | 0.21 | 0.19 | 0.20 | 0.18 | 0.18 | 0.26 | 0.20 |
| Olfr1424 | 0.08 | 0.09 | 0.11 | 0.11 | 0.13 | 0.15 | 0.06 | 0.05 | 0.07 | 0.05 |
| Olfr1425 | 0.25 | 0.28 | 0.23 | 0.28 | 0.26 | 0.28 | 0.23 | 0.21 | 0.26 | 0.22 |
| Olfr1426 | 0.44 | 0.43 | 0.25 | 0.29 | 0.27 | 0.28 | 0.21 | 0.19 | 0.22 | 0.21 |
| Olfr1427 | 0.23 | 0.26 | 0.22 | 0.19 | 0.25 | 0.25 | 0.16 | 0.17 | 0.17 | 0.16 |
| Olfr1428 | 0.34 | 0.41 | 0.26 | 0.29 | 0.27 | 0.27 | 0.24 | 0.23 | 0.27 | 0.29 |
| Olfr143 | 0.24 | 0.26 | 0.23 | 0.24 | 0.23 | 0.26 | 0.21 | 0.17 | 0.21 | 0.18 |
| Olfr1431 | 0.20 | 0.25 | 0.14 | 0.15 | 0.12 | 0.22 | 0.11 | 0.07 | 0.09 | 0.07 |
| Olfr1434 | 0.05 | 0.08 | 0.04 | 0.04 | 0.07 | 0.06 | 0.00 | 0.00 | 0.00 | 0.01 |
| Olfr1436 | 0.14 | 0.18 | 0.07 | 0.06 | 0.16 | 0.16 | 0.03 | 0.04 | 0.02 | NA |
| Olfr1437 | 0.23 | 0.20 | 0.17 | 0.18 | 0.13 | 0.13 | 0.15 | 0.13 | 0.12 | 0.11 |
| Olfr1440 | 0.22 | 0.25 | 0.19 | 0.21 | 0.21 | 0.22 | 0.18 | 0.17 | 0.22 | 0.21 |
| Olfr1441 | 0.20 | 0.24 | 0.16 | 0.17 | 0.18 | 0.21 | 0.16 | 0.13 | 0.18 | 0.17 |
| Olfr1442 | 0.17 | 0.21 | 0.12 | 0.14 | 0.18 | 0.20 | 0.10 | 0.10 | 0.13 | 0.10 |
| Olfr1443 | 0.08 | 0.08 | 0.05 | 0.07 | 0.07 | 0.08 | 0.03 | 0.02 | 0.06 | 0.03 |
| Olfr1444 | 0.28 | 0.30 | 0.20 | 0.20 | 0.13 | 0.16 | 0.13 | 0.12 | 0.13 | 0.14 |
| Olfr1445 | 0.20 | 0.19 | 0.18 | 0.20 | 0.17 | 0.18 | 0.15 | 0.13 | 0.14 | 0.14 |
| Olfr1446 | 0.11 | 0.10 | 0.10 | 0.11 | 0.13 | 0.12 | 0.08 | 0.06 | 0.08 | 0.07 |
| Olfr1447 | 0.11 | 0.14 | 0.13 | 0.18 | 0.14 | 0.18 | 0.07 | 0.06 | 0.06 | 0.06 |
| Olfr1448 | 0.06 | 0.06 | 0.06 | 0.07 | 0.07 | 0.09 | 0.05 | 0.04 | 0.05 | 0.04 |
| Olfr1449 | 0.28 | 0.29 | 0.21 | 0.22 | 0.23 | 0.28 | 0.21 | 0.19 | 0.19 | 0.16 |
| Olfr145 | 0.34 | 0.36 | 0.19 | 0.21 | 0.19 | 0.23 | 0.19 | 0.18 | 0.20 | 0.18 |
| Olfr1450 | 0.22 | 0.30 | 0.20 | 0.20 | 0.20 | 0.24 | 0.15 | 0.15 | 0.16 | 0.16 |
| Olfr1451 | 0.10 | 0.17 | 0.10 | 0.12 | 0.10 | 0.11 | 0.07 | 0.07 | 0.11 | 0.05 |
| Olfr1453 | 0.14 | 0.18 | 0.13 | 0.17 | 0.17 | 0.19 | 0.10 | 0.10 | 0.09 | 0.11 |
| Olfr1454 | 0.12 | 0.15 | 0.08 | 0.08 | 0.07 | 0.10 | 0.06 | 0.04 | 0.05 | 0.02 |
| Olfr1457 | 0.05 | 0.10 | 0.07 | 0.10 | 0.07 | 0.10 | 0.02 | 0.01 | 0.02 | 0.02 |
| Olfr1459 | 0.11 | 0.09 | 0.13 | 0.10 | 0.09 | 0.10 | 0.07 | 0.06 | 0.08 | 0.07 |
| Olfr146 | 0.11 | 0.15 | 0.15 | 0.15 | 0.12 | 0.13 | 0.10 | 0.09 | 0.10 | 0.08 |
| Olfr1461 | 0.32 | 0.42 | 0.16 | 0.17 | 0.21 | 0.20 | 0.13 | 0.13 | 0.17 | 0.11 |
| Olfr1462 | 0.07 | 0.06 | 0.05 | 0.07 | 0.08 | 0.07 | 0.03 | 0.03 | 0.04 | 0.04 |
| Olfr1463 | 0.26 | 0.22 | 0.19 | 0.20 | 0.18 | 0.19 | 0.23 | 0.20 | 0.25 | 0.23 |
| Olfr1465 | 0.32 | 0.34 | 0.25 | 0.28 | 0.28 | 0.31 | 0.19 | 0.19 | 0.22 | 0.20 |
| Olfr1466 | 0.18 | 0.18 | 0.14 | 0.14 | 0.11 | 0.12 | 0.08 | 0.08 | 0.10 | 0.10 |
| Olfr1467 | 0.20 | 0.20 | 0.12 | 0.10 | 0.11 | 0.12 | 0.10 | 0.09 | 0.08 | 0.08 |
| Olfr1469 | 0.18 | 0.21 | 0.10 | 0.12 | 0.09 | 0.12 | 0.08 | 0.09 | 0.09 | 0.08 |
| Olfr147 | 0.44 | 0.49 | 0.35 | 0.36 | 0.34 | 0.34 | 0.29 | 0.28 | 0.32 | 0.29 |
| Olfr1471 | 0.09 | 0.16 | 0.04 | 0.06 | 0.05 | 0.07 | 0.09 | 0.05 | 0.03 | 0.02 |
| Olfr1472 | 0.10 | 0.19 | 0.08 | 0.11 | 0.10 | 0.12 | 0.08 | 0.05 | 0.10 | 0.07 |
| Olfr1474 | 0.14 | 0.19 | 0.08 | 0.12 | 0.13 | 0.13 | 0.12 | 0.10 | 0.10 | 0.09 |
| Olfr1475 | 0.13 | 0.15 | 0.09 | 0.14 | 0.10 | 0.20 | 0.03 | 0.03 | 0.04 | 0.03 |
| Olfr1477 | 0.11 | 0.17 | NA | 0.01 | 0.06 | 0.06 | NA | NA | NA | NA |
| Olfr148 | 0.28 | 0.33 | 0.20 | 0.23 | 0.23 | 0.23 | 0.18 | 0.17 | 0.20 | 0.20 |
| Olfr1480 | 0.18 | 0.19 | 0.13 | 0.14 | 0.16 | 0.18 | 0.10 | 0.09 | 0.11 | 0.07 |
| Olfr1484 | 0.20 | 0.19 | 0.11 | 0.09 | 0.14 | 0.16 | 0.11 | 0.10 | 0.06 | 0.07 |
| Olfr1487 | 0.02 | 0.04 | 0.10 | 0.10 | 0.16 | 0.21 | 0.02 | 0.02 | 0.04 | 0.05 |
| Olfr1489 | 0.12 | 0.15 | 0.11 | 0.11 | 0.10 | 0.09 | 0.09 | 0.08 | 0.08 | 0.07 |
| Olfr149 | 0.48 | 0.53 | 0.43 | 0.44 | 0.39 | 0.39 | 0.34 | 0.33 | 0.38 | 0.34 |
| Olfr1490 | 0.29 | 0.28 | 0.20 | 0.25 | 0.23 | 0.24 | 0.19 | 0.19 | 0.23 | 0.21 |
| Olfr1491 | 0.30 | 0.33 | 0.27 | 0.27 | 0.28 | 0.32 | 0.24 | 0.22 | 0.29 | 0.26 |
| Olfr1494 | 0.10 | 0.14 | 0.08 | 0.10 | 0.14 | 0.19 | 0.07 | 0.06 | 0.10 | 0.05 |
| Olfr1495 | 0.29 | 0.34 | 0.23 | 0.26 | 0.23 | 0.26 | 0.20 | 0.21 | 0.24 | 0.26 |
| Olfr1496 | 0.06 | 0.07 | 0.07 | 0.05 | 0.05 | 0.06 | 0.05 | 0.04 | 0.04 | 0.03 |
| Olfr1497 | 0.40 | 0.43 | 0.33 | 0.39 | 0.40 | 0.44 | 0.32 | 0.30 | 0.38 | 0.33 |
| Olfr1499 | 0.18 | 0.25 | 0.11 | 0.14 | 0.16 | 0.16 | 0.12 | 0.09 | 0.11 | 0.11 |
| Olfr15 | 0.20 | 0.17 | 0.18 | 0.17 | 0.14 | 0.16 | 0.15 | 0.14 | 0.15 | 0.18 |
| Olfr150 | 0.25 | 0.27 | 0.20 | 0.25 | 0.20 | 0.25 | 0.18 | 0.19 | 0.20 | 0.18 |
| Olfr1500 | 0.29 | 0.30 | 0.23 | 0.26 | 0.27 | 0.26 | 0.21 | 0.20 | 0.24 | 0.22 |
| Olfr1501 | 0.11 | 0.14 | 0.06 | 0.11 | 0.11 | 0.18 | 0.05 | 0.04 | 0.08 | 0.05 |
| Olfr1502 | 0.39 | 0.48 | 0.29 | 0.31 | 0.32 | 0.35 | 0.23 | 0.22 | 0.27 | 0.24 |
| Olfr1504 | 0.25 | 0.28 | 0.12 | 0.17 | 0.15 | 0.17 | 0.11 | 0.10 | 0.11 | 0.11 |
| Olfr1505 | 0.27 | 0.27 | 0.19 | 0.23 | 0.23 | 0.25 | 0.14 | 0.11 | 0.24 | 0.17 |
| Olfr1506 | 0.30 | 0.31 | 0.21 | 0.24 | 0.25 | 0.28 | 0.25 | 0.24 | 0.22 | 0.20 |
| Olfr1507 | 0.22 | 0.23 | 0.09 | 0.10 | 0.15 | 0.13 | 0.10 | 0.07 | 0.10 | 0.09 |
| Olfr1508 | 0.27 | 0.34 | 0.21 | 0.24 | 0.21 | 0.21 | 0.21 | 0.19 | 0.22 | 0.18 |
| Olfr1509 | 0.18 | 0.22 | 0.12 | 0.14 | 0.14 | 0.13 | 0.12 | 0.11 | 0.12 | 0.13 |
| Olfr151 | 0.11 | 0.09 | 0.11 | 0.13 | 0.13 | 0.11 | 0.13 | 0.10 | 0.12 | 0.10 |
| Olfr1510 | 0.51 | 0.52 | 0.38 | 0.36 | 0.35 | 0.35 | 0.34 | 0.32 | 0.40 | 0.34 |
| Olfr1511 | 0.39 | 0.37 | 0.23 | 0.22 | 0.23 | 0.25 | 0.20 | 0.19 | 0.27 | 0.22 |
| Olfr1512 | 0.19 | 0.19 | 0.19 | 0.17 | 0.18 | 0.24 | 0.15 | 0.14 | 0.15 | 0.16 |
| Olfr1513 | 0.19 | 0.19 | 0.19 | 0.17 | 0.18 | 0.24 | 0.15 | 0.14 | 0.15 | 0.16 |
| Olfr152 | 0.20 | 0.30 | 0.18 | 0.23 | 0.26 | 0.25 | 0.13 | 0.12 | 0.17 | 0.15 |
| Olfr153 | 0.16 | 0.24 | 0.10 | 0.15 | 0.15 | 0.16 | 0.11 | 0.11 | 0.11 | 0.11 |
| Olfr1532-ps1 | 0.48 | 0.49 | 0.37 | 0.40 | 0.36 | 0.37 | 0.34 | 0.32 | 0.37 | 0.36 |
| Olfr1535 | 0.33 | 0.36 | 0.26 | 0.29 | 0.28 | 0.32 | 0.23 | 0.20 | 0.24 | 0.25 |
| Olfr1537 | 0.19 | 0.17 | 0.13 | 0.16 | 0.12 | 0.11 | 0.14 | 0.15 | 0.08 | 0.06 |
| Olfr154 | 0.10 | 0.18 | 0.08 | 0.10 | 0.13 | 0.11 | 0.14 | 0.11 | 0.10 | 0.10 |
| Olfr155 | 0.35 | 0.33 | 0.25 | 0.30 | 0.31 | 0.33 | 0.23 | 0.22 | 0.29 | 0.26 |
| Olfr156 | 0.18 | 0.18 | 0.14 | 0.15 | 0.16 | 0.16 | 0.12 | 0.11 | 0.16 | 0.14 |
| Olfr157 | 0.41 | 0.44 | 0.37 | 0.39 | 0.42 | 0.45 | 0.30 | 0.30 | 0.33 | 0.31 |
| Olfr159 | 0.26 | 0.25 | 0.24 | 0.23 | 0.20 | 0.23 | 0.21 | 0.19 | 0.21 | 0.21 |
| Olfr16 | 0.30 | 0.30 | 0.27 | 0.27 | 0.25 | 0.28 | 0.22 | 0.20 | 0.27 | 0.22 |
| Olfr160 | 0.23 | 0.25 | 0.23 | 0.25 | 0.25 | 0.28 | 0.19 | 0.19 | 0.21 | 0.20 |
| Olfr161 | 0.36 | 0.41 | 0.31 | 0.32 | 0.28 | 0.30 | 0.21 | 0.20 | 0.26 | 0.24 |
| Olfr164 | 0.20 | 0.20 | 0.14 | 0.14 | 0.16 | 0.17 | 0.13 | 0.12 | 0.20 | 0.13 |
| Olfr165 | 0.24 | 0.29 | 0.20 | 0.20 | 0.21 | 0.23 | 0.18 | 0.15 | 0.19 | 0.15 |
| Olfr166 | 0.25 | 0.32 | 0.24 | 0.22 | 0.30 | 0.41 | 0.24 | 0.22 | 0.22 | 0.17 |
| Olfr167 | 0.30 | 0.29 | 0.11 | 0.16 | 0.17 | 0.16 | 0.12 | 0.11 | 0.13 | 0.10 |
| Olfr168 | 0.21 | 0.26 | 0.14 | 0.14 | 0.19 | 0.20 | 0.12 | 0.11 | 0.15 | 0.12 |
| Olfr169 | 0.43 | 0.47 | 0.28 | 0.32 | 0.31 | 0.36 | 0.30 | 0.28 | 0.31 | 0.27 |
| Olfr17 | 0.23 | 0.22 | 0.19 | 0.24 | 0.23 | 0.27 | 0.19 | 0.18 | 0.22 | 0.19 |
| Olfr170 | 0.43 | 0.47 | 0.28 | 0.32 | 0.31 | 0.36 | 0.30 | 0.28 | 0.31 | 0.27 |
| Olfr171 | 0.43 | 0.47 | 0.28 | 0.32 | 0.31 | 0.36 | 0.30 | 0.28 | 0.31 | 0.27 |
| Olfr172 | 0.22 | 0.29 | 0.10 | 0.15 | 0.23 | 0.15 | 0.18 | 0.11 | 0.17 | 0.14 |
| Olfr173 | 0.07 | 0.13 | 0.08 | 0.11 | 0.13 | 0.08 | 0.07 | 0.05 | 0.10 | 0.05 |
| Olfr175-ps1 | 0.03 | 0.07 | 0.05 | 0.07 | 0.06 | 0.08 | 0.00 | NA | 0.01 | NA |
| Olfr176 | 0.01 | 0.05 | 0.04 | 0.04 | 0.10 | 0.08 | 0.03 | 0.01 | 0.04 | 0.02 |
| Olfr177 | 0.01 | 0.05 | 0.04 | 0.04 | 0.10 | 0.08 | 0.03 | 0.01 | 0.04 | 0.02 |
| Olfr178 | 0.06 | 0.08 | 0.03 | 0.11 | 0.08 | 0.12 | 0.00 | NA | 0.00 | NA |
| Olfr18 | 0.12 | 0.11 | 0.09 | 0.08 | 0.12 | 0.12 | 0.09 | 0.07 | 0.11 | 0.08 |
| Olfr180 | 0.22 | 0.20 | 0.22 | 0.23 | 0.24 | 0.24 | 0.20 | 0.18 | 0.20 | 0.19 |
| Olfr181 | 0.02 | 0.03 | 0.05 | 0.05 | 0.06 | 0.10 | 0.01 | 0.00 | 0.01 | 0.01 |
| Olfr183 | 0.04 | 0.08 | 0.05 | 0.06 | 0.08 | 0.09 | 0.02 | 0.02 | 0.07 | 0.04 |
| Olfr186 | 0.11 | 0.16 | 0.07 | 0.08 | 0.08 | 0.09 | 0.07 | 0.05 | 0.08 | 0.06 |
| Olfr187 | 0.09 | 0.10 | 0.08 | 0.07 | 0.10 | 0.09 | 0.04 | 0.02 | 0.05 | 0.06 |
| Olfr19 | 0.26 | 0.27 | 0.25 | 0.25 | 0.24 | 0.26 | 0.21 | 0.18 | 0.20 | 0.19 |
| Olfr190 | NA | 0.01 | NA | 0.02 | 0.07 | 0.04 | NA | NA | 0.01 | 0.00 |
| Olfr191 | 0.11 | 0.14 | 0.06 | 0.10 | 0.09 | 0.08 | 0.05 | 0.05 | 0.02 | 0.02 |
| Olfr192 | NA | 0.06 | NA | 0.01 | 0.04 | 0.06 | NA | 0.01 | NA | NA |
| Olfr193 | 0.00 | NA | 0.02 | 0.04 | 0.07 | 0.08 | 0.00 | NA | NA | NA |
| Olfr194 | 0.09 | 0.08 | 0.07 | 0.10 | 0.08 | 0.12 | 0.08 | 0.08 | 0.04 | 0.04 |
| Olfr195 | 0.15 | 0.12 | 0.11 | 0.11 | 0.15 | 0.17 | 0.06 | 0.07 | 0.07 | 0.09 |
| Olfr196 | 0.01 | 0.04 | NA | 0.03 | 0.02 | 0.02 | NA | NA | NA | NA |
| Olfr197 | 0.22 | 0.22 | 0.18 | 0.24 | 0.18 | 0.21 | 0.20 | 0.16 | 0.16 | 0.16 |
| Olfr198 | 0.30 | 0.27 | 0.13 | 0.15 | 0.27 | 0.28 | 0.13 | 0.12 | 0.12 | 0.11 |
| Olfr199 | 0.07 | 0.10 | 0.06 | 0.09 | 0.10 | 0.10 | 0.05 | 0.05 | 0.04 | 0.02 |
| Olfr2 | 0.11 | 0.13 | 0.09 | 0.11 | 0.11 | 0.12 | 0.09 | 0.09 | 0.09 | 0.07 |
| Olfr20 | 0.22 | 0.23 | 0.19 | 0.23 | 0.20 | 0.21 | 0.19 | 0.18 | 0.20 | 0.15 |
| Olfr201 | 0.10 | 0.15 | 0.10 | 0.15 | 0.10 | 0.13 | 0.06 | 0.06 | 0.05 | 0.07 |
| Olfr202 | 0.18 | 0.17 | 0.14 | 0.15 | 0.14 | 0.16 | 0.15 | 0.13 | 0.16 | 0.15 |
| Olfr203 | NA | NA | 0.07 | 0.05 | 0.03 | 0.04 | 0.01 | NA | NA | NA |
| Olfr204 | 0.11 | 0.12 | 0.08 | 0.12 | 0.11 | 0.11 | 0.09 | 0.08 | 0.10 | 0.08 |
| Olfr205 | 0.11 | 0.12 | 0.08 | 0.12 | 0.11 | 0.11 | 0.09 | 0.08 | 0.10 | 0.08 |
| Olfr206 | 0.00 | 0.05 | 0.08 | 0.07 | 0.08 | 0.11 | 0.01 | 0.00 | 0.03 | NA |
| Olfr207 | 0.11 | 0.09 | 0.14 | 0.12 | 0.14 | 0.17 | 0.11 | 0.10 | 0.11 | 0.11 |
| Olfr208 | 0.11 | 0.09 | 0.14 | 0.12 | 0.14 | 0.17 | 0.11 | 0.10 | 0.11 | 0.11 |
| Olfr209 | 0.11 | 0.09 | 0.14 | 0.12 | 0.14 | 0.17 | 0.11 | 0.10 | 0.11 | 0.11 |
| Olfr211 | 0.30 | 0.31 | 0.27 | 0.28 | 0.24 | 0.26 | 0.21 | 0.19 | 0.26 | 0.22 |
| Olfr212 | 0.30 | 0.36 | 0.19 | 0.19 | 0.17 | 0.18 | 0.15 | 0.14 | 0.19 | 0.21 |
| Olfr213 | 0.28 | 0.33 | 0.24 | 0.28 | 0.20 | 0.24 | 0.22 | 0.21 | 0.24 | 0.25 |
| Olfr214 | 0.37 | 0.37 | 0.30 | 0.32 | 0.30 | 0.29 | 0.29 | 0.25 | 0.30 | 0.27 |
| Olfr215 | 0.33 | 0.34 | 0.27 | 0.28 | 0.27 | 0.30 | 0.24 | 0.24 | 0.29 | 0.28 |
| Olfr218 | 0.16 | 0.15 | 0.14 | 0.14 | 0.13 | 0.17 | 0.11 | 0.09 | 0.12 | 0.11 |
| Olfr220 | 0.34 | 0.36 | 0.26 | 0.30 | 0.29 | 0.29 | 0.24 | 0.22 | 0.25 | 0.26 |
| Olfr221 | 0.34 | 0.36 | 0.28 | 0.33 | 0.33 | 0.33 | 0.26 | 0.24 | 0.29 | 0.27 |
| Olfr222 | 0.40 | 0.42 | 0.34 | 0.37 | 0.36 | 0.39 | 0.34 | 0.31 | 0.36 | 0.34 |
| Olfr223 | 0.29 | 0.31 | 0.20 | 0.22 | 0.20 | 0.21 | 0.18 | 0.16 | 0.20 | 0.16 |
| Olfr224 | 0.05 | 0.07 | 0.03 | 0.05 | 0.10 | 0.05 | 0.06 | 0.05 | 0.04 | 0.05 |
| Olfr225 | 0.32 | 0.33 | 0.30 | 0.30 | 0.30 | 0.30 | 0.29 | 0.25 | 0.28 | 0.32 |
| Olfr228 | 0.02 | 0.03 | 0.03 | 0.03 | 0.08 | 0.07 | 0.03 | 0.03 | 0.05 | 0.03 |
| Olfr229 | 0.01 | 0.04 | 0.02 | 0.06 | 0.05 | 0.04 | 0.01 | 0.01 | 0.02 | 0.02 |
| Olfr23 | 0.23 | 0.20 | 0.19 | 0.20 | 0.19 | 0.23 | 0.16 | 0.15 | 0.16 | 0.18 |
| Olfr231 | 0.38 | 0.36 | 0.29 | 0.32 | 0.30 | 0.34 | 0.26 | 0.24 | 0.28 | 0.27 |
| Olfr235 | 0.23 | 0.20 | 0.17 | 0.18 | 0.13 | 0.13 | 0.15 | 0.13 | 0.12 | 0.11 |
| Olfr237-ps1 | 0.12 | 0.17 | 0.10 | 0.11 | 0.12 | 0.17 | 0.12 | 0.10 | 0.13 | 0.10 |
| Olfr239 | 0.79 | 0.86 | 0.52 | 0.55 | 0.55 | 0.58 | 0.51 | 0.49 | 0.62 | 0.57 |
| Olfr24 | 0.22 | 0.25 | 0.19 | 0.20 | 0.23 | 0.28 | 0.18 | 0.17 | 0.20 | 0.18 |
| Olfr242 | 0.08 | 0.09 | 0.08 | 0.11 | 0.10 | 0.13 | 0.07 | 0.06 | 0.05 | 0.06 |
| Olfr243 | NA | NA | 0.03 | 0.04 | 0.04 | 0.10 | NA | 0.02 | 0.02 | 0.03 |
| Olfr247 | 0.46 | 0.48 | 0.29 | 0.31 | 0.28 | 0.31 | 0.28 | 0.26 | 0.28 | 0.23 |
| Olfr248 | 0.24 | 0.25 | 0.11 | 0.15 | 0.20 | 0.19 | 0.12 | 0.10 | 0.09 | 0.10 |
| Olfr25 | 0.15 | 0.13 | 0.09 | 0.11 | 0.11 | 0.12 | 0.12 | 0.08 | 0.09 | 0.07 |
| Olfr250 | 0.18 | 0.24 | 0.13 | 0.17 | 0.15 | 0.15 | 0.13 | 0.11 | 0.13 | 0.12 |
| Olfr251 | 0.17 | 0.20 | 0.13 | 0.16 | 0.17 | 0.20 | 0.13 | 0.11 | 0.13 | 0.11 |
| Olfr255 | 0.22 | 0.23 | 0.19 | 0.23 | 0.20 | 0.21 | 0.19 | 0.18 | 0.20 | 0.15 |
| Olfr257 | 0.33 | 0.36 | 0.29 | 0.32 | 0.31 | 0.33 | 0.26 | 0.24 | 0.30 | 0.30 |
| Olfr259 | 0.08 | 0.11 | 0.05 | 0.06 | 0.09 | 0.07 | 0.02 | 0.01 | 0.03 | 0.02 |
| Olfr26 | 0.21 | 0.22 | 0.17 | 0.16 | 0.16 | 0.18 | 0.15 | 0.14 | 0.14 | 0.13 |
| Olfr262 | 0.22 | 0.23 | 0.17 | 0.20 | 0.17 | 0.15 | 0.14 | 0.13 | 0.15 | 0.12 |
| Olfr263 | 0.08 | 0.05 | 0.06 | 0.10 | 0.13 | 0.08 | 0.05 | 0.05 | 0.05 | 0.04 |
| Olfr266 | 0.26 | 0.24 | 0.21 | 0.20 | 0.20 | 0.20 | 0.17 | 0.17 | 0.18 | 0.18 |
| Olfr267 | 0.43 | 0.37 | 0.30 | 0.32 | 0.30 | 0.28 | 0.29 | 0.28 | 0.32 | 0.31 |
| Olfr27 | 0.08 | 0.09 | 0.08 | 0.11 | 0.10 | 0.13 | 0.07 | 0.06 | 0.05 | 0.06 |
| Olfr270 | 0.09 | 0.13 | 0.09 | 0.11 | 0.10 | 0.11 | 0.08 | 0.08 | 0.14 | 0.11 |
| Olfr272 | 0.24 | 0.26 | 0.21 | 0.22 | 0.27 | 0.29 | 0.19 | 0.19 | 0.20 | 0.21 |
| Olfr273 | 0.23 | 0.21 | 0.19 | 0.18 | 0.20 | 0.22 | 0.16 | 0.15 | 0.16 | 0.18 |
| Olfr275 | 0.15 | 0.22 | 0.16 | 0.17 | 0.19 | 0.21 | 0.20 | 0.16 | 0.15 | 0.17 |
| Olfr279 | 0.34 | 0.27 | 0.29 | 0.30 | 0.28 | 0.30 | 0.30 | 0.28 | 0.36 | 0.35 |
| Olfr281 | 0.32 | 0.36 | 0.23 | 0.28 | 0.27 | 0.30 | 0.25 | 0.25 | 0.27 | 0.27 |
| Olfr282 | 0.49 | 0.48 | 0.37 | 0.40 | 0.38 | 0.41 | 0.36 | 0.33 | 0.41 | 0.36 |
| Olfr283 | 0.32 | 0.35 | 0.27 | 0.30 | 0.31 | 0.34 | 0.24 | 0.23 | 0.28 | 0.28 |
| Olfr284 | 0.35 | 0.37 | 0.28 | 0.29 | 0.29 | 0.32 | 0.27 | 0.25 | 0.36 | 0.34 |
| Olfr285 | 0.33 | 0.36 | 0.29 | 0.32 | 0.31 | 0.33 | 0.26 | 0.24 | 0.30 | 0.30 |
| Olfr286 | 0.25 | 0.26 | 0.20 | 0.22 | 0.25 | 0.28 | 0.20 | 0.19 | 0.22 | 0.22 |
| Olfr288 | 0.29 | 0.38 | 0.21 | 0.21 | 0.22 | 0.25 | 0.20 | 0.16 | 0.20 | 0.14 |
| Olfr29-ps1 | 0.33 | 0.35 | 0.29 | 0.30 | 0.30 | 0.33 | 0.25 | 0.26 | 0.28 | 0.27 |
| Olfr290 | 0.15 | 0.13 | 0.04 | 0.07 | 0.07 | 0.06 | 0.04 | 0.04 | 0.05 | 0.03 |
| Olfr291 | 0.12 | 0.13 | 0.07 | 0.07 | 0.10 | 0.10 | 0.07 | 0.06 | 0.05 | 0.08 |
| Olfr292 | 0.14 | 0.19 | 0.08 | 0.09 | 0.15 | 0.16 | 0.07 | 0.04 | 0.07 | 0.05 |
| Olfr293 | 0.18 | 0.20 | 0.18 | 0.16 | 0.22 | 0.24 | 0.12 | 0.12 | 0.13 | 0.15 |
| Olfr294 | 0.09 | 0.12 | 0.05 | 0.12 | 0.07 | 0.08 | 0.10 | 0.09 | 0.09 | 0.10 |
| Olfr295 | 0.09 | 0.14 | 0.04 | 0.05 | 0.06 | 0.06 | 0.03 | 0.02 | 0.02 | 0.03 |
| Olfr297 | 0.17 | 0.17 | 0.14 | 0.16 | 0.17 | 0.17 | 0.11 | 0.09 | 0.13 | 0.11 |
| Olfr298 | 0.08 | 0.08 | 0.09 | 0.12 | 0.10 | 0.14 | 0.07 | 0.06 | 0.08 | 0.05 |
| Olfr299 | 0.03 | 0.04 | NA | NA | NA | NA | NA | NA | 0.01 | NA |
| Olfr3 | 0.28 | 0.31 | 0.25 | 0.23 | 0.22 | 0.24 | 0.20 | 0.20 | 0.23 | 0.22 |
| Olfr30 | 0.41 | 0.39 | 0.33 | 0.32 | 0.35 | 0.36 | 0.31 | 0.30 | 0.38 | 0.36 |
| Olfr301 | 0.10 | 0.13 | 0.06 | 0.12 | 0.09 | 0.11 | 0.04 | 0.04 | 0.07 | 0.06 |
| Olfr303 | 0.15 | 0.20 | 0.13 | 0.13 | 0.17 | 0.22 | 0.12 | 0.12 | 0.17 | 0.14 |
| Olfr304 | 0.12 | 0.18 | 0.09 | 0.12 | 0.08 | 0.08 | 0.07 | 0.05 | 0.11 | 0.05 |
| Olfr305 | 0.21 | 0.22 | 0.18 | 0.21 | 0.16 | 0.22 | 0.15 | 0.14 | 0.17 | 0.13 |
| Olfr307 | 0.23 | 0.24 | 0.22 | 0.21 | 0.22 | 0.24 | 0.15 | 0.15 | 0.21 | 0.18 |
| Olfr308 | 0.12 | 0.13 | 0.11 | 0.11 | 0.14 | 0.15 | 0.09 | 0.07 | 0.07 | 0.09 |
| Olfr309 | 0.22 | 0.26 | 0.19 | 0.23 | 0.20 | 0.26 | 0.21 | 0.19 | 0.22 | 0.19 |
| Olfr31 | 0.33 | 0.37 | 0.26 | 0.28 | 0.25 | 0.28 | 0.23 | 0.23 | 0.26 | 0.24 |
| Olfr310 | 0.09 | 0.13 | 0.05 | 0.08 | 0.05 | 0.04 | 0.07 | 0.06 | 0.08 | 0.05 |
| Olfr311 | 0.19 | 0.18 | 0.16 | 0.19 | 0.16 | 0.18 | 0.14 | 0.13 | 0.16 | 0.19 |
| Olfr312 | 0.23 | 0.24 | 0.19 | 0.23 | 0.25 | 0.24 | 0.17 | 0.17 | 0.21 | 0.21 |
| Olfr313 | 0.28 | 0.33 | 0.22 | 0.22 | 0.25 | 0.24 | 0.18 | 0.17 | 0.19 | 0.19 |
| Olfr314 | 0.58 | 0.57 | 0.49 | 0.48 | 0.44 | 0.49 | 0.38 | 0.38 | 0.50 | 0.46 |
| Olfr315 | 0.40 | 0.40 | 0.33 | 0.33 | 0.32 | 0.34 | 0.30 | 0.30 | 0.38 | 0.37 |
| Olfr316 | 0.50 | 0.52 | 0.45 | 0.41 | 0.42 | 0.43 | 0.36 | 0.31 | 0.36 | 0.39 |
| Olfr317 | 0.34 | 0.36 | 0.28 | 0.29 | 0.32 | 0.31 | 0.29 | 0.26 | 0.31 | 0.30 |
| Olfr318 | 0.19 | 0.21 | 0.18 | 0.17 | 0.22 | 0.27 | 0.18 | 0.16 | 0.21 | 0.18 |
| Olfr319 | 0.25 | 0.23 | 0.20 | 0.24 | 0.26 | 0.26 | 0.18 | 0.14 | 0.20 | 0.22 |
| Olfr32 | 0.18 | 0.15 | 0.12 | 0.09 | 0.12 | 0.15 | 0.11 | 0.08 | 0.10 | 0.09 |
| Olfr320 | 0.55 | 0.55 | 0.30 | 0.38 | 0.30 | 0.32 | 0.32 | 0.30 | 0.32 | 0.30 |
| Olfr322 | 0.38 | 0.39 | 0.36 | 0.35 | 0.36 | 0.39 | 0.34 | 0.31 | 0.41 | 0.38 |
| Olfr323 | 0.15 | 0.17 | 0.13 | 0.17 | 0.18 | 0.18 | 0.13 | 0.12 | 0.18 | 0.15 |
| Olfr324 | 0.26 | 0.26 | 0.19 | 0.24 | 0.24 | 0.23 | 0.21 | 0.20 | 0.25 | 0.22 |
| Olfr325 | 0.16 | 0.20 | 0.13 | 0.16 | 0.16 | 0.16 | 0.09 | 0.08 | 0.11 | 0.10 |
| Olfr328 | 0.42 | 0.42 | 0.31 | 0.34 | 0.30 | 0.32 | 0.28 | 0.24 | 0.31 | 0.28 |
| Olfr329-ps | 0.34 | 0.37 | 0.30 | 0.29 | 0.27 | 0.26 | 0.27 | 0.26 | 0.35 | 0.31 |
| Olfr33 | 0.29 | 0.22 | 0.19 | 0.24 | 0.23 | 0.29 | 0.19 | 0.18 | 0.21 | 0.21 |
| Olfr330 | 0.36 | 0.30 | 0.21 | 0.24 | 0.31 | 0.28 | 0.27 | 0.24 | 0.30 | 0.28 |
| Olfr331 | 0.34 | 0.37 | 0.30 | 0.29 | 0.27 | 0.26 | 0.27 | 0.26 | 0.35 | 0.31 |
| Olfr332 | 0.35 | 0.40 | 0.25 | 0.29 | 0.27 | 0.28 | 0.27 | 0.24 | 0.32 | 0.27 |
| Olfr338 | 0.17 | 0.18 | 0.05 | 0.11 | 0.17 | 0.14 | 0.08 | 0.07 | 0.08 | 0.09 |
| Olfr339 | 0.07 | 0.07 | 0.06 | 0.08 | 0.12 | 0.10 | 0.07 | 0.04 | 0.03 | 0.03 |
| Olfr340 | 0.05 | 0.06 | 0.04 | 0.10 | 0.09 | 0.11 | 0.06 | 0.04 | 0.06 | 0.03 |
| Olfr341 | 0.23 | 0.26 | 0.20 | 0.22 | 0.20 | 0.23 | 0.18 | 0.16 | 0.16 | 0.16 |
| Olfr342 | 0.08 | 0.10 | 0.09 | 0.13 | 0.13 | 0.14 | 0.12 | 0.10 | 0.11 | 0.08 |
| Olfr344 | 0.29 | 0.31 | 0.12 | 0.17 | 0.18 | 0.20 | 0.11 | 0.09 | 0.10 | 0.08 |
| Olfr345 | 0.04 | 0.06 | 0.08 | 0.07 | 0.08 | 0.12 | 0.09 | 0.05 | 0.05 | 0.05 |
| Olfr346 | 0.17 | 0.23 | 0.13 | 0.15 | 0.15 | 0.15 | 0.12 | 0.12 | 0.12 | 0.09 |
| Olfr347 | 0.17 | 0.21 | 0.12 | 0.15 | 0.16 | 0.16 | 0.11 | 0.11 | 0.12 | 0.09 |
| Olfr348 | 0.15 | 0.15 | 0.11 | 0.14 | 0.19 | 0.19 | 0.11 | 0.10 | 0.10 | 0.08 |
| Olfr350 | 0.13 | 0.16 | 0.11 | 0.11 | 0.13 | 0.17 | 0.10 | 0.09 | 0.11 | 0.08 |
| Olfr351 | 0.30 | 0.29 | 0.23 | 0.26 | 0.25 | 0.31 | 0.20 | 0.19 | 0.22 | 0.21 |
| Olfr352 | 0.24 | 0.25 | 0.19 | 0.21 | 0.18 | 0.20 | 0.17 | 0.15 | 0.16 | 0.13 |
| Olfr353 | 0.17 | 0.17 | 0.17 | 0.20 | 0.17 | 0.18 | 0.13 | 0.12 | 0.14 | 0.13 |
| Olfr354 | 0.25 | 0.30 | 0.19 | 0.20 | 0.23 | 0.26 | 0.20 | 0.17 | 0.21 | 0.18 |
| Olfr355 | 0.11 | 0.19 | 0.12 | 0.10 | 0.13 | 0.13 | 0.11 | 0.09 | 0.11 | 0.08 |
| Olfr356 | 0.25 | 0.26 | 0.18 | 0.19 | 0.20 | 0.21 | 0.16 | 0.15 | 0.17 | 0.17 |
| Olfr357 | 0.19 | 0.26 | 0.16 | 0.17 | 0.18 | 0.18 | 0.12 | 0.12 | 0.14 | 0.11 |
| Olfr358 | 0.10 | 0.11 | 0.09 | 0.13 | 0.11 | 0.11 | 0.13 | 0.11 | 0.07 | 0.07 |
| Olfr360 | 0.10 | 0.11 | 0.09 | 0.13 | 0.11 | 0.11 | 0.13 | 0.11 | 0.07 | 0.07 |
| Olfr361 | 0.36 | 0.38 | 0.25 | 0.18 | 0.21 | 0.20 | 0.16 | 0.13 | 0.19 | 0.18 |
| Olfr362 | 0.31 | 0.31 | 0.29 | 0.29 | 0.28 | 0.30 | 0.30 | 0.27 | 0.32 | 0.30 |
| Olfr365 | 0.34 | 0.35 | 0.28 | 0.30 | 0.29 | 0.33 | 0.26 | 0.25 | 0.31 | 0.29 |
| Olfr366 | 0.25 | 0.28 | 0.23 | 0.24 | 0.21 | 0.21 | 0.18 | 0.18 | 0.22 | 0.20 |
| Olfr367-ps | 0.30 | 0.36 | 0.25 | 0.20 | 0.24 | 0.21 | 0.21 | 0.20 | 0.25 | 0.24 |
| Olfr368 | 0.39 | 0.40 | 0.39 | 0.38 | 0.37 | 0.42 | 0.31 | 0.30 | 0.38 | 0.36 |
| Olfr370 | 0.43 | 0.40 | 0.31 | 0.34 | 0.35 | 0.36 | 0.29 | 0.27 | 0.31 | 0.30 |
| Olfr371 | 0.31 | 0.35 | 0.26 | 0.31 | 0.27 | 0.29 | 0.26 | 0.23 | 0.28 | 0.26 |
| Olfr372 | 0.44 | 0.43 | 0.32 | 0.36 | 0.38 | 0.41 | 0.32 | 0.31 | 0.36 | 0.34 |
| Olfr373 | 0.48 | 0.42 | 0.27 | 0.34 | 0.32 | 0.32 | 0.27 | 0.25 | 0.30 | 0.30 |
| Olfr374 | 0.28 | 0.29 | 0.23 | 0.22 | 0.23 | 0.25 | 0.19 | 0.19 | 0.24 | 0.23 |
| Olfr376 | 0.24 | 0.28 | 0.19 | 0.22 | 0.21 | 0.22 | 0.16 | 0.13 | 0.18 | 0.17 |
| Olfr378 | 0.11 | 0.13 | 0.05 | 0.08 | 0.09 | 0.08 | 0.06 | 0.05 | 0.07 | 0.06 |
| Olfr38 | 0.28 | 0.32 | 0.26 | 0.27 | 0.28 | 0.28 | 0.21 | 0.19 | 0.26 | 0.22 |
| Olfr380 | 0.08 | 0.07 | 0.04 | 0.05 | 0.06 | 0.08 | 0.05 | 0.04 | 0.05 | 0.04 |
| Olfr381 | 0.13 | 0.15 | 0.16 | 0.17 | 0.16 | 0.16 | 0.13 | 0.12 | 0.15 | 0.14 |
| Olfr382 | 0.20 | 0.22 | 0.13 | 0.18 | 0.17 | 0.15 | 0.12 | 0.11 | 0.15 | 0.14 |
| Olfr384 | 0.14 | 0.14 | 0.07 | 0.11 | 0.11 | 0.12 | 0.07 | 0.06 | 0.08 | 0.08 |
| Olfr385 | 0.09 | 0.15 | 0.05 | 0.07 | 0.09 | 0.09 | 0.07 | 0.06 | 0.06 | 0.04 |
| Olfr389 | 0.22 | 0.24 | 0.13 | 0.17 | 0.16 | 0.16 | 0.13 | 0.12 | 0.12 | 0.11 |
| Olfr39 | 0.34 | 0.37 | 0.26 | 0.26 | 0.27 | 0.29 | 0.22 | 0.20 | 0.24 | 0.21 |
| Olfr390 | 0.11 | 0.13 | 0.10 | 0.11 | 0.12 | 0.15 | 0.11 | 0.09 | 0.10 | 0.09 |
| Olfr391-ps | 0.22 | 0.23 | 0.19 | 0.23 | 0.20 | 0.21 | 0.19 | 0.18 | 0.20 | 0.15 |
| Olfr392 | 0.05 | 0.02 | 0.05 | 0.06 | 0.05 | 0.06 | 0.04 | 0.04 | 0.03 | 0.03 |
| Olfr393 | 0.15 | 0.18 | 0.12 | 0.12 | 0.13 | 0.14 | 0.09 | 0.06 | 0.10 | 0.09 |
| Olfr394 | 0.19 | 0.21 | 0.15 | 0.17 | 0.20 | 0.21 | 0.13 | 0.12 | 0.15 | 0.13 |
| Olfr395 | 0.22 | 0.23 | 0.19 | 0.23 | 0.20 | 0.21 | 0.19 | 0.18 | 0.20 | 0.15 |
| Olfr397 | 0.16 | 0.15 | 0.12 | 0.16 | 0.16 | 0.19 | 0.10 | 0.10 | 0.14 | 0.11 |
| Olfr398 | 0.45 | 0.45 | 0.36 | 0.39 | 0.38 | 0.39 | 0.33 | 0.30 | 0.35 | 0.31 |
| Olfr399 | 0.34 | 0.36 | 0.28 | 0.28 | 0.27 | 0.31 | 0.22 | 0.19 | 0.22 | 0.23 |
| Olfr401 | 0.64 | 0.66 | 0.43 | 0.49 | 0.44 | 0.47 | 0.34 | 0.36 | 0.43 | 0.41 |
| Olfr402 | 0.35 | 0.35 | 0.29 | 0.28 | 0.27 | 0.28 | 0.24 | 0.22 | 0.27 | 0.24 |
| Olfr403 | 0.21 | 0.21 | 0.18 | 0.20 | 0.16 | 0.20 | 0.20 | 0.19 | 0.20 | 0.17 |
| Olfr406 | 0.32 | 0.34 | 0.23 | 0.27 | 0.23 | 0.26 | 0.26 | 0.23 | 0.23 | 0.23 |
| Olfr410 | 0.23 | 0.23 | 0.17 | 0.20 | 0.19 | 0.18 | 0.18 | 0.17 | 0.18 | 0.17 |
| Olfr411 | 0.47 | 0.46 | 0.38 | 0.40 | 0.36 | 0.41 | 0.35 | 0.34 | 0.37 | 0.33 |
| Olfr412 | 0.38 | 0.40 | 0.35 | 0.38 | 0.35 | 0.39 | 0.31 | 0.28 | 0.36 | 0.33 |
| Olfr414 | 0.35 | 0.32 | 0.28 | 0.30 | 0.30 | 0.31 | 0.25 | 0.25 | 0.30 | 0.30 |
| Olfr417 | 0.41 | 0.45 | 0.33 | 0.41 | 0.36 | 0.39 | 0.32 | 0.32 | 0.32 | 0.31 |
| Olfr418 | 0.23 | 0.26 | 0.20 | 0.21 | 0.22 | 0.25 | 0.17 | 0.16 | 0.20 | 0.18 |
| Olfr419 | 0.30 | 0.35 | 0.26 | 0.28 | 0.25 | 0.26 | 0.24 | 0.23 | 0.26 | 0.24 |
| Olfr420 | 0.23 | 0.27 | 0.22 | 0.22 | 0.20 | 0.22 | 0.21 | 0.20 | 0.23 | 0.20 |
| Olfr421-ps1 | 0.18 | 0.29 | 0.13 | 0.18 | 0.17 | 0.20 | 0.14 | 0.13 | 0.13 | 0.11 |
| Olfr424 | 0.34 | 0.32 | 0.23 | 0.30 | 0.29 | 0.36 | 0.22 | 0.19 | 0.24 | 0.24 |
| Olfr426 | 0.23 | 0.28 | 0.18 | 0.23 | 0.19 | 0.19 | 0.14 | 0.13 | 0.15 | 0.14 |
| Olfr427 | 0.23 | 0.28 | 0.18 | 0.23 | 0.19 | 0.19 | 0.14 | 0.13 | 0.15 | 0.14 |
| Olfr429 | 0.25 | 0.21 | 0.20 | 0.22 | 0.22 | 0.26 | 0.18 | 0.15 | 0.17 | 0.19 |
| Olfr43 | 0.09 | 0.09 | 0.07 | 0.09 | 0.07 | 0.07 | 0.03 | 0.03 | 0.05 | 0.03 |
| Olfr430 | 0.17 | 0.17 | 0.10 | 0.14 | 0.14 | 0.15 | 0.12 | 0.13 | 0.13 | 0.11 |
| Olfr432 | 0.18 | 0.19 | 0.19 | 0.20 | 0.19 | 0.15 | 0.14 | 0.13 | 0.23 | 0.23 |
| Olfr433 | 0.40 | 0.42 | 0.28 | 0.37 | 0.38 | 0.32 | 0.28 | 0.26 | 0.41 | 0.38 |
| Olfr434 | 0.22 | 0.23 | 0.21 | 0.25 | 0.21 | 0.25 | 0.18 | 0.17 | 0.25 | 0.24 |
| Olfr435 | 0.36 | 0.39 | 0.29 | 0.29 | 0.28 | 0.33 | 0.27 | 0.27 | 0.33 | 0.31 |
| Olfr437 | 0.28 | 0.30 | 0.21 | 0.27 | 0.23 | 0.25 | 0.20 | 0.20 | 0.21 | 0.21 |
| Olfr44 | 0.09 | 0.09 | 0.07 | 0.10 | 0.09 | 0.10 | 0.06 | 0.07 | 0.06 | 0.04 |
| Olfr441 | 0.31 | 0.32 | 0.19 | 0.25 | 0.25 | 0.25 | 0.21 | 0.22 | 0.25 | 0.24 |
| Olfr444 | 0.31 | 0.29 | 0.30 | 0.32 | 0.31 | 0.32 | 0.23 | 0.24 | 0.29 | 0.32 |
| Olfr446 | 0.28 | 0.35 | 0.16 | 0.20 | 0.25 | 0.25 | 0.13 | 0.13 | 0.15 | 0.15 |
| Olfr447 | 0.31 | 0.31 | 0.25 | 0.26 | 0.25 | 0.24 | 0.23 | 0.23 | 0.28 | 0.24 |
| Olfr448 | 0.29 | 0.29 | 0.25 | 0.25 | 0.22 | 0.27 | 0.18 | 0.20 | 0.22 | 0.23 |
| Olfr449 | 0.24 | 0.24 | 0.22 | 0.23 | 0.23 | 0.23 | 0.18 | 0.16 | 0.17 | 0.20 |
| Olfr45 | 0.42 | 0.45 | 0.31 | 0.26 | 0.29 | 0.26 | 0.28 | 0.27 | 0.27 | 0.26 |
| Olfr450 | 0.29 | 0.31 | 0.26 | 0.27 | 0.25 | 0.25 | 0.21 | 0.20 | 0.21 | 0.19 |
| Olfr452 | 0.05 | 0.08 | 0.09 | 0.11 | 0.06 | 0.05 | 0.06 | 0.04 | 0.05 | 0.02 |
| Olfr453 | 0.25 | 0.24 | 0.22 | 0.24 | 0.21 | 0.24 | 0.19 | 0.18 | 0.21 | 0.20 |
| Olfr455 | 0.25 | 0.23 | 0.21 | 0.22 | 0.23 | 0.24 | 0.19 | 0.18 | 0.22 | 0.23 |
| Olfr456 | 0.38 | 0.39 | 0.33 | 0.35 | 0.30 | 0.31 | 0.27 | 0.26 | 0.32 | 0.28 |
| Olfr457 | 0.38 | 0.39 | 0.33 | 0.35 | 0.30 | 0.31 | 0.27 | 0.26 | 0.32 | 0.28 |
| Olfr458 | 0.42 | 0.47 | 0.34 | 0.40 | 0.31 | 0.34 | 0.29 | 0.26 | 0.33 | 0.29 |
| Olfr459 | 0.34 | 0.36 | 0.27 | 0.27 | 0.29 | 0.29 | 0.25 | 0.23 | 0.26 | 0.27 |
| Olfr46 | 0.42 | 0.45 | 0.31 | 0.26 | 0.29 | 0.26 | 0.28 | 0.27 | 0.27 | 0.26 |
| Olfr460 | 0.32 | 0.31 | 0.17 | 0.20 | 0.19 | 0.20 | 0.15 | 0.13 | 0.16 | 0.15 |
| Olfr461 | 0.24 | 0.21 | 0.13 | 0.14 | 0.13 | 0.16 | 0.12 | 0.12 | 0.12 | 0.10 |
| Olfr462 | 0.50 | 0.55 | 0.41 | 0.44 | 0.45 | 0.51 | 0.39 | 0.37 | 0.43 | 0.42 |
| Olfr463 | 0.38 | 0.39 | 0.29 | 0.32 | 0.29 | 0.33 | 0.28 | 0.28 | 0.34 | 0.33 |
| Olfr464 | 0.39 | 0.44 | 0.33 | 0.37 | 0.35 | 0.36 | 0.30 | 0.29 | 0.35 | 0.32 |
| Olfr466 | 0.18 | 0.20 | 0.11 | 0.16 | 0.16 | 0.15 | 0.11 | 0.09 | 0.10 | 0.10 |
| Olfr467 | 0.41 | 0.37 | 0.22 | 0.24 | 0.26 | 0.30 | 0.25 | 0.22 | 0.24 | 0.22 |
| Olfr469 | 0.24 | 0.29 | 0.14 | 0.11 | 0.16 | 0.19 | 0.10 | 0.11 | 0.13 | 0.12 |
| Olfr47 | 0.25 | 0.25 | 0.21 | 0.23 | 0.25 | 0.28 | 0.18 | 0.17 | 0.21 | 0.21 |
| Olfr470 | 0.17 | 0.18 | 0.09 | 0.12 | 0.11 | 0.11 | 0.09 | 0.07 | 0.12 | 0.09 |
| Olfr472 | NA | 0.03 | NA | 0.01 | 0.00 | 0.03 | NA | NA | NA | NA |
| Olfr473 | 0.16 | 0.17 | 0.12 | 0.13 | 0.15 | 0.16 | 0.11 | 0.10 | 0.10 | 0.09 |
| Olfr474 | 0.04 | 0.07 | 0.02 | 0.05 | 0.04 | 0.08 | 0.03 | 0.04 | 0.04 | 0.03 |
| Olfr476 | 0.18 | 0.21 | 0.15 | 0.19 | 0.19 | 0.20 | 0.16 | 0.16 | 0.15 | 0.15 |
| Olfr477 | 0.31 | 0.38 | 0.25 | 0.27 | 0.25 | 0.26 | 0.25 | 0.23 | 0.25 | 0.23 |
| Olfr478 | 0.11 | 0.13 | 0.11 | 0.14 | 0.14 | 0.16 | 0.11 | 0.10 | 0.11 | 0.09 |
| Olfr479 | 0.38 | 0.42 | 0.27 | 0.32 | 0.27 | 0.30 | 0.26 | 0.24 | 0.24 | 0.22 |
| Olfr48 | 0.42 | 0.45 | 0.34 | 0.39 | 0.36 | 0.39 | 0.34 | 0.32 | 0.38 | 0.36 |
| Olfr480 | 0.01 | 0.03 | 0.05 | 0.04 | 0.05 | 0.07 | 0.02 | 0.01 | 0.01 | 0.02 |
| Olfr481 | 0.19 | 0.19 | 0.18 | 0.18 | 0.19 | 0.18 | 0.17 | 0.14 | 0.20 | 0.17 |
| Olfr482 | 0.25 | 0.27 | 0.11 | 0.16 | 0.20 | 0.23 | 0.14 | 0.13 | 0.13 | 0.11 |
| Olfr483 | 0.23 | 0.22 | 0.15 | 0.21 | 0.20 | 0.22 | 0.17 | 0.16 | 0.18 | 0.16 |
| Olfr484 | 0.25 | 0.26 | 0.16 | 0.20 | 0.20 | 0.20 | 0.15 | 0.16 | 0.13 | 0.13 |
| Olfr485 | 0.41 | 0.37 | 0.27 | 0.25 | 0.30 | 0.28 | 0.30 | 0.28 | 0.27 | 0.23 |
| Olfr486 | 0.24 | 0.29 | 0.19 | 0.20 | 0.19 | 0.23 | 0.20 | 0.17 | 0.20 | 0.18 |
| Olfr487 | 0.24 | 0.29 | 0.19 | 0.20 | 0.19 | 0.23 | 0.20 | 0.17 | 0.20 | 0.18 |
| Olfr488 | 0.18 | 0.24 | 0.09 | 0.12 | 0.12 | 0.13 | 0.15 | 0.14 | 0.14 | 0.10 |
| Olfr49 | 0.21 | 0.28 | 0.16 | 0.15 | 0.19 | 0.19 | 0.16 | 0.12 | 0.17 | 0.17 |
| Olfr490 | 0.12 | 0.21 | 0.03 | 0.08 | 0.04 | 0.05 | 0.03 | 0.00 | NA | NA |
| Olfr491 | 0.21 | 0.24 | 0.20 | 0.20 | 0.18 | 0.19 | 0.17 | 0.16 | 0.18 | 0.15 |
| Olfr492 | 0.22 | 0.21 | 0.12 | 0.15 | 0.18 | 0.16 | 0.16 | 0.14 | 0.11 | 0.11 |
| Olfr493 | 0.09 | 0.13 | 0.10 | 0.09 | 0.13 | 0.10 | 0.12 | 0.09 | 0.09 | 0.10 |
| Olfr494 | 0.26 | 0.27 | 0.14 | 0.18 | 0.17 | 0.19 | 0.16 | 0.14 | 0.17 | 0.17 |
| Olfr495 | 0.15 | 0.28 | 0.12 | 0.18 | 0.16 | 0.15 | 0.11 | 0.10 | 0.12 | 0.10 |
| Olfr497 | 0.18 | 0.24 | 0.11 | 0.13 | 0.13 | 0.14 | 0.11 | 0.12 | 0.12 | 0.08 |
| Olfr498 | 0.13 | 0.15 | 0.13 | 0.15 | 0.13 | 0.14 | 0.13 | 0.10 | 0.07 | 0.08 |
| Olfr5 | 0.30 | 0.29 | 0.26 | 0.27 | 0.24 | 0.26 | 0.24 | 0.23 | 0.32 | 0.27 |
| Olfr50 | 0.16 | 0.20 | 0.12 | 0.10 | 0.09 | 0.15 | 0.06 | 0.05 | 0.09 | 0.05 |
| Olfr502 | 0.18 | 0.19 | 0.18 | 0.20 | 0.18 | 0.19 | 0.14 | 0.15 | 0.15 | 0.14 |
| Olfr503 | 0.28 | 0.32 | 0.23 | 0.24 | 0.22 | 0.25 | 0.20 | 0.20 | 0.23 | 0.22 |
| Olfr504 | 0.21 | 0.25 | 0.11 | 0.16 | 0.17 | 0.16 | 0.08 | 0.07 | 0.09 | 0.08 |
| Olfr506 | 0.25 | 0.27 | 0.11 | 0.16 | 0.20 | 0.23 | 0.14 | 0.13 | 0.13 | 0.11 |
| Olfr507 | 0.25 | 0.27 | 0.11 | 0.16 | 0.20 | 0.23 | 0.14 | 0.13 | 0.13 | 0.11 |
| Olfr508 | 0.17 | 0.16 | 0.14 | 0.15 | 0.18 | 0.21 | 0.12 | 0.11 | 0.16 | 0.11 |
| Olfr509 | 0.13 | 0.24 | 0.08 | 0.11 | 0.13 | 0.13 | 0.05 | 0.05 | 0.09 | 0.09 |
| Olfr51 | 0.30 | 0.32 | 0.27 | 0.31 | 0.28 | 0.34 | 0.32 | 0.30 | 0.30 | 0.24 |
| Olfr510 | 0.10 | 0.17 | 0.11 | 0.10 | 0.18 | 0.19 | 0.10 | 0.12 | 0.11 | 0.10 |
| Olfr512 | 0.10 | 0.17 | 0.04 | 0.11 | 0.09 | 0.10 | 0.03 | 0.03 | 0.02 | 0.03 |
| Olfr513 | 0.34 | 0.39 | 0.24 | 0.30 | 0.30 | 0.33 | 0.22 | 0.21 | 0.23 | 0.20 |
| Olfr514 | 0.37 | 0.40 | 0.26 | 0.29 | 0.30 | 0.30 | 0.23 | 0.21 | 0.25 | 0.25 |
| Olfr516 | 0.06 | 0.13 | 0.08 | 0.07 | 0.10 | 0.07 | 0.04 | 0.03 | 0.12 | 0.07 |
| Olfr517 | 0.27 | 0.28 | 0.21 | 0.27 | 0.25 | 0.21 | 0.18 | 0.15 | 0.22 | 0.18 |
| Olfr518 | 0.23 | 0.28 | 0.09 | 0.14 | 0.13 | 0.15 | 0.14 | 0.10 | 0.12 | 0.09 |
| Olfr519 | 0.23 | 0.28 | 0.09 | 0.14 | 0.13 | 0.15 | 0.14 | 0.10 | 0.12 | 0.09 |
| Olfr52 | 0.23 | 0.24 | 0.18 | 0.22 | 0.24 | 0.29 | 0.19 | 0.19 | 0.21 | 0.19 |
| Olfr520 | 0.25 | 0.27 | 0.20 | 0.25 | 0.23 | 0.24 | 0.18 | 0.17 | 0.20 | 0.21 |
| Olfr521 | 0.14 | 0.23 | 0.16 | 0.16 | 0.20 | 0.26 | 0.15 | 0.10 | 0.18 | 0.13 |
| Olfr522 | 0.45 | 0.45 | 0.36 | 0.38 | 0.35 | 0.36 | 0.32 | 0.30 | 0.36 | 0.38 |
| Olfr523 | 0.42 | 0.42 | 0.32 | 0.35 | 0.29 | 0.30 | 0.30 | 0.29 | 0.34 | 0.31 |
| Olfr524 | 0.51 | 0.52 | 0.47 | 0.47 | 0.43 | 0.49 | 0.39 | 0.39 | 0.48 | 0.46 |
| Olfr525 | 0.31 | 0.35 | 0.22 | 0.23 | 0.26 | 0.27 | 0.19 | 0.19 | 0.24 | 0.23 |
| Olfr527 | 0.27 | 0.26 | 0.29 | 0.31 | 0.30 | 0.30 | 0.23 | 0.24 | 0.25 | 0.29 |
| Olfr53 | 0.42 | 0.45 | 0.31 | 0.26 | 0.29 | 0.26 | 0.28 | 0.27 | 0.27 | 0.26 |
| Olfr530 | 0.42 | 0.43 | 0.32 | 0.36 | 0.31 | 0.36 | 0.30 | 0.28 | 0.32 | 0.28 |
| Olfr531 | 0.14 | 0.13 | 0.19 | 0.18 | 0.20 | 0.29 | 0.15 | 0.15 | 0.19 | 0.19 |
| Olfr532 | 0.33 | 0.36 | 0.24 | 0.33 | 0.27 | 0.32 | 0.24 | 0.22 | 0.26 | 0.25 |
| Olfr533 | 0.42 | 0.43 | 0.32 | 0.36 | 0.31 | 0.36 | 0.30 | 0.28 | 0.32 | 0.28 |
| Olfr535 | 0.44 | 0.46 | 0.40 | 0.40 | 0.38 | 0.42 | 0.37 | 0.38 | 0.41 | 0.36 |
| Olfr536 | 0.42 | 0.43 | 0.32 | 0.36 | 0.31 | 0.36 | 0.30 | 0.28 | 0.32 | 0.28 |
| Olfr538 | 0.42 | 0.45 | 0.31 | 0.26 | 0.29 | 0.26 | 0.28 | 0.27 | 0.27 | 0.26 |
| Olfr539 | 0.42 | 0.45 | 0.31 | 0.26 | 0.29 | 0.26 | 0.28 | 0.27 | 0.27 | 0.26 |
| Olfr54 | 0.58 | 0.55 | 0.40 | 0.45 | 0.42 | 0.43 | 0.37 | 0.35 | 0.43 | 0.38 |
| Olfr541 | 0.42 | 0.45 | 0.31 | 0.26 | 0.29 | 0.26 | 0.28 | 0.27 | 0.27 | 0.26 |
| Olfr543 | 0.45 | 0.47 | 0.37 | 0.37 | 0.41 | 0.40 | 0.34 | 0.33 | 0.41 | 0.37 |
| Olfr544 | 0.20 | 0.20 | 0.22 | 0.22 | 0.23 | 0.23 | 0.16 | 0.15 | 0.22 | 0.23 |
| Olfr545 | 0.50 | 0.49 | 0.43 | 0.48 | 0.42 | 0.43 | 0.38 | 0.38 | 0.45 | 0.40 |
| Olfr547 | 0.20 | 0.19 | 0.16 | 0.17 | 0.17 | 0.19 | 0.12 | 0.12 | 0.18 | 0.16 |
| Olfr549 | 0.42 | 0.44 | 0.30 | 0.35 | 0.35 | 0.35 | 0.28 | 0.26 | 0.33 | 0.31 |
| Olfr55 | 0.51 | 0.58 | 0.27 | 0.27 | 0.31 | 0.31 | 0.25 | 0.24 | 0.31 | 0.27 |
| Olfr550 | 0.38 | 0.41 | 0.34 | 0.34 | 0.33 | 0.35 | 0.30 | 0.28 | 0.36 | 0.33 |
| Olfr551 | 0.22 | 0.19 | 0.20 | 0.23 | 0.22 | 0.23 | 0.19 | 0.19 | 0.21 | 0.19 |
| Olfr552 | 0.26 | 0.30 | 0.25 | 0.26 | 0.22 | 0.22 | 0.19 | 0.19 | 0.24 | 0.19 |
| Olfr553 | 0.23 | 0.21 | 0.19 | 0.20 | 0.20 | 0.19 | 0.19 | 0.18 | 0.23 | 0.18 |
| Olfr554 | 0.25 | 0.27 | 0.21 | 0.21 | 0.19 | 0.22 | 0.22 | 0.20 | 0.20 | 0.24 |
| Olfr555 | 0.27 | 0.25 | 0.27 | 0.28 | 0.28 | 0.32 | 0.23 | 0.21 | 0.24 | 0.21 |
| Olfr556 | 0.23 | 0.26 | 0.20 | 0.20 | 0.22 | 0.23 | 0.19 | 0.17 | 0.20 | 0.20 |
| Olfr557 | 0.21 | 0.25 | 0.17 | 0.21 | 0.16 | 0.18 | 0.16 | 0.15 | 0.19 | 0.17 |
| Olfr558 | 0.29 | 0.31 | 0.22 | 0.28 | 0.25 | 0.27 | 0.24 | 0.24 | 0.25 | 0.24 |
| Olfr559 | 0.31 | 0.37 | 0.10 | 0.17 | 0.21 | 0.20 | 0.16 | 0.14 | 0.13 | 0.12 |
| Olfr56 | 0.45 | 0.55 | 0.37 | 0.78 | 0.35 | 0.52 | 0.53 | 0.30 | 0.26 | 0.21 |
| Olfr560 | 0.11 | 0.11 | 0.08 | 0.10 | 0.10 | 0.13 | 0.08 | 0.07 | 0.11 | 0.08 |
| Olfr561 | 0.13 | 0.15 | 0.13 | 0.16 | 0.14 | 0.14 | 0.14 | 0.13 | 0.13 | 0.12 |
| Olfr564 | 0.16 | 0.17 | 0.10 | 0.10 | 0.10 | 0.07 | 0.07 | 0.07 | 0.10 | 0.07 |
| Olfr566 | 0.53 | 0.54 | 0.37 | 0.38 | 0.40 | 0.45 | 0.31 | 0.29 | 0.39 | 0.29 |
| Olfr568 | 0.20 | 0.22 | 0.18 | 0.18 | 0.21 | 0.24 | 0.17 | 0.18 | 0.15 | 0.16 |
| Olfr569 | 0.27 | 0.30 | 0.18 | 0.24 | 0.22 | 0.23 | 0.17 | 0.17 | 0.21 | 0.16 |
| Olfr57 | 0.15 | 0.32 | 0.09 | 0.11 | 0.15 | 0.21 | 0.08 | 0.06 | 0.09 | 0.06 |
| Olfr570 | 0.14 | 0.14 | 0.14 | 0.14 | 0.12 | 0.13 | 0.11 | 0.11 | 0.13 | 0.09 |
| Olfr571 | 0.28 | 0.32 | 0.21 | 0.27 | 0.25 | 0.26 | 0.25 | 0.22 | 0.24 | 0.22 |
| Olfr572 | 0.30 | 0.30 | 0.23 | 0.24 | 0.21 | 0.26 | 0.19 | 0.19 | 0.23 | 0.20 |
| Olfr574 | 0.24 | 0.26 | 0.17 | 0.22 | 0.16 | 0.17 | 0.16 | 0.14 | 0.17 | 0.17 |
| Olfr575 | 0.21 | 0.23 | 0.15 | 0.19 | 0.18 | 0.21 | 0.17 | 0.15 | 0.18 | 0.16 |
| Olfr576 | 0.24 | 0.27 | 0.21 | 0.26 | 0.18 | 0.21 | 0.20 | 0.17 | 0.20 | 0.18 |
| Olfr577 | 0.27 | 0.30 | 0.22 | 0.25 | 0.22 | 0.25 | 0.21 | 0.21 | 0.24 | 0.21 |
| Olfr578 | 0.10 | 0.12 | 0.12 | 0.12 | 0.13 | 0.15 | 0.08 | 0.09 | 0.10 | 0.08 |
| Olfr58 | 0.03 | 0.08 | 0.02 | 0.06 | 0.10 | 0.11 | 0.03 | 0.00 | 0.01 | NA |
| Olfr582 | 0.40 | 0.41 | 0.26 | 0.26 | 0.28 | 0.29 | 0.25 | 0.23 | 0.26 | 0.25 |
| Olfr583 | 0.10 | 0.10 | 0.07 | 0.09 | 0.13 | 0.13 | 0.06 | 0.05 | 0.06 | 0.06 |
| Olfr584 | 0.21 | 0.21 | 0.15 | 0.15 | 0.19 | 0.18 | 0.15 | 0.13 | 0.15 | 0.14 |
| Olfr585 | 0.10 | 0.10 | 0.07 | 0.09 | 0.13 | 0.13 | 0.06 | 0.05 | 0.06 | 0.06 |
| Olfr586 | 0.34 | 0.36 | 0.24 | 0.27 | 0.22 | 0.25 | 0.23 | 0.20 | 0.22 | 0.21 |
| Olfr589 | 0.22 | 0.29 | 0.21 | 0.20 | 0.27 | 0.27 | 0.19 | 0.17 | 0.19 | 0.22 |
| Olfr59 | 0.34 | 0.36 | 0.27 | 0.27 | 0.25 | 0.28 | 0.20 | 0.19 | 0.27 | 0.26 |
| Olfr591 | 0.15 | 0.16 | 0.13 | 0.18 | 0.18 | 0.21 | 0.16 | 0.15 | 0.13 | 0.12 |
| Olfr592 | 0.13 | 0.19 | 0.09 | 0.12 | 0.14 | 0.15 | 0.12 | 0.10 | 0.09 | 0.08 |
| Olfr593 | 0.26 | 0.26 | 0.20 | 0.20 | 0.20 | 0.24 | 0.17 | 0.14 | 0.18 | 0.15 |
| Olfr594 | 0.37 | 0.38 | 0.21 | 0.24 | 0.23 | 0.21 | 0.18 | 0.16 | 0.22 | 0.19 |
| Olfr596 | 0.22 | 0.21 | 0.17 | 0.18 | 0.19 | 0.20 | 0.17 | 0.17 | 0.14 | 0.14 |
| Olfr597 | 0.08 | 0.05 | 0.02 | 0.03 | 0.19 | 0.07 | 0.04 | 0.03 | 0.05 | 0.02 |
| Olfr598 | 0.15 | 0.14 | 0.21 | 0.24 | 0.26 | 0.32 | 0.16 | 0.16 | 0.22 | 0.20 |
| Olfr599 | 0.19 | 0.23 | 0.12 | 0.15 | 0.14 | 0.18 | 0.15 | 0.12 | 0.16 | 0.12 |
| Olfr6 | 0.24 | 0.26 | 0.24 | 0.22 | 0.25 | 0.26 | 0.21 | 0.20 | 0.24 | 0.25 |
| Olfr60 | 0.16 | 0.20 | 0.14 | 0.11 | 0.19 | 0.22 | 0.07 | 0.07 | 0.14 | 0.11 |
| Olfr600 | 0.18 | 0.17 | 0.13 | 0.16 | 0.15 | 0.17 | 0.11 | 0.10 | 0.13 | 0.13 |
| Olfr601 | 0.36 | 0.36 | 0.34 | 0.33 | 0.33 | 0.31 | 0.27 | 0.26 | 0.32 | 0.29 |
| Olfr603 | 0.22 | 0.30 | 0.17 | 0.20 | 0.20 | 0.20 | 0.14 | 0.12 | 0.19 | 0.16 |
| Olfr605 | 0.30 | 0.34 | 0.26 | 0.25 | 0.22 | 0.29 | 0.21 | 0.23 | 0.30 | 0.23 |
| Olfr606 | 0.12 | 0.13 | 0.10 | 0.11 | 0.11 | 0.11 | 0.13 | 0.11 | 0.11 | 0.09 |
| Olfr607 | 0.13 | 0.12 | 0.15 | 0.18 | 0.18 | 0.22 | 0.13 | 0.12 | 0.15 | 0.16 |
| Olfr608 | 0.38 | 0.37 | 0.31 | 0.36 | 0.30 | 0.33 | 0.26 | 0.25 | 0.29 | 0.30 |
| Olfr609 | 0.34 | 0.37 | 0.31 | 0.35 | 0.31 | 0.35 | 0.27 | 0.26 | 0.30 | 0.26 |
| Olfr61 | 0.42 | 0.45 | 0.31 | 0.26 | 0.29 | 0.26 | 0.28 | 0.27 | 0.27 | 0.26 |
| Olfr610 | 0.36 | 0.38 | 0.27 | 0.32 | 0.32 | 0.34 | 0.27 | 0.26 | 0.29 | 0.28 |
| Olfr611 | 0.20 | 0.25 | 0.23 | 0.25 | 0.20 | 0.19 | 0.17 | 0.13 | 0.18 | 0.14 |
| Olfr612 | 0.15 | 0.20 | 0.12 | 0.15 | 0.14 | 0.19 | 0.09 | 0.09 | 0.14 | 0.09 |
| Olfr613 | 0.16 | 0.19 | 0.16 | 0.18 | 0.16 | 0.19 | 0.11 | 0.11 | 0.11 | 0.12 |
| Olfr615 | 0.19 | 0.22 | 0.19 | 0.25 | 0.20 | 0.22 | 0.14 | 0.13 | 0.17 | 0.14 |
| Olfr616 | 0.22 | 0.24 | 0.16 | 0.20 | 0.20 | 0.24 | 0.17 | 0.15 | 0.17 | 0.14 |
| Olfr617 | 0.22 | 0.22 | 0.11 | 0.17 | 0.14 | 0.11 | 0.12 | 0.10 | 0.11 | 0.09 |
| Olfr618 | 0.12 | 0.09 | 0.07 | 0.09 | 0.07 | 0.10 | 0.08 | 0.07 | 0.05 | 0.04 |
| Olfr619 | 0.11 | 0.13 | 0.10 | 0.11 | 0.14 | 0.13 | 0.11 | 0.10 | 0.09 | 0.07 |
| Olfr62 | 0.24 | 0.25 | 0.20 | 0.24 | 0.23 | 0.26 | 0.20 | 0.16 | 0.20 | 0.21 |
| Olfr620 | 0.13 | 0.14 | 0.15 | 0.14 | 0.18 | 0.16 | 0.18 | 0.16 | 0.17 | 0.15 |
| Olfr622 | 0.17 | 0.24 | 0.13 | 0.14 | 0.18 | 0.18 | 0.16 | 0.15 | 0.15 | 0.11 |
| Olfr623 | 0.25 | 0.25 | 0.15 | 0.22 | 0.20 | 0.22 | 0.12 | 0.12 | 0.12 | 0.13 |
| Olfr624 | 0.04 | 0.02 | 0.07 | 0.06 | 0.12 | 0.11 | 0.17 | 0.05 | 0.06 | 0.03 |
| Olfr628 | 0.08 | 0.10 | 0.06 | 0.08 | 0.08 | 0.06 | 0.02 | 0.02 | 0.03 | 0.01 |
| Olfr629 | 0.13 | 0.11 | 0.15 | 0.14 | 0.16 | 0.18 | 0.09 | 0.09 | 0.13 | 0.10 |
| Olfr63 | 0.42 | 0.44 | 0.35 | 0.35 | 0.35 | 0.39 | 0.31 | 0.30 | 0.32 | 0.33 |
| Olfr630 | 0.18 | 0.17 | 0.08 | 0.10 | 0.15 | 0.17 | 0.08 | 0.06 | 0.07 | 0.06 |
| Olfr631 | 0.15 | 0.18 | 0.09 | 0.08 | 0.11 | 0.09 | 0.06 | 0.05 | 0.08 | 0.08 |
| Olfr632 | 0.37 | 0.38 | 0.32 | 0.33 | 0.31 | 0.33 | 0.29 | 0.26 | 0.32 | 0.31 |
| Olfr633 | 0.28 | 0.31 | 0.25 | 0.25 | 0.24 | 0.25 | 0.23 | 0.24 | 0.26 | 0.28 |
| Olfr635 | 0.28 | 0.30 | 0.19 | 0.24 | 0.23 | 0.27 | 0.23 | 0.20 | 0.22 | 0.20 |
| Olfr638 | 0.32 | 0.32 | 0.28 | 0.26 | 0.26 | 0.31 | 0.24 | 0.24 | 0.27 | 0.24 |
| Olfr639 | 0.22 | 0.23 | 0.16 | 0.17 | 0.19 | 0.21 | 0.14 | 0.13 | 0.14 | 0.15 |
| Olfr64 | 0.20 | 0.22 | 0.17 | 0.19 | 0.21 | 0.26 | 0.16 | 0.15 | 0.18 | 0.15 |
| Olfr640 | 0.19 | 0.25 | 0.18 | 0.23 | 0.16 | 0.19 | 0.14 | 0.13 | 0.19 | 0.13 |
| Olfr641 | 0.28 | 0.34 | 0.23 | 0.23 | 0.20 | 0.22 | 0.22 | 0.21 | 0.20 | 0.17 |
| Olfr642 | 0.34 | 0.33 | 0.24 | 0.26 | 0.23 | 0.24 | 0.25 | 0.23 | 0.23 | 0.22 |
| Olfr643 | 0.13 | 0.12 | 0.07 | 0.10 | 0.07 | 0.09 | 0.07 | 0.06 | 0.08 | 0.08 |
| Olfr644 | 0.31 | 0.28 | 0.20 | 0.22 | 0.21 | 0.21 | 0.20 | 0.19 | 0.18 | 0.18 |
| Olfr645 | 0.27 | 0.29 | 0.27 | 0.28 | 0.24 | 0.25 | 0.25 | 0.22 | 0.24 | 0.22 |
| Olfr646 | 0.37 | 0.34 | 0.28 | 0.30 | 0.30 | 0.33 | 0.26 | 0.27 | 0.27 | 0.26 |
| Olfr648 | 0.24 | 0.27 | 0.22 | 0.25 | 0.24 | 0.24 | 0.20 | 0.20 | 0.24 | 0.22 |
| Olfr649 | 0.19 | 0.23 | 0.18 | 0.21 | 0.17 | 0.22 | 0.16 | 0.15 | 0.16 | 0.15 |
| Olfr65 | 0.19 | 0.19 | 0.11 | 0.13 | 0.13 | 0.19 | 0.10 | 0.08 | 0.12 | 0.12 |
| Olfr651 | 0.22 | 0.30 | 0.19 | 0.24 | 0.23 | 0.25 | 0.21 | 0.20 | 0.22 | 0.22 |
| Olfr652 | 0.23 | 0.27 | 0.18 | 0.23 | 0.22 | 0.24 | 0.22 | 0.19 | 0.19 | 0.19 |
| Olfr653 | 0.32 | 0.35 | 0.26 | 0.30 | 0.30 | 0.31 | 0.23 | 0.22 | 0.25 | 0.23 |
| Olfr654 | 0.16 | 0.24 | 0.14 | 0.18 | 0.15 | 0.16 | 0.14 | 0.13 | 0.13 | 0.11 |
| Olfr655 | 0.15 | 0.17 | 0.19 | 0.16 | 0.17 | 0.17 | 0.17 | 0.15 | 0.17 | 0.16 |
| Olfr656 | 0.35 | 0.33 | 0.28 | 0.27 | 0.28 | 0.31 | 0.24 | 0.23 | 0.28 | 0.24 |
| Olfr657 | 0.28 | 0.27 | 0.23 | 0.25 | 0.23 | 0.27 | 0.22 | 0.21 | 0.23 | 0.20 |
| Olfr658 | 0.35 | 0.39 | 0.30 | 0.34 | 0.35 | 0.38 | 0.28 | 0.26 | 0.35 | 0.30 |
| Olfr659 | 0.22 | 0.25 | 0.16 | 0.18 | 0.17 | 0.18 | 0.14 | 0.12 | 0.13 | 0.13 |
| Olfr66 | 0.13 | 0.11 | 0.08 | 0.13 | 0.11 | 0.14 | 0.09 | 0.08 | 0.09 | 0.09 |
| Olfr661 | 0.28 | 0.28 | 0.19 | 0.19 | 0.20 | 0.23 | 0.17 | 0.15 | 0.18 | 0.16 |
| Olfr663 | 0.17 | 0.18 | 0.14 | 0.16 | 0.14 | 0.17 | 0.16 | 0.14 | 0.15 | 0.14 |
| Olfr665 | 0.11 | 0.13 | 0.12 | 0.13 | 0.09 | 0.11 | 0.05 | 0.06 | 0.07 | 0.05 |
| Olfr666 | 0.37 | 0.34 | 0.27 | 0.30 | 0.23 | 0.28 | 0.20 | 0.19 | 0.23 | 0.18 |
| Olfr667 | 0.28 | 0.28 | 0.20 | 0.24 | 0.23 | 0.25 | 0.19 | 0.18 | 0.22 | 0.18 |
| Olfr668 | 0.11 | 0.13 | 0.09 | 0.11 | 0.12 | 0.13 | 0.08 | 0.06 | 0.10 | 0.09 |
| Olfr669 | 0.29 | 0.29 | 0.26 | 0.28 | 0.26 | 0.27 | 0.25 | 0.21 | 0.27 | 0.26 |
| Olfr67 | 0.08 | 0.09 | 0.09 | 0.12 | 0.07 | 0.07 | 0.05 | 0.03 | 0.05 | 0.04 |
| Olfr670 | 0.23 | 0.23 | 0.17 | 0.19 | 0.20 | 0.24 | 0.22 | 0.21 | 0.21 | 0.18 |
| Olfr671 | 0.18 | 0.23 | 0.15 | 0.13 | 0.12 | 0.14 | 0.15 | 0.13 | 0.16 | 0.15 |
| Olfr672 | 0.26 | 0.27 | 0.14 | 0.16 | 0.14 | 0.15 | 0.13 | 0.10 | 0.13 | 0.13 |
| Olfr675 | 0.09 | 0.09 | 0.11 | 0.13 | 0.13 | 0.13 | 0.08 | 0.07 | 0.10 | 0.08 |
| Olfr676 | 0.11 | 0.12 | 0.09 | 0.11 | 0.11 | 0.11 | 0.11 | 0.10 | 0.10 | 0.08 |
| Olfr677 | 0.14 | 0.18 | 0.10 | 0.15 | 0.13 | 0.15 | 0.11 | 0.10 | 0.11 | 0.08 |
| Olfr678 | 0.44 | 0.46 | 0.34 | 0.37 | 0.37 | 0.40 | 0.31 | 0.30 | 0.35 | 0.32 |
| Olfr679 | 0.13 | 0.16 | 0.10 | 0.15 | 0.15 | 0.16 | 0.12 | 0.09 | 0.09 | 0.09 |
| Olfr68 | 0.19 | 0.20 | 0.15 | 0.18 | 0.17 | 0.14 | 0.15 | 0.12 | 0.14 | 0.11 |
| Olfr681 | 0.13 | 0.16 | 0.10 | 0.15 | 0.15 | 0.16 | 0.12 | 0.09 | 0.09 | 0.09 |
| Olfr683 | 0.16 | 0.20 | 0.14 | 0.17 | 0.15 | 0.13 | 0.11 | 0.10 | 0.11 | 0.15 |
| Olfr684 | 0.19 | 0.19 | 0.11 | 0.14 | 0.21 | 0.23 | 0.11 | 0.09 | 0.10 | 0.07 |
| Olfr685 | 0.21 | 0.24 | 0.21 | 0.20 | 0.20 | 0.25 | 0.20 | 0.18 | 0.19 | 0.20 |
| Olfr686 | 0.33 | 0.33 | 0.26 | 0.28 | 0.25 | 0.26 | 0.23 | 0.22 | 0.30 | 0.27 |
| Olfr688 | 0.26 | 0.25 | 0.16 | 0.18 | 0.20 | 0.24 | 0.15 | 0.14 | 0.15 | 0.15 |
| Olfr689 | 0.34 | 0.35 | 0.24 | 0.27 | 0.27 | 0.28 | 0.22 | 0.21 | 0.24 | 0.22 |
| Olfr69 | 0.08 | 0.08 | 0.07 | 0.03 | 0.08 | 0.08 | 0.03 | 0.02 | 0.02 | 0.02 |
| Olfr690 | 0.31 | 0.36 | 0.26 | 0.26 | 0.29 | 0.29 | 0.21 | 0.21 | 0.24 | 0.22 |
| Olfr691 | 0.22 | 0.24 | 0.22 | 0.25 | 0.24 | 0.26 | 0.20 | 0.19 | 0.23 | 0.21 |
| Olfr692 | 0.31 | 0.33 | 0.25 | 0.26 | 0.26 | 0.25 | 0.23 | 0.23 | 0.24 | 0.24 |
| Olfr693 | 0.25 | 0.26 | 0.20 | 0.24 | 0.22 | 0.23 | 0.20 | 0.19 | 0.23 | 0.23 |
| Olfr694 | 0.25 | 0.25 | 0.20 | 0.23 | 0.21 | 0.21 | 0.19 | 0.18 | 0.20 | 0.19 |
| Olfr695 | 0.17 | 0.19 | 0.14 | 0.15 | 0.17 | 0.14 | 0.12 | 0.12 | 0.13 | 0.15 |
| Olfr697 | 0.25 | 0.28 | 0.22 | 0.25 | 0.25 | 0.25 | 0.23 | 0.23 | 0.22 | 0.21 |
| Olfr698 | 0.26 | 0.34 | 0.23 | 0.28 | 0.23 | 0.24 | 0.20 | 0.18 | 0.22 | 0.21 |
| Olfr699 | 0.18 | 0.22 | 0.16 | 0.20 | 0.19 | 0.21 | 0.16 | 0.15 | 0.17 | 0.15 |
| Olfr70 | 0.37 | 0.37 | 0.32 | 0.32 | 0.31 | 0.32 | 0.30 | 0.28 | 0.35 | 0.33 |
| Olfr700 | 0.27 | 0.30 | 0.22 | 0.23 | 0.23 | 0.27 | 0.17 | 0.19 | 0.18 | 0.18 |
| Olfr701 | 0.15 | 0.15 | 0.11 | 0.15 | 0.14 | 0.19 | 0.07 | 0.06 | 0.09 | 0.09 |
| Olfr702 | 0.15 | 0.17 | 0.09 | 0.12 | 0.14 | 0.14 | 0.07 | 0.07 | 0.08 | 0.06 |
| Olfr703 | 0.25 | 0.28 | 0.18 | 0.20 | 0.20 | 0.19 | 0.16 | 0.14 | 0.18 | 0.17 |
| Olfr704 | 0.15 | 0.16 | 0.15 | 0.19 | 0.15 | 0.18 | 0.11 | 0.12 | 0.15 | 0.09 |
| Olfr705 | 0.15 | 0.16 | 0.13 | 0.16 | 0.15 | 0.18 | 0.13 | 0.12 | 0.17 | 0.13 |
| Olfr706 | 0.27 | 0.29 | 0.21 | 0.19 | 0.22 | 0.20 | 0.20 | 0.18 | 0.17 | 0.19 |
| Olfr707 | 0.10 | 0.13 | 0.09 | 0.16 | 0.24 | 0.20 | 0.10 | 0.08 | 0.13 | 0.09 |
| Olfr71 | 0.51 | 0.52 | 0.44 | 0.47 | 0.47 | 0.49 | 0.42 | 0.40 | 0.51 | 0.48 |
| Olfr710 | 0.27 | 0.28 | 0.22 | 0.24 | 0.24 | 0.27 | 0.23 | 0.22 | 0.24 | 0.22 |
| Olfr711 | 0.27 | 0.28 | 0.23 | 0.25 | 0.24 | 0.23 | 0.20 | 0.19 | 0.27 | 0.24 |
| Olfr713 | 0.34 | 0.36 | 0.31 | 0.31 | 0.31 | 0.36 | 0.23 | 0.23 | 0.27 | 0.26 |
| Olfr714 | 0.29 | 0.20 | 0.22 | 0.21 | 0.23 | 0.24 | 0.23 | 0.23 | 0.31 | 0.26 |
| Olfr715 | 0.16 | 0.19 | 0.06 | 0.11 | 0.09 | 0.10 | 0.10 | 0.08 | 0.09 | 0.08 |
| Olfr716 | 0.20 | 0.21 | 0.16 | 0.19 | 0.17 | 0.16 | 0.14 | 0.13 | 0.16 | 0.15 |
| Olfr720 | 0.24 | 0.28 | 0.15 | 0.19 | 0.18 | 0.21 | 0.18 | 0.16 | 0.19 | 0.17 |
| Olfr722 | 0.02 | 0.09 | 0.03 | 0.06 | 0.05 | 0.08 | 0.01 | NA | 0.01 | 0.00 |
| Olfr723 | 0.35 | 0.33 | 0.24 | 0.26 | 0.28 | 0.26 | 0.22 | 0.22 | 0.22 | 0.22 |
| Olfr724 | 0.35 | 0.33 | 0.24 | 0.26 | 0.28 | 0.26 | 0.22 | 0.22 | 0.22 | 0.22 |
| Olfr725 | 0.15 | 0.20 | 0.14 | 0.14 | 0.19 | 0.18 | 0.09 | 0.07 | 0.14 | 0.11 |
| Olfr726 | 0.16 | 0.17 | 0.11 | 0.14 | 0.16 | 0.17 | 0.05 | 0.06 | 0.10 | 0.07 |
| Olfr727 | 0.06 | 0.07 | 0.06 | 0.07 | 0.11 | 0.09 | 0.08 | 0.05 | 0.06 | 0.05 |
| Olfr728 | 0.15 | 0.17 | 0.13 | 0.14 | 0.16 | 0.16 | 0.15 | 0.14 | 0.16 | 0.12 |
| Olfr729 | 0.13 | 0.13 | 0.08 | 0.09 | 0.12 | 0.13 | 0.09 | 0.07 | 0.05 | 0.08 |
| Olfr73 | 0.25 | 0.27 | 0.15 | 0.19 | 0.22 | 0.25 | 0.16 | 0.14 | 0.20 | 0.19 |
| Olfr730 | 0.19 | 0.22 | 0.16 | 0.16 | 0.20 | 0.21 | 0.14 | 0.13 | 0.12 | 0.12 |
| Olfr731 | 0.08 | 0.10 | 0.09 | 0.10 | 0.16 | 0.16 | 0.11 | 0.10 | 0.11 | 0.07 |
| Olfr732 | 0.10 | 0.14 | 0.09 | 0.12 | 0.10 | 0.10 | 0.13 | 0.12 | 0.10 | 0.08 |
| Olfr733 | 0.14 | 0.14 | 0.12 | 0.12 | 0.10 | 0.09 | 0.13 | 0.10 | 0.16 | 0.09 |
| Olfr734 | 0.22 | 0.23 | 0.21 | 0.20 | 0.19 | 0.17 | 0.19 | 0.17 | 0.30 | 0.22 |
| Olfr735 | 0.20 | 0.23 | 0.18 | 0.18 | 0.18 | 0.16 | 0.14 | 0.14 | 0.24 | 0.16 |
| Olfr736 | 0.25 | 0.28 | 0.22 | 0.25 | 0.19 | 0.21 | 0.20 | 0.18 | 0.23 | 0.20 |
| Olfr738 | 0.24 | 0.28 | 0.20 | 0.22 | 0.22 | 0.25 | 0.19 | 0.19 | 0.17 | 0.18 |
| Olfr739 | 0.05 | 0.14 | 0.02 | 0.03 | 0.01 | 0.03 | 0.01 | NA | 0.03 | NA |
| Olfr74 | 0.20 | 0.20 | 0.16 | 0.18 | 0.17 | 0.15 | 0.13 | 0.13 | 0.17 | 0.14 |
| Olfr740 | 0.21 | 0.21 | 0.18 | 0.21 | 0.19 | 0.20 | 0.14 | 0.12 | 0.19 | 0.17 |
| Olfr741 | 0.23 | 0.23 | 0.17 | 0.21 | 0.24 | 0.24 | 0.15 | 0.15 | 0.17 | 0.16 |
| Olfr742 | 0.07 | 0.17 | 0.04 | 0.06 | 0.07 | 0.04 | 0.02 | 0.01 | 0.00 | NA |
| Olfr743 | 0.21 | 0.29 | 0.23 | 0.20 | 0.19 | 0.20 | 0.11 | 0.10 | 0.12 | 0.10 |
| Olfr744 | 0.31 | 0.25 | 0.24 | 0.29 | 0.30 | 0.29 | 0.26 | 0.21 | 0.25 | 0.26 |
| Olfr745 | 0.36 | 0.38 | 0.26 | 0.28 | 0.28 | 0.28 | 0.26 | 0.23 | 0.27 | 0.26 |
| Olfr746 | 0.14 | 0.15 | 0.10 | 0.10 | 0.14 | 0.16 | 0.08 | 0.08 | 0.06 | 0.06 |
| Olfr747 | 0.03 | 0.08 | 0.03 | 0.07 | 0.13 | 0.13 | 0.02 | 0.03 | 0.02 | 0.02 |
| Olfr748 | 0.13 | 0.15 | 0.06 | 0.08 | 0.11 | 0.12 | 0.09 | 0.07 | 0.11 | 0.05 |
| Olfr749 | 0.06 | 0.06 | 0.06 | 0.07 | 0.09 | 0.10 | 0.03 | 0.03 | 0.03 | 0.04 |
| Olfr750 | 0.38 | 0.40 | 0.27 | 0.28 | 0.31 | 0.34 | 0.26 | 0.25 | 0.29 | 0.27 |
| Olfr76 | 0.30 | 0.35 | 0.22 | 0.22 | 0.22 | 0.25 | 0.18 | 0.19 | 0.24 | 0.25 |
| Olfr761 | 0.15 | 0.22 | 0.15 | 0.17 | 0.15 | 0.15 | 0.10 | 0.10 | 0.11 | 0.09 |
| Olfr763 | 0.31 | 0.30 | 0.19 | 0.20 | 0.21 | 0.23 | 0.17 | 0.15 | 0.18 | 0.18 |
| Olfr765 | 0.35 | 0.33 | 0.27 | 0.29 | 0.24 | 0.25 | 0.24 | 0.20 | 0.26 | 0.28 |
| Olfr767 | 0.51 | 0.50 | 0.31 | 0.36 | 0.30 | 0.35 | 0.53 | 0.53 | 0.56 | 0.58 |
| Olfr768 | 0.02 | NA | 0.02 | 0.05 | 0.07 | 0.10 | 0.01 | 0.00 | 0.04 | 0.02 |
| Olfr769 | 0.28 | 0.29 | 0.17 | 0.22 | 0.22 | 0.25 | 0.14 | 0.14 | 0.18 | 0.16 |
| Olfr77 | 0.16 | 0.20 | 0.09 | 0.11 | 0.11 | 0.11 | 0.09 | 0.08 | 0.11 | 0.09 |
| Olfr770 | 0.30 | 0.30 | 0.22 | 0.25 | 0.22 | 0.24 | 0.23 | 0.21 | 0.23 | 0.22 |
| Olfr771 | 0.11 | 0.12 | 0.05 | 0.08 | 0.12 | 0.12 | 0.05 | 0.05 | 0.09 | 0.08 |
| Olfr772 | 0.07 | 0.08 | 0.06 | 0.08 | 0.06 | 0.08 | 0.05 | 0.04 | 0.04 | 0.04 |
| Olfr773 | 0.17 | 0.18 | 0.14 | 0.11 | 0.15 | 0.16 | 0.15 | 0.12 | 0.09 | 0.10 |
| Olfr774 | 0.35 | 0.37 | 0.27 | 0.30 | 0.28 | 0.29 | 0.28 | 0.25 | 0.28 | 0.25 |
| Olfr775 | 0.13 | 0.15 | 0.08 | 0.12 | 0.15 | 0.14 | 0.11 | 0.10 | 0.09 | 0.08 |
| Olfr776 | 0.15 | 0.24 | 0.05 | 0.07 | 0.09 | 0.17 | 0.07 | 0.04 | 0.04 | 0.01 |
| Olfr777 | 0.08 | 0.10 | 0.06 | 0.07 | 0.10 | 0.08 | 0.06 | 0.07 | 0.06 | 0.07 |
| Olfr78 | 0.11 | 0.11 | 0.08 | 0.10 | 0.10 | 0.13 | 0.08 | 0.07 | 0.11 | 0.08 |
| Olfr780 | 0.10 | 0.14 | 0.11 | 0.16 | 0.12 | 0.14 | 0.10 | 0.09 | 0.12 | 0.11 |
| Olfr781 | 0.09 | 0.10 | 0.06 | 0.10 | 0.08 | 0.09 | 0.04 | 0.04 | 0.06 | 0.04 |
| Olfr782 | 0.12 | 0.18 | 0.08 | 0.15 | 0.18 | 0.20 | 0.09 | 0.07 | 0.11 | 0.10 |
| Olfr784 | 0.25 | 0.34 | 0.16 | 0.19 | 0.21 | 0.27 | 0.13 | 0.14 | 0.16 | 0.15 |
| Olfr786 | 0.19 | 0.27 | 0.10 | 0.12 | 0.09 | 0.08 | 0.07 | 0.07 | 0.08 | 0.08 |
| Olfr787 | 0.04 | 0.07 | 0.03 | 0.01 | 0.11 | 0.13 | 0.06 | 0.05 | 0.02 | 0.02 |
| Olfr788 | 0.35 | 0.34 | 0.23 | 0.22 | 0.21 | 0.22 | 0.15 | 0.15 | 0.20 | 0.17 |
| Olfr790 | 0.26 | 0.29 | 0.21 | 0.27 | 0.22 | 0.24 | 0.16 | 0.16 | 0.20 | 0.16 |
| Olfr791 | 0.14 | 0.17 | 0.11 | 0.13 | 0.08 | 0.11 | 0.06 | 0.06 | 0.10 | 0.09 |
| Olfr792 | 0.06 | 0.04 | 0.03 | 0.10 | 0.09 | 0.04 | 0.06 | 0.04 | 0.04 | 0.05 |
| Olfr794 | 0.23 | 0.22 | 0.17 | 0.18 | 0.20 | 0.19 | 0.13 | 0.12 | 0.15 | 0.16 |
| Olfr796 | 0.42 | 0.45 | 0.32 | 0.34 | 0.35 | 0.36 | 0.27 | 0.25 | 0.31 | 0.30 |
| Olfr798 | 0.03 | 0.12 | 0.08 | 0.07 | 0.15 | 0.14 | 0.04 | 0.03 | 0.02 | 0.04 |
| Olfr799 | 0.12 | 0.21 | 0.12 | 0.12 | 0.13 | 0.14 | 0.08 | 0.07 | 0.09 | 0.08 |
| Olfr8 | 0.13 | 0.16 | 0.03 | 0.06 | 0.06 | 0.08 | 0.01 | 0.01 | 0.04 | 0.01 |
| Olfr800 | 0.08 | 0.10 | 0.06 | 0.07 | 0.10 | 0.08 | 0.06 | 0.07 | 0.06 | 0.07 |
| Olfr801 | 0.25 | 0.28 | 0.14 | 0.18 | 0.19 | 0.19 | 0.11 | 0.12 | 0.16 | 0.12 |
| Olfr802 | 0.14 | 0.21 | 0.11 | 0.08 | 0.10 | 0.10 | 0.08 | 0.07 | 0.07 | 0.06 |
| Olfr803 | 0.12 | 0.16 | 0.10 | 0.08 | 0.12 | 0.10 | 0.02 | 0.05 | 0.02 | 0.03 |
| Olfr804 | 0.12 | 0.18 | 0.08 | 0.15 | 0.18 | 0.20 | 0.09 | 0.07 | 0.11 | 0.10 |
| Olfr805 | 0.12 | 0.15 | 0.10 | 0.10 | 0.10 | 0.12 | 0.08 | 0.07 | 0.08 | 0.07 |
| Olfr806 | 0.06 | 0.13 | NA | NA | 0.04 | 0.04 | NA | NA | NA | NA |
| Olfr807 | 0.39 | 0.40 | 0.31 | 0.33 | 0.33 | 0.36 | 0.28 | 0.26 | 0.30 | 0.30 |
| Olfr808 | 0.15 | 0.21 | 0.14 | 0.20 | 0.17 | 0.19 | 0.14 | 0.13 | 0.15 | 0.13 |
| Olfr809 | 0.07 | 0.08 | 0.04 | 0.07 | 0.07 | 0.08 | 0.01 | 0.03 | 0.05 | 0.03 |
| Olfr810 | 0.32 | 0.33 | 0.21 | 0.23 | 0.21 | 0.24 | 0.19 | 0.18 | 0.22 | 0.21 |
| Olfr811 | 0.24 | 0.25 | 0.21 | 0.23 | 0.24 | 0.25 | 0.13 | 0.15 | 0.17 | 0.15 |
| Olfr812 | 0.25 | 0.29 | 0.17 | 0.19 | 0.16 | 0.18 | 0.15 | 0.13 | 0.14 | 0.14 |
| Olfr813 | 0.12 | 0.15 | 0.10 | 0.13 | 0.08 | 0.07 | 0.06 | 0.07 | 0.08 | 0.08 |
| Olfr814 | 0.03 | 0.07 | 0.06 | 0.05 | 0.08 | 0.14 | NA | NA | 0.01 | 0.01 |
| Olfr815 | 0.06 | 0.07 | 0.02 | 0.04 | 0.05 | 0.08 | 0.00 | NA | 0.02 | 0.00 |
| Olfr816 | 0.08 | 0.13 | 0.07 | 0.07 | 0.09 | 0.10 | 0.04 | 0.04 | 0.04 | 0.03 |
| Olfr818 | 0.20 | 0.30 | 0.13 | 0.13 | 0.15 | 0.16 | 0.09 | 0.09 | 0.10 | 0.07 |
| Olfr819 | 0.46 | 0.48 | 0.29 | 0.31 | 0.28 | 0.31 | 0.28 | 0.26 | 0.28 | 0.23 |
| Olfr820 | 0.10 | 0.14 | 0.12 | 0.10 | 0.09 | 0.10 | 0.05 | 0.05 | 0.05 | 0.05 |
| Olfr821 | 0.14 | 0.14 | 0.12 | 0.11 | 0.13 | 0.13 | 0.12 | 0.10 | 0.11 | 0.10 |
| Olfr822 | 0.14 | 0.14 | 0.11 | 0.13 | 0.11 | 0.11 | 0.07 | 0.07 | 0.08 | 0.06 |
| Olfr823 | 0.10 | 0.19 | 0.05 | 0.08 | 0.09 | 0.08 | 0.07 | 0.04 | 0.04 | 0.05 |
| Olfr824 | 0.10 | 0.19 | 0.05 | 0.08 | 0.09 | 0.08 | 0.07 | 0.04 | 0.04 | 0.05 |
| Olfr825 | 0.22 | 0.25 | 0.15 | 0.18 | 0.21 | 0.21 | 0.13 | 0.11 | 0.13 | 0.13 |
| Olfr826 | 0.07 | 0.11 | 0.07 | 0.06 | 0.09 | 0.08 | 0.01 | NA | 0.03 | 0.00 |
| Olfr827 | 0.13 | 0.13 | 0.06 | 0.08 | 0.09 | 0.12 | 0.07 | 0.06 | 0.07 | 0.06 |
| Olfr828 | 0.13 | 0.17 | 0.09 | 0.13 | 0.13 | 0.13 | 0.07 | 0.07 | 0.07 | 0.07 |
| Olfr829 | 0.13 | 0.17 | 0.12 | 0.14 | 0.15 | 0.18 | 0.11 | 0.09 | 0.13 | 0.10 |
| Olfr830 | 0.22 | 0.20 | 0.11 | 0.17 | 0.15 | 0.18 | 0.13 | 0.10 | 0.13 | 0.10 |
| Olfr832 | 0.04 | 0.04 | 0.03 | 0.07 | 0.07 | 0.07 | 0.02 | 0.02 | 0.04 | 0.03 |
| Olfr834 | 0.00 | NA | 0.02 | 0.04 | 0.08 | 0.12 | NA | NA | 0.01 | NA |
| Olfr835 | 0.23 | 0.28 | 0.17 | 0.20 | 0.25 | 0.26 | 0.15 | 0.13 | 0.16 | 0.18 |
| Olfr836 | 0.13 | 0.23 | 0.05 | 0.07 | 0.06 | 0.06 | 0.03 | 0.01 | 0.02 | 0.04 |
| Olfr837 | 0.01 | 0.03 | 0.02 | 0.03 | 0.07 | 0.09 | 0.02 | NA | 0.02 | 0.02 |
| Olfr843 | 0.17 | 0.19 | 0.14 | 0.17 | 0.15 | 0.15 | 0.17 | 0.15 | 0.12 | 0.12 |
| Olfr845 | 0.20 | 0.20 | 0.22 | 0.19 | 0.19 | 0.20 | 0.14 | 0.12 | 0.14 | 0.13 |
| Olfr846 | NA | 0.02 | 0.02 | 0.03 | 0.04 | 0.05 | 0.01 | NA | 0.02 | 0.01 |
| Olfr847 | 0.11 | 0.14 | 0.02 | 0.10 | 0.10 | 0.11 | 0.03 | 0.03 | 0.04 | 0.05 |
| Olfr849 | 0.08 | 0.11 | 0.05 | 0.04 | 0.03 | 0.05 | 0.01 | 0.01 | NA | 0.00 |
| Olfr850 | 0.28 | 0.34 | 0.20 | 0.22 | 0.19 | 0.21 | 0.17 | 0.14 | 0.17 | 0.14 |
| Olfr851 | 0.17 | 0.20 | 0.13 | 0.14 | 0.16 | 0.17 | 0.08 | 0.08 | 0.08 | 0.11 |
| Olfr853 | 0.11 | 0.14 | 0.08 | 0.13 | 0.11 | 0.11 | 0.07 | 0.06 | 0.09 | 0.07 |
| Olfr854 | 0.10 | 0.14 | 0.06 | 0.10 | 0.11 | 0.11 | 0.05 | 0.06 | 0.06 | 0.05 |
| Olfr855 | 0.09 | 0.15 | 0.07 | 0.10 | 0.13 | 0.15 | 0.09 | 0.09 | 0.08 | 0.09 |
| Olfr856-ps1 | 0.26 | 0.25 | 0.18 | 0.21 | 0.16 | 0.21 | 0.15 | 0.12 | 0.14 | 0.13 |
| Olfr857 | 0.11 | 0.14 | 0.11 | 0.15 | 0.10 | 0.16 | 0.08 | 0.07 | 0.07 | 0.04 |
| Olfr859 | 0.05 | 0.04 | 0.01 | 0.11 | 0.07 | 0.07 | 0.03 | 0.03 | 0.02 | 0.04 |
| Olfr860 | 0.03 | 0.06 | 0.07 | 0.07 | 0.08 | 0.09 | 0.05 | 0.04 | 0.05 | 0.06 |
| Olfr862 | 0.12 | 0.13 | 0.13 | 0.14 | 0.15 | 0.22 | 0.10 | 0.08 | 0.10 | 0.08 |
| Olfr866 | 0.09 | 0.13 | 0.12 | 0.12 | 0.13 | 0.11 | 0.07 | 0.06 | 0.10 | 0.07 |
| Olfr867 | 0.26 | 0.28 | 0.20 | 0.18 | 0.24 | 0.21 | 0.19 | 0.17 | 0.22 | 0.19 |
| Olfr868 | 0.04 | 0.06 | 0.05 | 0.07 | 0.07 | 0.07 | 0.03 | 0.02 | 0.03 | 0.03 |
| Olfr869 | 0.03 | 0.05 | 0.03 | 0.06 | 0.11 | 0.09 | 0.03 | 0.03 | 0.06 | 0.03 |
| Olfr870 | 0.25 | 0.25 | 0.14 | 0.18 | 0.17 | 0.20 | 0.14 | 0.14 | 0.17 | 0.17 |
| Olfr871 | 0.23 | 0.25 | 0.20 | 0.21 | 0.19 | 0.25 | 0.14 | 0.13 | 0.16 | 0.14 |
| Olfr872 | 0.09 | 0.10 | 0.02 | 0.02 | 0.09 | 0.09 | 0.01 | 0.01 | 0.00 | 0.01 |
| Olfr873 | 0.20 | 0.22 | 0.06 | 0.11 | 0.09 | 0.08 | 0.06 | 0.05 | 0.08 | 0.07 |
| Olfr874 | 0.32 | 0.37 | 0.24 | 0.27 | 0.26 | 0.25 | 0.22 | 0.22 | 0.25 | 0.24 |
| Olfr875 | 0.26 | 0.32 | 0.16 | 0.18 | 0.21 | 0.24 | 0.14 | 0.15 | 0.17 | 0.15 |
| Olfr876 | 0.20 | 0.23 | 0.19 | 0.19 | 0.17 | 0.21 | 0.15 | 0.14 | 0.18 | 0.14 |
| Olfr877 | 0.18 | 0.23 | 0.11 | 0.15 | 0.13 | 0.14 | 0.12 | 0.09 | 0.12 | 0.09 |
| Olfr878 | 0.25 | 0.28 | 0.21 | 0.24 | 0.19 | 0.21 | 0.18 | 0.17 | 0.20 | 0.18 |
| Olfr881 | 0.14 | 0.12 | 0.06 | 0.08 | 0.10 | 0.11 | 0.09 | 0.08 | 0.07 | 0.07 |
| Olfr883 | 0.12 | 0.13 | 0.06 | 0.08 | 0.09 | 0.12 | 0.08 | 0.06 | 0.05 | 0.04 |
| Olfr884 | 0.12 | 0.13 | 0.06 | 0.08 | 0.09 | 0.12 | 0.08 | 0.06 | 0.05 | 0.04 |
| Olfr885 | 0.12 | 0.13 | 0.06 | 0.08 | 0.09 | 0.12 | 0.08 | 0.06 | 0.05 | 0.04 |
| Olfr887 | 0.12 | 0.13 | 0.06 | 0.08 | 0.09 | 0.12 | 0.08 | 0.06 | 0.05 | 0.04 |
| Olfr888 | 0.10 | 0.10 | 0.05 | 0.07 | 0.05 | 0.06 | 0.06 | 0.05 | 0.07 | 0.05 |
| Olfr889 | 0.06 | 0.08 | 0.03 | 0.07 | 0.12 | 0.13 | 0.03 | 0.04 | 0.03 | 0.04 |
| Olfr890 | 0.03 | 0.05 | 0.04 | 0.06 | 0.07 | 0.07 | 0.03 | 0.04 | 0.05 | 0.03 |
| Olfr891 | 0.16 | 0.18 | 0.14 | 0.12 | 0.13 | 0.12 | 0.11 | 0.09 | 0.09 | 0.08 |
| Olfr893 | 0.08 | 0.10 | 0.07 | 0.05 | 0.07 | 0.09 | 0.07 | 0.05 | 0.05 | 0.05 |
| Olfr894 | 0.09 | 0.06 | 0.05 | 0.07 | 0.06 | 0.10 | 0.06 | 0.05 | 0.07 | 0.04 |
| Olfr895 | 0.05 | 0.12 | 0.05 | 0.08 | 0.07 | 0.07 | 0.04 | 0.04 | 0.05 | 0.02 |
| Olfr898 | 0.32 | 0.35 | 0.18 | 0.20 | 0.21 | 0.19 | 0.19 | 0.18 | 0.21 | 0.17 |
| Olfr899 | 0.18 | 0.24 | 0.13 | 0.17 | 0.15 | 0.15 | 0.13 | 0.11 | 0.13 | 0.12 |
| Olfr9 | 0.23 | 0.27 | 0.10 | 0.13 | 0.16 | 0.16 | 0.09 | 0.10 | 0.13 | 0.14 |
| Olfr90 | 0.30 | 0.31 | 0.21 | 0.25 | 0.17 | 0.18 | 0.17 | 0.16 | 0.21 | 0.16 |
| Olfr900 | 0.17 | 0.20 | 0.13 | 0.16 | 0.17 | 0.20 | 0.13 | 0.11 | 0.13 | 0.11 |
| Olfr901 | 0.30 | 0.27 | 0.22 | 0.24 | 0.22 | 0.23 | 0.19 | 0.17 | 0.22 | 0.20 |
| Olfr902 | 0.22 | 0.26 | 0.19 | 0.20 | 0.23 | 0.25 | 0.14 | 0.14 | 0.17 | 0.16 |
| Olfr904 | 0.10 | 0.14 | 0.06 | 0.07 | 0.08 | 0.09 | 0.03 | 0.02 | 0.04 | 0.03 |
| Olfr905 | 0.15 | 0.19 | 0.01 | 0.06 | 0.09 | 0.10 | 0.03 | 0.02 | 0.06 | 0.03 |
| Olfr906 | 0.14 | 0.21 | 0.10 | 0.12 | 0.15 | 0.13 | 0.10 | 0.08 | 0.11 | 0.07 |
| Olfr907 | 0.11 | 0.11 | 0.08 | 0.12 | 0.10 | 0.09 | 0.08 | 0.07 | 0.09 | 0.08 |
| Olfr908 | 0.12 | 0.15 | 0.05 | 0.06 | 0.11 | 0.14 | 0.07 | 0.06 | 0.09 | 0.04 |
| Olfr91 | 0.63 | 0.63 | 0.43 | 0.46 | 0.45 | 0.46 | 0.38 | 0.38 | 0.44 | 0.43 |
| Olfr910 | 0.03 | 0.05 | 0.04 | 0.08 | 0.04 | 0.03 | 0.02 | 0.02 | 0.04 | 0.01 |
| Olfr911-ps1 | 0.08 | 0.12 | 0.07 | 0.08 | 0.07 | 0.07 | 0.06 | 0.04 | 0.04 | 0.02 |
| Olfr912 | 0.09 | 0.17 | 0.06 | 0.08 | 0.08 | 0.12 | 0.02 | 0.02 | 0.04 | 0.03 |
| Olfr913 | 0.19 | 0.22 | 0.09 | 0.13 | 0.15 | 0.18 | 0.09 | 0.07 | 0.07 | 0.09 |
| Olfr914 | 0.18 | 0.23 | 0.10 | 0.14 | 0.13 | 0.19 | 0.10 | 0.11 | 0.14 | 0.11 |
| Olfr915 | 0.19 | 0.22 | 0.14 | 0.15 | 0.17 | 0.19 | 0.12 | 0.12 | 0.13 | 0.10 |
| Olfr916 | 0.20 | 0.28 | 0.07 | 0.12 | 0.07 | 0.08 | 0.05 | 0.04 | 0.07 | 0.05 |
| Olfr917 | 0.17 | 0.15 | 0.14 | 0.18 | 0.13 | 0.13 | 0.13 | 0.09 | 0.16 | 0.14 |
| Olfr918 | 0.15 | 0.10 | 0.08 | 0.10 | 0.09 | 0.10 | 0.07 | 0.05 | 0.09 | 0.08 |
| Olfr919 | 0.25 | 0.27 | 0.20 | 0.25 | 0.20 | 0.25 | 0.18 | 0.19 | 0.20 | 0.18 |
| Olfr92 | 0.47 | 0.49 | 0.31 | 0.36 | 0.32 | 0.36 | 0.27 | 0.25 | 0.36 | 0.29 |
| Olfr920 | 0.61 | 0.58 | 0.39 | 0.55 | 0.34 | 0.36 | 0.21 | 0.27 | 0.28 | 0.21 |
| Olfr921 | 0.14 | 0.13 | 0.07 | 0.12 | 0.08 | 0.11 | 0.06 | 0.05 | 0.05 | 0.05 |
| Olfr922 | 0.24 | 0.26 | 0.21 | 0.19 | 0.17 | 0.19 | 0.14 | 0.14 | 0.20 | 0.16 |
| Olfr923 | 0.26 | 0.28 | 0.20 | 0.20 | 0.22 | 0.24 | 0.17 | 0.15 | 0.17 | 0.17 |
| Olfr924 | 0.02 | 0.06 | 0.04 | 0.04 | 0.03 | 0.04 | NA | NA | 0.01 | NA |
| Olfr926 | 0.05 | 0.05 | 0.03 | 0.04 | 0.06 | 0.05 | 0.01 | 0.01 | 0.03 | 0.02 |
| Olfr93 | 0.33 | 0.33 | 0.21 | 0.21 | 0.28 | 0.30 | 0.18 | 0.19 | 0.28 | 0.25 |
| Olfr930 | 0.19 | 0.21 | 0.09 | 0.15 | 0.12 | 0.11 | 0.09 | 0.08 | 0.08 | 0.07 |
| Olfr933 | 0.24 | 0.23 | 0.18 | 0.21 | 0.27 | 0.26 | 0.15 | 0.14 | 0.18 | 0.17 |
| Olfr934 | 0.14 | 0.15 | 0.07 | 0.09 | 0.13 | 0.12 | 0.10 | 0.08 | 0.08 | 0.08 |
| Olfr935 | 0.18 | 0.23 | 0.14 | 0.18 | 0.17 | 0.19 | 0.10 | 0.09 | 0.12 | 0.10 |
| Olfr936 | 0.08 | 0.13 | 0.02 | 0.05 | 0.04 | 0.04 | NA | NA | 0.03 | NA |
| Olfr937 | 0.14 | 0.13 | 0.07 | 0.14 | 0.10 | 0.12 | 0.09 | 0.08 | 0.07 | 0.06 |
| Olfr938 | 0.21 | 0.22 | 0.14 | 0.16 | 0.13 | 0.12 | 0.10 | 0.09 | 0.12 | 0.10 |
| Olfr94 | 0.22 | 0.23 | 0.23 | 0.24 | 0.23 | 0.25 | 0.24 | 0.22 | 0.28 | 0.30 |
| Olfr943 | 0.08 | 0.13 | 0.07 | 0.09 | 0.12 | 0.10 | 0.08 | 0.06 | 0.08 | 0.06 |
| Olfr944 | 0.09 | 0.10 | 0.03 | 0.07 | 0.07 | 0.08 | 0.06 | 0.05 | 0.06 | 0.03 |
| Olfr945 | 0.07 | 0.05 | 0.06 | 0.07 | 0.06 | 0.06 | 0.04 | 0.03 | 0.05 | 0.04 |
| Olfr947-ps1 | 0.09 | 0.08 | 0.09 | 0.11 | 0.12 | 0.13 | 0.06 | 0.06 | 0.08 | 0.06 |
| Olfr948 | 0.04 | 0.07 | 0.04 | 0.02 | 0.05 | 0.07 | 0.02 | 0.02 | NA | NA |
| Olfr95 | 0.39 | 0.37 | 0.30 | 0.29 | 0.34 | 0.37 | 0.29 | 0.28 | 0.37 | 0.36 |
| Olfr951 | 0.12 | 0.10 | 0.11 | 0.10 | 0.13 | 0.14 | 0.10 | 0.09 | 0.09 | 0.09 |
| Olfr952 | 0.12 | 0.13 | 0.05 | 0.07 | 0.02 | 0.05 | 0.03 | 0.01 | 0.04 | 0.03 |
| Olfr954 | 0.12 | 0.10 | 0.11 | 0.10 | 0.13 | 0.14 | 0.10 | 0.09 | 0.09 | 0.09 |
| Olfr955 | 0.18 | 0.19 | 0.13 | 0.11 | 0.11 | 0.13 | 0.11 | 0.09 | 0.10 | 0.09 |
| Olfr957 | 0.18 | 0.19 | 0.12 | 0.13 | 0.16 | 0.18 | 0.11 | 0.11 | 0.10 | 0.09 |
| Olfr958 | 0.33 | 0.36 | 0.24 | 0.26 | 0.27 | 0.34 | 0.25 | 0.23 | 0.26 | 0.25 |
| Olfr959 | 0.34 | 0.33 | 0.29 | 0.29 | 0.26 | 0.29 | 0.23 | 0.21 | 0.27 | 0.25 |
| Olfr96 | 0.30 | 0.32 | 0.25 | 0.27 | 0.22 | 0.22 | 0.21 | 0.20 | 0.24 | 0.26 |
| Olfr960 | 0.24 | 0.24 | 0.17 | 0.14 | 0.15 | 0.19 | 0.10 | 0.07 | 0.10 | 0.13 |
| Olfr961 | 0.13 | 0.14 | 0.10 | 0.14 | 0.12 | 0.12 | 0.08 | 0.06 | 0.08 | 0.07 |
| Olfr963 | 0.11 | 0.19 | 0.14 | 0.11 | 0.14 | 0.15 | 0.14 | 0.11 | 0.10 | 0.13 |
| Olfr965 | 0.15 | 0.19 | 0.18 | 0.18 | 0.15 | 0.17 | 0.17 | 0.15 | 0.18 | 0.16 |
| Olfr967 | 0.16 | 0.19 | 0.19 | 0.20 | 0.18 | 0.23 | 0.18 | 0.15 | 0.16 | 0.15 |
| Olfr968 | 0.07 | 0.10 | 0.04 | 0.05 | 0.04 | 0.03 | 0.04 | 0.03 | 0.02 | 0.01 |
| Olfr969 | 0.12 | 0.12 | 0.08 | 0.07 | 0.10 | 0.11 | 0.07 | 0.06 | 0.06 | 0.04 |
| Olfr97 | 0.12 | 0.18 | 0.14 | 0.15 | 0.14 | 0.20 | 0.07 | 0.07 | 0.24 | 0.19 |
| Olfr970 | 0.09 | 0.12 | 0.04 | 0.09 | 0.05 | 0.09 | 0.04 | 0.03 | 0.03 | 0.02 |
| Olfr971 | 0.15 | 0.16 | 0.12 | 0.11 | 0.13 | 0.14 | 0.09 | 0.09 | 0.09 | 0.09 |
| Olfr972 | 0.12 | 0.16 | 0.07 | 0.08 | 0.06 | 0.09 | 0.04 | 0.03 | 0.04 | 0.04 |
| Olfr974 | 0.32 | 0.31 | 0.27 | 0.30 | 0.29 | 0.33 | 0.24 | 0.23 | 0.30 | 0.25 |
| Olfr975 | 0.38 | 0.43 | 0.31 | 0.33 | 0.33 | 0.37 | 0.28 | 0.27 | 0.32 | 0.30 |
| Olfr976 | 0.23 | 0.24 | 0.18 | 0.20 | 0.18 | 0.21 | 0.16 | 0.17 | 0.19 | 0.17 |
| Olfr978 | 0.37 | 0.38 | 0.25 | 0.29 | 0.29 | 0.30 | 0.24 | 0.22 | 0.28 | 0.26 |
| Olfr979 | 0.22 | 0.24 | 0.18 | 0.20 | 0.16 | 0.15 | 0.17 | 0.16 | 0.18 | 0.19 |
| Olfr98 | 0.12 | 0.18 | 0.14 | 0.15 | 0.14 | 0.20 | 0.07 | 0.07 | 0.24 | 0.19 |
| Olfr980 | 0.28 | 0.25 | 0.22 | 0.24 | 0.26 | 0.27 | 0.18 | 0.19 | 0.20 | 0.17 |
| Olfr981 | 0.27 | 0.26 | 0.25 | 0.25 | 0.21 | 0.24 | 0.22 | 0.21 | 0.27 | 0.25 |
| Olfr982 | 0.46 | 0.48 | 0.40 | 0.44 | 0.39 | 0.42 | 0.34 | 0.34 | 0.39 | 0.38 |
| Olfr983 | 0.12 | 0.13 | 0.06 | 0.08 | 0.09 | 0.12 | 0.08 | 0.06 | 0.05 | 0.04 |
| Olfr984 | 0.37 | 0.40 | 0.31 | 0.34 | 0.36 | 0.39 | 0.30 | 0.28 | 0.34 | 0.32 |
| Olfr985 | 0.26 | 0.25 | 0.20 | 0.18 | 0.20 | 0.21 | 0.19 | 0.18 | 0.17 | 0.19 |
| Olfr986 | 0.30 | 0.31 | 0.24 | 0.26 | 0.25 | 0.24 | 0.22 | 0.21 | 0.22 | 0.21 |
| Olfr987 | 0.11 | 0.13 | 0.12 | 0.11 | 0.10 | 0.12 | 0.09 | 0.07 | 0.10 | 0.07 |
| Olfr988 | 0.24 | 0.26 | 0.15 | 0.22 | 0.15 | 0.17 | 0.13 | 0.12 | 0.15 | 0.08 |
| Olfr99 | 0.64 | 0.63 | 0.42 | 0.48 | 0.36 | 0.42 | 0.18 | 0.16 | 0.36 | 0.25 |
| Olfr992 | 0.12 | 0.11 | 0.11 | 0.19 | 0.10 | 0.10 | 0.11 | 0.09 | 0.12 | 0.11 |
| Olfr993 | 0.17 | 0.10 | 0.11 | 0.11 | 0.19 | 0.17 | 0.03 | 0.03 | 0.07 | 0.04 |
| Olfr994 | 0.08 | 0.12 | 0.08 | 0.11 | 0.11 | 0.07 | 0.07 | 0.07 | 0.08 | 0.06 |
| Olfr995 | 0.09 | 0.08 | 0.03 | 0.06 | 0.07 | 0.06 | 0.02 | 0.02 | 0.03 | 0.02 |
| Olfr996 | 0.03 | 0.07 | 0.04 | 0.06 | 0.09 | 0.13 | 0.05 | 0.04 | 0.03 | 0.04 |
| Olfr998 | 0.33 | 0.37 | 0.19 | 0.19 | 0.21 | 0.22 | 0.19 | 0.17 | 0.19 | 0.17 |

| | Lung | Red | | MLN | | | | |
|---|---|---|---|---|---|---|---|---|
| | CD11b − | Pulp | Mo.6C + | DCs | Microglia | Mo.6C − | M0.6C + | M0.6C − |
| | Mφ | Mφ | II − .B1 | CD4+ | Mφ | II − .B1 | II + .B1 | II + .B1 |
| Gapdh | 1.00 | 1.00 | 1.00 | 1.00 | 1.00 | 1.00 | 1.00 | 1.00 |
| Rtp1 | 0.24 | 0.30 | 0.27 | 0.33 | 0.31 | 0.30 | 0.28 | 0.33 |
| Rtp2 | 0.32 | 0.35 | 0.31 | 0.37 | 0.35 | 0.38 | 0.36 | 0.39 |
| Reep3 | 0.718 | 0.693 | 0.71 | 0.716 | 0.601 | 0.53 | 0.689 | 0.576 |
| Cnga1 | 0.107 | 0.131 | 0.115 | 0.135 | 0.138 | 0.107 | 0.13 | 0.139 |
| Cnga2 | 0.153 | 0.156 | 0.145 | 0.199 | 0.185 | 0.169 | 0.15 | 0.17 |
| Cnga3 | 0.231 | 0.263 | 0.228 | 0.331 | 0.303 | 0.268 | 0.263 | 0.3 |
| Cnga4 | 0.196 | 0.24 | 0.209 | 0.23 | 0.242 | 0.264 | 0.207 | 0.265 |
| Cngb1 | 0.257 | 0.27 | 0.286 | 0.253 | 0.252 | 0.28 | 0.244 | 0.235 |
| Acy3 | 0.408 | 0.404 | 0.313 | 0.384 | 0.385 | 0.389 | 0.341 | 0.39 |
| Gna1 | 0.15 | 0.17 | 0.20 | 0.18 | 0.19 | 0.22 | 0.21 | 0.22 |
| Olfr1 | 0.19 | 0.24 | 0.21 | 0.26 | 0.19 | 0.28 | 0.21 | 0.29 |
| Olfr10 | 0.20 | 0.28 | 0.25 | 0.26 | 0.25 | 0.28 | 0.22 | 0.20 |
| Olfr100 | 0.13 | 0.15 | 0.15 | 0.19 | 0.20 | 0.18 | 0.21 | 0.19 |
| Olfr1000 | 0.07 | 0.08 | 0.07 | 0.07 | 0.10 | 0.09 | 0.08 | 0.14 |
| Olfr1002 | 0.02 | 0.04 | 0.03 | 0.04 | 0.02 | 0.06 | 0.05 | 0.10 |
| Olfr1006 | 0.15 | 0.20 | 0.18 | 0.20 | 0.22 | 0.21 | 0.18 | 0.23 |
| Olfr1008 | 0.07 | 0.11 | 0.10 | 0.13 | 0.10 | 0.12 | 0.07 | 0.15 |
| Olfr1009 | 0.15 | 0.15 | 0.15 | 0.21 | 0.21 | 0.20 | 0.18 | 0.21 |
| Olfr101 | 0.15 | 0.18 | 0.18 | 0.21 | 0.19 | 0.20 | 0.20 | 0.19 |
| Olfr1010 | 0.18 | 0.21 | 0.19 | 0.25 | 0.23 | 0.25 | 0.22 | 0.30 |
| Olfr1012 | 0.20 | 0.24 | 0.23 | 0.26 | 0.25 | 0.24 | 0.22 | 0.24 |
| Olfr1013 | 0.05 | 0.08 | 0.07 | 0.08 | 0.07 | 0.09 | 0.06 | 0.07 |
| Olfr1014 | 0.05 | 0.07 | 0.06 | 0.08 | 0.06 | 0.09 | 0.08 | 0.10 |
| Olfr1015 | 0.18 | 0.23 | 0.22 | 0.23 | 0.23 | 0.25 | 0.25 | 0.25 |
| Olfr1016 | 0.20 | 0.24 | 0.23 | 0.29 | 0.26 | 0.25 | 0.23 | 0.24 |
| Olfr1018 | 0.15 | 0.15 | 0.15 | 0.17 | 0.17 | 0.19 | 0.19 | 0.17 |
| Olfr1019 | 0.17 | 0.19 | 0.19 | 0.19 | 0.20 | 0.21 | 0.21 | 0.18 |
| Olfr102 | 0.13 | 0.15 | 0.15 | 0.19 | 0.20 | 0.18 | 0.21 | 0.19 |
| Olfr1020 | 0.05 | 0.06 | 0.06 | 0.09 | 0.07 | 0.07 | 0.05 | 0.11 |
| Olfr1022 | 0.06 | 0.09 | 0.08 | 0.11 | 0.11 | 0.11 | 0.09 | 0.13 |
| Olfr1023 | 0.10 | 0.10 | 0.10 | 0.14 | 0.13 | 0.15 | 0.13 | 0.18 |
| Olfr1024 | 0.15 | 0.18 | 0.17 | 0.16 | 0.18 | 0.21 | 0.20 | 0.20 |
| Olfr1026 | 0.09 | 0.12 | 0.10 | 0.14 | 0.13 | 0.13 | 0.14 | 0.13 |
| Olfr1028 | 0.08 | 0.09 | 0.09 | 0.10 | 0.12 | 0.13 | 0.10 | 0.20 |
| Olfr1029 | 0.12 | 0.18 | 0.18 | 0.18 | 0.23 | 0.20 | 0.24 | 0.25 |
| Olfr103 | 0.14 | 0.14 | 0.12 | 0.13 | 0.14 | 0.16 | 0.12 | 0.15 |
| Olfr1030 | 0.07 | 0.11 | 0.08 | 0.14 | 0.13 | 0.11 | 0.10 | 0.10 |
| Olfr1031 | 0.08 | 0.08 | 0.08 | 0.09 | 0.09 | 0.11 | 0.07 | 0.12 |
| Olfr1032 | 0.12 | 0.14 | 0.13 | 0.13 | 0.15 | 0.15 | 0.16 | 0.19 |
| Olfr1033 | 0.03 | 0.03 | 0.03 | 0.03 | 0.04 | 0.05 | 0.03 | 0.08 |
| Olfr1034 | 0.14 | 0.20 | 0.16 | 0.17 | 0.16 | 0.17 | 0.17 | 0.16 |
| Olfr1036 | 0.14 | 0.20 | 0.16 | 0.17 | 0.16 | 0.17 | 0.17 | 0.16 |
| Olfr1037 | 0.03 | 0.04 | 0.04 | 0.05 | 0.05 | 0.05 | 0.05 | 0.10 |
| Olfr1038-ps | 0.06 | 0.10 | 0.07 | 0.09 | 0.09 | 0.11 | 0.09 | 0.11 |
| Olfr1039 | NA | NA | 0.00 | 0.02 | 0.01 | 0.03 | 0.01 | 0.03 |
| Olfr1040 | 0.16 | 0.19 | 0.18 | 0.18 | 0.17 | 0.21 | 0.19 | 0.19 |
| Olfr1042 | 0.20 | 0.26 | 0.24 | 0.27 | 0.25 | 0.23 | 0.25 | 0.20 |
| Olfr1043 | 0.15 | 0.20 | 0.18 | 0.19 | 0.22 | 0.21 | 0.18 | 0.16 |
| Olfr1044 | 0.10 | 0.13 | 0.12 | 0.12 | 0.14 | 0.15 | 0.15 | 0.17 |
| Olfr1045 | 0.10 | 0.12 | 0.12 | 0.12 | 0.13 | 0.13 | 0.15 | 0.16 |
| Olfr1046 | NA | 0.02 | 0.00 | 0.02 | 0.02 | 0.02 | 0.03 | 0.04 |
| Olfr1047 | NA | NA | NA | NA | −0.01 | NA | 0.00 | 0.03 |
| Olfr1048 | 0.11 | 0.11 | 0.11 | 0.14 | 0.14 | 0.13 | 0.12 | 0.21 |
| Olfr1049 | NA | NA | NA | NA | −0.02 | 0.01 | 0.03 | 0.12 |
| Olfr1051 | NA | NA | NA | NA | −0.02 | 0.01 | NA | 0.07 |
| Olfr1052 | 0.07 | 0.08 | 0.08 | 0.09 | 0.11 | 0.08 | 0.12 | 0.14 |
| Olfr1053 | 0.13 | 0.18 | 0.17 | 0.22 | 0.18 | 0.17 | 0.17 | 0.28 |
| Olfr1054 | 0.15 | 0.20 | 0.15 | 0.20 | 0.17 | 0.20 | 0.17 | 0.23 |
| Olfr1055 | 0.07 | 0.08 | 0.08 | 0.08 | 0.09 | 0.11 | 0.09 | 0.13 |
| Olfr1056 | 0.13 | 0.14 | 0.15 | 0.15 | 0.15 | 0.15 | 0.13 | 0.19 |
| Olfr1057 | 0.12 | 0.14 | 0.12 | 0.15 | 0.15 | 0.17 | 0.16 | 0.18 |
| Olfr1058 | 0.05 | 0.07 | 0.05 | 0.06 | 0.08 | 0.06 | 0.08 | 0.06 |
| Olfr106-ps | 0.01 | 0.03 | 0.01 | 0.01 | 0.02 | 0.04 | 0.06 | 0.06 |
| Olfr1061 | NA | NA | NA | 0.01 | 0.01 | NA | 0.03 | 0.09 |
| Olfr1062 | 0.11 | 0.15 | 0.11 | 0.17 | 0.13 | 0.11 | 0.09 | 0.18 |
| Olfr1065 | 0.05 | 0.07 | 0.05 | 0.06 | 0.08 | 0.06 | 0.08 | 0.06 |
| Olfr1066 | NA | NA | NA | 0.02 | 0.01 | 0.00 | 0.01 | 0.03 |
| Olfr107 | 0.04 | 0.06 | 0.04 | 0.07 | 0.07 | 0.07 | 0.06 | 0.16 |
| Olfr1076 | 0.06 | 0.09 | 0.06 | 0.04 | 0.08 | 0.08 | 0.08 | 0.07 |
| Olfr1077-ps1 | 0.18 | 0.20 | 0.19 | 0.21 | 0.21 | 0.21 | 0.20 | 0.27 |
| Olfr1079 | 0.03 | 0.06 | 0.06 | 0.07 | 0.07 | 0.08 | 0.08 | 0.07 |
| Olfr108 | 0.03 | 0.07 | 0.07 | 0.08 | 0.06 | 0.09 | 0.09 | 0.07 |
| Olfr1080 | NA | 0.01 | 0.01 | 0.04 | 0.03 | 0.01 | 0.04 | 0.08 |
| Olfr1082 | NA | NA | NA | NA | 0.00 | 0.01 | 0.04 | 0.02 |
| Olfr1084 | NA | NA | NA | NA | 0.00 | NA | NA | 0.09 |
| Olfr1085 | 0.13 | 0.15 | 0.16 | 0.17 | 0.19 | 0.23 | 0.19 | 0.25 |
| Olfr1086 | 0.17 | 0.22 | 0.20 | 0.22 | 0.19 | 0.24 | 0.24 | 0.22 |
| Olfr1087 | 0.09 | 0.11 | 0.10 | 0.11 | 0.13 | 0.11 | 0.11 | 0.12 |
| Olfr1089 | 0.08 | 0.12 | 0.09 | 0.11 | 0.08 | 0.12 | 0.15 | 0.13 |
| Olfr109 | 0.23 | 0.28 | 0.24 | 0.28 | 0.25 | 0.28 | 0.28 | 0.25 |
| Olfr1090 | 0.06 | 0.09 | 0.09 | 0.10 | 0.09 | 0.09 | 0.07 | 0.10 |
| Olfr1093 | 0.17 | 0.17 | 0.17 | 0.19 | 0.17 | 0.20 | 0.20 | 0.24 |
| Olfr1094 | 0.06 | 0.11 | 0.08 | 0.10 | 0.10 | 0.08 | 0.07 | 0.17 |
| Olfr1095 | 0.00 | 0.03 | 0.04 | 0.06 | 0.05 | 0.09 | 0.04 | 0.08 |
| Olfr1097 | 0.04 | 0.08 | 0.06 | 0.07 | 0.07 | 0.09 | 0.10 | 0.06 |
| Olfr1098 | 0.06 | 0.07 | 0.06 | 0.06 | 0.07 | 0.07 | 0.07 | 0.12 |
| Olfr1099 | 0.12 | 0.18 | 0.18 | 0.16 | 0.19 | 0.18 | 0.17 | 0.14 |
| Olfr11 | 0.21 | 0.25 | 0.24 | 0.28 | 0.24 | 0.27 | 0.24 | 0.32 |
| Olfr110 | 0.15 | 0.19 | 0.16 | 0.21 | 0.25 | 0.19 | 0.18 | 0.18 |
| Olfr1100 | 0.02 | 0.04 | 0.03 | 0.04 | 0.04 | 0.05 | 0.05 | 0.06 |
| Olfr1101 | 0.08 | 0.11 | 0.10 | 0.11 | 0.11 | 0.17 | 0.14 | 0.17 |
| Olfr1102 | 0.00 | 0.01 | 0.02 | 0.02 | 0.02 | 0.03 | 0.04 | 0.01 |
| Olfr1104 | 0.13 | 0.15 | 0.12 | 0.17 | 0.15 | 0.17 | 0.18 | 0.13 |
| Olfr1105 | 0.18 | 0.21 | 0.20 | 0.23 | 0.21 | 0.25 | 0.24 | 0.18 |
| Olfr1106 | 0.07 | 0.10 | 0.08 | 0.10 | 0.10 | 0.11 | 0.11 | 0.12 |
| Olfr1107 | 0.02 | 0.04 | 0.02 | 0.04 | 0.06 | 0.03 | 0.03 | 0.06 |
| Olfr1109 | 0.01 | 0.05 | 0.04 | 0.07 | 0.06 | 0.06 | 0.07 | 0.19 |
| Olfr111 | 0.18 | 0.21 | 0.10 | 0.36 | 0.37 | 0.14 | 0.15 | 0.16 |
| Olfr1110 | 0.07 | 0.12 | 0.12 | 0.15 | 0.09 | 0.13 | 0.16 | 0.16 |
| Olfr1111 | 0.11 | 0.12 | 0.11 | 0.13 | 0.13 | 0.12 | 0.13 | 0.14 |
| Olfr1112 | 0.03 | 0.05 | 0.04 | 0.07 | 0.08 | 0.06 | 0.08 | 0.05 |
| Olfr1113 | 0.07 | 0.14 | 0.11 | 0.14 | 0.14 | 0.11 | 0.13 | 0.12 |
| Olfr1115 | 0.16 | 0.20 | 0.18 | 0.20 | 0.19 | 0.22 | 0.20 | 0.22 |
| Olfr1116 | 0.01 | 0.02 | 0.02 | 0.03 | 0.02 | 0.02 | 0.03 | 0.08 |
| Olfr1118 | 0.11 | 0.16 | 0.14 | 0.14 | 0.15 | 0.16 | 0.15 | 0.20 |
| Olfr112 | 0.02 | 0.05 | 0.04 | 0.03 | 0.05 | 0.04 | 0.05 | 0.08 |
| Olfr1120 | 0.10 | 0.14 | 0.12 | 0.16 | 0.15 | 0.16 | 0.16 | 0.20 |
| Olfr1121 | 0.08 | 0.13 | 0.09 | 0.11 | 0.12 | 0.10 | 0.09 | 0.15 |
| Olfr1122 | 0.16 | 0.19 | 0.15 | 0.18 | 0.18 | 0.18 | 0.18 | 0.20 |
| Olfr1123 | 0.05 | 0.07 | 0.05 | 0.08 | 0.08 | 0.07 | 0.07 | 0.06 |
| Olfr1124 | NA | 0.03 | 0.01 | 0.01 | 0.02 | 0.03 | 0.04 | 0.04 |
| Olfr1126 | 0.16 | 0.20 | 0.18 | 0.21 | 0.20 | 0.22 | 0.19 | 0.20 |
| Olfr1128 | 0.17 | 0.23 | 0.20 | 0.25 | 0.25 | 0.23 | 0.21 | 0.27 |
| Olfr1129 | 0.05 | 0.08 | 0.06 | 0.10 | 0.09 | 0.09 | 0.08 | 0.14 |
| Olfr113 | 0.21 | 0.25 | 0.24 | 0.20 | 0.25 | 0.28 | 0.24 | 0.26 |
| Olfr1130 | 0.05 | 0.10 | 0.06 | 0.10 | 0.08 | 0.10 | 0.10 | 0.14 |
| Olfr1131 | 0.07 | 0.11 | 0.10 | 0.11 | 0.11 | 0.10 | 0.14 | 0.21 |
| Olfr1132 | 0.15 | 0.14 | 0.15 | 0.18 | 0.17 | 0.18 | 0.16 | 0.25 |
| Olfr1133 | 0.17 | 0.20 | 0.19 | 0.20 | 0.20 | 0.22 | 0.20 | 0.25 |
| Olfr1134 | 0.03 | 0.04 | 0.02 | 0.05 | 0.04 | 0.09 | 0.06 | 0.15 |
| Olfr1135 | 0.03 | 0.06 | 0.06 | 0.06 | 0.06 | 0.05 | 0.07 | 0.07 |
| Olfr1136 | 0.11 | 0.13 | 0.12 | 0.14 | 0.14 | 0.15 | 0.15 | 0.16 |
| Olfr1137 | 0.13 | 0.14 | 0.15 | 0.15 | 0.19 | 0.17 | 0.18 | 0.23 |
| Olfr1138 | 0.11 | 0.14 | 0.13 | 0.15 | 0.15 | 0.15 | 0.13 | 0.16 |
| Olfr114 | 0.10 | 0.14 | 0.10 | 0.14 | 0.13 | 0.16 | 0.13 | 0.23 |
| Olfr1140 | 0.10 | 0.11 | 0.09 | 0.13 | 0.12 | 0.14 | 0.13 | 0.13 |
| Olfr1141 | 0.05 | 0.09 | 0.06 | 0.08 | 0.08 | 0.07 | 0.08 | 0.09 |
| Olfr1143 | 0.06 | 0.08 | 0.08 | 0.08 | 0.07 | 0.09 | 0.09 | 0.07 |
| Olfr1145 | 0.10 | 0.13 | 0.10 | 0.11 | 0.11 | 0.13 | 0.14 | 0.14 |
| Olfr1148 | 0.35 | 0.35 | 0.32 | 0.33 | 0.34 | 0.32 | 0.29 | 0.23 |
| Olfr115 | 0.18 | 0.21 | 0.19 | 0.21 | 0.20 | 0.20 | 0.20 | 0.18 |
| Olfr1151 | 0.09 | 0.15 | 0.12 | 0.13 | 0.13 | 0.11 | 0.14 | 0.16 |
| Olfr1152 | 0.31 | 0.38 | 0.37 | 0.35 | 0.35 | 0.39 | 0.31 | 0.35 |
| Olfr1153 | 0.13 | 0.15 | 0.14 | 0.18 | 0.18 | 0.17 | 0.19 | 0.17 |
| Olfr1154 | 0.07 | 0.10 | 0.08 | 0.11 | 0.11 | 0.11 | 0.13 | 0.22 |
| Olfr1155 | 0.25 | 0.29 | 0.27 | 0.29 | 0.29 | 0.31 | 0.30 | 0.33 |
| Olfr1156 | 0.17 | 0.20 | 0.21 | 0.23 | 0.22 | 0.23 | 0.22 | 0.20 |
| Olfr1157 | 0.14 | 0.16 | 0.12 | 0.16 | 0.16 | 0.17 | 0.15 | 0.17 |
| Olfr1158 | 0.09 | 0.11 | 0.08 | 0.12 | 0.12 | 0.13 | 0.11 | 0.11 |
| Olfr116 | 0.07 | 0.09 | 0.08 | 0.13 | 0.14 | 0.10 | 0.09 | 0.16 |
| Olfr1160 | NA | 0.00 | 0.01 | 0.03 | 0.00 | NA | 0.09 | 0.08 |
| Olfr1161 | 0.09 | 0.11 | 0.08 | 0.11 | 0.11 | 0.10 | 0.10 | 0.16 |
| Olfr1162 | 0.20 | 0.23 | 0.21 | 0.25 | 0.22 | 0.25 | 0.26 | 0.25 |
| Olfr1163 | 0.20 | 0.23 | 0.21 | 0.25 | 0.22 | 0.25 | 0.26 | 0.25 |
| Olfr1164 | 0.09 | 0.07 | 0.09 | 0.10 | 0.07 | 0.13 | 0.09 | 0.15 |
| Olfr1166 | 0.07 | 0.11 | 0.08 | 0.15 | 0.10 | 0.12 | 0.10 | 0.15 |
| Olfr1167 | 0.02 | 0.01 | 0.03 | 0.03 | 0.03 | 0.04 | 0.04 | 0.04 |
| Olfr1168 | 0.09 | 0.12 | 0.11 | 0.13 | 0.13 | 0.14 | 0.13 | 0.20 |
| Olfr117 | 0.09 | 0.13 | 0.11 | 0.10 | 0.13 | 0.12 | 0.14 | 0.18 |
| Olfr1170 | 0.08 | 0.12 | 0.09 | 0.11 | 0.12 | 0.10 | 0.08 | 0.11 |
| Olfr1173 | 0.05 | 0.08 | 0.06 | 0.06 | 0.06 | 0.08 | 0.09 | 0.11 |
| Olfr1174-ps | 0.57 | 0.69 | 0.61 | 0.72 | 0.78 | 0.67 | 0.61 | 0.59 |
| Olfr1176 | 0.05 | 0.07 | 0.07 | 0.09 | 0.08 | 0.08 | 0.09 | 0.13 |
| Olfr1178 | 0.06 | 0.07 | 0.06 | 0.08 | 0.09 | 0.08 | 0.07 | 0.09 |
| Olfr1179 | 0.15 | 0.17 | 0.17 | 0.16 | 0.21 | 0.21 | 0.23 | 0.21 |
| Olfr118 | 0.12 | 0.12 | 0.10 | 0.16 | 0.14 | 0.10 | 0.16 | 0.30 |
| Olfr1180 | 0.06 | 0.08 | 0.07 | 0.09 | 0.08 | 0.08 | 0.08 | 0.14 |
| Olfr1181 | 0.03 | 0.04 | 0.02 | 0.04 | 0.04 | 0.09 | 0.08 | 0.10 |
| Olfr1182 | 0.09 | 0.09 | 0.10 | 0.13 | 0.09 | 0.14 | 0.15 | 0.19 |
| Olfr1183 | 0.01 | 0.03 | NA | 0.03 | 0.10 | 0.03 | 0.03 | 0.08 |
| Olfr1184 | 0.04 | 0.05 | 0.06 | 0.08 | 0.08 | 0.08 | 0.07 | 0.05 |
| Olfr1186 | 0.01 | 0.04 | 0.02 | 0.05 | 0.05 | 0.06 | 0.05 | 0.06 |
| Olfr1188 | 0.12 | 0.13 | 0.11 | 0.18 | 0.16 | 0.14 | 0.16 | 0.15 |
| Olfr1189 | 0.06 | 0.07 | 0.07 | 0.09 | 0.10 | 0.10 | 0.07 | 0.10 |
| Olfr119 | 0.08 | 0.12 | 0.10 | 0.11 | 0.14 | 0.12 | 0.12 | 0.18 |
| Olfr1193 | 0.08 | 0.10 | 0.11 | 0.08 | 0.10 | 0.12 | 0.05 | 0.11 |
| Olfr1195 | 0.03 | 0.04 | 0.04 | 0.08 | 0.07 | 0.08 | 0.07 | 0.04 |
| Olfr1196 | 0.14 | 0.18 | 0.14 | 0.18 | 0.21 | 0.18 | 0.17 | 0.20 |
| Olfr1197 | 0.14 | 0.18 | 0.14 | 0.18 | 0.21 | 0.18 | 0.17 | 0.20 |
| Olfr1198 | 0.04 | 0.05 | 0.04 | 0.04 | 0.04 | 0.04 | 0.07 | 0.11 |
| Olfr1199 | 0.01 | NA | 0.02 | 0.03 | 0.01 | 0.02 | 0.02 | 0.08 |
| Olfr12 | 0.24 | 0.27 | 0.25 | 0.31 | 0.27 | 0.29 | 0.27 | 0.25 |
| Olfr120 | 0.08 | 0.16 | 0.11 | 0.13 | 0.14 | 0.14 | 0.14 | 0.11 |
| Olfr1200 | 0.20 | 0.23 | 0.20 | 0.26 | 0.24 | 0.26 | 0.22 | 0.20 |
| Olfr1201 | 0.12 | 0.13 | 0.13 | 0.15 | 0.16 | 0.12 | 0.15 | 0.19 |
| Olfr1202 | 0.05 | 0.08 | 0.05 | 0.08 | 0.09 | 0.09 | 0.07 | 0.11 |
| Olfr1204 | 0.07 | 0.06 | 0.06 | 0.09 | 0.06 | 0.06 | 0.09 | 0.15 |
| Olfr1205 | 0.12 | 0.13 | 0.13 | 0.15 | 0.16 | 0.12 | 0.15 | 0.19 |
| Olfr1206 | 0.12 | 0.13 | 0.13 | 0.15 | 0.16 | 0.12 | 0.15 | 0.19 |
| Olfr1208 | 0.06 | 0.09 | 0.10 | 0.08 | 0.08 | 0.11 | 0.08 | 0.14 |
| Olfr1209 | 0.14 | 0.17 | 0.15 | 0.18 | 0.16 | 0.20 | 0.19 | 0.18 |
| Olfr121 | 0.09 | 0.14 | 0.11 | 0.14 | 0.13 | 0.12 | 0.10 | 0.20 |
| Olfr1211 | 0.08 | 0.09 | 0.08 | 0.10 | 0.09 | 0.11 | 0.10 | 0.11 |
| Olfr1212 | 0.00 | 0.02 | 0.02 | 0.03 | 0.04 | 0.04 | 0.05 | 0.05 |
| Olfr1213 | 0.06 | 0.09 | 0.07 | 0.09 | 0.10 | 0.07 | 0.08 | 0.10 |
| Olfr1214 | 0.04 | 0.06 | 0.04 | 0.03 | 0.05 | 0.04 | 0.05 | 0.12 |
| Olfr1215 | 0.05 | 0.10 | 0.07 | 0.08 | 0.09 | 0.12 | 0.10 | 0.15 |
| Olfr1216 | 0.05 | 0.06 | 0.04 | 0.05 | 0.03 | 0.04 | 0.10 | 0.09 |
| Olfr1217 | 0.00 | 0.03 | 0.01 | 0.04 | 0.04 | 0.02 | 0.03 | 0.06 |
| Olfr1218 | 0.05 | 0.07 | 0.05 | 0.08 | 0.08 | 0.07 | 0.08 | 0.12 |
| Olfr1219 | 0.07 | 0.09 | 0.09 | 0.12 | 0.13 | 0.10 | 0.11 | 0.12 |
| Olfr122 | 0.19 | 0.24 | 0.22 | 0.26 | 0.30 | 0.29 | 0.24 | 0.29 |
| Olfr1220 | 0.06 | 0.06 | 0.05 | 0.07 | 0.08 | 0.09 | 0.09 | 0.09 |
| Olfr1221 | NA | 0.02 | NA | 0.00 | 0.00 | 0.00 | 0.02 | 0.04 |
| Olfr1222 | 0.19 | 0.23 | 0.22 | 0.22 | 0.30 | 0.25 | 0.27 | 0.22 |
| Olfr1223 | 0.07 | 0.10 | 0.08 | 0.07 | 0.09 | 0.11 | 0.07 | 0.13 |
| Olfr1225 | 0.10 | 0.15 | 0.13 | 0.15 | 0.12 | 0.16 | 0.15 | 0.20 |
| Olfr1226 | 0.13 | 0.19 | 0.15 | 0.18 | 0.18 | 0.16 | 0.19 | 0.20 |
| Olfr1228 | 0.01 | 0.04 | 0.04 | 0.05 | 0.05 | 0.04 | 0.05 | 0.06 |
| Olfr1229 | 0.30 | 0.33 | 0.30 | 0.38 | 0.37 | 0.35 | 0.32 | 0.38 |
| Olfr123 | 0.18 | 0.25 | 0.23 | 0.23 | 0.23 | 0.30 | 0.24 | 0.25 |
| Olfr1230 | 0.15 | 0.20 | 0.19 | 0.18 | 0.17 | 0.22 | 0.16 | 0.19 |
| Olfr1231 | 0.11 | 0.16 | 0.13 | 0.15 | 0.17 | 0.16 | 0.14 | 0.17 |
| Olfr1232 | 0.03 | 0.04 | 0.02 | 0.03 | 0.03 | 0.04 | 0.08 | 0.11 |
| Olfr1233 | 0.13 | 0.16 | 0.14 | 0.17 | 0.14 | 0.19 | 0.14 | 0.14 |
| Olfr1234 | 0.09 | 0.10 | 0.10 | 0.11 | 0.12 | 0.14 | 0.10 | 0.15 |
| Olfr1238 | 0.13 | 0.14 | 0.14 | 0.15 | 0.19 | 0.16 | 0.13 | 0.18 |
| Olfr1239 | 0.15 | 0.21 | 0.17 | 0.19 | 0.21 | 0.24 | 0.19 | 0.21 |
| Olfr124 | 0.13 | 0.17 | 0.17 | 0.19 | 0.19 | 0.17 | 0.18 | 0.21 |
| Olfr1240 | 0.09 | 0.09 | 0.09 | 0.13 | 0.10 | 0.12 | 0.12 | 0.16 |
| Olfr1241 | 0.08 | 0.12 | 0.08 | 0.11 | 0.11 | 0.12 | 0.10 | 0.14 |
| Olfr1242 | 0.11 | 0.13 | 0.14 | 0.13 | 0.13 | 0.16 | 0.15 | 0.17 |
| Olfr1243 | 0.06 | 0.07 | 0.07 | 0.09 | 0.06 | 0.07 | 0.07 | 0.09 |
| Olfr1245 | 0.01 | 0.05 | 0.06 | 0.07 | 0.06 | 0.09 | 0.06 | 0.16 |
| Olfr1246 | NA | 0.02 | 0.01 | 0.02 | 0.02 | 0.02 | 0.01 | 0.04 |
| Olfr1247 | 0.05 | 0.07 | 0.06 | 0.09 | 0.07 | 0.07 | 0.08 | 0.13 |
| Olfr1248 | NA | NA | 0.01 | NA | −0.02 | 0.00 | NA | 0.02 |
| Olfr1249 | 0.09 | 0.13 | 0.12 | 0.16 | 0.16 | 0.13 | 0.15 | 0.18 |
| Olfr125 | 0.03 | 0.05 | 0.05 | 0.04 | 0.05 | 0.05 | 0.04 | 0.08 |
| Olfr1250 | 0.01 | 0.05 | 0.03 | 0.02 | 0.04 | 0.04 | 0.04 | 0.07 |
| Olfr1251 | NA | NA | NA | NA | −0.02 | 0.02 | NA | 0.02 |
| Olfr1252 | 0.10 | 0.14 | 0.11 | 0.12 | 0.15 | 0.15 | 0.14 | 0.14 |
| Olfr1253 | 0.02 | 0.05 | NA | NA | 0.04 | 0.03 | 0.05 | 0.09 |
| Olfr1254 | NA | NA | NA | 0.01 | 0.00 | 0.01 | 0.00 | 0.06 |
| Olfr1255 | 0.11 | 0.14 | 0.14 | 0.14 | 0.15 | 0.15 | 0.16 | 0.19 |
| Olfr1256 | 0.13 | 0.17 | 0.15 | 0.18 | 0.17 | 0.17 | 0.18 | 0.17 |
| Olfr1257 | 0.04 | 0.04 | 0.04 | 0.05 | 0.09 | 0.06 | 0.07 | 0.09 |
| Olfr1258 | 0.14 | 0.16 | 0.15 | 0.19 | 0.17 | 0.18 | 0.17 | 0.19 |
| Olfr1259 | 0.13 | 0.15 | 0.15 | 0.20 | 0.15 | 0.17 | 0.15 | 0.16 |
| Olfr126 | 0.09 | 0.09 | 0.08 | 0.10 | 0.11 | 0.09 | 0.11 | 0.13 |
| Olfr1260 | 0.18 | 0.23 | 0.21 | 0.21 | 0.22 | 0.25 | 0.25 | 0.21 |
| Olfr1261 | 0.02 | 0.03 | 0.02 | 0.05 | 0.04 | 0.05 | 0.07 | 0.11 |
| Olfr1262 | 0.16 | 0.20 | 0.17 | 0.22 | 0.22 | 0.21 | 0.22 | 0.27 |
| Olfr1263 | 0.11 | 0.15 | 0.12 | 0.18 | 0.15 | 0.14 | 0.17 | 0.12 |
| Olfr1264 | 0.13 | 0.16 | 0.13 | 0.19 | 0.17 | 0.17 | 0.17 | 0.16 |
| Olfr1265 | 0.08 | 0.11 | 0.07 | 0.13 | 0.14 | 0.10 | 0.13 | 0.10 |
| Olfr1269 | 0.08 | 0.11 | 0.07 | 0.13 | 0.14 | 0.10 | 0.13 | 0.10 |
| Olfr127 | 0.09 | 0.09 | 0.08 | 0.10 | 0.11 | 0.09 | 0.11 | 0.13 |
| Olfr1270 | 0.08 | 0.10 | 0.08 | 0.12 | 0.12 | 0.12 | 0.13 | 0.16 |
| Olfr1271 | 0.09 | 0.12 | 0.09 | 0.10 | 0.09 | 0.12 | 0.12 | 0.17 |
| Olfr1272 | 0.24 | 0.29 | 0.24 | 0.30 | 0.30 | 0.28 | 0.26 | 0.30 |
| Olfr1273-ps | NA | 0.01 | 0.01 | 0.01 | 0.03 | 0.01 | 0.02 | 0.08 |
| Olfr1274-ps | 0.14 | 0.18 | 0.20 | 0.19 | 0.15 | 0.14 | 0.19 | 0.19 |
| Olfr1275 | 0.08 | 0.10 | 0.08 | 0.09 | 0.13 | 0.12 | 0.10 | 0.11 |
| Olfr1276 | 0.16 | 0.18 | 0.15 | 0.19 | 0.18 | 0.20 | 0.20 | 0.21 |
| Olfr1277 | 0.20 | 0.22 | 0.20 | 0.25 | 0.23 | 0.29 | 0.20 | 0.21 |
| Olfr1278 | 0.10 | 0.11 | 0.09 | 0.12 | 0.11 | 0.12 | 0.12 | 0.11 |
| Olfr1279 | 0.22 | 0.27 | 0.23 | 0.27 | 0.26 | 0.27 | 0.28 | 0.28 |
| Olfr128 | 0.09 | 0.09 | 0.08 | 0.10 | 0.11 | 0.09 | 0.11 | 0.13 |
| Olfr1280 | 0.07 | 0.07 | 0.06 | 0.07 | 0.07 | 0.08 | 0.07 | 0.10 |
| Olfr1281 | 0.03 | 0.02 | 0.03 | 0.05 | 0.03 | 0.06 | 0.04 | 0.10 |
| Olfr1282 | 0.07 | 0.07 | 0.06 | 0.07 | 0.07 | 0.08 | 0.07 | 0.10 |
| Olfr1283 | 0.07 | 0.07 | 0.06 | 0.07 | 0.07 | 0.08 | 0.07 | 0.10 |
| Olfr1284 | 0.23 | 0.28 | 0.27 | 0.28 | 0.26 | 0.27 | 0.28 | 0.28 |
| Olfr1286 | 0.07 | 0.07 | 0.06 | 0.07 | 0.07 | 0.08 | 0.07 | 0.10 |
| Olfr1287 | 0.07 | 0.07 | 0.06 | 0.07 | 0.07 | 0.08 | 0.07 | 0.10 |
| Olfr1288 | 0.04 | 0.05 | 0.05 | 0.07 | 0.06 | 0.04 | 0.04 | 0.14 |
| Olfr1289 | 0.04 | 0.07 | 0.05 | 0.07 | 0.06 | 0.06 | 0.07 | 0.11 |
| Olfr129 | 0.21 | 0.27 | 0.26 | 0.26 | 0.27 | 0.25 | 0.27 | 0.28 |
| Olfr1290 | 0.05 | 0.10 | 0.06 | 0.09 | 0.11 | 0.06 | 0.11 | 0.12 |
| Olfr1294 | 0.09 | 0.12 | 0.09 | 0.11 | 0.11 | 0.12 | 0.11 | 0.11 |
| Olfr1295 | 0.07 | 0.10 | 0.10 | 0.11 | 0.11 | 0.13 | 0.11 | 0.09 |
| Olfr1297 | 0.04 | 0.07 | 0.05 | 0.07 | 0.08 | 0.07 | 0.07 | 0.09 |
| Olfr1298 | 0.05 | 0.06 | 0.07 | 0.06 | 0.07 | 0.10 | 0.11 | 0.06 |
| Olfr1299 | 0.06 | 0.11 | 0.06 | 0.11 | 0.09 | 0.11 | 0.10 | 0.14 |
| Olfr13 | 0.24 | 0.31 | 0.25 | 0.32 | 0.29 | 0.28 | 0.30 | 0.27 |
| Olfr130 | 0.09 | 0.11 | 0.11 | 0.11 | 0.13 | 0.10 | 0.11 | 0.15 |
| Olfr1300-ps1 | 0.04 | 0.04 | 0.05 | 0.07 | 0.07 | 0.04 | 0.06 | 0.08 |
| Olfr1301 | 0.13 | 0.16 | 0.15 | 0.18 | 0.15 | 0.18 | 0.17 | 0.16 |
| Olfr1302 | 0.02 | 0.02 | 0.03 | 0.04 | 0.02 | 0.02 | 0.02 | 0.05 |
| Olfr1303 | 0.07 | 0.10 | 0.07 | 0.09 | 0.11 | 0.12 | 0.08 | 0.13 |
| Olfr1305 | 0.09 | 0.11 | 0.09 | 0.11 | 0.13 | 0.12 | 0.14 | 0.14 |
| Olfr1306 | NA | NA | NA | 0.00 | 0.02 | 0.01 | 0.01 | 0.05 |
| Olfr1307 | 0.08 | 0.15 | 0.10 | 0.11 | 0.12 | 0.16 | 0.11 | 0.17 |
| Olfr1308 | NA | 0.00 | 0.00 | 0.03 | 0.01 | 0.01 | 0.03 | 0.05 |
| Olfr1309 | 0.14 | 0.17 | 0.17 | 0.16 | 0.17 | 0.17 | 0.17 | 0.14 |
| Olfr131 | 0.12 | 0.14 | 0.12 | 0.16 | 0.14 | 0.15 | 0.13 | 0.15 |
| Olfr1310 | 0.09 | 0.12 | 0.10 | 0.16 | 0.13 | 0.13 | 0.13 | 0.16 |
| Olfr1311 | 0.12 | 0.15 | 0.12 | 0.18 | 0.13 | 0.16 | 0.17 | 0.14 |
| Olfr1312 | 0.01 | 0.06 | 0.03 | 0.01 | 0.04 | 0.06 | 0.02 | 0.06 |
| Olfr1313 | 0.09 | 0.12 | 0.10 | 0.12 | 0.13 | 0.11 | 0.15 | 0.14 |
| Olfr1314 | 0.09 | 0.10 | 0.09 | 0.11 | 0.10 | 0.09 | 0.11 | 0.11 |
| Olfr1316 | 0.07 | 0.09 | 0.09 | 0.10 | 0.12 | 0.11 | 0.12 | 0.12 |
| Olfr1317 | 0.03 | 0.05 | 0.05 | 0.08 | 0.06 | 0.05 | 0.06 | 0.14 |
| Olfr1318 | 0.06 | 0.11 | 0.09 | 0.11 | 0.11 | 0.10 | 0.08 | 0.11 |
| Olfr132 | 0.12 | 0.14 | 0.13 | 0.17 | 0.15 | 0.20 | 0.15 | 0.18 |
| Olfr1320 | 0.15 | 0.20 | 0.17 | 0.20 | 0.19 | 0.21 | 0.19 | 0.20 |
| Olfr1321 | 0.11 | 0.12 | 0.13 | 0.14 | 0.15 | 0.17 | 0.11 | 0.17 |
| Olfr1322 | 0.20 | 0.21 | 0.21 | 0.22 | 0.22 | 0.25 | 0.25 | 0.21 |
| Olfr1323 | 0.10 | 0.14 | 0.11 | 0.12 | 0.12 | 0.15 | 0.12 | 0.13 |
| Olfr1324 | 0.18 | 0.24 | 0.20 | 0.21 | 0.23 | 0.22 | 0.23 | 0.24 |
| Olfr1325 | 0.07 | 0.09 | 0.08 | 0.12 | 0.09 | 0.11 | 0.09 | 0.13 |
| Olfr1328 | 0.17 | 0.21 | 0.21 | 0.26 | 0.23 | 0.23 | 0.24 | 0.22 |
| Olfr1329 | 0.17 | 0.21 | 0.21 | 0.26 | 0.23 | 0.23 | 0.24 | 0.22 |
| Olfr133 | 0.01 | 0.04 | 0.04 | 0.06 | 0.04 | 0.06 | 0.05 | 0.05 |
| Olfr1330 | 0.17 | 0.21 | 0.21 | 0.26 | 0.23 | 0.23 | 0.24 | 0.22 |
| Olfr1331 | 0.13 | 0.20 | 0.16 | 0.21 | 0.23 | 0.21 | 0.22 | 0.22 |
| Olfr1333 | 0.17 | 0.21 | 0.21 | 0.26 | 0.23 | 0.23 | 0.24 | 0.22 |
| Olfr1335 | 0.17 | 0.21 | 0.21 | 0.26 | 0.23 | 0.23 | 0.24 | 0.22 |
| Olfr1336 | 0.23 | 0.28 | 0.24 | 0.28 | 0.26 | 0.29 | 0.23 | 0.26 |
| Olfr1337 | 0.17 | 0.21 | 0.21 | 0.26 | 0.23 | 0.23 | 0.24 | 0.22 |
| Olfr1338 | 0.17 | 0.21 | 0.21 | 0.26 | 0.23 | 0.23 | 0.24 | 0.22 |
| Olfr1339 | 0.30 | 0.34 | 0.32 | 0.36 | 0.34 | 0.36 | 0.36 | 0.30 |
| Olfr134 | NA | NA | NA | 0.03 | 0.01 | 0.00 | 0.01 | 0.04 |
| Olfr1340 | 0.26 | 0.30 | 0.26 | 0.36 | 0.33 | 0.29 | 0.29 | 0.36 |
| Olfr1341 | 0.12 | 0.16 | 0.14 | 0.15 | 0.18 | 0.18 | 0.17 | 0.19 |
| Olfr1342 | 0.23 | 0.25 | 0.26 | 0.32 | 0.26 | 0.30 | 0.29 | 0.24 |
| Olfr1344 | 0.28 | 0.32 | 0.28 | 0.32 | 0.32 | 0.35 | 0.31 | 0.32 |
| Olfr1346 | 0.32 | 0.37 | 0.32 | 0.42 | 0.41 | 0.38 | 0.36 | 0.45 |
| Olfr1347 | 0.37 | 0.46 | 0.38 | 0.46 | 0.48 | 0.48 | 0.40 | 0.47 |
| Olfr1348 | 0.26 | 0.26 | 0.24 | 0.29 | 0.28 | 0.32 | 0.29 | 0.27 |
| Olfr1349 | 0.36 | 0.41 | 0.37 | 0.44 | 0.43 | 0.42 | 0.41 | 0.41 |
| Olfr135 | 0.02 | 0.04 | 0.01 | 0.06 | 0.05 | 0.04 | 0.06 | 0.07 |
| Olfr1350 | 0.24 | 0.27 | 0.24 | 0.30 | 0.28 | 0.29 | 0.26 | 0.28 |
| Olfr1351 | 0.13 | 0.15 | 0.17 | 0.18 | 0.15 | 0.16 | 0.18 | 0.17 |
| Olfr1352 | 0.10 | 0.12 | 0.10 | 0.15 | 0.12 | 0.11 | 0.14 | 0.16 |
| Olfr1353 | 0.09 | 0.11 | 0.10 | 0.12 | 0.11 | 0.13 | 0.12 | 0.14 |
| Olfr1354 | 0.16 | 0.19 | 0.18 | 0.21 | 0.21 | 0.21 | 0.19 | 0.24 |
| Olfr1355 | 0.16 | 0.19 | 0.18 | 0.21 | 0.21 | 0.21 | 0.19 | 0.24 |
| Olfr1356 | 0.08 | 0.10 | 0.09 | 0.11 | 0.11 | 0.12 | 0.09 | 0.14 |
| Olfr1357 | 0.20 | 0.23 | 0.20 | 0.22 | 0.23 | 0.24 | 0.20 | 0.29 |
| Olfr1359 | 0.22 | 0.29 | 0.25 | 0.28 | 0.29 | 0.28 | 0.28 | 0.28 |
| Olfr136 | 0.03 | 0.04 | 0.04 | 0.04 | 0.07 | 0.06 | 0.07 | 0.09 |
| Olfr1360 | 0.22 | 0.29 | 0.25 | 0.28 | 0.29 | 0.28 | 0.28 | 0.28 |
| Olfr1361 | 0.25 | 0.27 | 0.25 | 0.30 | 0.27 | 0.28 | 0.23 | 0.29 |
| Olfr1362 | 0.31 | 0.35 | 0.33 | 0.34 | 0.36 | 0.35 | 0.30 | 0.33 |
| Olfr1364 | 0.29 | 0.31 | 0.30 | 0.33 | 0.32 | 0.30 | 0.33 | 0.31 |
| Olfr1366 | 0.24 | 0.27 | 0.28 | 0.27 | 0.26 | 0.30 | 0.32 | 0.27 |
| Olfr1367 | 0.11 | 0.11 | 0.11 | 0.15 | 0.13 | 0.10 | 0.12 | 0.22 |
| Olfr1368 | 0.08 | 0.09 | 0.08 | 0.10 | 0.08 | 0.11 | 0.14 | 0.15 |
| Olfr137 | 0.22 | 0.24 | 0.22 | 0.25 | 0.25 | 0.25 | 0.21 | 0.25 |
| Olfr1370 | 0.16 | 0.19 | 0.19 | 0.20 | 0.18 | 0.18 | 0.21 | 0.23 |
| Olfr1371 | 0.24 | 0.22 | 0.18 | 0.28 | 0.25 | 0.22 | 0.20 | 0.20 |
| Olfr1372-ps1 | 0.13 | 0.12 | 0.10 | 0.14 | 0.16 | 0.12 | 0.14 | 0.18 |
| Olfr1373 | 0.24 | 0.22 | 0.18 | 0.28 | 0.25 | 0.22 | 0.20 | 0.20 |
| Olfr1377 | 0.17 | 0.25 | 0.21 | 0.26 | 0.20 | 0.22 | 0.21 | 0.26 |
| Olfr1378 | 0.24 | 0.26 | 0.26 | 0.27 | 0.27 | 0.26 | 0.27 | 0.31 |
| Olfr138 | NA | NA | NA | NA | 0.01 | 0.01 | 0.02 | NA |
| Olfr1380 | 0.24 | 0.22 | 0.18 | 0.28 | 0.25 | 0.22 | 0.20 | 0.20 |
| Olfr1381 | 0.24 | 0.22 | 0.18 | 0.28 | 0.25 | 0.22 | 0.20 | 0.20 |
| Olfr1382 | 0.24 | 0.22 | 0.18 | 0.28 | 0.25 | 0.22 | 0.20 | 0.20 |
| Olfr1383 | 0.20 | 0.26 | 0.20 | 0.27 | 0.29 | 0.24 | 0.21 | 0.33 |
| Olfr1384 | 0.16 | 0.22 | 0.18 | 0.23 | 0.22 | 0.23 | 0.21 | 0.19 |
| Olfr1385 | 0.18 | 0.22 | 0.19 | 0.22 | 0.20 | 0.20 | 0.22 | 0.23 |
| Olfr1386 | 0.18 | 0.22 | 0.19 | 0.22 | 0.20 | 0.20 | 0.22 | 0.23 |
| Olfr1387 | 0.15 | 0.19 | 0.15 | 0.20 | 0.17 | 0.20 | 0.14 | 0.17 |
| Olfr1388 | 0.25 | 0.32 | 0.28 | 0.32 | 0.31 | 0.30 | 0.29 | 0.35 |
| Olfr1389 | 0.21 | 0.26 | 0.26 | 0.25 | 0.25 | 0.30 | 0.27 | 0.27 |
| Olfr139 | 0.28 | 0.34 | 0.28 | 0.35 | 0.35 | 0.35 | 0.34 | 0.35 |
| Olfr1390 | 0.25 | 0.25 | 0.27 | 0.28 | 0.29 | 0.28 | 0.30 | 0.25 |
| Olfr1391 | 0.18 | 0.21 | 0.17 | 0.21 | 0.21 | 0.24 | 0.22 | 0.21 |
| Olfr1392 | 0.23 | 0.28 | 0.25 | 0.29 | 0.29 | 0.28 | 0.27 | 0.36 |
| Olfr1393 | 0.29 | 0.38 | 0.34 | 0.39 | 0.33 | 0.37 | 0.36 | 0.35 |
| Olfr1394 | 0.38 | 0.43 | 0.41 | 0.46 | 0.46 | 0.45 | 0.43 | 0.46 |
| Olfr1395 | 0.41 | 0.46 | 0.42 | 0.49 | 0.47 | 0.46 | 0.46 | 0.44 |
| Olfr1396 | 0.20 | 0.24 | 0.25 | 0.30 | 0.25 | 0.28 | 0.29 | 0.19 |
| Olfr140 | 0.21 | 0.30 | 0.27 | 0.30 | 0.27 | 0.31 | 0.26 | 0.27 |
| Olfr1402 | 0.26 | 0.32 | 0.30 | 0.32 | 0.31 | 0.33 | 0.29 | 0.35 |
| Olfr1404 | 0.08 | 0.11 | 0.09 | 0.14 | 0.12 | 0.10 | 0.12 | 0.11 |
| Olfr1406 | 0.09 | 0.13 | 0.13 | 0.13 | 0.13 | 0.14 | 0.09 | 0.14 |
| Olfr1408 | 0.14 | 0.16 | 0.17 | 0.17 | 0.18 | 0.21 | 0.20 | 0.21 |
| Olfr141 | 0.09 | 0.10 | 0.11 | 0.12 | 0.10 | 0.11 | 0.12 | 0.13 |
| Olfr1410 | 0.37 | 0.45 | 0.40 | 0.42 | 0.40 | 0.46 | 0.41 | 0.42 |
| Olfr1411 | 0.21 | 0.28 | 0.24 | 0.30 | 0.25 | 0.27 | 0.25 | 0.29 |
| Olfr1412 | 0.30 | 0.32 | 0.29 | 0.33 | 0.29 | 0.35 | 0.30 | 0.25 |
| Olfr1413 | 0.30 | 0.35 | 0.34 | 0.37 | 0.34 | 0.37 | 0.36 | 0.31 |
| Olfr1414 | 0.32 | 0.37 | 0.36 | 0.39 | 0.39 | 0.40 | 0.37 | 0.35 |
| Olfr1415 | 0.32 | 0.37 | 0.36 | 0.39 | 0.39 | 0.40 | 0.37 | 0.35 |
| Olfr1416 | 0.32 | 0.37 | 0.36 | 0.39 | 0.39 | 0.40 | 0.37 | 0.35 |
| Olfr1417 | 0.23 | 0.29 | 0.27 | 0.29 | 0.32 | 0.33 | 0.30 | 0.29 |
| Olfr1418 | 0.18 | 0.23 | 0.23 | 0.19 | 0.24 | 0.18 | 0.22 | 0.23 |
| Olfr1419 | 0.22 | 0.22 | 0.24 | 0.26 | 0.25 | 0.26 | 0.21 | 0.23 |
| Olfr142 | 0.06 | 0.11 | 0.10 | 0.13 | 0.12 | 0.13 | 0.11 | 0.15 |
| Olfr1420 | 0.20 | 0.23 | 0.21 | 0.22 | 0.21 | 0.22 | 0.23 | 0.26 |
| Olfr1423 | 0.17 | 0.22 | 0.19 | 0.23 | 0.23 | 0.22 | 0.22 | 0.22 |
| Olfr1424 | 0.04 | 0.09 | 0.05 | 0.05 | 0.08 | 0.09 | 0.09 | 0.10 |
| Olfr1425 | 0.19 | 0.25 | 0.22 | 0.27 | 0.22 | 0.25 | 0.24 | 0.25 |
| Olfr1426 | 0.19 | 0.24 | 0.20 | 0.22 | 0.21 | 0.22 | 0.19 | 0.28 |
| Olfr1427 | 0.17 | 0.19 | 0.16 | 0.19 | 0.19 | 0.21 | 0.17 | 0.24 |
| Olfr1428 | 0.25 | 0.30 | 0.25 | 0.31 | 0.28 | 0.28 | 0.26 | 0.27 |
| Olfr143 | 0.16 | 0.21 | 0.19 | 0.19 | 0.19 | 0.21 | 0.20 | 0.23 |
| Olfr1431 | 0.07 | 0.10 | 0.10 | 0.10 | 0.10 | 0.11 | 0.08 | 0.12 |
| Olfr1434 | NA | NA | 0.02 | 0.03 | 0.04 | 0.03 | 0.06 | 0.12 |
| Olfr1436 | 0.00 | 0.05 | NA | 0.01 | 0.03 | 0.03 | 0.05 | 0.03 |
| Olfr1437 | 0.09 | 0.15 | 0.14 | 0.14 | 0.11 | 0.13 | 0.17 | 0.17 |
| Olfr1440 | 0.20 | 0.24 | 0.21 | 0.22 | 0.21 | 0.24 | 0.22 | 0.27 |
| Olfr1441 | 0.16 | 0.18 | 0.15 | 0.17 | 0.18 | 0.21 | 0.18 | 0.17 |
| Olfr1442 | 0.10 | 0.11 | 0.11 | 0.11 | 0.12 | 0.14 | 0.12 | 0.13 |
| Olfr1443 | 0.02 | 0.04 | 0.04 | 0.06 | 0.05 | 0.07 | 0.05 | 0.06 |
| Olfr1444 | 0.12 | 0.14 | 0.15 | 0.18 | 0.15 | 0.16 | 0.15 | 0.16 |
| Olfr1445 | 0.10 | 0.15 | 0.14 | 0.16 | 0.15 | 0.16 | 0.14 | 0.13 |
| Olfr1446 | 0.05 | 0.07 | 0.07 | 0.06 | 0.10 | 0.08 | 0.06 | 0.10 |
| Olfr1447 | 0.06 | 0.07 | 0.07 | 0.09 | 0.09 | 0.09 | 0.11 | 0.12 |
| Olfr1448 | 0.02 | 0.04 | 0.03 | 0.05 | 0.07 | 0.05 | 0.07 | 0.08 |
| Olfr1449 | 0.19 | 0.20 | 0.21 | 0.20 | 0.21 | 0.22 | 0.24 | 0.27 |
| Olfr145 | 0.16 | 0.20 | 0.18 | 0.23 | 0.21 | 0.22 | 0.20 | 0.19 |
| Olfr1450 | 0.14 | 0.16 | 0.17 | 0.16 | 0.17 | 0.18 | 0.17 | 0.18 |
| Olfr1451 | 0.05 | 0.08 | 0.06 | 0.08 | 0.07 | 0.07 | 0.06 | 0.08 |
| Olfr1453 | 0.08 | 0.13 | 0.10 | 0.13 | 0.13 | 0.12 | 0.13 | 0.20 |
| Olfr1454 | 0.03 | 0.06 | 0.04 | 0.07 | 0.05 | 0.04 | 0.05 | 0.06 |
| Olfr1457 | 0.01 | 0.03 | 0.02 | 0.03 | 0.03 | 0.01 | 0.02 | 0.05 |
| Olfr1459 | 0.05 | 0.06 | 0.08 | 0.07 | 0.07 | 0.08 | 0.05 | 0.10 |
| Olfr146 | 0.07 | 0.10 | 0.07 | 0.10 | 0.08 | 0.10 | 0.09 | 0.12 |
| Olfr1461 | 0.12 | 0.15 | 0.13 | 0.18 | 0.12 | 0.17 | 0.17 | 0.15 |
| Olfr1462 | 0.03 | 0.05 | 0.04 | 0.04 | 0.08 | 0.04 | 0.08 | 0.08 |
| Olfr1463 | 0.22 | 0.21 | 0.23 | 0.25 | 0.24 | 0.22 | 0.23 | 0.22 |
| Olfr1465 | 0.19 | 0.21 | 0.21 | 0.24 | 0.21 | 0.22 | 0.19 | 0.29 |
| Olfr1466 | 0.07 | 0.11 | 0.09 | 0.13 | 0.09 | 0.13 | 0.15 | 0.11 |
| Olfr1467 | 0.07 | 0.10 | 0.08 | 0.10 | 0.11 | 0.10 | 0.10 | 0.17 |
| Olfr1469 | 0.06 | 0.10 | 0.08 | 0.10 | 0.10 | 0.11 | 0.10 | 0.10 |
| Olfr147 | 0.27 | 0.33 | 0.30 | 0.34 | 0.31 | 0.34 | 0.31 | 0.36 |
| Olfr1471 | 0.03 | 0.04 | 0.04 | 0.03 | 0.05 | 0.07 | 0.04 | 0.10 |
| Olfr1472 | 0.06 | 0.07 | 0.09 | 0.08 | 0.09 | 0.09 | 0.09 | 0.11 |
| Olfr1474 | 0.08 | 0.10 | 0.08 | 0.13 | 0.12 | 0.10 | 0.11 | 0.16 |
| Olfr1475 | 0.01 | 0.04 | 0.04 | 0.06 | 0.07 | 0.06 | 0.04 | 0.14 |
| Olfr1477 | NA | NA | NA | NA | −0.01 | 0.01 | NA | 0.05 |
| Olfr148 | 0.15 | 0.21 | 0.18 | 0.20 | 0.23 | 0.21 | 0.19 | 0.24 |
| Olfr1480 | 0.07 | 0.10 | 0.10 | 0.11 | 0.10 | 0.09 | 0.10 | 0.12 |
| Olfr1484 | 0.07 | 0.09 | 0.07 | 0.10 | 0.08 | 0.10 | 0.07 | 0.08 |
| Olfr1487 | 0.02 | 0.05 | 0.04 | 0.05 | 0.07 | 0.10 | 0.04 | 0.09 |
| Olfr1489 | 0.06 | 0.09 | 0.06 | 0.09 | 0.09 | 0.09 | 0.10 | 0.11 |
| Olfr149 | 0.33 | 0.41 | 0.36 | 0.38 | 0.39 | 0.42 | 0.38 | 0.40 |
| Olfr1490 | 0.18 | 0.23 | 0.20 | 0.23 | 0.20 | 0.24 | 0.20 | 0.22 |
| Olfr1491 | 0.22 | 0.26 | 0.25 | 0.31 | 0.30 | 0.31 | 0.26 | 0.21 |
| Olfr1494 | 0.08 | 0.08 | 0.07 | 0.08 | 0.09 | 0.08 | 0.10 | 0.11 |
| Olfr1495 | 0.20 | 0.26 | 0.21 | 0.28 | 0.28 | 0.27 | 0.25 | 0.33 |
| Olfr1496 | 0.03 | 0.03 | 0.03 | 0.05 | 0.05 | 0.04 | 0.05 | 0.05 |
| Olfr1497 | 0.31 | 0.33 | 0.31 | 0.38 | 0.34 | 0.36 | 0.33 | 0.39 |
| Olfr1499 | 0.10 | 0.11 | 0.11 | 0.15 | 0.11 | 0.12 | 0.14 | 0.16 |
| Olfr15 | 0.13 | 0.17 | 0.13 | 0.20 | 0.17 | 0.19 | 0.18 | 0.23 |
| Olfr150 | 0.15 | 0.23 | 0.20 | 0.21 | 0.20 | 0.25 | 0.22 | 0.24 |
| Olfr1500 | 0.19 | 0.25 | 0.21 | 0.24 | 0.23 | 0.25 | 0.22 | 0.30 |
| Olfr1501 | 0.04 | 0.08 | 0.06 | 0.08 | 0.08 | 0.10 | 0.08 | 0.16 |
| Olfr1502 | 0.22 | 0.27 | 0.24 | 0.26 | 0.26 | 0.26 | 0.27 | 0.28 |
| Olfr1504 | 0.09 | 0.13 | 0.09 | 0.13 | 0.12 | 0.13 | 0.11 | 0.18 |
| Olfr1505 | 0.16 | 0.19 | 0.16 | 0.20 | 0.22 | 0.24 | 0.19 | 0.18 |
| Olfr1506 | 0.17 | 0.23 | 0.23 | 0.27 | 0.23 | 0.25 | 0.21 | 0.23 |
| Olfr1507 | 0.07 | 0.10 | 0.08 | 0.09 | 0.13 | 0.11 | 0.13 | 0.13 |
| Olfr1508 | 0.18 | 0.22 | 0.18 | 0.22 | 0.22 | 0.27 | 0.21 | 0.18 |
| Olfr1509 | 0.09 | 0.11 | 0.10 | 0.14 | 0.10 | 0.15 | 0.11 | 0.16 |
| Olfr151 | 0.09 | 0.13 | 0.10 | 0.13 | 0.12 | 0.14 | 0.13 | 0.13 |
| Olfr1510 | 0.31 | 0.42 | 0.34 | 0.44 | 0.41 | 0.40 | 0.36 | 0.40 |
| Olfr1511 | 0.19 | 0.28 | 0.24 | 0.26 | 0.28 | 0.25 | 0.24 | 0.29 |
| Olfr1512 | 0.12 | 0.16 | 0.13 | 0.17 | 0.16 | 0.16 | 0.16 | 0.21 |
| Olfr1513 | 0.12 | 0.16 | 0.13 | 0.17 | 0.16 | 0.16 | 0.16 | 0.21 |
| Olfr152 | 0.12 | 0.15 | 0.14 | 0.14 | 0.21 | 0.16 | 0.17 | 0.23 |
| Olfr153 | 0.07 | 0.10 | 0.09 | 0.12 | 0.13 | 0.10 | 0.09 | 0.15 |
| Olfr1532-ps1 | 0.31 | 0.39 | 0.35 | 0.40 | 0.38 | 0.41 | 0.38 | 0.39 |
| Olfr1535 | 0.22 | 0.29 | 0.26 | 0.26 | 0.25 | 0.26 | 0.26 | 0.24 |
| Olfr1537 | 0.08 | 0.16 | 0.10 | 0.10 | 0.10 | 0.08 | 0.14 | 0.08 |
| Olfr154 | 0.08 | 0.11 | 0.08 | 0.10 | 0.10 | 0.11 | 0.10 | 0.15 |
| Olfr155 | 0.24 | 0.25 | 0.24 | 0.28 | 0.27 | 0.26 | 0.27 | 0.30 |
| Olfr156 | 0.12 | 0.16 | 0.12 | 0.18 | 0.17 | 0.19 | 0.13 | 0.17 |
| Olfr157 | 0.28 | 0.35 | 0.32 | 0.33 | 0.35 | 0.36 | 0.34 | 0.37 |
| Olfr159 | 0.20 | 0.21 | 0.20 | 0.24 | 0.20 | 0.23 | 0.22 | 0.24 |
| Olfr16 | 0.21 | 0.24 | 0.23 | 0.24 | 0.22 | 0.26 | 0.23 | 0.26 |
| Olfr160 | 0.18 | 0.22 | 0.19 | 0.22 | 0.18 | 0.22 | 0.21 | 0.22 |
| Olfr161 | 0.22 | 0.27 | 0.25 | 0.27 | 0.26 | 0.28 | 0.28 | 0.26 |
| Olfr164 | 0.09 | 0.13 | 0.13 | 0.18 | 0.16 | 0.14 | 0.16 | 0.15 |
| Olfr165 | 0.13 | 0.17 | 0.16 | 0.19 | 0.21 | 0.20 | 0.17 | 0.23 |
| Olfr166 | 0.20 | 0.21 | 0.15 | 0.23 | 0.18 | 0.15 | 0.17 | 0.23 |
| Olfr167 | 0.09 | 0.13 | 0.12 | 0.13 | 0.15 | 0.13 | 0.11 | 0.10 |
| Olfr168 | 0.09 | 0.14 | 0.12 | 0.15 | 0.15 | 0.12 | 0.14 | 0.18 |
| Olfr169 | 0.26 | 0.32 | 0.25 | 0.33 | 0.34 | 0.30 | 0.32 | 0.33 |
| Olfr17 | 0.16 | 0.21 | 0.18 | 0.20 | 0.21 | 0.24 | 0.22 | 0.27 |
| Olfr170 | 0.26 | 0.32 | 0.25 | 0.33 | 0.34 | 0.30 | 0.32 | 0.33 |
| Olfr171 | 0.26 | 0.32 | 0.25 | 0.33 | 0.34 | 0.30 | 0.32 | 0.33 |
| Olfr172 | 0.11 | 0.18 | 0.19 | 0.15 | 0.15 | 0.17 | 0.23 | 0.19 |
| Olfr173 | 0.04 | 0.07 | 0.09 | 0.07 | 0.05 | 0.08 | 0.10 | 0.13 |
| Olfr175-ps1 | NA | NA | 0.00 | 0.01 | 0.01 | 0.01 | 0.02 | 0.05 |
| Olfr176 | 0.02 | 0.03 | 0.03 | 0.03 | 0.05 | 0.05 | 0.06 | 0.07 |
| Olfr177 | 0.02 | 0.03 | 0.03 | 0.03 | 0.05 | 0.05 | 0.06 | 0.07 |
| Olfr178 | NA | 0.02 | 0.01 | 0.04 | −0.01 | 0.03 | 0.00 | 0.09 |
| Olfr18 | 0.06 | 0.08 | 0.06 | 0.10 | 0.09 | 0.10 | 0.07 | 0.14 |
| Olfr180 | 0.18 | 0.21 | 0.19 | 0.21 | 0.18 | 0.27 | 0.22 | 0.20 |
| Olfr181 | 0.00 | 0.01 | 0.01 | 0.04 | 0.00 | 0.01 | 0.02 | 0.06 |
| Olfr183 | 0.04 | 0.06 | 0.04 | 0.05 | 0.07 | 0.05 | 0.08 | 0.08 |
| Olfr186 | 0.04 | 0.06 | 0.06 | 0.09 | 0.09 | 0.08 | 0.10 | 0.13 |
| Olfr187 | 0.06 | 0.04 | 0.06 | 0.08 | 0.07 | 0.08 | 0.10 | 0.07 |
| Olfr19 | 0.19 | 0.24 | 0.21 | 0.23 | 0.22 | 0.21 | 0.22 | 0.19 |
| Olfr190 | NA | 0.01 | NA | 0.01 | 0.01 | NA | 0.03 | 0.09 |
| Olfr191 | 0.02 | 0.04 | 0.03 | 0.04 | 0.03 | 0.02 | 0.05 | 0.06 |
| Olfr192 | NA | NA | NA | 0.01 | 0.01 | NA | 0.02 | 0.01 |
| Olfr193 | NA | NA | NA | 0.00 | 0.00 | NA | NA | 0.02 |
| Olfr194 | 0.04 | 0.06 | 0.04 | 0.06 | 0.08 | 0.06 | 0.08 | 0.13 |
| Olfr195 | 0.06 | 0.08 | 0.08 | 0.09 | 0.07 | 0.10 | 0.11 | 0.16 |
| Olfr196 | NA | NA | NA | NA | −0.01 | NA | NA | NA |
| Olfr197 | 0.16 | 0.17 | 0.15 | 0.18 | 0.15 | 0.22 | 0.18 | 0.28 |
| Olfr198 | 0.09 | 0.11 | 0.12 | 0.12 | 0.16 | 0.15 | 0.16 | 0.17 |
| Olfr199 | 0.03 | 0.04 | 0.03 | 0.04 | 0.04 | 0.06 | 0.05 | 0.06 |
| Olfr2 | 0.06 | 0.07 | 0.08 | 0.12 | 0.09 | 0.13 | 0.11 | 0.15 |
| Olfr20 | 0.14 | 0.18 | 0.17 | 0.21 | 0.19 | 0.19 | 0.18 | 0.17 |
| Olfr201 | 0.05 | 0.06 | 0.06 | 0.09 | 0.08 | 0.07 | 0.09 | 0.08 |
| Olfr202 | 0.12 | 0.17 | 0.15 | 0.18 | 0.16 | 0.18 | 0.14 | 0.25 |
| Olfr203 | NA | NA | NA | NA | 0.00 | 0.03 | 0.01 | 0.04 |
| Olfr204 | 0.06 | 0.10 | 0.08 | 0.11 | 0.13 | 0.09 | 0.10 | 0.11 |
| Olfr205 | 0.06 | 0.10 | 0.08 | 0.11 | 0.13 | 0.09 | 0.10 | 0.11 |
| Olfr206 | NA | 0.03 | 0.02 | 0.02 | 0.02 | 0.02 | 0.03 | 0.09 |
| Olfr207 | 0.10 | 0.12 | 0.07 | 0.11 | 0.12 | 0.11 | 0.07 | 0.15 |
| Olfr208 | 0.10 | 0.12 | 0.07 | 0.11 | 0.12 | 0.11 | 0.07 | 0.15 |
| Olfr209 | 0.10 | 0.12 | 0.07 | 0.11 | 0.12 | 0.11 | 0.07 | 0.15 |
| Olfr211 | 0.21 | 0.25 | 0.22 | 0.26 | 0.24 | 0.28 | 0.26 | 0.24 |
| Olfr212 | 0.17 | 0.20 | 0.17 | 0.20 | 0.21 | 0.22 | 0.23 | 0.19 |
| Olfr213 | 0.23 | 0.27 | 0.22 | 0.27 | 0.26 | 0.23 | 0.25 | 0.25 |
| Olfr214 | 0.25 | 0.31 | 0.27 | 0.30 | 0.29 | 0.32 | 0.29 | 0.29 |
| Olfr215 | 0.23 | 0.25 | 0.23 | 0.31 | 0.29 | 0.27 | 0.29 | 0.33 |
| Olfr218 | 0.09 | 0.11 | 0.09 | 0.11 | 0.11 | 0.13 | 0.12 | 0.08 |
| Olfr220 | 0.24 | 0.26 | 0.26 | 0.26 | 0.26 | 0.25 | 0.26 | 0.24 |
| Olfr221 | 0.25 | 0.30 | 0.29 | 0.33 | 0.30 | 0.30 | 0.30 | 0.35 |
| Olfr222 | 0.32 | 0.38 | 0.33 | 0.39 | 0.41 | 0.38 | 0.38 | 0.39 |
| Olfr223 | 0.16 | 0.20 | 0.18 | 0.22 | 0.22 | 0.22 | 0.22 | 0.29 |
| Olfr224 | 0.03 | 0.02 | 0.02 | 0.08 | 0.02 | 0.05 | 0.06 | 0.07 |
| Olfr225 | 0.27 | 0.31 | 0.26 | 0.32 | 0.30 | 0.31 | 0.30 | 0.26 |
| Olfr228 | 0.02 | 0.04 | 0.02 | 0.04 | 0.02 | 0.03 | 0.06 | 0.12 |
| Olfr229 | 0.03 | 0.03 | 0.02 | 0.04 | 0.02 | 0.00 | 0.02 | 0.04 |
| Olfr23 | 0.17 | 0.20 | 0.15 | 0.18 | 0.18 | 0.20 | 0.16 | 0.19 |
| Olfr231 | 0.24 | 0.30 | 0.26 | 0.30 | 0.28 | 0.30 | 0.25 | 0.30 |
| Olfr235 | 0.09 | 0.15 | 0.14 | 0.14 | 0.11 | 0.13 | 0.17 | 0.17 |
| Olfr237-ps1 | 0.08 | 0.13 | 0.09 | 0.14 | 0.15 | 0.11 | 0.09 | 0.13 |
| Olfr239 | 0.50 | 0.62 | 0.56 | 0.63 | 0.59 | 0.60 | 0.54 | 0.65 |
| Olfr24 | 0.17 | 0.22 | 0.19 | 0.22 | 0.19 | 0.22 | 0.20 | 0.25 |
| Olfr242 | 0.04 | 0.07 | 0.06 | 0.08 | 0.09 | 0.07 | 0.11 | 0.09 |
| Olfr243 | NA | NA | NA | NA | 0.00 | 0.04 | NA | 0.04 |
| Olfr247 | 0.22 | 0.27 | 0.24 | 0.26 | 0.27 | 0.31 | 0.26 | 0.28 |
| Olfr248 | 0.08 | 0.10 | 0.09 | 0.11 | 0.11 | 0.13 | 0.08 | 0.12 |
| Olfr25 | 0.06 | 0.10 | 0.07 | 0.10 | 0.12 | 0.09 | 0.09 | 0.15 |
| Olfr250 | 0.11 | 0.15 | 0.13 | 0.14 | 0.14 | 0.17 | 0.16 | 0.14 |
| Olfr251 | 0.11 | 0.15 | 0.12 | 0.16 | 0.15 | 0.16 | 0.13 | 0.12 |
| Olfr255 | 0.14 | 0.18 | 0.17 | 0.21 | 0.19 | 0.19 | 0.18 | 0.17 |
| Olfr257 | 0.27 | 0.30 | 0.27 | 0.32 | 0.32 | 0.30 | 0.30 | 0.26 |
| Olfr259 | 0.00 | 0.02 | 0.02 | 0.03 | 0.04 | 0.04 | 0.01 | 0.06 |
| Olfr26 | 0.13 | 0.16 | 0.14 | 0.16 | 0.14 | 0.14 | 0.13 | 0.16 |
| Olfr262 | 0.12 | 0.17 | 0.14 | 0.17 | 0.16 | 0.19 | 0.16 | 0.16 |
| Olfr263 | 0.02 | 0.04 | 0.04 | 0.04 | 0.05 | 0.04 | 0.07 | 0.12 |
| Olfr266 | 0.16 | 0.18 | 0.18 | 0.17 | 0.19 | 0.25 | 0.19 | 0.19 |
| Olfr267 | 0.32 | 0.34 | 0.30 | 0.35 | 0.32 | 0.34 | 0.30 | 0.32 |
| Olfr27 | 0.04 | 0.07 | 0.06 | 0.08 | 0.09 | 0.07 | 0.11 | 0.09 |
| Olfr270 | 0.09 | 0.11 | 0.10 | 0.12 | 0.13 | 0.16 | 0.14 | 0.14 |
| Olfr272 | 0.19 | 0.20 | 0.19 | 0.24 | 0.21 | 0.23 | 0.19 | 0.24 |
| Olfr273 | 0.14 | 0.16 | 0.15 | 0.19 | 0.18 | 0.21 | 0.16 | 0.16 |
| Olfr275 | 0.15 | 0.19 | 0.17 | 0.18 | 0.18 | 0.18 | 0.17 | 0.17 |
| Olfr279 | 0.27 | 0.32 | 0.30 | 0.35 | 0.36 | 0.36 | 0.31 | 0.38 |
| Olfr281 | 0.25 | 0.28 | 0.25 | 0.32 | 0.27 | 0.28 | 0.26 | 0.32 |
| Olfr282 | 0.34 | 0.40 | 0.35 | 0.43 | 0.41 | 0.40 | 0.40 | 0.38 |
| Olfr283 | 0.24 | 0.29 | 0.27 | 0.27 | 0.32 | 0.27 | 0.28 | 0.28 |
| Olfr284 | 0.27 | 0.31 | 0.30 | 0.39 | 0.32 | 0.35 | 0.34 | 0.37 |
| Olfr285 | 0.27 | 0.30 | 0.27 | 0.32 | 0.32 | 0.30 | 0.30 | 0.26 |
| Olfr286 | 0.19 | 0.22 | 0.18 | 0.23 | 0.22 | 0.19 | 0.22 | 0.24 |
| Olfr288 | 0.15 | 0.16 | 0.22 | 0.22 | 0.15 | 0.24 | 0.22 | 0.19 |
| Olfr29-ps1 | 0.25 | 0.30 | 0.29 | 0.32 | 0.28 | 0.29 | 0.28 | 0.27 |
| Olfr290 | 0.04 | 0.05 | 0.03 | 0.04 | 0.05 | 0.08 | 0.05 | 0.07 |
| Olfr291 | 0.06 | 0.09 | 0.04 | 0.07 | 0.08 | 0.06 | 0.07 | 0.09 |
| Olfr292 | 0.05 | 0.06 | 0.05 | 0.08 | 0.08 | 0.11 | 0.06 | 0.08 |
| Olfr293 | 0.11 | 0.14 | 0.12 | 0.13 | 0.13 | 0.13 | 0.12 | 0.15 |
| Olfr294 | 0.07 | 0.11 | 0.10 | 0.10 | 0.11 | 0.10 | 0.13 | 0.17 |
| Olfr295 | 0.00 | 0.02 | 0.01 | 0.02 | 0.02 | 0.05 | 0.01 | 0.06 |
| Olfr297 | 0.08 | 0.10 | 0.11 | 0.13 | 0.11 | 0.12 | 0.16 | 0.14 |
| Olfr298 | 0.06 | 0.08 | 0.07 | 0.09 | 0.08 | 0.09 | 0.07 | 0.08 |
| Olfr299 | NA | NA | NA | NA | −0.04 | NA | NA | 0.03 |
| Olfr3 | 0.18 | 0.25 | 0.20 | 0.25 | 0.21 | 0.25 | 0.22 | 0.25 |
| Olfr30 | 0.31 | 0.36 | 0.30 | 0.44 | 0.40 | 0.36 | 0.34 | 0.37 |
| Olfr301 | 0.05 | 0.06 | 0.06 | 0.07 | 0.07 | 0.08 | 0.07 | 0.15 |
| Olfr303 | 0.11 | 0.15 | 0.11 | 0.15 | 0.19 | 0.17 | 0.14 | 0.21 |
| Olfr304 | 0.06 | 0.07 | 0.07 | 0.10 | 0.09 | 0.10 | 0.11 | 0.16 |
| Olfr305 | 0.12 | 0.15 | 0.17 | 0.18 | 0.19 | 0.22 | 0.19 | 0.21 |
| Olfr307 | 0.15 | 0.18 | 0.16 | 0.21 | 0.20 | 0.18 | 0.20 | 0.29 |
| Olfr308 | 0.07 | 0.09 | 0.08 | 0.07 | 0.10 | 0.11 | 0.10 | 0.11 |
| Olfr309 | 0.19 | 0.21 | 0.21 | 0.22 | 0.21 | 0.21 | 0.21 | 0.22 |
| Olfr31 | 0.23 | 0.26 | 0.24 | 0.28 | 0.28 | 0.30 | 0.27 | 0.27 |
| Olfr310 | 0.05 | 0.04 | 0.04 | 0.09 | 0.08 | 0.10 | 0.10 | 0.13 |
| Olfr311 | 0.14 | 0.15 | 0.14 | 0.20 | 0.17 | 0.19 | 0.17 | 0.20 |
| Olfr312 | 0.19 | 0.22 | 0.18 | 0.24 | 0.23 | 0.23 | 0.20 | 0.25 |
| Olfr313 | 0.16 | 0.20 | 0.19 | 0.20 | 0.21 | 0.20 | 0.17 | 0.17 |
| Olfr314 | 0.41 | 0.49 | 0.43 | 0.53 | 0.48 | 0.50 | 0.47 | 0.46 |
| Olfr315 | 0.30 | 0.32 | 0.32 | 0.38 | 0.38 | 0.37 | 0.35 | 0.34 |
| Olfr316 | 0.36 | 0.37 | 0.37 | 0.42 | 0.42 | 0.41 | 0.37 | 0.31 |
| Olfr317 | 0.27 | 0.30 | 0.29 | 0.33 | 0.28 | 0.31 | 0.30 | 0.29 |
| Olfr318 | 0.18 | 0.19 | 0.16 | 0.20 | 0.20 | 0.22 | 0.21 | 0.22 |
| Olfr319 | 0.19 | 0.25 | 0.19 | 0.24 | 0.25 | 0.24 | 0.21 | 0.22 |
| Olfr32 | 0.08 | 0.09 | 0.09 | 0.09 | 0.09 | 0.12 | 0.09 | 0.09 |
| Olfr320 | 0.26 | 0.31 | 0.32 | 0.35 | 0.32 | 0.36 | 0.31 | 0.33 |
| Olfr322 | 0.33 | 0.34 | 0.35 | 0.39 | 0.38 | 0.38 | 0.38 | 0.43 |
| Olfr323 | 0.12 | 0.15 | 0.13 | 0.19 | 0.16 | 0.14 | 0.13 | 0.23 |
| Olfr324 | 0.20 | 0.24 | 0.20 | 0.27 | 0.29 | 0.26 | 0.27 | 0.22 |
| Olfr325 | 0.08 | 0.11 | 0.09 | 0.14 | 0.15 | 0.11 | 0.11 | 0.16 |
| Olfr328 | 0.24 | 0.28 | 0.27 | 0.33 | 0.32 | 0.32 | 0.31 | 0.37 |
| Olfr329-ps | 0.26 | 0.32 | 0.29 | 0.34 | 0.29 | 0.33 | 0.30 | 0.29 |
| Olfr33 | 0.16 | 0.22 | 0.18 | 0.23 | 0.22 | 0.26 | 0.20 | 0.25 |
| Olfr330 | 0.25 | 0.30 | 0.27 | 0.29 | 0.26 | 0.29 | 0.28 | 0.30 |
| Olfr331 | 0.26 | 0.32 | 0.29 | 0.34 | 0.29 | 0.33 | 0.30 | 0.29 |
| Olfr332 | 0.24 | 0.33 | 0.27 | 0.35 | 0.34 | 0.32 | 0.30 | 0.31 |
| Olfr338 | 0.04 | 0.05 | 0.04 | 0.07 | 0.10 | 0.10 | 0.08 | 0.14 |
| Olfr339 | 0.02 | 0.03 | 0.04 | 0.03 | 0.04 | 0.07 | 0.05 | 0.11 |
| Olfr340 | 0.01 | 0.05 | 0.03 | 0.04 | 0.06 | 0.06 | 0.02 | 0.11 |
| Olfr341 | 0.13 | 0.17 | 0.15 | 0.21 | 0.21 | 0.20 | 0.18 | 0.17 |
| Olfr342 | 0.06 | 0.10 | 0.09 | 0.09 | 0.10 | 0.11 | 0.12 | 0.18 |
| Olfr344 | 0.08 | 0.12 | 0.07 | 0.08 | 0.12 | 0.13 | 0.11 | 0.13 |
| Olfr345 | 0.05 | 0.07 | 0.06 | 0.09 | 0.04 | 0.06 | 0.06 | 0.10 |
| Olfr346 | 0.08 | 0.11 | 0.09 | 0.12 | 0.12 | 0.11 | 0.11 | 0.14 |
| Olfr347 | 0.10 | 0.12 | 0.09 | 0.15 | 0.13 | 0.15 | 0.13 | 0.14 |
| Olfr348 | 0.07 | 0.09 | 0.09 | 0.11 | 0.12 | 0.13 | 0.11 | 0.11 |
| Olfr350 | 0.08 | 0.12 | 0.12 | 0.13 | 0.11 | 0.11 | 0.12 | 0.13 |
| Olfr351 | 0.21 | 0.25 | 0.21 | 0.25 | 0.23 | 0.26 | 0.24 | 0.24 |
| Olfr352 | 0.12 | 0.16 | 0.15 | 0.20 | 0.16 | 0.18 | 0.15 | 0.20 |
| Olfr353 | 0.11 | 0.15 | 0.13 | 0.13 | 0.14 | 0.17 | 0.14 | 0.20 |
| Olfr354 | 0.16 | 0.20 | 0.20 | 0.22 | 0.19 | 0.24 | 0.19 | 0.21 |
| Olfr355 | 0.08 | 0.08 | 0.08 | 0.11 | 0.10 | 0.08 | 0.07 | 0.11 |
| Olfr356 | 0.14 | 0.16 | 0.18 | 0.17 | 0.17 | 0.16 | 0.19 | 0.16 |
| Olfr357 | 0.11 | 0.14 | 0.13 | 0.16 | 0.14 | 0.15 | 0.14 | 0.15 |
| Olfr358 | 0.07 | 0.10 | 0.08 | 0.11 | 0.10 | 0.11 | 0.10 | 0.07 |
| Olfr360 | 0.07 | 0.10 | 0.08 | 0.11 | 0.10 | 0.11 | 0.10 | 0.07 |
| Olfr361 | 0.18 | 0.19 | 0.17 | 0.23 | 0.23 | 0.21 | 0.19 | 0.24 |
| Olfr362 | 0.26 | 0.30 | 0.28 | 0.33 | 0.30 | 0.31 | 0.29 | 0.30 |
| Olfr365 | 0.24 | 0.28 | 0.28 | 0.34 | 0.33 | 0.33 | 0.31 | 0.30 |
| Olfr366 | 0.19 | 0.22 | 0.21 | 0.26 | 0.24 | 0.25 | 0.22 | 0.24 |
| Olfr367-ps | 0.24 | 0.27 | 0.25 | 0.29 | 0.26 | 0.32 | 0.28 | 0.27 |
| Olfr368 | 0.33 | 0.36 | 0.34 | 0.38 | 0.39 | 0.37 | 0.34 | 0.38 |
| Olfr370 | 0.26 | 0.30 | 0.27 | 0.34 | 0.33 | 0.32 | 0.28 | 0.32 |
| Olfr371 | 0.24 | 0.28 | 0.27 | 0.30 | 0.28 | 0.31 | 0.28 | 0.32 |
| Olfr372 | 0.32 | 0.37 | 0.33 | 0.39 | 0.36 | 0.39 | 0.34 | 0.39 |
| Olfr373 | 0.27 | 0.35 | 0.28 | 0.36 | 0.37 | 0.37 | 0.33 | 0.43 |
| Olfr374 | 0.21 | 0.25 | 0.21 | 0.22 | 0.28 | 0.26 | 0.24 | 0.27 |
| Olfr376 | 0.15 | 0.19 | 0.18 | 0.19 | 0.19 | 0.22 | 0.19 | 0.20 |
| Olfr378 | 0.05 | 0.05 | 0.05 | 0.08 | 0.07 | 0.08 | 0.09 | 0.12 |
| Olfr38 | 0.22 | 0.24 | 0.23 | 0.25 | 0.23 | 0.27 | 0.23 | 0.27 |
| Olfr380 | 0.02 | 0.06 | 0.03 | 0.08 | 0.07 | 0.07 | 0.05 | 0.13 |
| Olfr381 | 0.11 | 0.15 | 0.15 | 0.15 | 0.16 | 0.15 | 0.12 | 0.19 |
| Olfr382 | 0.12 | 0.15 | 0.13 | 0.15 | 0.15 | 0.14 | 0.17 | 0.20 |
| Olfr384 | 0.05 | 0.09 | 0.06 | 0.09 | 0.09 | 0.10 | 0.10 | 0.09 |
| Olfr385 | 0.04 | 0.05 | 0.05 | 0.06 | 0.06 | 0.09 | 0.06 | 0.09 |
| Olfr389 | 0.11 | 0.14 | 0.14 | 0.15 | 0.13 | 0.14 | 0.11 | 0.16 |
| Olfr39 | 0.19 | 0.24 | 0.23 | 0.24 | 0.25 | 0.26 | 0.24 | 0.28 |
| Olfr390 | 0.09 | 0.12 | 0.10 | 0.11 | 0.11 | 0.14 | 0.10 | 0.08 |
| Olfr391-ps | 0.14 | 0.18 | 0.17 | 0.21 | 0.19 | 0.19 | 0.18 | 0.17 |
| Olfr392 | 0.03 | 0.05 | 0.04 | 0.04 | 0.05 | 0.07 | 0.06 | 0.09 |
| Olfr393 | 0.05 | 0.07 | 0.08 | 0.07 | 0.08 | 0.11 | 0.10 | 0.08 |
| Olfr394 | 0.11 | 0.14 | 0.11 | 0.15 | 0.16 | 0.14 | 0.11 | 0.15 |
| Olfr395 | 0.14 | 0.18 | 0.17 | 0.21 | 0.19 | 0.19 | 0.18 | 0.17 |
| Olfr397 | 0.10 | 0.14 | 0.11 | 0.12 | 0.12 | 0.12 | 0.15 | 0.16 |
| Olfr398 | 0.31 | 0.36 | 0.32 | 0.35 | 0.35 | 0.35 | 0.33 | 0.35 |
| Olfr399 | 0.21 | 0.26 | 0.23 | 0.27 | 0.25 | 0.24 | 0.23 | 0.24 |
| Olfr401 | 0.37 | 0.44 | 0.40 | 0.48 | 0.47 | 0.42 | 0.39 | 0.41 |
| Olfr402 | 0.21 | 0.28 | 0.24 | 0.28 | 0.25 | 0.26 | 0.24 | 0.28 |
| Olfr403 | 0.17 | 0.21 | 0.16 | 0.23 | 0.20 | 0.23 | 0.21 | 0.22 |
| Olfr406 | 0.20 | 0.24 | 0.20 | 0.28 | 0.25 | 0.26 | 0.26 | 0.26 |
| Olfr410 | 0.15 | 0.18 | 0.16 | 0.18 | 0.22 | 0.20 | 0.16 | 0.20 |
| Olfr411 | 0.32 | 0.38 | 0.34 | 0.44 | 0.38 | 0.39 | 0.36 | 0.38 |
| Olfr412 | 0.29 | 0.35 | 0.31 | 0.34 | 0.34 | 0.36 | 0.34 | 0.34 |
| Olfr414 | 0.25 | 0.26 | 0.26 | 0.29 | 0.29 | 0.29 | 0.28 | 0.30 |
| Olfr417 | 0.30 | 0.34 | 0.33 | 0.35 | 0.36 | 0.32 | 0.34 | 0.35 |
| Olfr418 | 0.15 | 0.19 | 0.17 | 0.20 | 0.19 | 0.20 | 0.20 | 0.22 |
| Olfr419 | 0.24 | 0.27 | 0.23 | 0.27 | 0.29 | 0.29 | 0.28 | 0.22 |
| Olfr420 | 0.19 | 0.22 | 0.23 | 0.25 | 0.23 | 0.22 | 0.23 | 0.20 |
| Olfr421-ps1 | 0.10 | 0.13 | 0.13 | 0.14 | 0.15 | 0.16 | 0.18 | 0.21 |
| Olfr424 | 0.19 | 0.25 | 0.23 | 0.26 | 0.24 | 0.26 | 0.21 | 0.28 |
| Olfr426 | 0.11 | 0.15 | 0.15 | 0.14 | 0.14 | 0.17 | 0.16 | 0.14 |
| Olfr427 | 0.11 | 0.15 | 0.15 | 0.14 | 0.14 | 0.17 | 0.16 | 0.14 |
| Olfr429 | 0.16 | 0.21 | 0.17 | 0.24 | 0.23 | 0.21 | 0.22 | 0.19 |
| Olfr43 | 0.01 | 0.06 | 0.06 | 0.06 | 0.07 | 0.05 | 0.08 | 0.10 |
| Olfr430 | 0.09 | 0.13 | 0.13 | 0.13 | 0.11 | 0.13 | 0.09 | 0.12 |
| Olfr432 | 0.15 | 0.18 | 0.18 | 0.20 | 0.16 | 0.18 | 0.19 | 0.17 |
| Olfr433 | 0.29 | 0.31 | 0.28 | 0.33 | 0.31 | 0.29 | 0.27 | 0.31 |
| Olfr434 | 0.20 | 0.20 | 0.20 | 0.23 | 0.24 | 0.24 | 0.20 | 0.26 |
| Olfr435 | 0.29 | 0.32 | 0.29 | 0.35 | 0.33 | 0.33 | 0.30 | 0.35 |
| Olfr437 | 0.19 | 0.23 | 0.21 | 0.24 | 0.23 | 0.26 | 0.21 | 0.24 |
| Olfr44 | 0.03 | 0.05 | 0.05 | 0.06 | 0.06 | 0.05 | 0.05 | 0.05 |
| Olfr441 | 0.21 | 0.25 | 0.24 | 0.27 | 0.25 | 0.27 | 0.23 | 0.23 |
| Olfr444 | 0.24 | 0.29 | 0.26 | 0.33 | 0.32 | 0.31 | 0.35 | 0.38 |
| Olfr446 | 0.14 | 0.16 | 0.13 | 0.17 | 0.20 | 0.20 | 0.20 | 0.24 |
| Olfr447 | 0.22 | 0.28 | 0.23 | 0.29 | 0.25 | 0.25 | 0.26 | 0.25 |
| Olfr448 | 0.20 | 0.24 | 0.20 | 0.24 | 0.23 | 0.25 | 0.19 | 0.27 |
| Olfr449 | 0.17 | 0.16 | 0.18 | 0.20 | 0.21 | 0.22 | 0.22 | 0.18 |
| Olfr45 | 0.22 | 0.29 | 0.24 | 0.31 | 0.27 | 0.30 | 0.28 | 0.26 |
| Olfr450 | 0.19 | 0.24 | 0.22 | 0.23 | 0.21 | 0.26 | 0.24 | 0.20 |
| Olfr452 | 0.05 | 0.05 | 0.05 | 0.05 | 0.03 | 0.05 | 0.10 | 0.07 |
| Olfr453 | 0.18 | 0.20 | 0.18 | 0.21 | 0.21 | 0.20 | 0.23 | 0.20 |
| Olfr455 | 0.20 | 0.21 | 0.22 | 0.22 | 0.23 | 0.24 | 0.24 | 0.23 |
| Olfr456 | 0.25 | 0.34 | 0.31 | 0.33 | 0.32 | 0.34 | 0.29 | 0.36 |
| Olfr457 | 0.25 | 0.34 | 0.31 | 0.33 | 0.32 | 0.34 | 0.29 | 0.36 |
| Olfr458 | 0.26 | 0.33 | 0.31 | 0.37 | 0.32 | 0.36 | 0.31 | 0.38 |
| Olfr459 | 0.22 | 0.27 | 0.22 | 0.27 | 0.24 | 0.28 | 0.27 | 0.28 |
| Olfr46 | 0.22 | 0.29 | 0.24 | 0.31 | 0.27 | 0.30 | 0.28 | 0.26 |
| Olfr460 | 0.14 | 0.16 | 0.14 | 0.18 | 0.17 | 0.17 | 0.16 | 0.16 |
| Olfr461 | 0.10 | 0.12 | 0.09 | 0.16 | 0.10 | 0.15 | 0.10 | 0.13 |
| Olfr462 | 0.37 | 0.48 | 0.40 | 0.46 | 0.46 | 0.42 | 0.43 | 0.42 |
| Olfr463 | 0.27 | 0.31 | 0.28 | 0.38 | 0.36 | 0.35 | 0.35 | 0.31 |
| Olfr464 | 0.29 | 0.34 | 0.32 | 0.37 | 0.39 | 0.36 | 0.35 | 0.37 |
| Olfr466 | 0.08 | 0.11 | 0.09 | 0.11 | 0.13 | 0.12 | 0.09 | 0.11 |
| Olfr467 | 0.21 | 0.27 | 0.23 | 0.25 | 0.24 | 0.28 | 0.24 | 0.24 |
| Olfr469 | 0.09 | 0.11 | 0.11 | 0.13 | 0.15 | 0.13 | 0.16 | 0.20 |
| Olfr47 | 0.16 | 0.20 | 0.17 | 0.20 | 0.23 | 0.21 | 0.24 | 0.24 |
| Olfr470 | 0.09 | 0.10 | 0.08 | 0.11 | 0.12 | 0.13 | 0.10 | 0.15 |
| Olfr472 | NA | 0.00 | NA | NA | −0.01 | NA | 0.01 | 0.02 |
| Olfr473 | 0.08 | 0.10 | 0.09 | 0.10 | 0.14 | 0.12 | 0.11 | 0.14 |
| Olfr474 | 0.02 | 0.04 | 0.03 | 0.05 | 0.03 | 0.03 | 0.06 | 0.08 |
| Olfr476 | 0.14 | 0.18 | 0.16 | 0.17 | 0.15 | 0.22 | 0.16 | 0.19 |
| Olfr477 | 0.22 | 0.25 | 0.23 | 0.28 | 0.24 | 0.26 | 0.25 | 0.26 |
| Olfr478 | 0.08 | 0.11 | 0.10 | 0.10 | 0.11 | 0.14 | 0.11 | 0.16 |
| Olfr479 | 0.24 | 0.25 | 0.24 | 0.25 | 0.26 | 0.25 | 0.25 | 0.24 |
| Olfr48 | 0.34 | 0.38 | 0.37 | 0.39 | 0.38 | 0.39 | 0.37 | 0.39 |
| Olfr480 | 0.02 | 0.01 | 0.02 | 0.01 | 0.03 | 0.03 | 0.01 | 0.06 |
| Olfr481 | 0.15 | 0.16 | 0.16 | 0.17 | 0.17 | 0.18 | 0.16 | 0.20 |
| Olfr482 | 0.08 | 0.12 | 0.12 | 0.14 | 0.15 | 0.11 | 0.15 | 0.16 |
| Olfr483 | 0.15 | 0.19 | 0.17 | 0.18 | 0.20 | 0.20 | 0.19 | 0.19 |
| Olfr484 | 0.13 | 0.14 | 0.15 | 0.14 | 0.16 | 0.14 | 0.16 | 0.18 |
| Olfr485 | 0.21 | 0.25 | 0.24 | 0.26 | 0.28 | 0.25 | 0.24 | 0.34 |
| Olfr486 | 0.15 | 0.19 | 0.17 | 0.20 | 0.19 | 0.20 | 0.18 | 0.26 |
| Olfr487 | 0.15 | 0.19 | 0.17 | 0.20 | 0.19 | 0.20 | 0.18 | 0.26 |
| Olfr488 | 0.08 | 0.13 | 0.11 | 0.12 | 0.12 | 0.15 | 0.13 | 0.14 |
| Olfr49 | 0.12 | 0.16 | 0.14 | 0.15 | 0.19 | 0.17 | 0.15 | 0.17 |
| Olfr490 | NA | 0.02 | NA | 0.02 | 0.00 | 0.00 | NA | 0.05 |
| Olfr491 | 0.15 | 0.20 | 0.15 | 0.19 | 0.17 | 0.19 | 0.15 | 0.20 |
| Olfr492 | 0.09 | 0.13 | 0.11 | 0.14 | 0.14 | 0.14 | 0.14 | 0.15 |
| Olfr493 | 0.07 | 0.09 | 0.09 | 0.12 | 0.09 | 0.11 | 0.11 | 0.18 |
| Olfr494 | 0.13 | 0.16 | 0.15 | 0.18 | 0.16 | 0.17 | 0.16 | 0.18 |
| Olfr495 | 0.09 | 0.11 | 0.13 | 0.11 | 0.11 | 0.14 | 0.09 | 0.15 |
| Olfr497 | 0.06 | 0.10 | 0.11 | 0.10 | 0.10 | 0.13 | 0.15 | 0.15 |
| Olfr498 | 0.08 | 0.10 | 0.09 | 0.11 | 0.11 | 0.12 | 0.12 | 0.13 |
| Olfr5 | 0.24 | 0.31 | 0.27 | 0.35 | 0.29 | 0.28 | 0.26 | 0.29 |
| Olfr50 | 0.06 | 0.09 | 0.11 | 0.10 | 0.14 | 0.08 | 0.14 | 0.05 |
| Olfr502 | 0.13 | 0.17 | 0.13 | 0.13 | 0.16 | 0.16 | 0.15 | 0.13 |
| Olfr503 | 0.21 | 0.29 | 0.23 | 0.25 | 0.26 | 0.27 | 0.26 | 0.27 |
| Olfr504 | 0.07 | 0.08 | 0.09 | 0.12 | 0.10 | 0.12 | 0.12 | 0.14 |
| Olfr506 | 0.08 | 0.12 | 0.12 | 0.14 | 0.15 | 0.11 | 0.15 | 0.16 |
| Olfr507 | 0.08 | 0.12 | 0.12 | 0.14 | 0.15 | 0.11 | 0.15 | 0.16 |
| Olfr508 | 0.11 | 0.18 | 0.13 | 0.13 | 0.20 | 0.17 | 0.14 | 0.16 |
| Olfr509 | 0.05 | 0.06 | 0.04 | 0.07 | 0.10 | 0.07 | 0.07 | 0.11 |
| Olfr51 | 0.24 | 0.26 | 0.26 | 0.27 | 0.27 | 0.26 | 0.27 | 0.31 |
| Olfr510 | 0.09 | 0.09 | 0.07 | 0.11 | 0.09 | 0.11 | 0.12 | 0.17 |
| Olfr512 | 0.02 | 0.04 | 0.05 | 0.05 | 0.07 | 0.06 | 0.04 | 0.11 |
| Olfr513 | 0.19 | 0.24 | 0.23 | 0.23 | 0.26 | 0.25 | 0.24 | 0.22 |
| Olfr514 | 0.21 | 0.28 | 0.25 | 0.28 | 0.24 | 0.30 | 0.27 | 0.27 |
| Olfr516 | 0.02 | 0.06 | 0.04 | 0.10 | 0.09 | 0.06 | 0.09 | 0.11 |
| Olfr517 | 0.18 | 0.21 | 0.20 | 0.24 | 0.22 | 0.22 | 0.19 | 0.28 |
| Olfr518 | 0.08 | 0.13 | 0.10 | 0.12 | 0.12 | 0.10 | 0.13 | 0.13 |
| Olfr519 | 0.08 | 0.13 | 0.10 | 0.12 | 0.12 | 0.10 | 0.13 | 0.13 |
| Olfr52 | 0.17 | 0.20 | 0.19 | 0.20 | 0.21 | 0.20 | 0.21 | 0.27 |
| Olfr520 | 0.18 | 0.22 | 0.21 | 0.22 | 0.25 | 0.24 | 0.24 | 0.27 |
| Olfr521 | 0.12 | 0.20 | 0.12 | 0.19 | 0.22 | 0.17 | 0.13 | 0.18 |
| Olfr522 | 0.33 | 0.37 | 0.34 | 0.36 | 0.36 | 0.39 | 0.37 | 0.36 |
| Olfr523 | 0.28 | 0.35 | 0.31 | 0.37 | 0.36 | 0.36 | 0.32 | 0.32 |
| Olfr524 | 0.40 | 0.48 | 0.43 | 0.49 | 0.48 | 0.48 | 0.46 | 0.45 |
| Olfr525 | 0.20 | 0.22 | 0.20 | 0.25 | 0.26 | 0.25 | 0.23 | 0.25 |
| Olfr527 | 0.23 | 0.26 | 0.26 | 0.29 | 0.26 | 0.32 | 0.26 | 0.24 |
| Olfr53 | 0.22 | 0.29 | 0.24 | 0.31 | 0.27 | 0.30 | 0.28 | 0.26 |
| Olfr530 | 0.27 | 0.33 | 0.28 | 0.34 | 0.32 | 0.36 | 0.30 | 0.27 |
| Olfr531 | 0.18 | 0.17 | 0.17 | 0.20 | 0.21 | 0.21 | 0.18 | 0.22 |
| Olfr532 | 0.22 | 0.26 | 0.24 | 0.28 | 0.27 | 0.28 | 0.24 | 0.26 |
| Olfr533 | 0.27 | 0.33 | 0.28 | 0.34 | 0.32 | 0.36 | 0.30 | 0.27 |
| Olfr535 | 0.34 | 0.40 | 0.35 | 0.42 | 0.40 | 0.39 | 0.38 | 0.37 |
| Olfr536 | 0.27 | 0.33 | 0.28 | 0.34 | 0.32 | 0.36 | 0.30 | 0.27 |
| Olfr538 | 0.22 | 0.29 | 0.24 | 0.31 | 0.27 | 0.30 | 0.28 | 0.26 |
| Olfr539 | 0.22 | 0.29 | 0.24 | 0.31 | 0.27 | 0.30 | 0.28 | 0.26 |
| Olfr54 | 0.35 | 0.43 | 0.39 | 0.46 | 0.42 | 0.43 | 0.40 | 0.43 |
| Olfr541 | 0.22 | 0.29 | 0.24 | 0.31 | 0.27 | 0.30 | 0.28 | 0.26 |
| Olfr543 | 0.35 | 0.36 | 0.36 | 0.39 | 0.38 | 0.41 | 0.38 | 0.35 |
| Olfr544 | 0.18 | 0.22 | 0.19 | 0.24 | 0.27 | 0.21 | 0.19 | 0.15 |
| Olfr545 | 0.38 | 0.44 | 0.40 | 0.44 | 0.46 | 0.45 | 0.43 | 0.47 |
| Olfr547 | 0.13 | 0.16 | 0.13 | 0.18 | 0.17 | 0.16 | 0.18 | 0.22 |
| Olfr549 | 0.26 | 0.32 | 0.29 | 0.35 | 0.31 | 0.35 | 0.33 | 0.33 |
| Olfr55 | 0.26 | 0.35 | 0.29 | 0.36 | 0.33 | 0.35 | 0.30 | 0.30 |
| Olfr550 | 0.32 | 0.35 | 0.31 | 0.38 | 0.36 | 0.39 | 0.34 | 0.37 |
| Olfr551 | 0.19 | 0.22 | 0.18 | 0.24 | 0.24 | 0.26 | 0.21 | 0.22 |
| Olfr552 | 0.21 | 0.22 | 0.20 | 0.25 | 0.20 | 0.22 | 0.25 | 0.23 |
| Olfr553 | 0.16 | 0.18 | 0.18 | 0.20 | 0.19 | 0.19 | 0.20 | 0.17 |
| Olfr554 | 0.20 | 0.22 | 0.21 | 0.24 | 0.23 | 0.25 | 0.24 | 0.23 |
| Olfr555 | 0.19 | 0.21 | 0.21 | 0.24 | 0.23 | 0.25 | 0.23 | 0.26 |
| Olfr556 | 0.16 | 0.20 | 0.18 | 0.21 | 0.20 | 0.22 | 0.19 | 0.22 |
| Olfr557 | 0.15 | 0.20 | 0.16 | 0.21 | 0.20 | 0.20 | 0.18 | 0.26 |
| Olfr558 | 0.23 | 0.25 | 0.22 | 0.26 | 0.26 | 0.28 | 0.25 | 0.24 |
| Olfr559 | 0.09 | 0.12 | 0.13 | 0.14 | 0.19 | 0.12 | 0.11 | 0.18 |
| Olfr56 | 0.23 | 0.39 | 0.47 | 0.28 | 0.20 | 0.34 | 0.46 | 0.49 |
| Olfr560 | 0.07 | 0.10 | 0.08 | 0.11 | 0.12 | 0.11 | 0.10 | 0.14 |
| Olfr561 | 0.10 | 0.15 | 0.13 | 0.14 | 0.14 | 0.13 | 0.13 | 0.13 |
| Olfr564 | 0.07 | 0.07 | 0.06 | 0.11 | 0.10 | 0.10 | 0.11 | 0.13 |
| Olfr566 | 0.24 | 0.34 | 0.35 | 0.40 | 0.38 | 0.40 | 0.38 | 0.46 |
| Olfr568 | 0.15 | 0.19 | 0.15 | 0.21 | 0.19 | 0.18 | 0.18 | 0.23 |
| Olfr569 | 0.16 | 0.20 | 0.16 | 0.21 | 0.21 | 0.24 | 0.21 | 0.23 |
| Olfr57 | 0.03 | 0.07 | 0.06 | 0.07 | 0.09 | 0.09 | 0.08 | 0.11 |
| Olfr570 | 0.10 | 0.10 | 0.12 | 0.12 | 0.12 | 0.15 | 0.15 | 0.13 |
| Olfr571 | 0.20 | 0.28 | 0.25 | 0.27 | 0.24 | 0.29 | 0.27 | 0.23 |
| Olfr572 | 0.17 | 0.22 | 0.20 | 0.23 | 0.21 | 0.22 | 0.22 | 0.23 |
| Olfr574 | 0.14 | 0.15 | 0.16 | 0.18 | 0.16 | 0.17 | 0.17 | 0.14 |
| Olfr575 | 0.17 | 0.15 | 0.16 | 0.15 | 0.17 | 0.19 | 0.14 | 0.18 |
| Olfr576 | 0.16 | 0.21 | 0.21 | 0.21 | 0.20 | 0.25 | 0.25 | 0.17 |
| Olfr577 | 0.20 | 0.24 | 0.24 | 0.24 | 0.23 | 0.29 | 0.20 | 0.21 |
| Olfr578 | 0.08 | 0.09 | 0.08 | 0.10 | 0.12 | 0.10 | 0.11 | 0.16 |
| Olfr58 | 0.00 | 0.01 | 0.01 | 0.00 | 0.01 | 0.01 | 0.02 | 0.07 |
| Olfr582 | 0.24 | 0.29 | 0.26 | 0.30 | 0.28 | 0.34 | 0.28 | 0.29 |
| Olfr583 | 0.04 | 0.05 | 0.04 | 0.06 | 0.06 | 0.08 | 0.07 | 0.13 |
| Olfr584 | 0.12 | 0.14 | 0.14 | 0.18 | 0.16 | 0.15 | 0.14 | 0.23 |
| Olfr585 | 0.04 | 0.05 | 0.04 | 0.06 | 0.06 | 0.08 | 0.07 | 0.13 |
| Olfr586 | 0.21 | 0.22 | 0.20 | 0.24 | 0.24 | 0.27 | 0.24 | 0.24 |
| Olfr589 | 0.18 | 0.20 | 0.20 | 0.21 | 0.21 | 0.22 | 0.23 | 0.28 |
| Olfr59 | 0.21 | 0.25 | 0.23 | 0.24 | 0.25 | 0.26 | 0.27 | 0.26 |
| Olfr591 | 0.09 | 0.11 | 0.12 | 0.09 | 0.13 | 0.12 | 0.10 | 0.14 |
| Olfr592 | 0.08 | 0.11 | 0.08 | 0.12 | 0.09 | 0.14 | 0.09 | 0.16 |
| Olfr593 | 0.13 | 0.18 | 0.14 | 0.17 | 0.17 | 0.15 | 0.16 | 0.19 |
| Olfr594 | 0.17 | 0.23 | 0.21 | 0.25 | 0.24 | 0.24 | 0.19 | 0.22 |
| Olfr596 | 0.12 | 0.16 | 0.14 | 0.17 | 0.18 | 0.16 | 0.17 | 0.17 |
| Olfr597 | NA | 0.03 | 0.06 | 0.03 | 0.05 | 0.07 | 0.05 | 0.11 |
| Olfr598 | 0.16 | 0.16 | 0.16 | 0.20 | 0.21 | 0.19 | 0.19 | 0.38 |
| Olfr599 | 0.13 | 0.14 | 0.10 | 0.15 | 0.16 | 0.18 | 0.14 | 0.18 |
| Olfr6 | 0.21 | 0.23 | 0.22 | 0.24 | 0.25 | 0.29 | 0.22 | 0.25 |
| Olfr60 | 0.10 | 0.11 | 0.08 | 0.12 | 0.14 | 0.14 | 0.13 | 0.17 |
| Olfr600 | 0.11 | 0.17 | 0.12 | 0.13 | 0.16 | 0.13 | 0.14 | 0.11 |
| Olfr601 | 0.26 | 0.31 | 0.29 | 0.33 | 0.32 | 0.31 | 0.31 | 0.35 |
| Olfr603 | 0.08 | 0.09 | 0.10 | 0.17 | 0.14 | 0.14 | 0.12 | 0.17 |
| Olfr605 | 0.21 | 0.24 | 0.19 | 0.33 | 0.26 | 0.27 | 0.26 | 0.25 |
| Olfr606 | 0.08 | 0.10 | 0.09 | 0.10 | 0.11 | 0.12 | 0.10 | 0.13 |
| Olfr607 | 0.13 | 0.14 | 0.16 | 0.15 | 0.17 | 0.19 | 0.15 | 0.19 |
| Olfr608 | 0.25 | 0.31 | 0.27 | 0.31 | 0.31 | 0.31 | 0.30 | 0.24 |
| Olfr609 | 0.24 | 0.29 | 0.27 | 0.29 | 0.28 | 0.29 | 0.28 | 0.28 |
| Olfr61 | 0.22 | 0.29 | 0.24 | 0.31 | 0.27 | 0.30 | 0.28 | 0.26 |
| Olfr610 | 0.25 | 0.30 | 0.30 | 0.32 | 0.30 | 0.31 | 0.32 | 0.32 |
| Olfr611 | 0.15 | 0.16 | 0.11 | 0.18 | 0.14 | 0.19 | 0.16 | 0.17 |
| Olfr612 | 0.09 | 0.10 | 0.11 | 0.14 | 0.11 | 0.13 | 0.11 | 0.16 |
| Olfr613 | 0.12 | 0.14 | 0.12 | 0.14 | 0.14 | 0.13 | 0.13 | 0.12 |
| Olfr615 | 0.13 | 0.14 | 0.13 | 0.16 | 0.15 | 0.16 | 0.16 | 0.21 |
| Olfr616 | 0.14 | 0.18 | 0.16 | 0.19 | 0.20 | 0.20 | 0.17 | 0.19 |
| Olfr617 | 0.08 | 0.13 | 0.09 | 0.11 | 0.11 | 0.10 | 0.11 | 0.11 |
| Olfr618 | 0.04 | 0.06 | 0.05 | 0.06 | 0.06 | 0.07 | 0.05 | 0.09 |
| Olfr619 | 0.07 | 0.08 | 0.08 | 0.10 | 0.10 | 0.09 | 0.09 | 0.08 |
| Olfr62 | 0.16 | 0.19 | 0.19 | 0.23 | 0.22 | 0.21 | 0.20 | 0.22 |
| Olfr620 | 0.13 | 0.17 | 0.14 | 0.18 | 0.16 | 0.21 | 0.14 | 0.17 |
| Olfr622 | 0.12 | 0.13 | 0.13 | 0.17 | 0.17 | 0.19 | 0.18 | 0.20 |
| Olfr623 | 0.13 | 0.11 | 0.10 | 0.12 | 0.14 | 0.14 | 0.09 | 0.17 |
| Olfr624 | 0.05 | 0.05 | 0.05 | 0.07 | 0.08 | 0.04 | 0.04 | 0.07 |
| Olfr628 | 0.02 | 0.02 | 0.02 | 0.02 | 0.04 | 0.02 | 0.01 | 0.07 |
| Olfr629 | 0.11 | 0.12 | 0.12 | 0.13 | 0.12 | 0.13 | 0.11 | 0.15 |
| Olfr63 | 0.30 | 0.31 | 0.30 | 0.36 | 0.36 | 0.34 | 0.34 | 0.31 |
| Olfr630 | 0.07 | 0.09 | 0.07 | 0.09 | 0.09 | 0.11 | 0.08 | 0.14 |
| Olfr631 | 0.05 | 0.07 | 0.06 | 0.08 | 0.07 | 0.10 | 0.06 | 0.09 |
| Olfr632 | 0.30 | 0.33 | 0.30 | 0.34 | 0.34 | 0.36 | 0.31 | 0.33 |
| Olfr633 | 0.24 | 0.27 | 0.24 | 0.32 | 0.25 | 0.28 | 0.29 | 0.28 |
| Olfr635 | 0.18 | 0.23 | 0.19 | 0.24 | 0.20 | 0.22 | 0.20 | 0.20 |
| Olfr638 | 0.23 | 0.28 | 0.25 | 0.27 | 0.28 | 0.31 | 0.30 | 0.27 |
| Olfr639 | 0.13 | 0.17 | 0.15 | 0.19 | 0.14 | 0.16 | 0.18 | 0.19 |
| Olfr64 | 0.13 | 0.15 | 0.13 | 0.19 | 0.15 | 0.16 | 0.16 | 0.17 |
| Olfr640 | 0.14 | 0.14 | 0.15 | 0.18 | 0.16 | 0.18 | 0.16 | 0.19 |
| Olfr641 | 0.18 | 0.22 | 0.19 | 0.24 | 0.22 | 0.21 | 0.23 | 0.22 |
| Olfr642 | 0.21 | 0.26 | 0.22 | 0.25 | 0.24 | 0.24 | 0.27 | 0.25 |
| Olfr643 | 0.06 | 0.10 | 0.08 | 0.09 | 0.11 | 0.10 | 0.12 | 0.13 |
| Olfr644 | 0.16 | 0.18 | 0.17 | 0.19 | 0.20 | 0.20 | 0.18 | 0.22 |
| Olfr645 | 0.23 | 0.28 | 0.24 | 0.27 | 0.26 | 0.29 | 0.25 | 0.26 |
| Olfr646 | 0.25 | 0.29 | 0.24 | 0.29 | 0.29 | 0.31 | 0.27 | 0.29 |
| Olfr648 | 0.19 | 0.23 | 0.21 | 0.24 | 0.24 | 0.24 | 0.22 | 0.23 |
| Olfr649 | 0.14 | 0.15 | 0.14 | 0.18 | 0.19 | 0.18 | 0.18 | 0.21 |
| Olfr65 | 0.09 | 0.11 | 0.09 | 0.11 | 0.11 | 0.11 | 0.13 | 0.19 |
| Olfr651 | 0.20 | 0.24 | 0.21 | 0.22 | 0.21 | 0.22 | 0.22 | 0.22 |
| Olfr652 | 0.19 | 0.22 | 0.20 | 0.22 | 0.20 | 0.24 | 0.21 | 0.20 |
| Olfr653 | 0.21 | 0.26 | 0.25 | 0.25 | 0.25 | 0.28 | 0.26 | 0.23 |
| Olfr654 | 0.12 | 0.18 | 0.12 | 0.15 | 0.13 | 0.17 | 0.12 | 0.13 |
| Olfr655 | 0.14 | 0.18 | 0.15 | 0.17 | 0.15 | 0.17 | 0.17 | 0.19 |
| Olfr656 | 0.22 | 0.30 | 0.25 | 0.26 | 0.28 | 0.28 | 0.27 | 0.27 |
| Olfr657 | 0.19 | 0.26 | 0.21 | 0.23 | 0.23 | 0.23 | 0.23 | 0.23 |
| Olfr658 | 0.29 | 0.36 | 0.31 | 0.35 | 0.34 | 0.32 | 0.33 | 0.36 |
| Olfr659 | 0.11 | 0.14 | 0.14 | 0.18 | 0.15 | 0.17 | 0.21 | 0.15 |
| Olfr66 | 0.07 | 0.11 | 0.10 | 0.09 | 0.09 | 0.14 | 0.11 | 0.10 |
| Olfr661 | 0.16 | 0.17 | 0.17 | 0.18 | 0.19 | 0.18 | 0.17 | 0.17 |
| Olfr663 | 0.13 | 0.16 | 0.16 | 0.16 | 0.18 | 0.14 | 0.16 | 0.17 |
| Olfr665 | 0.04 | 0.08 | 0.08 | 0.06 | 0.08 | 0.11 | 0.05 | 0.10 |
| Olfr666 | 0.18 | 0.24 | 0.21 | 0.21 | 0.21 | 0.26 | 0.22 | 0.21 |
| Olfr667 | 0.18 | 0.21 | 0.19 | 0.20 | 0.21 | 0.20 | 0.20 | 0.26 |
| Olfr668 | 0.07 | 0.11 | 0.07 | 0.10 | 0.10 | 0.13 | 0.09 | 0.10 |
| Olfr669 | 0.23 | 0.25 | 0.25 | 0.27 | 0.27 | 0.27 | 0.26 | 0.32 |
| Olfr67 | 0.02 | 0.04 | 0.04 | 0.07 | 0.04 | 0.07 | 0.08 | 0.12 |
| Olfr670 | 0.19 | 0.22 | 0.20 | 0.24 | 0.21 | 0.24 | 0.22 | 0.29 |
| Olfr671 | 0.11 | 0.17 | 0.15 | 0.17 | 0.20 | 0.14 | 0.16 | 0.15 |
| Olfr672 | 0.11 | 0.14 | 0.13 | 0.16 | 0.14 | 0.19 | 0.16 | 0.16 |
| Olfr675 | 0.06 | 0.09 | 0.08 | 0.09 | 0.09 | 0.05 | 0.11 | 0.11 |
| Olfr676 | 0.08 | 0.11 | 0.09 | 0.11 | 0.12 | 0.15 | 0.13 | 0.14 |
| Olfr677 | 0.09 | 0.09 | 0.11 | 0.14 | 0.12 | 0.13 | 0.11 | 0.13 |
| Olfr678 | 0.29 | 0.35 | 0.31 | 0.36 | 0.32 | 0.36 | 0.33 | 0.35 |
| Olfr679 | 0.07 | 0.09 | 0.09 | 0.09 | 0.11 | 0.12 | 0.09 | 0.12 |
| Olfr68 | 0.12 | 0.14 | 0.13 | 0.18 | 0.15 | 0.14 | 0.14 | 0.16 |
| Olfr681 | 0.07 | 0.09 | 0.09 | 0.09 | 0.11 | 0.12 | 0.09 | 0.12 |
| Olfr683 | 0.10 | 0.14 | 0.11 | 0.12 | 0.15 | 0.15 | 0.16 | 0.13 |
| Olfr684 | 0.09 | 0.12 | 0.09 | 0.14 | 0.14 | 0.15 | 0.15 | 0.19 |
| Olfr685 | 0.20 | 0.21 | 0.21 | 0.24 | 0.21 | 0.21 | 0.23 | 0.20 |
| Olfr686 | 0.24 | 0.27 | 0.24 | 0.27 | 0.28 | 0.27 | 0.25 | 0.26 |
| Olfr688 | 0.12 | 0.17 | 0.14 | 0.17 | 0.16 | 0.17 | 0.18 | 0.17 |
| Olfr689 | 0.20 | 0.26 | 0.20 | 0.25 | 0.26 | 0.24 | 0.25 | 0.28 |
| Olfr69 | 0.01 | 0.01 | 0.00 | 0.03 | 0.02 | 0.01 | 0.04 | 0.04 |
| Olfr690 | 0.20 | 0.26 | 0.24 | 0.28 | 0.26 | 0.24 | 0.25 | 0.25 |
| Olfr691 | 0.19 | 0.21 | 0.20 | 0.22 | 0.24 | 0.24 | 0.19 | 0.24 |
| Olfr692 | 0.24 | 0.27 | 0.26 | 0.29 | 0.27 | 0.30 | 0.27 | 0.25 |
| Olfr693 | 0.20 | 0.25 | 0.20 | 0.27 | 0.24 | 0.28 | 0.20 | 0.26 |
| Olfr694 | 0.17 | 0.21 | 0.16 | 0.19 | 0.21 | 0.23 | 0.18 | 0.19 |
| Olfr695 | 0.12 | 0.16 | 0.12 | 0.15 | 0.14 | 0.16 | 0.14 | 0.17 |
| Olfr697 | 0.22 | 0.25 | 0.21 | 0.25 | 0.22 | 0.25 | 0.22 | 0.22 |
| Olfr698 | 0.18 | 0.20 | 0.21 | 0.24 | 0.22 | 0.22 | 0.22 | 0.25 |
| Olfr699 | 0.14 | 0.20 | 0.17 | 0.18 | 0.18 | 0.21 | 0.21 | 0.16 |
| Olfr70 | 0.30 | 0.35 | 0.31 | 0.34 | 0.34 | 0.34 | 0.34 | 0.35 |
| Olfr700 | 0.18 | 0.20 | 0.20 | 0.23 | 0.22 | 0.20 | 0.22 | 0.21 |
| Olfr701 | 0.07 | 0.12 | 0.07 | 0.13 | 0.12 | 0.10 | 0.07 | 0.13 |
| Olfr702 | 0.05 | 0.09 | 0.07 | 0.09 | 0.10 | 0.10 | 0.08 | 0.12 |
| Olfr703 | 0.16 | 0.20 | 0.19 | 0.19 | 0.19 | 0.20 | 0.20 | 0.19 |
| Olfr704 | 0.11 | 0.15 | 0.12 | 0.16 | 0.17 | 0.14 | 0.18 | 0.14 |
| Olfr705 | 0.11 | 0.16 | 0.13 | 0.18 | 0.17 | 0.15 | 0.15 | 0.16 |
| Olfr706 | 0.18 | 0.19 | 0.19 | 0.21 | 0.20 | 0.21 | 0.18 | 0.24 |
| Olfr707 | 0.09 | 0.09 | 0.11 | 0.16 | 0.16 | 0.10 | 0.13 | 0.20 |
| Olfr71 | 0.43 | 0.49 | 0.46 | 0.51 | 0.46 | 0.48 | 0.50 | 0.53 |
| Olfr710 | 0.21 | 0.25 | 0.22 | 0.27 | 0.23 | 0.27 | 0.24 | 0.27 |
| Olfr711 | 0.22 | 0.25 | 0.24 | 0.28 | 0.25 | 0.27 | 0.24 | 0.32 |
| Olfr713 | 0.22 | 0.27 | 0.25 | 0.28 | 0.28 | 0.27 | 0.27 | 0.26 |
| Olfr714 | 0.22 | 0.26 | 0.25 | 0.25 | 0.28 | 0.24 | 0.24 | 0.27 |
| Olfr715 | 0.05 | 0.07 | 0.07 | 0.08 | 0.08 | 0.08 | 0.09 | 0.15 |
| Olfr716 | 0.14 | 0.15 | 0.13 | 0.18 | 0.17 | 0.16 | 0.16 | 0.13 |
| Olfr720 | 0.17 | 0.21 | 0.18 | 0.20 | 0.18 | 0.21 | 0.16 | 0.21 |
| Olfr722 | 0.00 | 0.01 | NA | 0.02 | 0.01 | NA | 0.01 | 0.06 |
| Olfr723 | 0.19 | 0.24 | 0.22 | 0.24 | 0.21 | 0.22 | 0.26 | 0.21 |
| Olfr724 | 0.19 | 0.24 | 0.22 | 0.24 | 0.21 | 0.22 | 0.26 | 0.21 |
| Olfr725 | 0.09 | 0.11 | 0.08 | 0.11 | 0.15 | 0.13 | 0.09 | 0.18 |
| Olfr726 | 0.04 | 0.08 | 0.05 | 0.08 | 0.08 | 0.10 | 0.08 | 0.16 |
| Olfr727 | 0.05 | 0.06 | 0.05 | 0.08 | 0.07 | 0.05 | 0.08 | 0.15 |
| Olfr728 | 0.10 | 0.14 | 0.13 | 0.16 | 0.15 | 0.19 | 0.15 | 0.15 |
| Olfr729 | 0.07 | 0.08 | 0.07 | 0.08 | 0.07 | 0.11 | 0.07 | 0.10 |
| Olfr73 | 0.14 | 0.18 | 0.16 | 0.21 | 0.23 | 0.26 | 0.17 | 0.20 |
| Olfr730 | 0.10 | 0.10 | 0.13 | 0.14 | 0.14 | 0.18 | 0.16 | 0.17 |
| Olfr731 | 0.08 | 0.10 | 0.07 | 0.09 | 0.10 | 0.10 | 0.08 | 0.12 |
| Olfr732 | 0.09 | 0.11 | 0.09 | 0.10 | 0.11 | 0.12 | 0.11 | 0.13 |
| Olfr733 | 0.08 | 0.12 | 0.10 | 0.09 | 0.10 | 0.14 | 0.12 | 0.12 |
| Olfr734 | 0.18 | 0.22 | 0.17 | 0.23 | 0.20 | 0.23 | 0.23 | 0.25 |
| Olfr735 | 0.14 | 0.17 | 0.17 | 0.21 | 0.17 | 0.17 | 0.18 | 0.20 |
| Olfr736 | 0.20 | 0.23 | 0.21 | 0.24 | 0.22 | 0.24 | 0.23 | 0.24 |
| Olfr738 | 0.17 | 0.18 | 0.19 | 0.18 | 0.16 | 0.20 | 0.20 | 0.24 |
| Olfr739 | NA | 0.03 | NA | NA | 0.01 | 0.05 | 0.01 | 0.01 |
| Olfr74 | 0.13 | 0.16 | 0.15 | 0.17 | 0.15 | 0.18 | 0.16 | 0.16 |
| Olfr740 | 0.14 | 0.17 | 0.15 | 0.19 | 0.19 | 0.20 | 0.18 | 0.24 |
| Olfr741 | 0.14 | 0.19 | 0.16 | 0.19 | 0.20 | 0.18 | 0.16 | 0.21 |
| Olfr742 | 0.01 | 0.02 | NA | 0.03 | 0.03 | 0.02 | 0.08 | 0.03 |
| Olfr743 | 0.08 | 0.11 | 0.09 | 0.12 | 0.12 | 0.14 | 0.11 | 0.20 |
| Olfr744 | 0.23 | 0.22 | 0.21 | 0.26 | 0.25 | 0.26 | 0.24 | 0.24 |
| Olfr745 | 0.24 | 0.27 | 0.25 | 0.30 | 0.26 | 0.30 | 0.30 | 0.27 |
| Olfr746 | 0.06 | 0.07 | 0.08 | 0.05 | 0.08 | 0.13 | 0.07 | 0.11 |
| Olfr747 | 0.02 | 0.04 | 0.01 | 0.03 | 0.05 | 0.03 | 0.07 | 0.06 |
| Olfr748 | 0.07 | 0.10 | 0.09 | 0.08 | 0.12 | 0.11 | 0.10 | 0.15 |
| Olfr749 | 0.03 | 0.05 | 0.03 | 0.04 | 0.03 | 0.06 | 0.04 | 0.06 |
| Olfr750 | 0.26 | 0.29 | 0.26 | 0.30 | 0.29 | 0.28 | 0.28 | 0.27 |
| Olfr76 | 0.21 | 0.27 | 0.21 | 0.24 | 0.23 | 0.24 | 0.21 | 0.21 |
| Olfr761 | 0.09 | 0.09 | 0.08 | 0.10 | 0.11 | 0.09 | 0.11 | 0.13 |
| Olfr763 | 0.16 | 0.20 | 0.15 | 0.20 | 0.23 | 0.19 | 0.18 | 0.25 |
| Olfr765 | 0.25 | 0.26 | 0.25 | 0.29 | 0.24 | 0.30 | 0.26 | 0.25 |
| Olfr767 | 0.53 | 0.54 | 0.47 | 0.62 | 0.57 | 0.51 | 0.42 | 0.46 |
| Olfr768 | 0.01 | 0.02 | 0.02 | 0.05 | 0.05 | 0.02 | 0.05 | 0.10 |
| Olfr769 | 0.13 | 0.17 | 0.14 | 0.20 | 0.19 | 0.21 | 0.15 | 0.19 |
| Olfr77 | 0.07 | 0.11 | 0.11 | 0.12 | 0.11 | 0.13 | 0.11 | 0.12 |
| Olfr770 | 0.19 | 0.26 | 0.22 | 0.27 | 0.21 | 0.24 | 0.22 | 0.22 |
| Olfr771 | 0.05 | 0.08 | 0.08 | 0.06 | 0.10 | 0.09 | 0.08 | 0.15 |
| Olfr772 | 0.04 | 0.08 | 0.05 | 0.07 | 0.08 | 0.09 | 0.08 | 0.05 |
| Olfr773 | 0.08 | 0.11 | 0.11 | 0.10 | 0.14 | 0.12 | 0.11 | 0.13 |
| Olfr774 | 0.22 | 0.26 | 0.23 | 0.26 | 0.26 | 0.30 | 0.26 | 0.27 |
| Olfr775 | 0.07 | 0.08 | 0.09 | 0.10 | 0.14 | 0.13 | 0.07 | 0.18 |
| Olfr776 | 0.01 | 0.04 | 0.01 | 0.03 | 0.04 | 0.06 | 0.02 | 0.03 |
| Olfr777 | 0.04 | 0.05 | 0.05 | 0.05 | 0.05 | 0.05 | 0.04 | 0.10 |
| Olfr78 | 0.07 | 0.10 | 0.08 | 0.11 | 0.12 | 0.11 | 0.10 | 0.14 |
| Olfr780 | 0.09 | 0.11 | 0.09 | 0.12 | 0.12 | 0.11 | 0.11 | 0.14 |
| Olfr781 | 0.03 | 0.05 | 0.03 | 0.05 | 0.07 | 0.06 | 0.06 | 0.15 |
| Olfr782 | 0.07 | 0.09 | 0.08 | 0.09 | 0.12 | 0.10 | 0.09 | 0.13 |
| Olfr784 | 0.12 | 0.18 | 0.16 | 0.15 | 0.17 | 0.18 | 0.20 | 0.22 |
| Olfr786 | 0.07 | 0.11 | 0.09 | 0.13 | 0.10 | 0.08 | 0.09 | 0.10 |
| Olfr787 | NA | 0.03 | 0.01 | 0.05 | 0.04 | 0.02 | 0.02 | 0.09 |
| Olfr788 | 0.15 | 0.18 | 0.17 | 0.20 | 0.20 | 0.20 | 0.23 | 0.21 |
| Olfr790 | 0.14 | 0.18 | 0.16 | 0.17 | 0.18 | 0.18 | 0.17 | 0.20 |
| Olfr791 | 0.08 | 0.07 | 0.08 | 0.08 | 0.11 | 0.08 | 0.11 | 0.13 |
| Olfr792 | 0.04 | 0.05 | 0.05 | 0.06 | 0.04 | 0.06 | 0.07 | 0.10 |
| Olfr794 | 0.13 | 0.15 | 0.13 | 0.16 | 0.15 | 0.15 | 0.15 | 0.18 |
| Olfr796 | 0.27 | 0.34 | 0.30 | 0.34 | 0.36 | 0.35 | 0.32 | 0.35 |
| Olfr798 | 0.02 | 0.04 | 0.05 | 0.05 | 0.06 | 0.05 | 0.03 | 0.14 |
| Olfr799 | 0.06 | 0.09 | 0.07 | 0.09 | 0.10 | 0.10 | 0.11 | 0.14 |
| Olfr8 | NA | 0.03 | 0.02 | 0.04 | 0.03 | 0.02 | 0.06 | 0.11 |
| Olfr800 | 0.04 | 0.05 | 0.05 | 0.05 | 0.05 | 0.05 | 0.04 | 0.10 |
| Olfr801 | 0.12 | 0.17 | 0.14 | 0.14 | 0.15 | 0.15 | 0.14 | 0.15 |
| Olfr802 | 0.05 | 0.08 | 0.06 | 0.08 | 0.08 | 0.07 | 0.09 | 0.08 |
| Olfr803 | 0.02 | 0.03 | 0.05 | 0.02 | 0.04 | 0.04 | 0.03 | 0.10 |
| Olfr804 | 0.07 | 0.09 | 0.08 | 0.09 | 0.12 | 0.10 | 0.09 | 0.13 |
| Olfr805 | 0.06 | 0.10 | 0.07 | 0.07 | 0.08 | 0.11 | 0.10 | 0.10 |
| Olfr806 | NA | NA | NA | NA | −0.03 | NA | 0.02 | NA |
| Olfr807 | 0.28 | 0.32 | 0.29 | 0.32 | 0.31 | 0.30 | 0.31 | 0.31 |
| Olfr808 | 0.11 | 0.11 | 0.12 | 0.15 | 0.12 | 0.15 | 0.15 | 0.13 |
| Olfr809 | 0.02 | 0.06 | 0.03 | 0.06 | 0.06 | 0.06 | 0.06 | 0.11 |
| Olfr810 | 0.19 | 0.25 | 0.20 | 0.22 | 0.23 | 0.26 | 0.25 | 0.25 |
| Olfr811 | 0.14 | 0.17 | 0.15 | 0.17 | 0.18 | 0.18 | 0.16 | 0.20 |
| Olfr812 | 0.12 | 0.15 | 0.14 | 0.14 | 0.15 | 0.17 | 0.16 | 0.14 |
| Olfr813 | 0.06 | 0.10 | 0.06 | 0.08 | 0.09 | 0.09 | 0.11 | 0.13 |
| Olfr814 | NA | 0.01 | 0.00 | 0.01 | 0.01 | NA | 0.05 | 0.08 |
| Olfr815 | NA | 0.01 | 0.00 | 0.01 | 0.02 | 0.03 | 0.04 | 0.06 |
| Olfr816 | 0.04 | 0.05 | 0.03 | 0.05 | 0.07 | 0.06 | 0.07 | 0.09 |
| Olfr818 | 0.08 | 0.09 | 0.09 | 0.10 | 0.10 | 0.10 | 0.10 | 0.11 |
| Olfr819 | 0.22 | 0.27 | 0.24 | 0.26 | 0.27 | 0.31 | 0.26 | 0.28 |
| Olfr820 | 0.05 | 0.06 | 0.06 | 0.09 | 0.08 | 0.07 | 0.07 | 0.07 |
| Olfr821 | 0.09 | 0.11 | 0.11 | 0.12 | 0.13 | 0.14 | 0.11 | 0.14 |
| Olfr822 | 0.05 | 0.09 | 0.07 | 0.09 | 0.08 | 0.09 | 0.10 | 0.13 |
| Olfr823 | 0.03 | 0.04 | 0.04 | 0.04 | 0.05 | 0.04 | 0.07 | 0.11 |
| Olfr824 | 0.03 | 0.04 | 0.04 | 0.04 | 0.05 | 0.04 | 0.07 | 0.11 |
| Olfr825 | 0.12 | 0.16 | 0.12 | 0.15 | 0.14 | 0.14 | 0.14 | 0.23 |
| Olfr826 | 0.00 | 0.01 | 0.02 | 0.05 | 0.06 | 0.02 | 0.04 | 0.11 |
| Olfr827 | 0.05 | 0.06 | 0.04 | 0.07 | 0.06 | 0.07 | 0.09 | 0.08 |
| Olfr828 | 0.08 | 0.09 | 0.10 | 0.12 | 0.11 | 0.07 | 0.12 | 0.14 |
| Olfr829 | 0.09 | 0.14 | 0.11 | 0.12 | 0.13 | 0.14 | 0.12 | 0.16 |
| Olfr830 | 0.09 | 0.12 | 0.12 | 0.12 | 0.10 | 0.15 | 0.16 | 0.12 |
| Olfr832 | 0.02 | 0.05 | 0.03 | 0.05 | 0.05 | 0.04 | 0.04 | 0.11 |
| Olfr834 | NA | 0.01 | 0.01 | 0.01 | 0.02 | 0.01 | NA | 0.08 |
| Olfr835 | 0.16 | 0.14 | 0.18 | 0.19 | 0.14 | 0.21 | 0.16 | 0.18 |
| Olfr836 | 0.01 | 0.05 | 0.02 | 0.03 | 0.02 | 0.05 | 0.09 | 0.12 |
| Olfr837 | NA | 0.02 | NA | 0.02 | 0.03 | 0.02 | 0.06 | 0.14 |
| Olfr843 | 0.09 | 0.14 | 0.13 | 0.12 | 0.14 | 0.14 | 0.15 | 0.14 |
| Olfr845 | 0.13 | 0.17 | 0.14 | 0.17 | 0.14 | 0.14 | 0.13 | 0.16 |
| Olfr846 | 0.00 | 0.03 | 0.01 | 0.01 | 0.03 | 0.03 | 0.04 | 0.08 |
| Olfr847 | 0.01 | 0.03 | 0.01 | 0.06 | 0.05 | 0.06 | 0.05 | 0.10 |
| Olfr849 | 0.00 | 0.03 | NA | 0.04 | 0.02 | 0.05 | 0.01 | 0.03 |
| Olfr850 | 0.16 | 0.18 | 0.21 | 0.20 | 0.17 | 0.19 | 0.19 | 0.15 |
| Olfr851 | 0.09 | 0.10 | 0.08 | 0.08 | 0.12 | 0.11 | 0.09 | 0.13 |
| Olfr853 | 0.05 | 0.08 | 0.06 | 0.07 | 0.09 | 0.08 | 0.10 | 0.12 |
| Olfr854 | 0.06 | 0.06 | 0.06 | 0.07 | 0.07 | 0.09 | 0.09 | 0.09 |
| Olfr855 | 0.06 | 0.08 | 0.09 | 0.07 | 0.08 | 0.11 | 0.11 | 0.16 |
| Olfr856-ps1 | 0.12 | 0.14 | 0.13 | 0.17 | 0.13 | 0.17 | 0.11 | 0.18 |
| Olfr857 | 0.03 | 0.05 | 0.04 | 0.05 | 0.10 | 0.09 | 0.09 | 0.08 |
| Olfr859 | 0.00 | NA | NA | 0.02 | 0.05 | 0.04 | 0.05 | 0.17 |
| Olfr860 | 0.05 | 0.04 | 0.05 | 0.07 | 0.06 | 0.05 | 0.09 | 0.11 |
| Olfr862 | 0.08 | 0.07 | 0.09 | 0.11 | 0.12 | 0.09 | 0.09 | 0.13 |
| Olfr866 | 0.06 | 0.08 | 0.09 | 0.09 | 0.07 | 0.09 | 0.10 | 0.09 |
| Olfr867 | 0.18 | 0.22 | 0.17 | 0.21 | 0.20 | 0.24 | 0.19 | 0.22 |
| Olfr868 | 0.01 | 0.04 | 0.02 | 0.06 | 0.04 | 0.06 | 0.05 | 0.07 |
| Olfr869 | 0.02 | 0.06 | 0.02 | 0.02 | 0.03 | 0.03 | 0.05 | 0.13 |
| Olfr870 | 0.13 | 0.16 | 0.14 | 0.18 | 0.19 | 0.17 | 0.13 | 0.20 |
| Olfr871 | 0.13 | 0.16 | 0.15 | 0.17 | 0.16 | 0.16 | 0.17 | 0.20 |
| Olfr872 | NA | 0.01 | 0.01 | 0.01 | 0.03 | 0.03 | 0.05 | 0.10 |
| Olfr873 | 0.07 | 0.08 | 0.06 | 0.08 | 0.09 | 0.09 | 0.08 | 0.11 |
| Olfr874 | 0.22 | 0.25 | 0.24 | 0.24 | 0.28 | 0.27 | 0.26 | 0.27 |
| Olfr875 | 0.15 | 0.14 | 0.16 | 0.16 | 0.16 | 0.18 | 0.19 | 0.16 |
| Olfr876 | 0.14 | 0.17 | 0.14 | 0.16 | 0.20 | 0.18 | 0.16 | 0.15 |
| Olfr877 | 0.07 | 0.10 | 0.07 | 0.13 | 0.13 | 0.13 | 0.11 | 0.15 |
| Olfr878 | 0.17 | 0.20 | 0.18 | 0.19 | 0.19 | 0.20 | 0.22 | 0.19 |
| Olfr881 | 0.04 | 0.07 | 0.05 | 0.09 | 0.08 | 0.08 | 0.09 | 0.08 |
| Olfr883 | 0.03 | 0.06 | 0.03 | 0.06 | 0.07 | 0.05 | 0.04 | 0.06 |
| Olfr884 | 0.03 | 0.06 | 0.03 | 0.06 | 0.07 | 0.05 | 0.04 | 0.06 |
| Olfr885 | 0.03 | 0.06 | 0.03 | 0.06 | 0.07 | 0.05 | 0.04 | 0.06 |
| Olfr887 | 0.03 | 0.06 | 0.03 | 0.06 | 0.07 | 0.05 | 0.04 | 0.06 |
| Olfr888 | 0.05 | 0.06 | 0.07 | 0.07 | 0.07 | 0.11 | 0.07 | 0.07 |
| Olfr889 | 0.02 | 0.04 | 0.03 | 0.04 | 0.05 | 0.03 | 0.05 | 0.10 |
| Olfr890 | 0.04 | 0.05 | 0.04 | 0.04 | 0.03 | 0.06 | 0.07 | 0.08 |
| Olfr891 | 0.07 | 0.10 | 0.09 | 0.11 | 0.11 | 0.09 | 0.10 | 0.15 |
| Olfr893 | 0.06 | 0.05 | 0.04 | 0.06 | 0.05 | 0.07 | 0.06 | 0.09 |
| Olfr894 | 0.05 | 0.07 | 0.04 | 0.05 | 0.06 | 0.07 | 0.06 | 0.09 |
| Olfr895 | 0.01 | 0.02 | 0.02 | 0.06 | 0.07 | 0.03 | 0.05 | 0.08 |
| Olfr898 | 0.14 | 0.20 | 0.19 | 0.21 | 0.18 | 0.21 | 0.21 | 0.17 |
| Olfr899 | 0.11 | 0.15 | 0.13 | 0.14 | 0.14 | 0.17 | 0.16 | 0.14 |
| Olfr9 | 0.09 | 0.12 | 0.14 | 0.14 | 0.14 | 0.14 | 0.11 | 0.14 |
| Olfr90 | 0.15 | 0.20 | 0.19 | 0.23 | 0.19 | 0.23 | 0.20 | 0.25 |
| Olfr900 | 0.11 | 0.15 | 0.12 | 0.16 | 0.15 | 0.16 | 0.13 | 0.12 |
| Olfr901 | 0.16 | 0.18 | 0.19 | 0.26 | 0.20 | 0.22 | 0.19 | 0.23 |
| Olfr902 | 0.13 | 0.14 | 0.15 | 0.17 | 0.17 | 0.21 | 0.19 | 0.22 |
| Olfr904 | 0.02 | 0.03 | 0.04 | 0.05 | 0.06 | 0.07 | 0.08 | 0.08 |
| Olfr905 | 0.01 | 0.05 | 0.05 | 0.07 | 0.04 | 0.05 | 0.08 | 0.10 |
| Olfr906 | 0.06 | 0.11 | 0.10 | 0.09 | 0.09 | 0.11 | 0.11 | 0.13 |
| Olfr907 | 0.07 | 0.07 | 0.07 | 0.10 | 0.09 | 0.10 | 0.09 | 0.09 |
| Olfr908 | 0.06 | 0.12 | 0.07 | 0.10 | 0.12 | 0.11 | 0.09 | 0.13 |
| Olfr91 | 0.40 | 0.47 | 0.41 | 0.46 | 0.46 | 0.47 | 0.43 | 0.50 |
| Olfr910 | 0.00 | 0.02 | 0.01 | 0.06 | 0.02 | 0.03 | 0.02 | 0.03 |
| Olfr911-ps1 | 0.04 | 0.05 | 0.04 | 0.05 | 0.05 | 0.04 | 0.06 | 0.10 |
| Olfr912 | 0.02 | 0.02 | 0.03 | 0.03 | 0.06 | 0.03 | 0.05 | 0.07 |
| Olfr913 | 0.06 | 0.11 | 0.08 | 0.09 | 0.09 | 0.09 | 0.10 | 0.14 |
| Olfr914 | 0.11 | 0.12 | 0.14 | 0.15 | 0.14 | 0.15 | 0.16 | 0.19 |
| Olfr915 | 0.10 | 0.13 | 0.13 | 0.14 | 0.14 | 0.12 | 0.12 | 0.16 |
| Olfr916 | 0.03 | 0.08 | 0.05 | 0.06 | 0.05 | 0.06 | 0.05 | 0.07 |
| Olfr917 | 0.12 | 0.16 | 0.11 | 0.16 | 0.16 | 0.15 | 0.14 | 0.13 |
| Olfr918 | 0.07 | 0.09 | 0.08 | 0.09 | 0.08 | 0.09 | 0.09 | 0.15 |
| Olfr919 | 0.15 | 0.23 | 0.20 | 0.21 | 0.20 | 0.25 | 0.22 | 0.24 |
| Olfr92 | 0.24 | 0.35 | 0.28 | 0.37 | 0.33 | 0.35 | 0.31 | 0.32 |
| Olfr920 | 0.30 | 0.27 | 0.34 | 0.29 | 0.21 | 0.21 | 0.29 | 0.26 |
| Olfr921 | 0.04 | 0.07 | 0.05 | 0.06 | 0.08 | 0.05 | 0.08 | 0.09 |
| Olfr922 | 0.17 | 0.16 | 0.16 | 0.19 | 0.16 | 0.19 | 0.20 | 0.17 |
| Olfr923 | 0.15 | 0.16 | 0.16 | 0.19 | 0.16 | 0.17 | 0.17 | 0.21 |
| Olfr924 | NA | NA | 0.01 | NA | 0.00 | 0.00 | 0.04 | 0.01 |
| Olfr926 | NA | 0.01 | 0.01 | 0.02 | 0.04 | 0.03 | 0.03 | 0.09 |
| Olfr93 | 0.20 | 0.26 | 0.21 | 0.28 | 0.24 | 0.26 | 0.20 | 0.22 |
| Olfr930 | 0.06 | 0.09 | 0.07 | 0.09 | 0.08 | 0.11 | 0.08 | 0.11 |
| Olfr933 | 0.14 | 0.18 | 0.17 | 0.17 | 0.19 | 0.19 | 0.20 | 0.23 |
| Olfr934 | 0.06 | 0.09 | 0.07 | 0.08 | 0.10 | 0.09 | 0.11 | 0.11 |
| Olfr935 | 0.10 | 0.11 | 0.11 | 0.13 | 0.12 | 0.11 | 0.15 | 0.17 |
| Olfr936 | NA | 0.02 | 0.01 | 0.01 | 0.02 | NA | 0.01 | 0.05 |
| Olfr937 | 0.06 | 0.07 | 0.06 | 0.07 | 0.08 | 0.10 | 0.07 | 0.09 |
| Olfr938 | 0.09 | 0.13 | 0.12 | 0.10 | 0.13 | 0.16 | 0.12 | 0.14 |
| Olfr94 | 0.24 | 0.26 | 0.24 | 0.28 | 0.29 | 0.26 | 0.27 | 0.26 |
| Olfr943 | 0.04 | 0.07 | 0.05 | 0.07 | 0.05 | 0.08 | 0.07 | 0.08 |
| Olfr944 | 0.02 | 0.06 | 0.04 | 0.06 | 0.05 | 0.05 | 0.04 | 0.06 |
| Olfr945 | 0.03 | 0.03 | 0.03 | 0.02 | 0.04 | 0.05 | 0.03 | 0.05 |
| Olfr947-ps1 | 0.05 | 0.07 | 0.06 | 0.06 | 0.06 | 0.09 | 0.07 | 0.10 |
| Olfr948 | NA | 0.00 | 0.01 | 0.04 | 0.04 | NA | 0.01 | 0.08 |
| Olfr95 | 0.31 | 0.34 | 0.31 | 0.40 | 0.38 | 0.40 | 0.37 | 0.35 |
| Olfr951 | 0.07 | 0.09 | 0.07 | 0.10 | 0.08 | 0.10 | 0.08 | 0.10 |
| Olfr952 | 0.02 | 0.03 | 0.02 | 0.03 | 0.03 | 0.04 | 0.03 | 0.06 |
| Olfr954 | 0.07 | 0.09 | 0.07 | 0.10 | 0.08 | 0.10 | 0.08 | 0.10 |
| Olfr955 | 0.08 | 0.09 | 0.09 | 0.11 | 0.09 | 0.12 | 0.11 | 0.15 |
| Olfr957 | 0.10 | 0.13 | 0.09 | 0.11 | 0.13 | 0.12 | 0.11 | 0.13 |
| Olfr958 | 0.23 | 0.23 | 0.24 | 0.26 | 0.23 | 0.27 | 0.25 | 0.23 |
| Olfr959 | 0.23 | 0.28 | 0.25 | 0.27 | 0.28 | 0.27 | 0.26 | 0.26 |
| Olfr96 | 0.22 | 0.28 | 0.23 | 0.28 | 0.26 | 0.29 | 0.24 | 0.27 |
| Olfr960 | 0.12 | 0.12 | 0.10 | 0.16 | 0.13 | 0.16 | 0.16 | 0.17 |
| Olfr961 | 0.06 | 0.10 | 0.06 | 0.09 | 0.09 | 0.10 | 0.10 | 0.15 |
| Olfr963 | 0.13 | 0.11 | 0.15 | 0.13 | 0.14 | 0.12 | 0.09 | 0.08 |
| Olfr965 | 0.13 | 0.14 | 0.15 | 0.16 | 0.13 | 0.17 | 0.13 | 0.17 |
| Olfr967 | 0.14 | 0.16 | 0.14 | 0.17 | 0.16 | 0.17 | 0.14 | 0.17 |
| Olfr968 | 0.00 | 0.03 | 0.03 | 0.01 | 0.00 | 0.03 | 0.03 | 0.05 |
| Olfr969 | 0.03 | 0.05 | 0.05 | 0.08 | 0.08 | 0.07 | 0.05 | 0.08 |
| Olfr97 | 0.06 | 0.17 | 0.09 | 0.15 | 0.12 | 0.10 | 0.11 | 0.15 |
| Olfr970 | 0.01 | 0.05 | 0.02 | 0.05 | 0.06 | 0.05 | 0.04 | 0.06 |
| Olfr971 | 0.08 | 0.11 | 0.08 | 0.09 | 0.10 | 0.10 | 0.10 | 0.09 |
| Olfr972 | 0.03 | 0.05 | 0.05 | 0.08 | 0.07 | 0.04 | 0.08 | 0.07 |
| Olfr974 | 0.23 | 0.27 | 0.24 | 0.30 | 0.30 | 0.28 | 0.28 | 0.29 |
| Olfr975 | 0.27 | 0.31 | 0.30 | 0.32 | 0.32 | 0.32 | 0.31 | 0.33 |
| Olfr976 | 0.16 | 0.19 | 0.18 | 0.21 | 0.19 | 0.23 | 0.21 | 0.25 |
| Olfr978 | 0.24 | 0.29 | 0.26 | 0.30 | 0.32 | 0.28 | 0.26 | 0.27 |
| Olfr979 | 0.17 | 0.22 | 0.18 | 0.21 | 0.20 | 0.21 | 0.20 | 0.23 |
| Olfr98 | 0.06 | 0.17 | 0.09 | 0.15 | 0.12 | 0.10 | 0.11 | 0.15 |
| Olfr980 | 0.16 | 0.19 | 0.20 | 0.21 | 0.22 | 0.23 | 0.21 | 0.23 |
| Olfr981 | 0.22 | 0.25 | 0.23 | 0.26 | 0.30 | 0.25 | 0.23 | 0.28 |
| Olfr982 | 0.35 | 0.41 | 0.34 | 0.42 | 0.42 | 0.45 | 0.37 | 0.44 |
| Olfr983 | 0.03 | 0.06 | 0.03 | 0.06 | 0.07 | 0.05 | 0.04 | 0.06 |
| Olfr984 | 0.29 | 0.33 | 0.32 | 0.36 | 0.34 | 0.37 | 0.36 | 0.35 |
| Olfr985 | 0.16 | 0.20 | 0.18 | 0.22 | 0.19 | 0.25 | 0.21 | 0.21 |
| Olfr986 | 0.19 | 0.21 | 0.19 | 0.22 | 0.23 | 0.22 | 0.20 | 0.27 |
| Olfr987 | 0.06 | 0.08 | 0.10 | 0.11 | 0.08 | 0.11 | 0.10 | 0.08 |
| Olfr988 | 0.10 | 0.12 | 0.12 | 0.14 | 0.13 | 0.13 | 0.12 | 0.16 |
| Olfr99 | 0.14 | 0.31 | 0.19 | 0.34 | 0.25 | 0.30 | 0.30 | 0.30 |
| Olfr992 | 0.08 | 0.11 | 0.11 | 0.11 | 0.11 | 0.16 | 0.17 | 0.13 |
| Olfr993 | 0.02 | 0.05 | 0.03 | 0.11 | 0.05 | 0.06 | 0.06 | 0.11 |
| Olfr994 | 0.06 | 0.06 | 0.05 | 0.08 | 0.08 | 0.07 | 0.04 | 0.14 |
| Olfr995 | 0.01 | 0.02 | 0.02 | 0.03 | 0.04 | 0.04 | 0.02 | 0.09 |
| Olfr996 | 0.04 | 0.03 | 0.05 | 0.05 | 0.04 | 0.04 | 0.07 | 0.13 |
| Olfr998 | 0.17 | 0.20 | 0.19 | 0.21 | 0.19 | 0.19 | 0.19 | 0.22 |

TABLE 2
Olfactory receptors and accessory genes expression in sorted vascular macrophages from Apoe −/− mice in
western diet

| | RPKM value | | RPKM value | | RPKM value |
|---|---|---|---|---|---|
| Gene name | Cut-off 0.05 | Gene name | Cut-off 0.05 | Gene name | Cut-off 0.05 |
| Rtp1 | 0.112 | Olfr108 | 0.09 | Olfr118 | 0.05 |
| Rtp2 | 0.106 | Olfr1082 | 0.15 | Olfr1180 | 0.05 |
| Reep3 | 11.44 | Olfr1085 | 0.17 | Olfr1183 | 0.07 |
| Cngal | 0.106 | Olfr1086 | 0.06 | Olfr1188 | 0.08 |
| Cnga2 | 0.12 | Olfr109 | 0.09 | Olfr1189 | 0.05 |
| Cnga3 | 0.147 | Olfr1090 | 0.47 | Olfr119 | 0.05 |
| Cnga4 | 0.157 | Olfr1094 | 0.08 | Olfr1193 | 0.07 |
| Cngb1 | 0.283 | Olfr1095 | 0.08 | Olfr1195 | 0.11 |
| Acy3 | 0.862 | Olfr110 | 0.27 | Olfr1196 | 0.05 |
| Gnal | 1.513 | Olfr1102 | 0.05 | Olfr1199 | 0.05 |
| Olfr1 | 0.09 | Olfr1104 | 0.11 | Olfr12 | 0.36 |
| Olfr10 | 0.08 | Olfr1107 | 0.06 | Olfr1200 | 0.09 |
| Olfr1000 | 0.23 | Olfr1109 | 0.19 | Olfr1201 | 0.19 |
| Olfr1006 | 0.09 | Olfr111 | 0.26 | Olfr1202 | 0.05 |
| Olfr1009 | 0.08 | Olfr1112 | 0.60 | Olfr1204 | 0.05 |
| Olfr101 | 0.10 | Olfr1120 | 0.09 | Olfr1205 | 0.05 |
| Olfr1010 | 0.28 | Olfr1121 | 0.11 | Olfr1206 | 0.14 |
| Olfr1012 | 0.07 | Olfr1122 | 0.21 | Olfr1208 | 0.07 |
| Olfr1014 | 0.09 | Olfr1124 | 0.30 | Olfr1211 | 0.07 |
| Olfr1015 | 0.05 | Olfr1128 | 0.05 | Olfr1213 | 0.10 |
| Olfr1016 | 0.37 | Olfr1131 | 0.05 | Olfr1214 | 0.07 |
| Olfr1019 | 0.06 | Olfr1132 | 0.11 | Olfr1215 | 0.09 |
| Olfr1020 | 0.08 | Olfr1133 | 0.11 | Olfr1216 | 0.07 |
| Olfr1022 | 0.07 | Olfr1134 | 0.05 | Olfr1218 | 0.11 |
| Olfr1023 | 0.10 | Olfr1135 | 0.08 | Olfr122 | 0.09 |
| Olfr1026 | 0.12 | Olfr1137 | 0.07 | Olfr1221 | 0.10 |
| Olfr1029 | 0.06 | Olfr1138 | 0.11 | Olfr1222 | 0.05 |
| Olfr103 | 0.06 | Olfr1143 | 0.09 | Olfr1223 | 0.07 |
| Olfr1030 | 0.28 | Olfr1145 | 0.23 | Olfr1226 | 0.07 |
| Olfr1031 | 0.05 | Olfr1148 | 0.11 | Olfr1228 | 0.05 |
| Olfr1032 | 0.06 | Olfr115 | 0.16 | Olfr123 | 0.50 |
| Olfr1033 | 3.64 | Olfr1152 | 0.23 | Olfr1230 | 0.06 |
| Olfr1037 | 0.07 | Olfr1156 | 0.09 | Olfr1231 | 0.09 |
| Olfr1042 | 0.10 | Olfr1157 | 0.06 | Olfr1233 | 0.07 |
| Olfr1043 | 0.08 | Olfr1161 | 0.12 | Olfr1238 | 0.11 |
| Olfr1044 | 0.06 | Olfr1162 | 0.06 | Olfr124 | 0.05 |
| Olfr1046 | 0.09 | Olfr1163 | 0.05 | Olfr1240 | 0.09 |
| Olfr1047 | 0.08 | Olfr1166 | 0.09 | Olfr1243 | 0.06 |
| Olfr1048 | 0.05 | Olfr1167 | 0.08 | Olfr1245 | 0.06 |
| Olfr1055 | 0.05 | Olfr1168 | 0.11 | Olfr1246 | 0.08 |
| Olfr1056 | 0.06 | Olfr117 | 0.12 | Olfr1248 | 0.05 |
| Olfr1065 | 0.07 | Olfr1173 | 0.05 | Olfr125 | 0.05 |
| Olfr1066 | 0.08 | Olfr1176 | 0.05 | Olfr1251 | 0.05 |
| Olfr1076 | 0.06 | Olfr1178 | 0.15 | Olfr1253 | 0.06 |
| Olfr1079 | 0.11 | Olfr1179 | 0.09 | Olfr1255 | 0.07 |
| Olfr1256 | 0.06 | Olfr134 | 0.08 | Olfr1412 | 0.20 |
| Olfr1257 | 0.07 | Olfr1340 | 0.07 | Olfr1413 | 0.23 |
| Olfr1258 | 0.15 | Olfr1341 | 0.17 | Olfr1414 | 0.52 |
| Olfr1259 | 0.09 | Olfr1342 | 0.09 | Olfr1415 | 0.06 |
| Olfr126 | 0.12 | Olfr1344 | 0.45 | Olfr1416 | 0.10 |
| Olfr1260 | 0.06 | Olfr1346 | 0.10 | Olfr1417 | 0.21 |
| Olfr1261 | 0.07 | Olfr1347 | 0.10 | Olfr1419 | 0.19 |
| Olfr1262 | 0.05 | Olfr1348 | 0.15 | Olfr142 | 0.13 |
| Olfr1264 | 0.13 | Olfr1349 | 0.17 | Olfr1420 | 0.98 |
| Olfr1265 | 0.06 | Olfr1350 | 0.19 | Olfr1424 | 0.08 |
| Olfr1269 | 0.25 | Olfr1351 | 0.12 | Olfr1426 | 0.06 |
| Olfr1270 | 0.07 | Olfr1353 | 0.05 | Olfr1427 | 0.09 |
| Olfr1271 | 0.05 | Olfr1355 | 0.08 | Olfr1428 | 0.15 |
| Olfr1272 | 0.05 | Olfr1356 | 0.40 | Olfr143 | 0.08 |
| Olfr1273-ps | 0.06 | Olfr1357 | 0.15 | Olfr1431 | 0.13 |
| Olfr1275 | 0.08 | Olfr1361 | 0.19 | Olfr1437 | 0.10 |
| Olfr1278 | 0.06 | Olfr1362 | 0.05 | Olfr1440 | 0.14 |
| Olfr128 | 0.09 | Olfr1364 | 0.06 | Olfr1441 | 0.06 |
| Olfr1282 | 0.05 | Olfr1366 | 0.13 | Olfr1442 | 0.08 |
| Olfr1284 | 0.13 | Olfr1367 | 0.06 | Olfr1443 | 0.05 |
| Olfr1289 | 0.06 | Olfr137 | 0.08 | Olfr1444 | 0.16 |
| Olfr129 | 0.07 | Olfr1370 | 0.06 | Olfr1445 | 0.09 |
| Olfr1290 | 0.07 | Olfr1372-ps1 | 0.10 | Olfr1446 | 0.14 |
| Olfr1299 | 0.08 | Olfr1377 | 0.07 | Olfr1447 | 0.06 |
| Olfr13 | 0.09 | Olfr1378 | 0.22 | Olfr1448 | 0.09 |
| Olfr1300-ps1 | 0.07 | Olfr138 | 0.07 | Olfr1449 | 0.15 |
| Olfr1302 | 0.06 | Olfr1380 | 0.05 | Olfr145 | 0.07 |
| Olfr1303 | 0.15 | Olfr1382 | 0.10 | Olfr1450 | 0.17 |
| Olfr1305 | 0.07 | Olfr1384 | 0.08 | Olfr1451 | 0.05 |
| Olfr1309 | 0.11 | Olfr1385 | 0.09 | Olfr1454 | 0.05 |
| Olfr131 | 0.11 | Olfr1386 | 0.05 | Olfr1461 | 0.05 |
| Olfr1311 | 0.05 | Olfr1387 | 0.12 | Olfr1462 | 0.08 |
| Olfr1312 | 0.06 | Olfr1388 | 0.12 | Olfr1463 | 0.05 |
| Olfr1313 | 0.05 | Olfr1389 | 0.32 | Olfr1465 | 0.07 |
| Olfr1316 | 0.05 | Olfr139 | 0.13 | Olfr1469 | 0.17 |
| Olfr1318 | 0.09 | Olfr1390 | 0.13 | Olfr147 | 0.09 |
| Olfr132 | 0.11 | Olfr1391 | 0.08 | Olfr149 | 0.20 |
| Olfr1321 | 0.07 | Olfr1392 | 0.17 | Olfr1490 | 0.12 |
| Olfr1322 | 0.22 | Olfr1393 | 0.12 | Olfr1491 | 0.06 |
| Olfr1328 | 0.10 | Olfr1394 | 0.19 | Olfr1494 | 0.10 |
| Olfr1329 | 0.05 | Olfr1395 | 0.17 | Olfr1495 | 0.11 |
| Olfr133 | 0.10 | Olfr1396 | 0.13 | Olfr1496 | 0.11 |
| Olfr1330 | 0.25 | Olfr140 | 0.09 | Olfr15 | 0.19 |
| Olfr1331 | 0.33 | Olfr1402 | 0.13 | Olfr150 | 0.10 |
| Olfr1335 | 0.11 | Olfr1404 | 0.14 | Olfr1500 | 0.27 |
| Olfr1336 | 0.15 | Olfr1406 | 0.24 | Olfr1501 | 0.21 |
| Olfr1337 | 0.17 | Olfr1408 | 0.17 | Olfr1504 | 0.05 |
| Olfr1338 | 0.23 | Olfr1410 | 0.16 | Olfr1505 | 0.12 |
| Olfr1339 | 0.16 | Olfr1411 | 0.19 | Olfr1507 | 0.24 |
| Olfr1508 | 0.07 | Olfr24 | 0.07 | Olfr330 | 0.12 |
| Olfr151 | 0.08 | Olfr248 | 0.05 | Olfr332 | 0.15 |
| Olfr1511 | 0.06 | Olfr25 | 0.05 | Olfr338 | 0.06 |
| Olfr1512 | 0.09 | Olfr259 | 0.06 | Olfr339 | 0.06 |
| Olfr1513 | 0.09 | Olfr26 | 0.12 | Olfr341 | 0.14 |
| Olfr153 | 0.05 | Olfr262 | 0.09 | Olfr342 | 0.07 |
| Olfr1535 | 0.09 | Olfr263 | 0.11 | Olfr344 | 0.06 |
| Olfr155 | 0.13 | Olfr266 | 0.05 | Olfr345 | 0.06 |
| Olfr156 | 0.17 | Olfr267 | 0.11 | Olfr346 | 0.11 |
| Olfr157 | 0.26 | Olfr272 | 0.11 | Olfr347 | 0.06 |
| Olfr159 | 0.16 | Olfr273 | 0.09 | Olfr350 | 0.18 |
| Olfr160 | 0.07 | Olfr279 | 0.20 | Olfr351 | 0.10 |
| Olfr161 | 0.15 | Olfr281 | 0.32 | Olfr352 | 0.12 |
| Olfr164 | 0.06 | Olfr282 | 0.24 | Olfr353 | 0.12 |
| Olfr165 | 0.08 | Olfr283 | 0.08 | Olfr354 | 0.13 |
| Olfr166 | 0.08 | Olfr284 | 0.16 | Olfr356 | 0.07 |
| Olfr167 | 0.09 | Olfr286 | 0.20 | Olfr357 | 0.08 |
| Olfr169 | 0.06 | Olfr287 | 0.11 | Olfr358 | 0.19 |
| Olfr17 | 0.46 | Olfr288 | 0.09 | Olfr360 | 0.06 |
| Olfr170 | 0.08 | Olfr29-ps1 | 0.10 | Olfr361 | 0.14 |
| Olfr172 | 0.08 | Olfr291 | 0.06 | Olfr362 | 0.09 |
| Olfr175-ps1 | 0.06 | Olfr294 | 0.06 | Olfr365 | 0.12 |
| Olfr179 | 0.15 | Olfr295 | 0.05 | Olfr366 | 0.20 |
| Olfr18 | 0.08 | Olfr299 | 0.07 | Olfr368 | 0.12 |
| Olfr187 | 0.09 | Olfr3 | 0.19 | Olfr370 | 0.16 |
| Olfr19 | 0.05 | Olfr30 | 0.25 | Olfr371 | 0.12 |
| Olfr190 | 0.05 | Olfr301 | 0.06 | Olfr372 | 0.14 |
| Olfr191 | 0.08 | Olfr303 | 0.11 | Olfr373 | 0.15 |
| Olfr196 | 0.13 | Olfr304 | 0.08 | Olfr374 | 0.28 |
| Olfr197 | 0.07 | Olfr308 | 0.05 | Olfr376 | 0.07 |
| Olfr198 | 0.05 | Olfr309 | 0.11 | Olfr378 | 0.12 |
| Olfr2 | 0.05 | Olfr31 | 0.25 | Olfr382 | 0.06 |
| Olfr202 | 0.11 | Olfr310 | 0.10 | Olfr384 | 0.05 |
| Olfr211 | 0.22 | Olfr311 | 0.13 | Olfr389 | 0.05 |
| Olfr212 | 0.13 | Olfr312 | 0.05 | Olfr39 | 0.88 |
| Olfr213 | 0.08 | Olfr313 | 0.13 | Olfr390 | 0.11 |
| Olfr214 | 0.10 | Olfr314 | 0.20 | Olfr393 | 0.05 |
| Olfr215 | 0.10 | Olfr315 | 0.22 | Olfr394 | 0.07 |
| Olfr218 | 0.11 | Olfr316 | 0.16 | Olfr395 | 0.10 |
| Olfr220 | 0.14 | Olfr317 | 0.15 | Olfr397 | 0.09 |
| Olfr221 | 0.15 | Olfr318 | 0.11 | Olfr398 | 0.17 |
| Olfr222 | 0.36 | Olfr319 | 0.12 | Olfr399 | 0.14 |
| Olfr223 | 0.21 | Olfr32 | 0.10 | Olfr401 | 0.05 |
| Olfr224 | 0.19 | Olfr320 | 0.14 | Olfr406-ps | 0.12 |
| Olfr225 | 0.12 | Olfr323 | 0.12 | Olfr411 | 0.21 |
| Olfr229 | 0.33 | Olfr324 | 0.10 | Olfr412 | 0.11 |
| Olfr23 | 0.64 | Olfr328 | 0.12 | Olfr414 | 0.08 |
| Olfr237-ps1 | 0.11 | Olfr329-ps | 0.06 | Olfr417 | 0.05 |
| Olfr239 | 0.06 | Olfr33 | 0.06 | Olfr418-ps1 | 0.23 |
| Olfr419 | 0.05 | Olfr498 | 0.20 | Olfr566 | 0.10 |
| Olfr424 | 0.24 | Olfr5 | 0.28 | Olfr568 | 0.07 |
| Olfr43 | 0.21 | Olfr50 | 0.48 | Olfr57 | 0.19 |
| Olfr432 | 0.37 | Olfr506 | 0.16 | Olfr571 | 0.14 |
| Olfr433 | 0.61 | Olfr507 | 0.14 | Olfr572 | 0.08 |
| Olfr434 | 0.05 | Olfr508 | 0.17 | Olfr575 | 0.05 |
| Olfr435 | 0.05 | Olfr509 | 0.09 | Olfr576 | 0.08 |
| Olfr437 | 0.11 | Olfr51 | 0.12 | Olfr577 | 0.16 |
| Olfr444 | 0.12 | Olfr510 | 0.15 | Olfr578 | 0.13 |
| Olfr446 | 0.14 | Olfr512 | 0.12 | Olfr58 | 0.08 |
| Olfr447 | 0.08 | Olfr513 | 0.14 | Olfr582 | 0.10 |
| Olfr448 | 0.10 | Olfr514 | 0.09 | Olfr584 | 0.09 |
| Olfr449 | 0.09 | Olfr517 | 0.14 | Olfr585 | 0.09 |
| Olfr450 | 0.09 | Olfr52 | 0.07 | Olfr589 | 0.09 |
| Olfr452 | 0.15 | Olfr520 | 0.11 | Olfr59 | 0.18 |
| Olfr453 | 0.07 | Olfr521 | 0.09 | Olfr592 | 0.07 |
| Olfr455 | 0.26 | Olfr522 | 0.15 | Olfr593 | 0.05 |
| Olfr456 | 0.42 | Olfr523 | 0.19 | Olfr594 | 0.06 |
| Olfr457 | 0.11 | Olfr524 | 0.15 | Olfr598 | 0.05 |
| Olfr458 | 0.16 | Olfr525 | 0.08 | Olfr6 | 0.08 |
| Olfr46 | 0.13 | Olfr527 | 0.15 | Olfr60 | 0.45 |
| Olfr460 | 0.10 | Olfr53 | 0.10 | Olfr600 | 0.09 |
| Olfr461 | 0.08 | Olfr530 | 0.12 | Olfr601 | 0.09 |
| Olfr462 | 0.10 | Olfr531 | 0.05 | Olfr603 | 0.06 |
| Olfr463 | 0.06 | Olfr532 | 0.11 | Olfr605 | 0.07 |
| Olfr464 | 0.14 | Olfr533 | 0.11 | Olfr606 | 0.06 |
| Olfr466 | 0.09 | Olfr535 | 0.25 | Olfr608 | 0.06 |
| Olfr467 | 0.13 | Olfr536 | 0.09 | Olfr609 | 0.14 |
| Olfr47 | 0.09 | Olfr538 | 0.14 | Olfr61 | 0.09 |
| Olfr470 | 0.18 | Olfr539 | 0.09 | Olfr610 | 0.07 |
| Olfr472 | 0.08 | Olfr54 | 0.19 | Olfr612 | 0.05 |
| Olfr473 | 0.18 | Olfr541 | 0.10 | Olfr613 | 30.74 |
| Olfr479 | 0.12 | Olfr543 | 0.54 | Olfr615 | 0.19 |
| Olfr48 | 0.07 | Olfr544 | 0.07 | Olfr617 | 0.17 |
| Olfr480 | 0.08 | Olfr545 | 0.16 | Olfr619 | 0.05 |
| Olfr481 | 0.10 | Olfr547 | 0.19 | Olfr62 | 0.15 |
| Olfr482 | 0.09 | Olfr549 | 0.24 | Olfr622 | 0.05 |
| Olfr483 | 0.08 | Olfr550 | 0.15 | Olfr623 | 0.05 |
| Olfr484 | 0.11 | Olfr551 | 0.34 | Olfr624 | 0.05 |
| Olfr485 | 0.26 | Olfr552 | 0.09 | Olfr629 | 0.08 |
| Olfr486 | 0.10 | Olfr553 | 0.05 | Olfr63 | 0.06 |
| Olfr487 | 0.06 | Olfr554 | 0.63 | Olfr630 | 0.05 |
| Olfr49 | 0.08 | Olfr555 | 0.09 | Olfr631 | 0.07 |
| Olfr490 | 0.05 | Olfr556 | 0.18 | Olfr632 | 0.26 |
| Olfr491 | 0.07 | Olfr557 | 0.05 | Olfr633 | 0.12 |
| Olfr493 | 0.08 | Olfr558 | 0.09 | Olfr635 | 0.10 |
| Olfr494 | 0.09 | Olfr559 | 0.17 | Olfr638 | 0.09 |
| Olfr495 | 0.08 | Olfr56 | 0.20 | Olfr639 | 0.15 |
| Olfr497 | 0.13 | Olfr561 | 0.15 | Olfr64 | 0.06 |
| Olfr640 | 0.18 | Olfr703 | 0.15 | Olfr801 | 0.22 |
| Olfr641 | 0.11 | Olfr704 | 0.11 | Olfr802 | 0.14 |
| Olfr642 | 0.11 | Olfr705 | 0.17 | Olfr803 | 0.05 |
| Olfr643 | 0.05 | Olfr706 | 0.19 | Olfr806 | 0.06 |
| Olfr644 | 0.06 | Olfr71 | 0.20 | Olfr808 | 0.22 |
| Olfr645 | 0.20 | Olfr710 | 0.31 | Olfr810 | 0.07 |
| Olfr646 | 0.18 | Olfr711 | 0.10 | Olfr812 | 0.05 |
| Olfr648 | 0.24 | Olfr714 | 0.17 | Olfr814 | 0.16 |
| Olfr649 | 0.09 | Olfr715 | 0.07 | Olfr816 | 0.43 |
| Olfr65 | 0.07 | Olfr716 | 0.11 | Olfr818 | 0.08 |
| Olfr651 | 0.16 | Olfr720 | 0.23 | Olfr822 | 0.10 |
| Olfr652 | 0.10 | Olfr73 | 0.11 | Olfr827 | 0.06 |
| Olfr653 | 0.19 | Olfr731 | 0.10 | Olfr829 | 0.07 |
| Olfr655 | 0.09 | Olfr733 | 0.05 | Olfr832 | 0.09 |
| Olfr656 | 0.05 | Olfr734 | 0.11 | Olfr834 | 0.06 |
| Olfr658 | 0.06 | Olfr735 | 0.08 | Olfr835 | 0.13 |
| Olfr659 | 0.14 | Olfr736 | 0.12 | Olfr836 | 0.17 |
| Olfr66 | 0.10 | Olfr738 | 0.26 | Olfr843 | 0.07 |
| Olfr661 | 0.08 | Olfr739 | 0.07 | Olfr854 | 0.11 |
| Olfr663 | 0.06 | Olfr74 | 0.11 | Olfr856-ps1 | 79.03 |
| Olfr665 | 0.07 | Olfr740 | 0.12 | Olfr857 | 0.23 |
| Olfr666 | 0.12 | Olfr741 | 0.08 | Olfr860 | 0.14 |
| Olfr667 | 0.05 | Olfr742 | 0.06 | Olfr866 | 0.06 |
| Olfr668 | 0.10 | Olfr744 | 0.08 | Olfr869 | 0.06 |
| Olfr669 | 0.44 | Olfr745 | 0.60 | Olfr871 | 0.05 |
| Olfr67 | 0.05 | Olfr747 | 0.09 | Olfr874 | 0.06 |
| Olfr670 | 0.08 | Olfr748 | 0.05 | Olfr875 | 0.12 |
| Olfr672 | 0.08 | Olfr749 | 0.08 | Olfr876 | 0.07 |
| Olfr676 | 0.44 | Olfr75-ps1 | 0.16 | Olfr877 | 0.12 |
| Olfr678 | 0.07 | Olfr750 | 0.21 | Olfr878 | 0.09 |
| Olfr683 | 0.05 | Olfr76 | 0.10 | Olfr881 | 0.15 |
| Olfr684 | 0.07 | Olfr763 | 0.13 | Olfr883 | 0.12 |
| Olfr685 | 0.20 | Olfr765 | 0.05 | Olfr884 | 0.06 |
| Olfr686 | 0.13 | Olfr767 | 0.07 | Olfr885 | 0.14 |
| Olfr688 | 0.15 | Olfr768 | 0.06 | Olfr887 | 0.06 |
| Olfr689 | 0.09 | Olfr769 | 0.05 | Olfr888 | 0.14 |
| Olfr690 | 0.06 | Olfr770 | 0.07 | Olfr891 | 0.06 |
| Olfr691 | 0.21 | Olfr772 | 0.06 | Olfr893 | 0.14 |
| Olfr692 | 0.18 | Olfr773 | 0.06 | Olfr894 | 0.05 |
| Olfr693 | 0.09 | Olfr777 | 0.05 | Olfr9 | 0.17 |
| Olfr694 | 0.08 | Olfr78 | 0.05 | Olfr90 | 0.10 |
| Olfr695 | 0.26 | Olfr782 | 0.05 | Olfr904 | 0.08 |
| Olfr697 | 0.06 | Olfr784 | 0.11 | Olfr905 | 0.09 |
| Olfr698 | 0.23 | Olfr786 | 0.15 | Olfr906 | 0.22 |
| Olfr699 | 0.07 | Olfr790 | 0.18 | Olfr907 | 0.09 |
| Olfr70 | 0.18 | Olfr791 | 0.12 | Olfr91 | 0.12 |
| Olfr700 | 0.32 | Olfr794 | 0.09 | Olfr911-ps1 | 0.17 |
| Olfr701 | 0.08 | Olfr796 | 0.12 | Olfr876 | 0.07 |
| Olfr702 | 0.10 | Olfr800 | 0.09 | Olfr877 | 0.12 |
| Olfr917 | 0.05 | Olfr914 | 0.09 | Olfr878 | 0.09 |
| Olfr92 | 0.19 | Olfr916 | 0.08 | Olfr881 | 0.15 |
| Olfr922 | 0.06 | Olfr917 | 0.05 | Olfr883 | 0.12 |
| Olfr923 | 0.17 | Olfr92 | 0.19 | Olfr884 | 0.06 |
| Olfr803 | 0.05 | Olfr922 | 0.06 | Olfr885 | 0.14 |
| Olfr806 | 0.06 | Olfr923 | 0.17 | Olfr887 | 0.06 |
| Olfr808 | 0.22 | Olfr924 | 0.05 | Olfr888 | 0.14 |
| Olfr810 | 0.07 | Olfr926 | 0.05 | Olfr891 | 0.06 |
| Olfr812 | 0.05 | Olfr930 | 0.06 | Olfr893 | 0.14 |
| Olfr814 | 0.16 | Olfr934 | 0.12 | Olfr894 | 0.05 |
| Olfr816 | 0.43 | Olfr935 | 0.05 | Olfr9 | 0.17 |
| Olfr818 | 0.08 | Olfr937 | 0.07 | Olfr90 | 0.10 |
| Olfr822 | 0.10 | Olfr94 | 0.15 | Olfr904 | 0.08 |
| Olfr827 | 0.06 | Olfr945 | 0.08 | Olfr905 | 0.09 |
| Olfr829 | 0.07 | Olfr948 | 0.05 | Olfr906 | 0.22 |
| Olfr832 | 0.09 | Olfr95 | 0.20 | Olfr907 | 0.09 |
| Olfr834 | 0.06 | Olfr951 | 0.11 | Olfr91 | 0.12 |
| Olfr835 | 0.13 | Olfr952 | 0.06 | Olfr911-ps1 | 0.17 |
| Olfr836 | 0.17 | Olfr957 | 0.10 | Olfr979 | 0.15 |
| Olfr843 | 0.07 | Olfr958 | 0.21 | Olfr98 | 0.19 |
| Olfr854 | 0.11 | Olfr959 | 0.13 | Olfr981 | 0.07 |
| Olfr856-ps1 | 79.03 | Olfr961 | 0.05 | Olfr982 | 0.10 |
| Olfr857 | 0.23 | Olfr963 | 0.07 | Olfr983 | 0.14 |
| Olfr860 | 0.14 | Olfr967 | 0.08 | Olfr984 | 0.07 |
| Olfr866 | 0.06 | Olfr969 | 0.06 | Olfr986 | 0.13 |
| Olfr869 | 0.06 | Olfr97 | 0.07 | Olfr988 | 0.05 |
| Olfr871 | 0.05 | Olfr971 | 0.12 | Olfr99 | 0.32 |
| Olfr874 | 0.06 | Olfr974 | 0.06 | Olfr996 | 0.08 |
| Olfr875 | 0.12 | Olfr975 | 0.12 | Olfr998 | 0.06 |

TABLE 3
Olfrs expressed in vascular macrophages in mice, along with corresponding human ortholog

| Mouse | Human orth. | Mouse | Human orth. | Mouse | Human orth. | Mouse | Human orth. |
|---|---|---|---|---|---|---|---|
| Olfr239 | OR10H1 | Olfr1461 | OR5B3 | Olfr694 | OR2AG1, OR2AG2 | Olfr1022 | OR5M1, OR5M10 |
| Olfr1174-ps | AP006437.1 | Olfr874 | OR8B12 | Olfr1028 | OR5M11 | Olfr1138 | OR5W2 |
| Olfr401 | OR3A1 | Olfr406 | OR1P1 | Olfr469 | OR5P2 | Olfr470 | OR5P2 |
| Olfr91 | OR2H1, OR2H2 | Olfr638 | OR51Q1 | Olfr486 | OR5P2 | Olfr1219 | OR4C15 |
| Olfr1029 | OR5M11 | Olfr460 | OR9A4 | Olfr487 | OR5P2 | Olfr430 | OR6N2, OR2A42 |
| Olfr314 | OR2T12 | Olfr1222 | OR4C15 | Olfr376 | OR1E1 | Olfr1038-ps | OR5R1, OR8J3 |
| Olfr320 | OR2AK2, OR2T4, | Olfr692 | OR52W1, OR2AG2 | Olfr720 | OR2T6 | Olfr1356 | OR7C2, OR2J2, |
| | OR2T5 | | | | | | OR2J3 |
| Olfr566 | OR51F1 | Olfr441 | OR2A14 | Olfr574 | OR51T1 | Olfr77 | OR7D4 |
| Olfr55 | OR10H1, OR52K2 | Olfr447 | OR2A25 | Olfr1423 | OR4D11 | Olfr715 | OR2D2 |
| Olfr1510 | OR10G2 | Olfr362 | OR1B1, ORIL6 | Olfr248 | OR10X1 | Olfr1211 | OR4C15 |
| Olfr316 | OR2AK2 | Olfr1016 | OR9G1, OR12D2 | Olfr1104 | OR8I2 | Olfr218 | OR10J3 |
| Olfr92 | OR2H1, OR2H2 | Olfr419 | OR10Z1 | Olfr1032 | OR5M3 | Olfr57 | OR7A10 |
| Olfr982 | OR10S1 | Olfr76 | OR5A1 | Olfr1136 | OR5W2 | Olfr495 | OR5P2 |
| Olfr398 | AC087498.1 | Olfr1491 | OR10Q1 | Olfr1264 | OR4C3 | Olfr808 | OR6C65 |
| Olfr522 | OR6Q1, OR10H2, | Olfr96 | OR11A1, OR10G7, | Olfr518 | OR10A3, OR10A6 | Olfr1352 | OR7A10, OR7A17, |
| | OR10H5 | | OR10G8 | | | | OR7A5 |
| Olfr147 | OR8B2 | Olfr211 | OR13A1 | Olfr519 | OR10A3 | Olfr393 | OR1E1 |
| Olfr678 | OR52E5 | Olfr986 | OR6X1 | Olfr835 | OR7G2 | Olfr631 | OR51M1 |
| Olfr1388 | OR2Y1 | Olfr1506 | OR4X2 | Olfr420 | OR6K2 | Olfr1202 | OR4C13 |
| Olfr1426 | OR4D10 | Olfr90 | OR2H1, OR2H2 | Olfr556 | OR52I1 | Olfr1097 | OR8H2, OR8H3 |
| Olfr169 | OR2AJ1 | Olfr167 | OR2L2 | Olfr1193 | OR4S2 | Olfr802 | OR6C1 |
| Olfr170 | OR2AJ1 | Olfr288 | OR10AD1 | Olfr1353 | OR7A10, OR7A17, | Olfr1241 | OR4A16, OR4A5, |
| | | | | | OR7A5 | | OR4A8 |
| Olfr171 | OR2AJ1, OR5H14, | Olfr1495 | OR10Q1 | Olfr410 | OR3A1 | Olfr292 | OR14C36 |
| | OR5H15 | | | | | | |
| Olfr370 | OR10K2, OR1E2, | Olfr1226 | OR4C15 | Olfr1255 | OR4C12 | Olfr677 | OR52E4 |
| | OR1E1 | | | | | | |
| Olfr267 | OR2K2 | Olfr558 | OR51E1 | Olfr1258 | OR4C46 | Olfr1270 | OR4B1 |
| Olfr796 | OR10P1, OR7A17, | Olfr448 | OR2A5 | Olfr1166 | OR5D13 | Olfr1436 | OR5AN1 |
| | OR7A5 | | | | | | |
| Olfr63 | OR10H3 | Olfr1217 | OR4C15 | Olfr309 | OR13G1 | Olfr746 | OR11H7 |
| Olfr328 | OR2T29, OR2DT4, | Olfr1490 | OR10W1 | Olfr659 | OR52N4 | Olfr384 | OR1E1, OR1E2 |
| | OR2T5 | | | | | | |
| Olfr1272 | OR4B1 | Olfr446 | OR2A12 | Olfr389 | OR1E1 | Olfr821 | OR6C74 |
| Olfr417 | OR10X1 | Olfr850 | OR7G2 | Olfr1057 | OR8J1 | Olfr937 | OR8G1 |
| Olfr485 | OR5P2 | Olfr641 | OR51I2 | Olfr979 | OR10G4 | Olfr836 | OR7G2 |
| Olfr222 | OR2B11 | Olfr137 | OR2J1 | Olfr1507 | OR4E1 | Olfr592 | OR52J3, OR10H4 |
| Olfr582 | OR52R1 | Olfr571 | ORS1S1 | Olfr262 | OR5AN1 | Olfr1289 | OR4F17, OR2A7 |
| Olfr1502 | OR9I1 | Olfr3 | OR1J1 | Olfr1387 | OR2Y1 | Olfr828 | OR7G2 |
| Olfr464 | OR4D1 | Olfr437 | OR2A2 | Olfr492 | OR5P2 | Olfr1023 | OR5M1 |
| Olfr1260 | OR4C5 | Olfr1444 | OR5B21 | Olfr830 | OR7G2 | Olfr1220 | OR4C15 |
| Olfr13 | OR2A4, OR4F21, | Olfr635 | OR51Q1 | Olfr1209 | OR4C16 | Olfr8 | OR7A10 |
| | OR4F29 | | | | | | |
| Olfr1511 | OR10G2 | Olfr1137 | OR5W2 | Olfr1242 | OR4A16, OR4A5, | Olfr679 | OR56A3 |
| | | | | | OR4A8 | | |
| Olfr95 | OR10C1 | Olfr102 | OR12D1 | Olfr168 | OR2L13 | Olfr681 | OR56A3 |
| Olfr750 | OR6S1 | Olfr1508 | OR4E1 | Olfr504 | OR56B1 | Olfr498 | OR5P2 |
| Olfr463 | OR4D2 | Olfr1238 | OR4A16, OR4A5, | Olfr557 | OR51D1 | Olfr1170 | OR5D13 |
| | | | OR4A8 | | | | |
| Olfr231 | OR6K6, OR10H2, | Olfr569 | OR52R1, OR7A17, | Olfr1153 | OR5W2 | Olfr1243 | OR4A16, OR4A5, |
| | OR10H5 | | OR7A5 | | | | OR4A8 |
| Olfr514 | OR10A3, OR10A6 | Olfr700 | OR2AG1, OR2AG2 | Olfr26 | OR8D1 | Olfr729 | OR4K5 |
| Olfr984 | OR4D5 | Olfr577 | OR51G2 | Olfr1431 | OR5AN1 | Olfr490 | OR5P2 |
| Olfr978 | OR10G4, OR10G7, | Olfr706 | OR2AG1, OR10A5 | Olfr1263 | OR4C45 | Olfr1058 | OR8K3 |
| | OR10G8 | | | | | | |
| Olfr666 | OR52N2, OR52E8 | Olfr1259 | OR4C12 | Olfr876 | OR8B12 | Olfr1065 | OR8K3 |
| Olfr161 | OR1F1, OR2L3, | Olfr1118 | OR10AG1 | Olfr382 | OR1E1, OR1E2 | Olfr1454 | OR5B3 |
| | OR2L5 | | | | | | |
| Olfr1076 | OR8K3 | Olfr981 | OR10G6 | Olfr873 | OR7E24 | Olfr1141 | OR5W2 |
| Olfr435 | OR2A1 | Olfr698 | OR2AG1, OR2AG2 | Olfr716 | OR2D3 | Olfr186 | OR5H1, OR7A17, |
| | | | | | | | OR7A5 |
| Olfr745 | OR11H6 | Olfr875 | OR8B12 | Olfr1467 | OR5B3 | Olfr1014 | OR9G1 |
| Olfr402 | OR3A1 | Olfr1099 | OR8H1 | Olfr74 | OR5D18 | Olfr191 | OR5H1, OR5H14, |
| | | | | | | | OR5H15 |
| Olfr788 | OR6C3 | Olfr1354 | OR7A10, OR7A17, | Olfr142 | OR4B1 | Olfr378 | OR1E2 |
| | | | OR7A5 | | | | |
| Olfr723 | OR4L1 | Olfr1355 | OR7A10 | Olfr786 | OR6C1 | Olfr2 | OR6A2 |
| Olfr724 | OR4L1 | Olfr552 | OR52K1 | Olfr357 | OR1Q1 | Olfr1199 | OR4C16 |
| Olfr765 | OR2AP1 | Olfr790 | OR6C75 | Olfr640 | OR51I1 | Olfr1451 | OR5B2 |
| Olfr1428 | OR4D6 | Olfr867 | OR7D4 | Olfr930 | OR8D1 | Olfr512 | OR10A3, OR10A6 |
| Olfr39 | OR7D4, OR1E2 | Olfr494 | OR5P2 | Olfr68 | OR52A5 | Olfr1020 | OR5AP2, OR5M10 |
| Olfr329-ps | OR2T29, OR2T4, | Olfr19 | OR7A10, OR5H14, | Olfr1249 | OR4A16, OR4A5, | Olfr1273-ps | OR4B1, OR4F4, |
| | OR2T5 | | OR5H15 | | OR4A8 | | OR4F5 |
| Olfr331 | OR2T29 | Olfr688 | OR56B4 | Olfr1499 | OR9I1 | Olfr385 | OR1E1, OR1E2 |
| Olfr220 | OR6Y1 | Olfr985 | OR8D4 | Olfr497 | OR5P2 | Olfr295 | OR14C36 |
| Olfr459 | OR9A2 | Olfr354 | OR1N2 | Olfr1140 | OR5W2 | Olfr1228 | OR4C15, OR4A5, |
| | | | | | | | OR4A8 |
| Olfr713 | OR10A2 | Olfr703 | OR2AG1, OR2AG2 | Olfr671 | OR52E6 | Olfr872 | OR7E24 |
| Olfr59 | OR1P1 | Olfr878 | OR8B4, OR2H2 | Olfr1303 | OR4F16/17, OR7A5 | Olfr944 | OR8G1 |
| Olfr145 | OR8B8 | Olfr697 | OR2AG1, OR2AG2 | Olfr1509 | OR4E2 | Olfr1116 | OR10AG1 |
| Olfr317 | OR2W3 | Olfr482 | OR5P2 | Olfr1469 | OR5B3, OR8B3 | Olfr936 | OR8G5 |
| Olfr689 | OR56B4 | Olfr506 | OR5P2 | Olfr466 | OR9H1P | Olfr943 | OR8G1 |
| Olfr365 | OR1L4 | Olfr507 | OR5P2, OR10A6 | Olfr502 | OR5P2 | Olfr849 | OR7G2 |
| Olfr1252 | OR4A16 | Olfr520 | OR2AT4 | Olfr1466 | OR5B3 | Olfr1066 | OR8K3 |
| Olfr31 | OR2T1, OR2T33, | Olfr554 | OR52M1, OR52I2 | Olfr1404 | OR10J3 | Olfr199 | OR5AC1 |
| | OR2T8 | | | | | | |
| Olfr958 | OR10D3 | Olfr484 | OR5P2 | Olfr32 | OR4B1 | Olfr1167 | OR5D13 |
| Olfr93 | OR2H1 | Olfr693 | OR2AG1, OR2AG2 | Olfr1442 | OR5B3 | Olfr196 | OR5H1 |

TABLE 4
Human olfactory receptors expressed in coronaries

| Gene | Controls NXT_A5 | Case NXT_B5 | GFOLD(0.01) | Gene_Name |
|---|---|---|---|---|
| ENSG00000181785.2 | 546.864638 | 578.635755 | 0 | OR5AS1 |
| ENSG00000176695.8 | 504.918616 | 563.419456 | 0 | OR4F17 |
| ENSG00000127530.3 | 386.176633 | 315.330905 | −0.162011 | OR7C1 |
| ENSG00000204695.4 | 377.706822 | 556.177204 | 0.210455 | OR14J1 |
| ENSG00000228198.3 | 276.882578 | 318.614617 | 0 | OR2M3 |
| ENSG00000172146.2 | 276.518697 | 346.4817 | 0 | OR1A1 |
| ENSG00000183444.10 | 276.319502 | 433.805203 | 0.274878 | OR7E38P |
| ENSG00000177201.2 | 254.799213 | 402.89834 | 0.270254 | OR2T12 |
| ENSG00000172519.9 | 214.260285 | 265.658244 | 0 | OR10H5 |
| ENSG00000203663.4 | 201.99031 | 291.13782 | 0.0984326 | OR2L2 |
| ENSG00000094661.3 | 181.6724 | 302.355662 | 0.299551 | OR1I1 |
| ENSG00000186723.4 | 169.65786 | 133.203895 | −0.0897745 | OR10H1 |
| ENSG00000172742.5 | 162.285486 | 202.345304 | 0 | OR4D9 |
| ENSG00000254593.1 | 162.27436 | 84.3381327 | −0.626424 | OR7E126P |
| ENSG00000187847.4 | 159.507692 | 254.762033 | 0.218945 | OR7E25P |
| ENSG00000255804.2 | 158.239868 | 112.673288 | −0.188318 | OR6J1 |
| ENSG00000170356.9 | 139.080996 | 69.1550135 | −0.640902 | OR2A20P |
| ENSG00000173285.4 | 138A41653 | 167.502134 | 0 | OR10K1 |
| ENSG00000228336.2 | 136.812705 | 176.281207 | 0 | OR9H1P |
| ENSG00000171561.4 | 133.862532 | 207.151682 | 0.140105 | OR2AT4 |
| ENSG00000172320.3 | 124.38252 | 173.357825 | 0 | OR5A1 |
| ENSG00000255303.2 | 122.229214 | 168.645339 | 0 | OR5BA1P |
| ENSG00000184166.2 | 121.129162 | 132.773611 | 0 | OR1D2 |
| ENSG00000170790.5 | 120.097289 | 80.0655486 | −0.232222 | OR10A2 |
| ENSG00000180785.9 | 110.761584 | 117.063217 | 0 | OR51E1 |
| ENSG00000188000.4 | 107.470536 | 136.721424 | 0 | OR7D2 |
| ENSG00000174667.4 | 105.063188 | 137.408601 | 0 | OR7D4 |
| ENSG00000170923.3 | 99.9626014 | 123.530422 | 0 | OR7G2 |
| ENSG00000284791.1 | 88.3624998 | 0 | −3.74841 | OR7E47P |
| ENSG00000183389.5 | 87.4286738 | 102.511116 | 0 | OR56A4 |
| ENSG00000274052.2 | 85.9523142 | 63.7066912 | 0 | OR7E156P |
| ENSG00000215160.3 | 85.0764391 | 268.369648 | 1.12898 | OR7E122P |
| ENSG00000188558.6 | 83.3979651 | 101.176283 | 0 | OR2G6 |
| ENSG00000203757.2 | 82.0528018 | 151.775593 | 0.314364 | OR6K3 |
| ENSG00000182070.5 | 76.7592432 | 131.555004 | 0.177862 | OR52A1 |
| ENSG00000232268.6 | 73.9029234 | 53.1062282 | −0.017257 | OR52I1 |
| ENSG00000176495.3 | 73.3445318 | 8.15428516 | −1.91019 | OR5AN1 |
| ENSG00000169208.2 | 72.063169 | 111.668453 | 0.00942992 | OR10G3 |
| ENSG00000221836.3 | 70.8155486 | 76.7516508 | 0 | OR2A5 |
| ENSG00000254737.2 | 69.2812575 | 162.790322 | 0.658625 | OR10G4 |
| ENSG00000185385.4 | 68.9845498 | 177.591567 | 0.789019 | OR7A17 |
| ENSG00000172324.5 | 68.9097609 | 71.4393367 | 0 | OR5A2 |
| ENSG00000197532.2 | 68.7974219 | 60.9833381 | 0 | OR6Y1 |
| ENSG00000229386.3 | 68.3171876 | 138.37922 | 0.422025 | OR8B9P |
| ENSG00000249646.2 | 67.5546022 | 71.1007876 | 0 | OR7E94P |
| ENSG00000176230.6 | 67.3145407 | 79.9467046 | 0 | OR4K17 |
| ENSG00000254752.2 | 65.1416483 | 71.0021782 | 0 | OR5M2P |
| ENSG00000219926.11 | 64.8293406 | 3.86557323 | −2.49098 | OR7E104P |
| ENSG00000196119.7 | 63.3775606 | 53.0251179 | 0 | OR8A1 |
| ENSG00000180437.5 | 62.9029845 | 0 | −3.2517 | OR6K4P |
| ENSG00000168158.3 | 60.0437651 | 0 | −3.18163 | OR2C1 |
| ENSG00000227892.1 | 60.0437207 | 0 | −3.18163 | OR5P4P |
| ENSG00000170929.6 | 59.4290287 | 92.9168677 | 0 | OR1M1 |
| ENSG00000169327.5 | 54.8306032 | 81.0759544 | 0 | OR5AU1 |
| ENSG00000226413.2 | 51.4660582 | 0 | −2.9484 | OR8T1P |
| ENSG00000205409.3 | 50.5129918 | 0 | −2.9484 | OR52E6 |
| ENSG00000170683.6 | 49.5791167 | 67.8787387 | 0 | OR10A3 |
| ENSG00000196184.9 | 49.1793067 | 85.5376168 | 0.0905611 | OR10J1 |
| ENSG00000182652.3 | 48.8846845 | 6.12415162 | −1.61798 | OR4Q3 |
| ENSG00000285474.1 | 47.6916295 | 0 | −2.86146 | OR7E154P |
| ENSG00000224402.2 | 45.8477711 | 63.8943019 | 0 | OR6D1P |
| ENSG00000236032.3 | 45.1332562 | 68.0523365 | 0 | OR5H14 |
| ENSG00000180090.5 | 44.6279381 | 4.59413216 | −1.66446 | OR3A1 |
| ENSG00000180934.6 | 44.2651434 | 72.6954164 | 0 | OR56A1 |
| ENSG00000272595.2 | 42.8095771 | 61.9533093 | 0 | OR10AH1P |
| ENSG00000176253.3 | 42.0160431 | 69.7616334 | 0 | OR4K13 |
| ENSG00000159961.2 | 39.9535248 | 43.73905 | 0 | OR3A3 |
| ENSG00000243896.4 | 38.6890368 | 25.9453507 | 0 | OR2A7 |
| ENSG00000182613.2 | 37.6769509 | 58.8546337 | 0 | OR2V2 |
| ENSG00000171180.2 | 36.0617331 | 71.5995097 | 0.221662 | OR2M4 |
| ENSG00000167332.8 | 30.0670845 | 85.1835567 | 0.686353 | OR51E2 |
| ENSG00000179626.4 | 29.3418447 | 46.5122723 | 0 | OR6C4 |
| ENSG00000284689.1 | 28.6439911 | 12.5393039 | −0.0918595 | OR7E84P |
| ENSG00000177535.8 | 28.6215207 | 36.0929581 | 0 | OR2B11 |
| ENSG00000204700.5 | 28.3794383 | 40.6400862 | 0 | OR2J2 |
| ENSG00000221882.3 | 28.3044513 | 16.4305549 | 0 | OR3A2 |
| ENSG00000187857.4 | 28.2646054 | 54.1032411 | 0.042119 | OR6C75 |
| ENSG00000188269.9 | 27.7235813 | 31.5140557 | 0 | OR7A5 |
| ENSG00000196778.3 | 26.6804545 | 51.5584451 | 0.0208175 | OR52K1 |
| ENSG00000237258.8 | 26.5021368 | 32.452784 | 0 | OR11A1 |
| ENSG00000182854.8 | 26.4952553 | 40.14141 | 0 | OR4F15 |
| ENSG00000196534.5 | 24.9196619 | 33.5016257 | 0 | OR9K1P |
| ENSG00000179615.2 | 24.3038141 | 41.804654 | 0 | OR2AP1 |
| ENSG00000224392.1 | 23.8268859 | 0 | −1.8117 | OR2G1P |
| ENSG00000197309.3 | 23.8268405 | 0 | −1.8117 | OR10D3 |
| ENSG00000236359.1 | 21.9207231 | 0 | −1.614 | OR51B8P |
| ENSG00000188324.4 | 21.7452412 | 14.9005208 | 0 | OR6C6 |
| ENSG00000213215.5 | 21.0860876 | 46.3028088 | 0.189909 | OR2F1 |
| ENSG00000181693.8 | 20.8014812 | 11.6145091 | 0 | OR8H1 |
| ENSG00000167355.8 | 20.4650197 | 40.3322461 | 0.0112981 | OR51B5 |
| ENSG00000197125.3 | 20.0233414 | 9.80312258 | 0 | OR8B8 |
| ENSG00000181767.3 | 19.4605494 | 27.3553558 | 0 | OR8H2 |
| ENSG00000276240.2 | 19.2968745 | 8.23140684 | 0 | OR4E1 |
| ENSG00000180974.4 | 18.5520959 | 46.8234928 | 0.340268 | OR52E4 |
| ENSG00000181001.2 | 18.4750523 | 36.4094962 | 0 | OR52N1 |
| ENSG00000186513.3 | 18.1084257 | 0 | −1.30024 | OR9Q2 |
| ENSG00000171496.4 | 18.0900614 | 9.22728509 | 0 | OR1L8 |
| ENSG00000177212.4 | 18.0687622 | 27.4919869 | 0 | OR2T33 |
| ENSG00000228307.2 | 17.9950181 | 12.3023386 | 0 | OR2S1P |
| ENSG00000228960.6 | 17.9127332 | 8.88412332 | 0 | OR2A9P |
| ENSG00000285405.1 | 17.8618733 | 0 | −1.30024 | OR11J6P |
| ENSG00000231861.2 | 17.1553819 | 0 | −1.21004 | OR5K2 |
| ENSG00000197786.3 | 17.1553619 | 0 | −1.21004 | OR5B17 |
| ENSG00000231548.2 | 16.1342986 | 24.5528768 | 0 | OR55B1P |
| ENSG00000221933.3 | 16.0915447 | 83.5895442 | 1.42166 | OR2A25 |
| ENSG00000284768.1 | 15.8708554 | 0 | −1.11392 | OR2A12 |
| ENSG00000197454.2 | 15.2810263 | 11.5155112 | 0 | OR2L5 |
| ENSG00000196242.9 | 15.0452579 | 12.2219147 | 0 | OR2C3 |
| ENSG00000196341.3 | 14.429841 | 13.1216065 | 0 | OR8D1 |
| ENSG00000196832.4 | 14.0244617 | 27.5769539 | 0 | OR11G2 |
| ENSG00000234485.5 | 13.6451259 | 1.89094436 | −0.900453 | OR7E46P |
| ENSG00000180658.4 | 13.4774257 | 4.35645584 | 0 | OR2A4 |
| ENSG00000176269.3 | 13.3430873 | 0 | −0.780893 | OR4F21 |
| ENSG00000174957.1 | 13.3302961 | 8.15766182 | 0 | OR5J2 |
| ENSG00000197428.3 | 12.687851 | 23.1221105 | 0 | OR51D1 |
| ENSG00000188124.3 | 12.6658524 | 19.3691531 | 0 | OR2AG2 |
| ENSG00000186119.8 | 12.3899635 | 0 | −0.65081 | OR5D18 |
| ENSG00000176798.2 | 11.8267594 | 20.955519 | 0 | OR51L1 |
| ENSG00000227152.6 | 11.4720377 | 16.2501062 | 0 | OR2T7 |
| ENSG00000177306.4 | 11.4603333 | 0 | −0.508248 | OR7E125P |
| ENSG00000184669.9 | 11.2959781 | 21.721789 | 0 | OR7E14P |
| ENSG00000181927.3 | 10.9751701 | 32.4867146 | 0.127801 | OR4P4 |
| ENSG00000181518.4 | 10.7557213 | 35.1191278 | 0.286733 | OR8D4 |
| ENSG00000141194.6 | 10.5631057 | 15.2371567 | 0 | OR4D1 |
| ENSG00000244292.1 | 10.4837954 | 0 | −0.350579 | OR9N1P |
| ENSG00000273423.1 | 10.4319367 | 7.66719819 | 0 | OR13I1P |
| ENSG00000176510.5 | 9.68307826 | 9.19509916 | 0 | OR10AC1 |
| ENSG00000205240.4 | 8.44623198 | 0 | −0.008352 | OR7E36P |
| ENSG00000233606.2 | 8.14883753 | 8.8199876 | 0 | OR6C66P |
| ENSG00000237988.5 | 7.9657598 | 58.3501724 | 1.27572 | OR2I1P |
| ENSG00000221888.4 | 7.80105605 | 3.0412225 | 0 | OR1C1 |
| ENSG00000279301.4 | 7.64688388 | 11.6049031 | 0 | OR2T11 |
| ENSG00000238243.3 | 7.62459069 | 0 | −0.008352 | OR2W3 |
| ENSG00000254613.2 | 6.9527864 | 7.83296348 | 0 | OR6M2P |
| ENSG00000187747.2 | 6.67155028 | 0 | 0 | OR52B6 |
| ENSG00000231049.2 | 6.53827785 | 7.00611683 | 0 | OR52B5P |
| ENSG00000272937.2 | 5.81627334 | 17.507293 | 0 | OR6U2P |
| ENSG00000166693.7 | 5.71844202 | 0 | 0 | OR5M13P |
| ENSG00000181214.7 | 5.71843666 | 0 | 0 | OR8G2P |
| ENSG00000254743.1 | 5.02054052 | 0 | 0 | OR10V3P |
| ENSG00000258822.2 | 5.01359415 | 3.06027254 | 0 | OR4K16P |
| ENSG00000167822.2 | 4.99342079 | 13.3152596 | 0 | OR8J3 |
| ENSG00000231237.1 | 4.90876873 | 5.2754809 | 0 | OR6K1P |
| ENSG00000197849.6 | 4.8214225 | 6.09046705 | 0 | OR8G1 |
| ENSG00000182634.8 | 4.76544301 | 0 | 0 | OR10G7 |
| ENSG00000175398.2 | 4.76538558 | 0 | 0 | OR10P1 |
| ENSG00000198128.4 | 4.76529329 | 0 | 0 | OR2L3 |
| ENSG00000136834.3 | 4.50869629 | 11.5209323 | 0 | OR1J1 |
| ENSG00000267450.1 | 4.28362022 | 1.31368778 | 0 | OR1AB1P |
| ENSG00000221970.2 | 4.21172164 | 6.36591094 | 0 | OR2A1 |
| ENSG00000180708.5 | 4.04281914 | 7.49534286 | 0 | OR10K2 |
| ENSG00000185372.3 | 3.86737836 | 9.5489069 | 0 | OR2V1 |
| ENSG00000237225.2 | 3.83277315 | 1.02230229 | 0 | OR13K1P |
| ENSG00000236083.2 | 3.78692704 | 8.20484906 | 0 | OR13E1P |
| ENSG00000273484.1 | 3.17139557 | 15.6655187 | 0.12071 | OR6R2P |
| ENSG00000176231.2 | 3.05605359 | 0 | 0 | OR10H4 |
| ENSG00000221813.4 | 3.00184947 | 15.2722523 | 0.00301549 | OR6B1 |
| ENSG00000165762.3 | 2.9285894 | 8.15410454 | 0 | OR4K2 |
| ENSG00000251491.2 | 2.90325628 | 0 | 0 | OR7E28P |
| ENSG00000279486.5 | 2.8894006 | 3.04534005 | 0 | OR2AG1 |
| ENSG00000231192.2 | 2.87103993 | 9.13098984 | 0 | OR5H1 |
| ENSG00000169488.6 | 2.85923931 | 0 | 0 | OR4K15 |
| ENSG00000282137.1 | 2.85923161 | 0 | 0 | OR4G3P |
| ENSG00000255203.1 | 2.85922295 | 0 | 0 | OR7E2P |
| ENSG00000183122.5 | 2.85921245 | 0 | 0 | OR2A3P |
| ENSG00000259324.2 | 2.85920839 | 0 | 0 | OR11K1P |
| ENSG00000189398.5 | 2.63024582 | 0 | 0 | OR7E12P |
| ENSG00000254948.1 | 2.22885391 | 1.08803581 | 0 | OR7E158P |
| ENSG00000235623.2 | 2.20667157 | 0 | 0 | OR7E110P |
| ENSG00000184698.5 | 2.13789161 | 28.4812714 | 1.3736 | OR51M1 |
| ENSG00000250640.2 | 1.91942484 | 0 | 0 | OR7E41P |
| ENSG00000204657.4 | 1.91137294 | 1.01858945 | 0 | OR2H2 |
| ENSG00000175619.3 | 1.90614891 | 0 | 0 | OR4B1 |
| ENSG00000172148.3 | 1.1695972 | 0 | 0 | OR7A2P |
| ENSG00000250561.2 | 1.10800264 | 9.22895742 | 0.00835202 | OR7E59P |
| ENSG00000285469.1 | 1.06276224 | 0 | 0 | OR7E161P |
| ENSG00000279000.3 | 0.9668797 | 11.050567 | 0.350579 | OR10A6 |
| ENSG00000254472.1 | 0.96558353 | 5.02852014 | 0 | OR4A49P |
| ENSG00000181074.4 | 0.96528631 | 28.167611 | 1.87204 | OR52N4 |
| ENSG00000186124.4 | 0.95922815 | 8.09787288 | 0 | OR9M1P |
| ENSG00000221977.2 | 0.95848729 | 4.0756067 | 0 | OR4E2 |
| ENSG00000181718.6 | 0.95630535 | 4.06116094 | 0 | OR5T2 |
| ENSG00000186509.4 | 0.95620445 | 1.01540285 | 0 | OR9Q1 |
| ENSG00000242525.1 | 0.95430062 | 1.01824823 | 0 | OR7E100P |
| ENSG00000176895.9 | 0.95308364 | 0 | 0 | OR51A7 |
| ENSG00000184140.6 | 0.95306147 | 0 | 0 | OR4F6 |
| ENSG00000225101.6 | 0.95302828 | 0 | 0 | OR52K3P |
| ENSG00000183909.6 | 0 | 41.7949942 | 2.52693 | OR4G2P |
| ENSG00000184022.4 | 0 | 37.6930047 | 2.36796 | OR2T10 |
| ENSG00000248553.1 | 0 | 36.6743329 | 2.32534 | OR52H2P |
| ENSG00000238228.1 | 0 | 35.6555844 | 2.28142 | OR7E7P |
| ENSG00000176904.3 | 0 | 30.5619742 | 2.03938 | OR51H1 |
| ENSG00000177174.1 | 0 | 27.5057615 | 1.87204 | OR14C36 |
| ENSG00000181780.4 | 0 | 27.5057426 | 1.87204 | OR5J1P |
| ENSG00000249633.1 | 0 | 22.4120845 | 1.54166 | OR52V1P |
| ENSG00000228914.2 | 0 | 18.3371754 | 1.21004 | OR1H1P |
| ENSG00000237610.1 | 0 | 16.2997032 | 1.01105 | OR4C50P |
| ENSG00000255030.1 | 0 | 14.2622616 | 0.780893 | OR8B5P |
| ENSG00000205495.1 | 0 | 13.2435191 | 0.65081 | OR52J3 |
| ENSG00000231319.3 | 0 | 12.2247952 | 0.508248 | OR2B3 |
| ENSG00000244623.1 | 0 | 11.5834438 | 0.508248 | OR2AE1 |
| ENSG00000279270.2 | 0 | 11.2060429 | 0.350579 | OR52R1 |
| ENSG00000197882.3 | 0 | 10.4088558 | 0 | OR7E13P |
| ENSG00000230655.3 | 0 | 9.36785945 | 0.00835202 | OR7E55P |
| ENSG00000214344.4 | 0 | 9.17599513 | 0.00835202 | OR4F13P |
| ENSG00000226288.2 | 0 | 9.16866232 | 0.00835202 | OR52I2 |
| ENSG00000203408.4 | 0 | 9.16859147 | 0.00835202 | OR6C71P |
| ENSG00000184478.7 | 0 | 8.14976718 | 0 | OR56A3 |
| ENSG00000258550.1 | 0 | 7.7890611 | 0 | OR7E106P |
| ENSG00000274360.2 | 0 | 7.13113873 | 0 | OR6W1P |
| ENSG00000280094.2 | 0 | 7.13113737 | 0 | OR1B1 |
| ENSG00000204701.2 | 0 | 5.09376099 | 0 | OR2J3 |
| ENSG00000166884.2 | 0 | 5.09365521 | 0 | OR4D6 |
| ENSG00000228547.5 | 0 | 4.52426088 | 0 | OR7E26P |
| ENSG00000230261.1 | 0 | 4.07491126 | 0 | OR52J2P |
| ENSG00000197938.5 | 0 | 4.0749112 | 0 | OR5H2 |
| ENSG00000273252.1 | 0 | 3.1590137 | 0 | OR7E39P |
| ENSG00000225980.4 | 0 | 3.06023742 | 0 | OR7E19P |
| ENSG00000232511.4 | 0 | 3.05624557 | 0 | OR2AH1P |
| ENSG00000232381.1 | 0 | 3.05620091 | 0 | OR52U1P |
| ENSG00000254848.1 | 0 | 3.05619803 | 0 | OR5BN1P |
| ENSG00000180409.3 | 0 | 3.05619094 | 0 | OR10AA1P |
| ENSG00000181009.5 | 0 | 3.05615923 | 0 | OR52N5 |
| ENSG00000277932.2 | 0 | 3.05612603 | 0 | OR52E5 |
| ENSG00000226537.2 | 0 | 2.08221903 | 0 | OR7E33P |
| ENSG00000171936.2 | 0 | 2.03745586 | 0 | OR10H3 |
| ENSG00000198601.3 | 0 | 1.01876029 | 0 | OR2M2 |
| ENSG00000174937.4 | 0 | 1.01875812 | 0 | OR5M3 |
| ENSG00000196099.4 | 0 | 1.01875546 | 0 | OR6M1 |
| ENSG00000172640.3 | 0 | 1.01875434 | 0 | OR10AD1 |
| ENSG00000189181.4 | 0 | 1.01871442 | 0 | OR14I1 |
| ENSG00000176951.6 | 0 | 1.01871387 | 0 | OR51N1P |
| ENSG00000257542.5 | 17.7745506 | 0 | −1.30024 | OR7E47P |
| ENSG00000221858.3 | 0 | 22.0390932 | 1.54166 | OR2A12 |

TABLE 5
Human olfactory receptors found in the Biobank of Karolinska
Endarterectomy (BiKE) dataset

| | | | | |
|---|---|---|---|---|
| OR5E1P | OR5AK4P | OR51M1 | OR7E111P | OR1J2 |
| OR2I1P | OR2AD1P | OR52A1 | OR1F1 | OR10D3 |
| OR10A3 | OR10C1 | OR6W1P | OR51I2 | OR2A20P |
| OR3A2 | OR51E2 | OR7A5 | OR2C1 | OR1D2 |
| OR5G1P | OR2A4 | OR7A17 | OR7E47P | OR10A4 |
| OR7E24 | OR2L1P | OR4A1P | OR3A3 | OR8B8 |
| OR2H4P | OR6A2 | OR10J1 | OR1F2P | OR1Q1 |
| OR2B4P | OR2F1 | OR2S2 | OR13C4 | OR1A1 |
| OR8G2 | OR51E1 | OR5L2 | OR52K3P | OR4D2 |
| OR5P2 | OR3A1 | OR52D1 | OR10H1 | OR5P3 |
| OR2K2 | OR51A10P | OR51I1 | OR2N1P | OR12D3 |
| OR2H1 | OR7D2 | OR51A1P | OR12D2 | OR2H2 |
| OR10A5 | OR1D5 | OR1G1 | OR7E12P | OR2B6 |
| OR4C1P | OR1C1 | OR51J1 | OR7E156P | OR8G1 |
| OR2B3 | OR8D1 | OR10D1P | OR7C2 | OR10H3 |
| OR1J4 | OR8D2 | OR4D1 | OR1E3 | OR5I1 |
| OR51B4 | OR51B6 | OR4F16 | OR1I1 | OR51B5 |
| OR7E19P | OR7C1 | OR51B2 | OR7E37P | OR5V1 |
| OR2L2 | OR14J1 | OR1E1 | OR7E104P | |

TABLE 6
Human olfactory receptors expressed in coronaries or found in BIKE dataset

| Gene | Controls NXT_A5 | Case NXT_B5 | GFOLD(0.01) | OR |
|---|---|---|---|---|
| ENSG00000221858.3 | 0 | 22.0390932 | 1.54166 | OR2A12 |
| ENSG00000184698.5 | 2.13789161 | 28.4812714 | 1.3736 | OR51M1 |
| ENSG00000185385.4 | 68.9845498 | 177.591567 | 0.789019 | OR7A17 |
| ENSG00000167332.8 | 30.0670845 | 85.1835567 | 0.686353 | OR51E2 |
| ENSG00000231319.3 | 0 | 12.2247952 | 0.508248 | OR2B3 |
| ENSG00000094661.3 | 181.6724 | 302.355662 | 0.299551 | OR1I1 |
| ENSG00000204695.4 | 377.706822 | 556.177204 | 0.210455 | OR14J1 |
| ENSG00000213215.5 | 21.0860876 | 46.3028088 | 0.189909 | OR2F1 |
| ENSG00000182070.5 | 76.7592432 | 131.555004 | 0.177862 | OR52A1 |
| ENSG00000203663.4 | 201.99031 | 291.13782 | 0.0984326 | OR2L2 |
| ENSG00000196184.9 | 49.1793067 | 85.5376168 | 0.0905611 | OR10J1 |
| ENSG00000167355.8 | 20.4650197 | 40.3322461 | 0.0112981 | OR51B5 |
| ENSG00000172146.2 | 276.518697 | 346.4817 | 0 | OR1A1 |
| ENSG00000184166.2 | 121.129162 | 132.773611 | 0 | OR1D2 |
| ENSG00000180785.9 | 110.761584 | 117.063217 | 0 | OR51E1 |
| ENSG00000188000.4 | 107.470536 | 136.721424 | 0 | OR7D2 |
| ENSG00000274052.2 | 85.9523142 | 63.7066912 | 0 | OR7E156P |
| ENSG00000170683.6 | 49.5791167 | 67.8787387 | 0 | OR10A3 |
| ENSG00000159961.2 | 39.9535248 | 43.73905 | 0 | OR3A3 |
| ENSG00000221882.3 | 28.3044513 | 16.4305549 | 0 | OR3A2 |
| ENSG00000188269.9 | 27.7235813 | 31.5140557 | 0 | OR7A5 |
| ENSG00000197125.3 | 20.0233414 | 9.80312258 | 0 | OR8B8 |
| ENSG00000196341.3 | 14.429841 | 13.1216065 | 0 | OR8D1 |
| ENSG00000180658.4 | 13.4774257 | 4.35645584 | 0 | OR2A4 |
| ENSG00000141194.6 | 10.5631057 | 15.2371567 | 0 | OR4D1 |
| ENSG00000221888.4 | 7.80105605 | 3.0412225 | 0 | OR1C1 |
| ENSG00000197849.6 | 4.8214225 | 6.09046705 | 0 | OR8G1 |
| ENSG00000189398.5 | 2.63024582 | 0 | 0 | OR7E12P |
| ENSG00000204657.4 | 1.91137294 | 1.01858945 | 0 | OR2H2 |
| ENSG00000225101.6 | 0.95302828 | 0 | 0 | OR52K3P |
| ENSG00000274360.2 | 0 | 7.13113873 | 0 | OR6W1P |
| ENSG00000225980.4 | 0 | 3.06023742 | 0 | OR7E19P |
| ENSG00000171936.2 | 0 | 2.03745586 | 0 | OR10H3 |
| ENSG00000186723.4 | 169.65786 | 133.203895 | −0.0897745 | OR10H1 |
| ENSG00000127530.3 | 386.176633 | 315.330905 | −0.162011 | OR7C1 |
| ENSG00000170356.9 | 139.080996 | 69.1550135 | −0.640902 | OR2A20P |
| ENSG00000284768.1 | 15.8708554 | 0 | −1.11392 | OR2A12 |
| ENSG00000257542.5 | 17.7745506 | 0 | −1.30024 | OR7E47P |
| ENSG00000180090.5 | 44.6279381 | 4.59413216 | −1.66446 | OR3A1 |
| ENSG00000197309.3 | 23.8268405 | 0 | −1.8117 | OR10D3 |
| ENSG00000219926.11 | 64.8293406 | 3.86557323 | −2.49098 | OR7E104P |
| ENSG00000168158.3 | 60.0437651 | 0 | −3.18163 | OR2C1 |
| ENSG00000284791.1 | 88.3624998 | 0 | −3.74841 | OR7E47P |

TABLE 7
Olfactory Receptor Screening-Top 17 expressed receptors with known
human orthologue and agonist

| | Mouse | | |
|---|---|---|---|
| | Ortholog | | |
| | (Ensemble | | |
| Gene Name | source) | Agonist | Known Antagonist |
| OR2B11 | Olfr222 | Coumarin | |
| OR3A1 | Olfr402 | helional, Lilial | Methylcinnamaldehyde, |
| | | | Hydrocinnamaldehyde, |
| | | | Bourgeonal |
| OR4D6 | Olfr1428 | b-ionone | |
| OR5A1 | Olfr76 | b-ionone | |
| OR7C1 | Olfr1352 | Androstadienone | |
| OR7D4 | Olfr39 | Androstenone, | |
| | | Androstadienone | |
| OR8D1 | Olfr26 | Caramel furanone | |
| OR10A6 | Olfr519 | 3-phenyl propyl | |
| | | propienate | |
| OR10G7 | Olfr978 | Eugenol | |
| OR10G9 | Olfr979 | Ethil Vanillin | |
| OR11A1 | Olfr96 | 2-ethyl-fencol | |
| OR11H6 | Olfr745 | Isovaleric Acid | |
| OR51E1 | Olfr558 | Nonanoic acid, | |
| | | Butyl | |
| | | butyryllactate, | |
| | | butyric acid, | |
| | | isovaleric acid, | |
| | | propionic acid | |
| OR4E2 | Olfr1509 | N-amyl acetate | |
| OR4D2 | Olfr463 | eugenol acetate | |
| OR10H1 | Olfr239 | sandalwood | |
| OR6A2 | Olfr2 | octanal | |

TABLE 8
Agonizing odorants: OLFR pairs

| Odorant | | Odorant | |
|---|---|---|---|
| (agonist) | Olfactory receptor | (agonist) | Olfactory receptor |
| Lyral | OLFR16 | vanillin | OR10G4 K295Q |
| azelaic acid | Olfr544 (human unknown) | 2-methoxy-4- | OR10G7 |
| | | methylphenol | |
| propionic acid | OLFR78 | banana | OR10G7 |
| isoeugenol | OR10A6 | eugenol | OR10G7 |
| androstenone | OR10A6 A117V/V140G/L287P | eugenol methyl | OR10G7 |
| | | ether | |
| 3-phenyl propyl | OR10A6 L287P | guaiacol | OR10G7 |
| propionate | | | |
| androstenone | OR10A6 L287P | jasmine | OR10G7 |
| 3-phenyl propyl | OR10A6 V140G/L287P | nutmeg | OR10G7 |
| propionate | | | |
| amyl laurate | OR10A6 V140G/L287P | spearmint | OR10G7 |
| androstadienone | OR10A6 V140G/L287P | eugenol | OR10G7 T13M |
| androstenone | OR10A6 V140G/L287P | eugenol | OR10G7 T90A |
| butyric acid | OR10A6 V140G/L287P | sandalwood | OR10H1 |
| isoeugenol | OR10A6 V140G/L287P | Santalol | OR10H1 |
| cinnamon | OR10AG1 | Sandranol | OR10H1 |
| eugenol acetate | OR10G3 | cinnamaldehyde | OR10H2 |
| vanillin | OR10G3 | geranyl acetate | OR10H2 |
| vanillin | OR10G3 S73G | cinnamaldehyde | OR10H2 L40Q |
| vanillin | OR10G4 | eugenol | OR10H5 |
| 2-methoxy-4- | OR10G4 | lyral | OR10J5 |
| methylphenol | A9V/M134V/V195E/R235G/K295Q | | |
| vanillin | OR10G4 | α-cedrene | OR10J5 |
| | A9V/M134V/V195E/R235G/K295Q | | |
| 2-decenal | OR14A2 | Lyral | OR10J5 |
| cis-3-hexen-1-ol | OR14J1 | lyral | OR10J5 R233W |
| (+)-carvone | OR1A1 | (−)-menthol | OR10X1 |
| coumarin | OR1C1 | 2-ethylfenchol | OR11A1 |
| linalool | OR1C1 | isovaleric acid | OR11H4 |
| geranyl acetate | OR1D2 | isovaleric acid | OR11H6 |
| bourgeonal | OR1D2 | eugenol acetate | OR13C3 G2C |
| eugenol | OR1D5 | sandalwood | OR14A16 Indel |
| isovaleric acid | OR1E1 | cyclohexyl | OR2A4/7 |
| | | salicylate | |
| cinnamon | OR1N1 | amyl butyrate | OR2AG1 |
| cinnamon | OR1N1 P18S | isoeugenol | OR2AT4 |
| linalool | OR1N2 | Sandranol | OR2AT4 |
| jasmine | OR1S2 | Brahmanol | OR2AT4 |
| jasmine | OR1S2 I46T | cinnamaldehyde | OR2B11 |
| geranyl acetate | OR2A25 | coffee difuran | OR2B11 |
| quinoline | OR2A25 | coumarin | OR2B11 |
| geranyl acetate | OR2A25 A209P | dicyclohexyl | OR2B11 |
| | | disulfide | |
| geranyl acetate | OR2A25 S75N | quinoline | OR2B11 |
| geranyl acetate | OR2A25 S75N/A209P | spearmint | OR2B11 |
| quinoline | OR2A25 S75N/A209P | cis-3-hexen-1- | OR2J1 L12I |
| | | ol | |
| cinnamaldehyde | OR2B11 I130S/V198M | androstenone | OR2J2 |
| coffee difuran | OR2B11 V198M | butyl | OR2J2 |
| | | anthranilate | |
| coumarin | OR2B11 V198M | cis-3-hexen-1- | OR2J2 |
| | | ol | |
| quinoline | OR2B11 V198M | isovaleric acid | OR8U8 |
| spearmint | OR2B11 V198M | cinnamaldehyde | OR9G1 |
| coffee difuran | OR2B11 V198M/T293I/D300G | ethyl vanillin | OR2J2 |
| coumarin | OR2B11 V198M/T293I/D300G | eugenol methyl | OR2J2 |
| | | ether | |
| quinoline | OR2B11 V198M/T293I/D300G | isoeugenol | OR2J2 |
| spearmint | OR2B11 V198M/T293I/D300G | phenyl | OR2J2 |
| | | acetaldehyde | |
| 1-octanethiol | OR2C1 | androstenone | OR2J2 T111A |
| cinnamaldehyde | OR2C1 | butyl | OR2J2 T111A |
| | | anthranilate | |
| phenyl | OR2C1 C149W | cis-3-hexen-1- | OR2J2 T111A |
| acetaldehyde | | ol | |
| thioglycolic acid | OR2C1 C149W | ethyl vanillin | OR2J2 T111A |
| cinnamaldehyde | OR2C1 | eugenol methyl | OR2J2 T111A |
| | G16S/C149W/C169Y/R229H | ether | |
| phenyl | OR2C1 | isoeugenol | OR2J2 T111A |
| acetaldehyde | G16S/C149W/C169Y/R229H | | |
| eugenol acetate | OR2F1 | phenyl | OR2J2 T111A |
| | | acetaldehyde | |
| α-cedrene | OR2H2 | ethyl vanillin | OR2J2 |
| | | | Y74H/T111A/V146A/T218A |
| aldehyde 13-13 | OR2H2 | isoeugenol | OR2J2 |
| | | | Y74H/T111A/V146A/T218A |
| cis-3-hexen-1-ol | OR2J3 I228V | phenyl | OR2J2 |
| | | acetaldehyde | Y74H/T111A/V146A/T218A |
| ethylene | OR2J3 I228V | cinnamaldehyde | OR2J3 |
| brassylate | | | |
| geranyl acetate | OR2J3 I228V | cis-3-hexen-1- | OR2J3 |
| | | ol | |
| cinnamaldehyde | OR2J3 I228V/M261I | geranyl acetate | OR2J3 |
| cis-3-hexen-1-ol | OR2J3 I228V/M261I | n-amyl acetate | OR2J3 |
| eugenol methyl | OR2J3 I228V/M261I | cinnamaldehyde | OR2J3 I228V |
| ether | | | |
| geranyl acetate | OR2J3 I228V/M261I | butyric acid | OR2T1 P132L |
| octyl aldehyde | OR2J3 I228V/M261I | sandalwood | OR2T1 P132L |
| spearmint | OR2J3 I228V/M261I | jasmine | OR2T6 N21D/C23G/S243A |
| cinnamaldehyde | OR2J3 R226Q/I228V/M261I | cis-3-hexen-1- | OR2W1 |
| | | ol | |
| cis-3-hexen-1-ol | OR2J3 R226Q/I228V/M261I | cis-3-hexen-1- | OR2W1 D296N |
| | | ol | |
| geranyl acetate | OR2J3 R226Q/I228V/M2611 | cis-3-hexen-1- | OR2W1 M81V |
| | | ol | |
| cinnamaldehyde | OR2J3 | cinnamon | OR3A1 |
| | T113A/R226Q/I228V/M261I | | |
| geranyl acetate | OR2M2 R220G/C235R | dihydrojasmone | OR3A1 |
| geraniol | OR2M7 | cinnamon | OR3A1 R125Q |
| geraniol | OR2M7 F35L/V78A/C178F | ethyl vanillin | OR3A1 R125Q |
| isoeugenol | OR2S2 | jasmine | OR4A4 |
| butyric acid | OR2T1 | isovaleric acid | OR4C12 V283L |
| butyric acid | OR2T1 I76V | PI-23472 | OR4D1 |
| Nonanoic acid | OR51E1 | eugenol acetate | OR4D2 |
| isovaleric acid | OR51E1 K299R | eugenol acetate | OR4D2 C97S/L187F |
| isovaleric acid | OR51E1 SUN | eugenol acetate | OR4D2 L29I |
| propionic acid | OR51E2 | androstenone | OR4F17 |
| β-ionone | OR51E2 | geranyl acetate | OR4L1 R52S |
| allyl | OR51L1 | jasmine | OR4N4 |
| phenylacetate | | | |
| allyl | OR51L1 I281M | butyl | OR4Q3 |
| phenylacetate | | anthranilate | |
| allyl | OR51L1 T196I/A207V | eugenol | OR4Q3 |
| phenylacetate | | | |
| eugenol | OR52B6 | eugenol | OR4Q3 F238L |
| | T36A/L90H/A146T/H149R/V267I | | |
| undecanal | OR56A4 | jasmine | OR4X2 |
| butyric acid | OR5AL1 | Troenan | OR51B4 |
| butyric acid | OR5AL1 Insertion | isononyl | OR51B5 |
| | | alcohol | |
| butyric acid | OR5D14 | butyric acid | OR51D1 |
| butyric acid | OR5D14 Q102L/S249A | isovaleric acid | OR51E1 |
| eugenol methyl | OR5K1 | androstadienone | OR7C1 V126I/E171K/S210P |
| ether | | | |
| TMT | OR5K1 | androstenone | OR7D4 |
| coumarin | OR5P3 | androstenone | OR7D4 P79L |
| eugenol methyl | OR6F1 F215L | androstenone | OR7D4 R88W/T133M |
| ether | | | |
| anisaldehyde | OR6P1 | butyric acid | OR7G2 V263A/F281V |
| phenyl | OR6P1 | (+)-carvone | OR8B3 |
| acetaldehyde | | | H20R/Q24R/V34I/M114I |
| Myrac | OR7A5 | (−)-carvone | OR8B3 |
| | | | H20R/Q24R/V34I/M114I |
| androstadienone | OR7C1 | r-carvone | OR8B3 |
| | | | H20R/Q24R/V34I/M114I |
| androstenone | OR7C1 | caramel | OR8D1 |
| | | furanone | |
| androstadienone | OR7C1 S99G/V126I/E171K/S210P | sandalwood | OR8D1 |
| eugenol acetate | OR9G1 | (+)-menthol | OR8K3 L122R |
| Coumarin | OR2B11 | (−)-menthol | OR8K3 L122R |
| helional, Lilial | OR3A1 | Ethil Vanillin | OR10G9 |
| b-ionone | OR4D6 | 2-ethyl-fencol | OR11A1 |
| b-ionone | OR5A1 | Isovaleric Acid | OR11H6 |
| Androstadienone | OR7C1 | Nonanoic acid, | OR51E1 |
| | | Butyl | |
| | | butyryllactate, | |
| | | butyric acid, | |
| | | isovaleric acid, | |
| | | propionic acid | |
| Androstenone, | OR7D4 | N-amyl acetate | OR4E2 |
| Androstadienone | | | |
| Caramel | OR8D1 | eugenol acetate | OR4D2 |
| furanone | | | |
| 3-phenyl propyl | OR10A6 | sandalwood | OR10H1 |
| propienate | | | |
| Eugenol | OR10G7 | octanal | OR6A2 |

TABLE 9
Antagonistic odorants: OLFR pairs

| Odorant (antagonistic) | Olfactory receptor |
|---|---|
| undecanal | OR17-4 |
| undecanal | OR1D2 |
| oxyphenylon | OR2AT4 |
| phenirat | OR2AT4 |
| methyl cinnamaldehyde | OR3A1 |
| hydrocinamaldehyde | OR3A1 |
| bourgeonal | OR3A1 |
| ethylhexanoic acid | OR51E1 |
| α-ionone | OR51E2 |
| octanoic acid | ORs6 |
| octanoic acid | ORs86 |

What is claimed is:

1. A method of modulating an immune response in a subject, comprising modulating expression or activity of one or more olfactory receptors (OLFR).

2. The method of claim 1, wherein the OLFR is expressed by a cell in vivo.

3. The method of claim 2, wherein the cell is an animal cell, with the proviso that the cell is not an olfactory cell.

4. The method of any one of claims 1-3, wherein the cell is a macrophage.

5. The method of claim 4, wherein the macrophage is a vascular macrophage.

6. The method of any one of claims 1-5, wherein the modulation comprises one or more of inhibiting, decreasing, reducing, suppressing, limiting or controlling the immune response by a method comprising administering to the subject an effective amount of an agent that inhibits the expression of or deactivates the OLFR.

7. The method of any one of claims 1-5, wherein the modulation comprises one or more of decreasing, reducing, inhibiting, suppressing, limiting or controlling an undesirable or aberrant immune response, immune disorder, inflammatory response or inflammation, by a method comprising administering to the subject an effective amount of an agent that inhibits the expression of or deactivates the OLFR.

8. The method of any one of claims 1-5, wherein the modulation comprises one or more of decreasing, reducing, inhibiting, suppressing, limiting or controlling an autoimmune response, disorder or disease in a subject, by a method comprising administering to the subject an effective amount of an agent that inhibits the expression of or deactivates the OLFR.

9. The method of claim 8, wherein the modulation comprises one or more of decreasing, reducing, inhibiting, suppressing, limiting or controlling an adverse symptom of the undesirable or aberrant immune response, immune disorder, inflammatory response or inflammation, or an adverse symptom of the autoimmune response, disorder or disease in the subject.

10. The method of claim 9, wherein the adverse symptom of the undesirable or aberrant immune response, immune disorder, inflammatory response or inflammation or an adverse symptom of the autoimmune response, disorder or disease is swelling, pain, rash, headache, fever, nausea, diarrhea, bloat, lethargy, skeletal joint stiffness or tissue or cell damage.

11. The method of claim 9 or 10, wherein the adverse symptom of the undesirable or aberrant immune response, immune disorder, inflammatory response or inflammation or the adverse symptom of the autoimmune response, disorder or disease is chronic or acute.

12. The method of any one of claims 8 to 11, wherein the immune disorder, inflammatory response, inflammation, autoimmune response disorder or autoimmune disease comprises rheumatoid arthritis, juvenile rheumatoid arthritis, osteoarthritis, psoriatic arthritis, diabetes mellitus, multiple sclerosis, encephalomyelitis, myasthenia gravis, systemic lupus erythematosus (SLE), autoimmune thyroiditis, atopic dermatitis, eczematous dermatitis, psoriasis, Sjogren's Syndrome, Crohn's disease, aphthous ulcer, iritis, conjunctivitis, keratoconjunctivitis, ulcerative colitis, inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common g chain (c) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (g5/14.1) deficiency, Hyper-IgM syndrome: X-linked (CD40 ligand deficiency) or non-X-linked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon g receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1 A/SAP deficiency), or hyper IgE syndrome.

13. The method of any one of claims 1-5, wherein the modulation comprises one or more of decreasing, reducing, inhibiting, suppressing, limiting or controlling an adverse cardiovascular event or cardiovascular disease by a method comprising administering to the subject an effective amount of an agent that inhibits the expression of or deactivates the OLFR.

14. The method of claim 13, wherein the adverse cardiovascular event or cardiovascular disease comprises coronary artery disease, peripheral artery disease, cerebrovascular disease, renal artery disease, stroke, myocardial infarction (heart attack), ischemic heart failure, transient ischemic attack or brain trauma, atherosclerosis, atherosclerotic plaque formation or elevated blood cholesterol.

15. The method of any one of claims 1-5, wherein the modulation comprises one or more of decreasing, reducing, inhibiting, suppressing, limiting or controlling atherosclerosis, by a method comprising administering to the subject an effective amount of an agent that inhibits the expression of or deactivates the OLFR.

16. The method of any one of claims 1-5, wherein the method comprises one or more of reducing or inhibiting in a subject viral, bacterial or fungal infection, by a method comprising administering to the subject an effective amount of an agent that increases the expression of or activates the OLFR.

17. The method of any one of claims 1-5, wherein the modulation comprises one or more of comprising decreasing, reducing, inhibiting, suppressing, limiting or controlling an adverse symptom of the neoplasia, neoplastic disorder, tumor, cancer or malignancy, metastasis of a neoplasia, tumor, cancer or malignancy to other sites, or formation or establishment of a metastatic neoplasia, neoplastic disorder, tumor, cancer or malignancy to other sites distal from a primary neoplasia, neoplastic disorder, tumor, cancer or malignancy, or viral, bacterial or fungal infection by a method comprising administering to the subject an effective amount of an agent that increases the expression of or activates the OLFR.

18. The method of claim 17, wherein the neoplasia, neoplastic disorder, tumor, cancer or malignancy treated is a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, lymphosarcoma, liposarcoma, osteosarcoma, chondrosarcoma, leiomyosarcoma, rhabdomyosarcoma, fibrosarcoma or melanoma; or a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, neoplastic disorder, tumor, cancer or malignancy.

19. The method of any one of claims 1-18, wherein the OLFR is an OLFR listed in any one of Tables 1-9, and FIGS. 2A and 2B.

20. The method of any one of claims 1-18, wherein the OLFR is an OLFR listed in Table 7.

21. The method of any one of claims 1-18, wherein the OLFR is selected from the group consisting of OR7C1, OR7D4, OR10A6, OR11H6, OR4E2, OR10H1, and OR6A2.

22. The method of any one of claims 6 to 21, wherein the agent is selected from the group of: a ligand or small molecule that binds to the OLFR or blocks the binding of the OLFR to the ligand or an agent that inhibits the expression of the OLFR by the cell.

23. The method of any preceding claim, wherein the agent is selected from the group of an antibody, fragment or mimetic that binds to OLFR or an OLFR ligand, an anti-OLFR gene silencing agent, octanal, heptanal, or a prodrug or solvate thereof.

24. The method of any one of claims 6-21, wherein the agent modulates the OLFR by modulating the trafficking of the OLFR to a plasma membrane of a cell.

25. The method of claim 24, wherein the agent is a protein selected from the group of receptor transporting protein 1 (RTP1), RTP2, receptor expression enhancing protein 1 (REEP1), aminoacylase 3 (Acy3), or guanine nucleotide-binding protein G(olf) subunit alpha (Gnal).

26. The method of claim 24, wherein the agent is a nucleotide encoding a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal).

27. The method of any of claims 16-26, wherein the immune response is stimulated by a method comprising administering an agent that increases expression or secretion of an inflammatory cytokine.

28. The method of claim 28, wherein the inflammatory cytokine is selected from the group consisting of tumor necrosis factor (TNF), C-C motif chemokine ligand 2 (CCL2), CCL4, CCL5, interleukin 6 (IL-6), IL-1B, IL-18, and nitric oxide synthase 2 (NOS2).

29. A method of suppressing an immune response in a subject in need thereof, the method comprising administering to the subject an agent that decreases the expression of or activity of an olfactory receptor (OLFR), thereby suppressing an immune response in the subject.

30. The method of claim 29, wherein the OLFR is an OLFR listed in any one of Tables 1-9.

31. The method of claim 29 or 30, wherein the OLFR is an OLFR listed in Table 7.

32. The method of any of claims 29-31, wherein the OLFR is selected from the group consisting of OR7C1, OR7D4, OR10A6, OR11H6, OR4E2, OR10H1, and OR6A2.

33. The method of any of claims 29-32, wherein the agent is a gene silencing agent.

34. The method of claim 23 or 33, wherein the gene silencing agent is selected from the group consisting of a RNA interference (RNAi) molecule, zinc finger nuclease, transcription activator-like effector nuclease (TALEN), and Clustered Regulatory Interspaced Short Palindromic Repeats (CRISPR) enzyme.

35. The method of claim 34, wherein the RNAi molecule is selected from the group consisting of a small interference RNA (siRNA), short hairpin RNA (shRNA) and microRNA (miRNA).

36. The method of any of claims 29-32, wherein the agent decreases the activity of the OLFR by inhibiting the binding of the OLFR with its ligand.

37. The method of claim 36, wherein the ligand is an OLFR ligand listed in Table 8.

38. The method of any of claims 29-32, wherein the agent is an OLFR antagonist.

39. The method of claim 38, wherein the OLFR antagonist is citral, undecanal, oxyphenylon, phenirat, methyl cinnamaldehyde, hydrocinamaldehyde, bourgeonal, ethylhexanoic acid, α-ionone, octanoic acid, a solvate or prodrug thereof.

40. The method of claim 38, wherein the OLFR antagonist is an antagonist listed in Table 9.

41. The method of any of claims 29-32, wherein the agent decreases the activity of the OLFR by inhibiting the trafficking of the OLFR to a plasma membrane of a cell.

42. The method of any of claims 29-32, wherein the agent is an antibody, fragment or mimetic thereof that binds to an OLFR.

43. A method of increasing an immune response in a subject in need thereof, the method comprising administering to the subject an agent that increases or promotes the trafficking of an olfactory receptor (OLFR) to a plasma membrane of a cell, thereby increasing an immune response in the subject.

44. A method of suppressing an immune response in a subject in need thereof, the method comprising administering to the subject an agent that inhibits the trafficking of an olfactory receptor (OLFR) to a plasma membrane of a cell, thereby suppressing an immune response in the subject.

45. A method of treating an autoimmune disease in a subject in need thereof, the method comprising administering to the subject an agent that decreases the expression of or activity of an olfactory receptor (OLFR).

46. A method of treating an autoimmune disease in a subject in need thereof, the method comprising administering to the subject an agent that inhibits the trafficking of an olfactory receptor (OLFR) to a plasma membrane of a cell.

47. A method of treating a cardiovascular disease in a subject in need thereof, the method comprising administering to the subject an agent that decreases the expression of or activity of an olfactory receptor (OLFR).

48. A method of a cardiovascular disease in a subject in need thereof, the method comprising administering to the subject an agent that inhibits the trafficking of an olfactory receptor (OLFR) to a plasma membrane of a cell.

49. A method of treating an infection in a subject in need thereof, the method comprising administering to the subject an agent that increases the expression of or activates an olfactory receptor (OLFR).

50. A method of treating an infection in a subject in need thereof, the method comprising administering to the subject an agent that increases or promotes the trafficking of an olfactory receptor (OLFR) to a plasma membrane of a cell.

51. The method of claim 49 or 50, wherein the infection is selected from the group consisting of a viral infection, bacterial infection, or fungal infection.

52. A method of treating cancer in a subject in need thereof, the method comprising administering to the subject an agent that increases the expression of or activates an olfactory receptor (OLFR).

53. A method of cancer in a subject in need thereof, the method comprising administering to the subject an agent that increases or promotes the trafficking of an olfactory receptor (OLFR) to a plasma membrane of a cell.

54. A method of modulating the expression or activity of one or more proteins involved in an immune response, the method comprising contacting a cell with an agent that modulates the expression or activity of an olfactory receptor (OLFR).

55. The method of claim 54, wherein the agent increases the expression of or activates the OLFR.

56. The method of claim 54, wherein the agent decreases the expression or activity of the OLFR.

57. A method of increasing the expression or activity of one or more proteins involved in an immune response, the method comprising contacting a cell with an agent that increases the expression of or activates an olfactory receptor (OLFR).

58. A method of decreasing the expression or activity of one or more proteins involved in an immune response, the method comprising contacting a cell with an agent that decreases the expression of or inhibits an olfactory receptor (OLFR).

59. A method of modulating the expression or activity of one or more proteins involved in an immune response, the method comprising contacting a cell with an agent that modulates the trafficking of an olfactory receptor (OLFR) to a plasma membrane of the cell.

60. The method of claim 59, wherein agent increases or promotes the trafficking of the OLFR to the plasma membrane of the cell.

61. The method of claim 59, wherein the agent decreases or inhibits the trafficking of the OLFR to the plasma membrane of a cell.

62. A method of increasing the expression or activity of one or more proteins involved in an immune response, the method comprising contacting a cell with an agent that increases or promotes the trafficking of an olfactory receptor (OLFR) to a plasma membrane of the cell.

63. A method of decreasing the expression or activity of one or more proteins involved in an immune response, the method comprising contacting a cell with an agent that decreases or inhibits the trafficking of an olfactory receptor (OLFR) to a plasma membrane of a cell.

64. The method of any of claims 44-48, 54, 56, 58, 59, 61, and 63, wherein the agent is a gene silencing agent.

65. The method of claim 64, wherein the gene silencing agent is selected from the group consisting of a RNA interference (RNAi) molecule, zinc finger nuclease, transcription activator-like effector nuclease (TALEN), and Clustered Regulatory Interspaced Short Palindromic Repeats (CRISPR) enzyme.

66. The method of claim 65, wherein the RNAi molecule is selected from the group consisting of a small interference RNA (siRNA), short hairpin RNA (shRNA) and microRNA (miRNA).

67. The method of any of claims 45, 47, 56, and 58, wherein the agent decreases the activity of the OLFR by inhibiting the binding of the OLFR with its ligand.

68. The method of claim 67, wherein the ligand is an OLFR ligand listed in Table 8.

69. The method of any of claims 44-48, 54, 56, 58, 59, 61, and 63, wherein the agent is an OLFR antagonist.

70. The method of claim 69, wherein the OLFR antagonist is citral, undecanal, oxyphenylon, phenirat, methyl cinnamaldehyde, hydrocinamaldehyde, bourgeonal, ethylhexanoic acid, α-ionone, octanoic acid, a solvate or prodrug thereof.

71. The method of claim 69, wherein the OLFR antagonist is an antagonist listed in Table 9.

72. The method of any of claims 45, 47, 56, and 58, wherein the agent decreases the activity of the OLFR by inhibiting the trafficking of the OLFR to a plasma membrane of a cell.

73. The method of any of claims 44-48, 54, 56, 58, 59, 61, and 63, wherein the agent is an antibody, fragment or mimetic thereof that binds to an OLFR.

74. The method of any of claims 43, 49-55, 57, 59, and 60, wherein the agent is an OLFR agonist.

75. The method of claim 74, wherein the OLFR agonist is an agonist listed in Table 8.

76. The method of claim 74, wherein the OLFR agonist is selected from the group consisting of octanal, coumarin, helional, lilial, b-ionone, androstenone, androstadienone, caramel furanone, 3-phenyl propyl propionate, eugenol, ethil vanillin, 2-ethyl-fencol, isovaleric acid, nonanoic acid, butyl butyryllactate, butyric acid, isovaleric acid, propionic acid, N-amyl acetate, eugenol acetate, sandalwood, S-(−)-citronellol, S-(−)-citronellal, (+)-carvine, (−) carvone, (+) carvone, linalool, bourgeonal, acetophenone, amyl butyrate, nonanethiol, allyl phenyl acetate, N-amyl acetate, muscone, isoeugenol, eugenol methyl ether, 1-hexanol, 1-heptanole, 1-octanol, celery ketone, anis aldehyde, (+)-menthol, vanillin, guaiacol, lyral, ethyl heptanoate, methyl octanoate, nonanal 1-nonanol, 2-nonanol, 3-octanone, 3-nonanone, decyl aldehyde.

77. The method of any of claims 43, 49-55, 57, 59, and 60, wherein the agent is octanal, heptanal, or a prodrug or solvate thereof.

78. The method of any of claims 43, 49-55, 57, 59, and 60, wherein the agent is a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal).

79. The method of any of claims 43, 49-55, 57, 59, and 60, wherein the agent comprises a nucleotide encoding a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal).

80. The method of any of claims 43-79, wherein the OLFR is an OLFR listed in any one of Tables 1-9 and FIGS. 2A-2B.

81. The method of any of claims 43-80, wherein the OLFR is an OLFR listed in Table 7.

82. The method of any of claims 43-81, wherein the OLFR is selected from the group consisting of OR7C1, OR7D4, OR10A6, OR11H6, OR4E2, OR10H1, and OR6A2.

83. The method of any of claims 43-82, wherein the OLFR is expressed by a cell in vivo.

84. The method of claim 83, wherein the cell is an animal cell, with the proviso that the cell is not an olfactory cell.

85. The method of claim 83 or 84, wherein the cell is a macrophage.

86. The method of claim 85, wherein the macrophage is a vascular macrophage.

87. The method of any one of claims 54-63, wherein the one or more proteins involved in an immune response is any of the proteins shown in FIGS. 16 and 17.

88. The method of any one of claims 54-63, wherein the one or more proteins involved in an immune response is a protein selected from CCL5 (C-C Motif Chemokine Ligand 5), Tnfrsf12a (Osteoprotegerin), Axin 1, Nadk, Ahr (Aryl hydrocarbon receptor), QDPR (quinoid dihydropteridine reductase), HGF (Hepatocyte Growth Factor), ADAM23, or Snap29.

89. The method of any of claims 6 to 53, wherein the administration is local or systemic.

90. The method of claim 89, wherein the administration comprises by a method comprising intravenously.

91. The method of any one of claims 6 to 53, 89, and 90, wherein the subject is a mammal.

92. The method of any one of claims 6 to 53, and 89-91, wherein the subject has or is predisposed to a disease or disorder involving an immune dysregulation.

93. The method of claim 91 or 92, wherein the subject is a human patient.

94. A kit comprising an agent that modulates the activity of an OLFR and instructions for use.

95. The kit of any claim 94, wherein the agent is a gene silencing agent.

96. The kit of claim 95, wherein the gene silencing agent is selected from the group consisting of a RNA interference (RNAi) molecule, zinc finger nuclease, transcription activator-like effector nuclease (TALEN), and Clustered Regulatory Interspaced Short Palindromic Repeats (CRISPR) enzyme.

97. The kit of claim 96, wherein the RNAi molecule is selected from the group consisting of a small interference RNA (siRNA), short hairpin RNA (shRNA) and microRNA (miRNA).

98. The kit of any of claims 94-97, wherein the agent modulates the activity of the OLFR by inhibiting the binding of the OLFR with its ligand.

99. The kit of claim 98, wherein the ligand is an OLFR ligand listed in Table 8.

100. The kit of claim 94, wherein the agent is an OLFR antagonist.

101. The kit of claim 100, wherein the OLFR antagonist is citral, undecanal, oxyphenylon, phenirat, methyl cinnamaldehyde, hydrocinamaldehyde, bourgeonal, ethylhexanoic acid, α-ionone, octanoic acid, a solvate or prodrug thereof.

102. The kit of claim 100, wherein the OLFR antagonist is an antagonist listed in Table 9.

103. The kit of claim 94, wherein the agent modulates the activity of the OLFR by inhibiting the trafficking of the OLFR to a plasma membrane of a cell.

104. The kit of claim 94, wherein the agent is an antibody, fragment or mimetic thereof that binds to an OLFR.

105. The kit of claim 94, wherein the agent is an OLFR agonist.

106. The kit of claim 105, wherein the OLFR agonist is an agonist listed in Table 8.

107. The kit of claim 105, wherein the OLFR agonist is selected from the group consisting of octanal, coumarin, helional, lilial, b-ionone, androstenone, androstadienone, caramel furanone, 3-phenyl propyl propionate, eugenol, ethil vanillin, 2-ethyl-fencol, isovaleric acid, nonanoic acid, butyl butyryllactate, butyric acid, isovaleric acid, propionic acid, N-amyl acetate, eugenol acetate, sandalwood, S-(−)-citronellol, S-(−)-citronellal, (+)-carvine, (−) carvone, (+) carvone, linalool, bourgeonal, acetophenone, amyl butyrate, nonanethiol, allyl phenyl acetate, N-amyl acetate, muscone, isoeugenol, eugenol methyl ether, 1-hexanol, 1-heptanole, 1-octanol, celery ketone, anis aldehyde, (+)-menthol, vanillin, guaiacol, lyral, ethyl heptanoate, methyl octanoate, nonanal 1-nonanol, 2-nonanol, 3-octanone, 3-nonanone, decyl aldehyde.

108. The kit of claim 94, wherein the agent is octanal, heptanal, or a prodrug or solvate thereof.

109. The kit of claim 94, wherein the agent is a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal).

110. The kit of claim 94, wherein the agent comprises a nucleotide encoding a protein selected from the group consisting of receptor transporting protein 1 (RTP1), RTP2, receptor expression enhancing protein 1 (REEP1), aminoacylase 3 (Acy3), and guanine nucleotide-binding protein G(olf) subunit alpha (Gnal).

111. The kit of any of claims 94-110, wherein the OLFR is an OLFR listed in any one of Tables 1-9 and FIGS. 2A-2B.

112. The kit of any of claims 94-111, wherein the OLFR is an OLFR listed in Table 7.

113. The kit of any of claims 94-112, wherein the OLFR is selected from the group consisting of OR7C1, OR7D4, OR10A6, OR11H6, OR4E2, OR10H1, and OR6A2.
